(19) United States
(12) Patent Application Publication
(10) Pub. No.: US 2006/0040957 A1
(43) Pub. Date: Feb. 23, 2006

(54) BICYCLIC PYRIMIDINE MATRIX METALLOPROTEINASE INHIBITORS

(76) Inventors: Richard Dennis Dyer, Ann Arbor, MI (US); William Glen Harter, Chelsea, MI (US); James Lester Hicks, Chelsea, MI (US); Adam Richard Johnson, Ann Arbor, MI (US); Jie Jack Li, Ann Arbor, MI (US); William Howard Roark, Ann Arbor, MI (US); Kevon Ray Shuler, Chelsea, MI (US)

Correspondence Address: WARNER-LAMBERT COMPANY, 2800 PLYMOUTH RD, ANN ARBOR, MI, 48105, US

(21) Appl. No.: 10/071,032

(22) Filed: Feb. 8, 2002

Related U.S. Application Data
(60) Provisional application No. 60/268,780, filed on Feb. 14, 2001.

(51) Int. Cl. A61K 31/519; C07D 498/04

(52) U.S. Cl. 514/259.2; 544/278

(57) ABSTRACT

Selective MMP-13 inhibitors are bicyclic pyrimidines of the Formula I 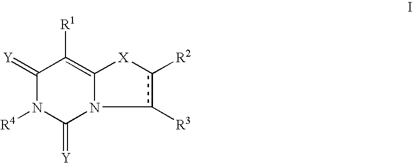
or a pharmaceutically acceptable salt thereof, wherein R¹ is hydrogen or alkyl; R², R³, and R⁴ include hydrogen, halo, alkyl, C≡C(CH₂)ₘ aryl, CO₂ alkyl, CO₂(CH₂)ₘ aryl, COHN alkyl, and CONH(CH₂)ₘ aryl; and X is O, S, SO, SO₂, CH₂, C═O, CHOH, NH, or NR⁵. A compound of Formula I, or a pharmaceutically acceptable salt thereof, is useful for treating cancer or arthritis.

### CROSS-REFERENCE TO RELATED APPLICATIONS

This application claims benefit of priority from U.S. provisional application No. 60/268,780, filed Feb. 14, 2001.

### FIELD OF THE INVENTION

This invention relates to a group of bicyclic pyrimidine derivatives which inhibit matrix metalloproteinase enzymes and thus are useful for treating diseases resulting from tissue breakdown, such as heart disease, multiple sclerosis, arthritis, atherosclerosis, and osteoporosis.

### BACKGROUND OF THE INVENTION

Matrix metalloproteinases (sometimes referred to as MMPs) are naturally-occurring enzymes found in most mammals. Over-expression and activation of MMPs or an imbalance between MMPs and inhibitors of MMPs have been suggested as factors in the pathogenesis of diseases characterized by the breakdown of extracellular matrix or connective tissues.

Stromelysin-1 and gelatinase A are members of the matrix metalloproteinases (MMP) family. Other members include fibroblast collagenase (MMP-1), neutrophil collagenase (MMP-8), gelatinase B (92 kDa gelatinase) (MMP-9), stromelysin-2 (MMP-10), stromelysin-3 (MMP-11), matrilysin (MMP-7), collagenase 3 (MMP-13), TNF-alpha converting enzyme (TACE), and other newly discovered membrane-associated matrix metalloproteinases (Sato H, Takino T, Okada Y, Cao J, Shinagawa A, Yamamoto E, and Seiki M., Nature, 1994;370:61-65). These enzymes have been implicated with a number of diseases which result from breakdown of connective tissue, including such diseases as rheumatoid arthritis, osteoarthritis, osteoporosis, periodontitis, multiple sclerosis, gingivitis, corneal epidermal and gastric ulceration, atherosclerosis, neointimal proliferation which leads to restenosis and ischemic heart failure, and tumor metastasis. A method for preventing and treating these and other diseases is now recognized to be by inhibiting metalloproteinase enzymes, thereby curtailing and/or eliminating the breakdown of connective tissues that results in the disease states.

The catalytic zinc in matrix metalloproteinases is typically the focal point for inhibitor design. The modification of substrates by introducing zinc chelating groups has generated potent inhibitors such as peptide hydroxamates and thiol-containing peptides. Peptide hydroxamates and the natural endogenous inhibitors of MMPs (TIMPs) have been used successfully to treat animal models of cancer and inflammation. MMP inhibitors have also been used to prevent and treat congestive heart failure and other cardiovascular diseases, U.S. Pat. No. 5,948,780.

A major limitation on the use of currently known MMP inhibitors is their lack of specificity for any particular enzyme. Recent data has established that specific MMP enzymes are associated with some diseases, with no effect on others. The MMPs are generally categorized based on their substrate specificity, and indeed the collagenase subfamily of MMP-1, MMP-8, and MMP-13 selectively cleave native interstitial collagens, and thus are associated only with diseases linked to such interstitial collagen tissue. This is evidenced by the recent discovery that MMP-13 alone is over expressed in breast carcinoma, while MMP-1 alone is over expressed in papillary carcinoma (see Chen et al., J. Am. Chem. Soc., 2000;122:9648-9654).

There appears to be few selective inhibitors of MMP-13 reported. A compound named WAY-170523 has been reported by Chen et al., supra., 2000, and a few other compounds are reported in PCT international patent application Number WO 01/63244 A1, as allegedly selective inhibitors of MMP-13. Further, U.S. Pat. No. 6,008,243 discloses inhibitors of MMP-13. However, no selective or nonselective inhibitor of MMP-13 has been approved and marketed for the treatment of any disease in any mammal. Accordingly, the need continues to find new low molecular weight compounds that are potent and selective MMP inhibitors, and that have an acceptable therapeutic index of toxicity/potency to make them amenable for use clinically in the prevention and treatment of the associated disease states. An object of this invention is to provide a group of selective MMP-13 inhibitor compounds characterized as being bicyclic pyrimidines.

### SUMMARY OF THE INVENTION

This invention provides a group of bicyclic pyrimidine compounds that are inhibitors of matrix metalloproteinase enzymes, and especially MMP-13. The invention is more particularly directed to compounds defined by Formula I 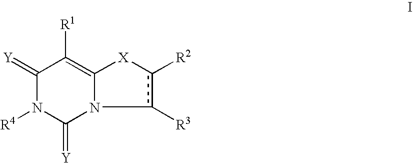
or a pharmaceutically acceptable salt thereof, wherein:
“- - - ” is absent or is a bond;
X is O, S, SO, SO₂, CH₂, C═O, CHOH, NH, or NR⁵;
Y is O or S; R¹ is H, (O)ₙC₁-C₆ alkyl, (O)ₙ substituted C₁-C₆ alkyl, NO₂, NR⁵R⁶, CHO, or halo;
R², R³, and R⁴ independently are hydrogen, halo, C₁-C₆ alkyl, substituted C₁-C₆ alkyl, C₂-C₆ alkenyl, substituted C₂-C₆ alkenyl, C₂-C₁₀ alkynyl, substituted C₂-C₁₀ alkynyl, (CH2)ₘ OH, (CH₂)ₘ OR⁵, (CH₂)ₘ cycloalkyl, (CH₂)ₘ substituted cycloalkyl, CHOH(CH₂)ₘ aryl, CHOH(CH₂)ₘ substituted aryl, CHOH(CH₂)ₘ heteroaryl, CHOH(CH₂)ₘ substituted heteroaryl, (CO₂)ₙ(CH₂)ₘ aryl, (CO₂)ₙ(CH₂)ₘ substituted aryl, (CO₂)ₙ(CH₂)ₘ heteroaryl, (CO₂)ₙ(CH₂)ₘ substituted heteroaryl, (CO₂)ₙ(CH₂)ₘ carbocycle, (CO₂)ₙ(CH₂)ₘ substituted carbocycle, (CO₂)ₙ(CH₂)ₘ heterocycle, (CO₂)ₙ(CH₂)ₘ substituted heterocycle, (CO₂)ₙ(CH₂)ₘ NR⁵R⁶, CH(C₁₋₆ alkyl)-aryl, (CH₂)ₘ N(H)C(═O)aryl, (CH₂)ₘ—S(O)₀₋₂—(CH₂)ₙ-aryl, CH(C₁-C₆ alkyl)-substituted aryl, (CH₂)ₘN(H)C(═O) substituted aryl, (CH₂)ₘ—S(O)₀₋₂—(CH₂)ₙ substituted aryl,

C(═O)N(R⁵)—(CH₂)ₘ aryl, C(═O)N(R⁵)—(CH₂)ₘ substituted aryl, C(═O)N(R⁵)—(CH₂)ₘ heteroaryl, C(═O)N(R⁵)—(CH₂)ₘ substituted heteroaryl, C≡C—(CH₂)ₘ aryl, C≡C—(CH₂)ₘ substituted aryl, C≡C—(CH₂)ₘ-heteroaryl, C≡C—(CH₂)ₘ substituted heteroaryl, C≡C—(CH₂)ₘ carbocycle, C≡C—(CH₂)ₘ substituted carbocycle, (CH₂)ₘ—O-aryl, (CH₂)ₘ—O-substituted aryl, (CH₂)ₘCOR⁵, 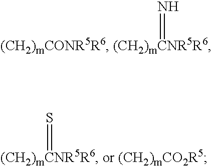
m is an integer from 0 to 6;
R⁵ and R⁶ independently are hydrogen, C₁-C₆ alkyl, substituted C₁-C₆ alkyl, (CH₂)ₘ aryl, (CH₂)ₘ substituted aryl, (CH₂)ₘ heteroaryl or (CH₂)ₘ substituted heteroaryl, or R⁵ and R⁶ are taken together with the nitrogen atom to which they are attached complete a 3- to 7-membered ring; containing carbon atoms, the nitrogen atom bearing R⁵ and R⁶, and optionally 1 or 2 heteroatoms independently selected form O, S, and NR², wherein R² is as defined above and;
n is 0 or 1; with the proviso that R² and R⁴ are not both selected from hydrogen and C₁-C₆ alkyl.

Another invention embodiment is compounds of Formula I, or a pharmaceutically acceptable salt thereof, wherein X is S, SO, or SO₂, and Y, R¹, R², R³, and R⁴ are as defined above.

Another invention embodiment is compounds of Formula I, or a pharmaceutically acceptable salt thereof, wherein R² and R⁴ are not H.

Another invention embodiment is compounds of Formula I, or a pharmaceutically acceptable salt thereof, wherein R³ is H or fluoro, and both R² and R⁴ are not H.

Another invention embodiment is compounds of Formula I, or a pharmaceutically acceptable salt thereof, that have R² equal CO₂ aryl or CO₂ heteroaryl, wherein aryl and heteroaryl may be unsubstituted or substituted.

Another invention embodiment is compounds of Formula II 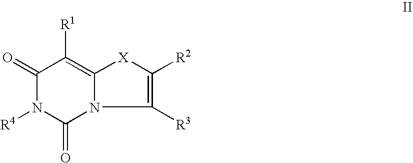
or a pharmaceutically acceptable salt thereof, wherein R¹, R², R³, R⁴, and X are as defined above.

Another invention embodiment is compounds of Formula II, or a pharmaceutically acceptable salt thereof, wherein R¹ is H or CH₃, R² is CO₂CH₂ aryl, CO₂CH₂ heteroaryl, CONHCH₂ aryl, or CONHCH₂ heteroaryl, wherein the aryl and heteroaryl groups are unsubstituted or substituted, and R³ is H or fluoro.

Also another invention embodiment is compounds of Formula II, or a pharmaceutically acceptable salt thereof, that are amides, i.e., compounds wherein R² is

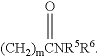

Another invention embodiment is compounds of Formula III 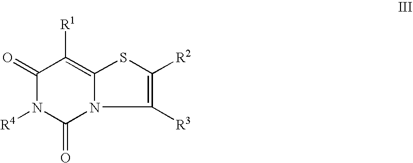
or a pharmaceutically acceptable salt thereof, wherein R¹, R², R³, and R⁴ are as defined above.

Another invention embodiment is compounds of Formula III, or a pharmaceutically acceptable salt thereof, where R² is CO₂CH₂ aryl, CO₂CH₂ heteroaryl, CONHCH₂ aryl, or CONHCH₂ heteroaryl, wherein the aryl and heteroaryl groups are unsubstituted or substituted, R³ is H, and R⁴ is CH₂ aryl, CH₂ substituted aryl, CH₂ heteroaryl, or CH₂ substituted heteroaryl.

Another invention embodiment is a compound of Formula III, or a pharmaceutically acceptable salt thereof, selected from:

6-Benzyl-8-methyl-5,7-dioxo-6,7-dihydro-5H-thiazolo[3,2-c]pyrimidine-2-carbothioic acid benzylamide; and

6-Benzyl-8-methyl-5,7-dioxo-6,7-dihydro-5H-thiazolo[3,2-c]pyrimidine-2-carbothioic acid 4-methoxy-benzylamide.

Another invention embodiment is a compound of Formula III, or a pharmaceutically acceptable salt thereof, named:

6-Benzyl-2-(3-phenyl-propionyl)-thiazolo[3,2-c]pyrimidine-5,7-dione;

6-Benzyl-8-methyl-5,7-dioxo-6,7-dihydro-5H-thiazolo[3,2-c]pyrimidine-2-carboxylic acid prop-2-ynylamide;

6-Benzyl-8-methyl-5,7-dioxo-6,7-dihydro-5H-thiazolo[3,2-c]pyrimidine-2-carboxylic acid(piperidin-4-ylmethyl)-amide hydrochloride;

Another invention embodiment is a compound of Formula III, or a pharmaceutically acceptable salt thereof, selected from:

6-Benzyl-2-(1-hydroxy-3-phenyl-allyl)-8-methyl-thiazolo[3,2-c]pyrimidine-5,7-dione;

6-Benzyl-2-(1-hydroxy-3-phenyl-prop-2-ynyl)-8-methyl-thiazolo[3,2-c]pyrimidine-5,7-dione;

6-Benzyl-2-(hydroxy-phenyl-methyl)-thiazolo[3,2-c]pyrimidine-5,7-dione; and

6-Benzyl-2-(1-hydroxy-3-phenyl-propyl)-thiazolo[3,2-c]pyrimidine-5,7-dione.

Another invention embodiment is compounds that have Formula IV 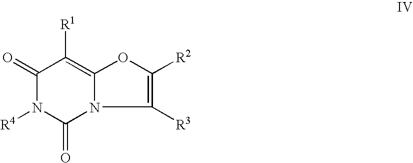
or a pharmaceutically acceptable salt thereof, wherein R¹, R², R³, and R⁴ are as defined above.

Another invention embodiment is a compound of Formula IV, or a pharmaceutically acceptable salt thereof, selected from:

6-Benzyl-8-methyl-5,7-dioxo-6,7-dihydro-5H-oxazolo[3,2-c]pyrimidine-2-carboxylic acid benzyl ester; and

6-Benzyl-5,7-dioxo-6,7-dihydro-5H-oxazolo[3,2-c]pyrimidine-2-carboxylic acid benzyl ester.

Another invention embodiment is a compound of Formula IV, or a pharmaceutically acceptable salt thereof, selected from:

6-Benzyl-5,7-dioxo-6,7-dihydro-5H-oxazolo[3,2-c]pyrimidine-2-carboxylic acid benzylamide;

6-Benzyl-8-methyl-5,7-dioxo-6,7-dihydro-5H-oxazolo[3,2-c]pyrimidine-2-carboxylic acid 4-methoxy-benzylamide;

6-Benzyl-8-methyl-5,7-dioxo-6,7-dihydro-5H-oxazolo[3,2-c]pyrimidine-2-carboxylic acid(pyridin-4-ylmethyl)-amide; and

6-Benzyl-8-methyl-5,7-dioxo-6,7-dihydro-5H-oxazolo[3,2-c]pyrimidine-2-carboxylic acid(benzo[1,3]dioxol-5-ylmethyl)-amide.

Another invention embodiment is compounds that have Formula V 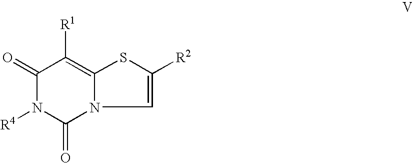
or a pharmaceutically acceptable salt thereof, wherein R¹ is hydrogen, (O)ₙC₁-C₆ alkyl, or (O)ₙ substituted C₁-C₆ alkyl, R² is CO₂(CH₂)ₘ aryl, CO₂(CH₂)ₘ substituted aryl,

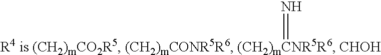

(CH₂)ₘ aryl, CHOH(CH₂)ₘ substituted aryl, CHOH(CH₂)ₘ heteroaryl, CHOH(CH₂)ₘ substituted aryl. Preferred compounds of Formula V are those wherein m is 0 or 1.

Another invention embodiment are 2,3-dihydro compounds of Formula VI: 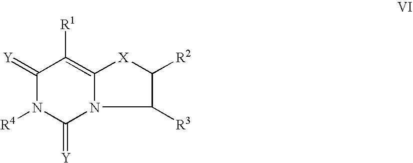
or a pharmaceutically acceptable salt thereof, wherein R¹, R², R³, R⁴, Y, and X are as defined above.

Another invention embodiment is a compound of Formula VI, or a pharmaceutically acceptable salt thereof, selected from:

6-Benzyl-8-methyl-5,7-dioxo-1,5,6,7-tetrahydro-imidazo[1,2-c]pyrimidine-2-carboxylic acid(benzo[1,3]dioxol-5-ylmethyl)-amide;

6-Benzyl-1,8-dimethyl-5,7-dioxo-1,5,6,7-tetrahydro-imidazo[1,2-c]pyrimidine-2-carboxylic acid(benzo[1,3]dioxol-5-ylmethyl)-amide;

6-Benzyl-1,8-dimethyl-5,7-dioxo-1,5,6,7-tetrahydro-imidazo[1,2-c]pyrimidine-2-carboxylic acid benzylamide;

6-Benzyl-1,8-dimethyl-5,7-dioxo-1,5,6,7-tetrahydro-imidazo[1,2-c]pyrimidine-2-carboxylic acid 4-methoxy-benzylamide;

6-Benzyl-1-methyl-5,7-dioxo-1,5,6,7-tetrahydro-imidazo[1,2-c]pyrimidine-2-carboxylic acid 4-methoxy-benzylamide;

6-(4-Methoxy-benzyl)-1-methyl-5,7-dioxo-1,5,6,7-tetrahydro-imidazo[1,2-c]pyrimidine-2-carboxylic acid 4-methoxy-benzylamide; and

6-(4-Methoxy-benzyl)-1,8-dimethyl-5,7-dioxo-1,5,6,7-tetrahydro-imidazo[1,2-c]pyrimidine-2-carboxylic acid(pyridin4-ylmethyl)-amide.

Another invention embodiment is a compound of Formula VI, or a pharmaceutically acceptable salt thereof, selected from:

6-Benzyl-5,7-dioxo-2,3,6,7-tetrahydro-5H-thiazolo[3,2-c]pyrimidine-2-carboxylic acid benzyl ester 2,3-Dihydroxypropionic acid benzyl ester;

6-Benzyl-5,7-dioxo-2,3,6,7-tetrahydro-5H-thiazolo[3,2-c]pyrimidine-2-carboxylic acid pyridin4-ylmethyl ester hydrochloride;

6-Benzyl-1,5,7-trioxo-1,2,3,5,6,7-hexahydro-1l⁴-thiazolo[3,2-c]pyrimidine-3-carboxylic acid benzyl ester; and

6-Benzyl-1,8-dimethyl-5,7-dioxo-1,5,6,7-tetrahydro-imidazo-[1,2-c]pyrimidine-2-carboxylic acid 4-methoxy-benzyl ester.

Another invention embodiment is a compound of Formula VI, or a pharmaceutically acceptable salt thereof, named 6-benzyl-3-ethoxy-2,3-dihydro-oxazolo[3,2-c]pyrimidine-5,7-dione.

Another invention embodiment is sulfoxides and sulfones of Formula VII 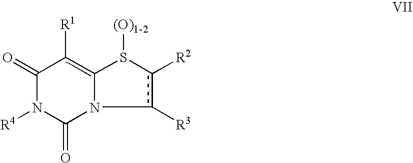
or a pharmaceutically acceptable salt thereof, wherein R¹, R², R³, and R⁴ are as defined above.

Another invention embodiment is a compound of Formula I of Formula VIII 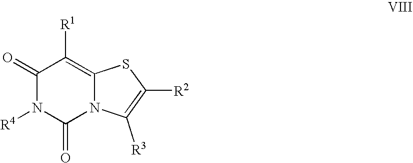
or a pharmaceutically acceptable salt thereof, wherein:
R¹ is H, CH₃, CH₂OH, or CHO;
R² is (CO₂)(CH₂)ₘ aryl, (CO₂)(CH₂)ₘ substituted aryl, (CO₂)(CH₂)ₘ heteroaryl, (CO₂)(CH₂)ₘ substituted heteroaryl, C(═O)N(R⁵)—(CH₂)ₘ-aryl, C(═O)N(R⁵)—(CH₂)ₘ substituted aryl, C(═O)N(R⁵)—(CH₂)ₘ heteroaryl, C(═O)N(R⁵)—(CH₂)ₘ substituted heteroaryl, C≡C—(CH₂)ₘ aryl, C≡C—(CH₂)ₘ substituted aryl, C≡C—(CH₂)ₘ heteroaryl, or C≡C—(CH₂)ₘ substituted heteroaryl, wherein R⁵ is hydrogen or methyl;
R³ is hydrogen or fluoro;
R⁴ is C₂-C₆ alkenyl, substituted C₂-C₆ alkenyl, C₁-C₆ alkyl, substituted C₁-C₆ alkyl, C₂-C₁₀ alkenyl, substituted C₂-C₁₀ alkynyl, (CH₂)ₘCOR⁵, (CH₂)ₘS(O)₀₋₂—(CH₂)ₙaryl, C(═O)N(R⁵)—(CH₂)ₘ aryl, (CH₂)ₘ—O-aryl, (CH₂)ₘS(O)₀₋₂—(CH₂)ₙ substituted aryl, C(═O)N(R⁵)—CH₂)ₘ substituted aryl, (CH₂)ₘ—O-substituted aryl, (CO₂)ₙ(CH₂)ₘ aryl, (CO₂)ₙ(CH₂)ₘ substituted aryl, (CO₂)ₙ(CH₂)ₘ heteroaryl, (CO₂)ₙ(CH₂)ₘ substituted heteroaryl, (CO₂)ₙ(CH₂)ₘ carbocycle, or (CO₂)ₙ(CH₂)ₘ substituted carbocycle, wherein
n is 0 or 1;
m is an integer of from 0 to 6; and
R⁵ is as defined above for Formula I.

Another invention embodiment is a compound of Formula VIII, or a pharmaceutically acceptable salt thereof, wherein:
R¹ is H or CH₃;
R² is C(═O)N(R⁵)—(CH₂)ₘ aryl, C(═O)N(R⁵)—(CH₂)ₘ substituted aryl, C(═O)N(R⁵)—(CH₂)ₘ heteroaryl, C(═O)N(R⁵)—(CH₂)ₘ substituted heteroaryl, C≡C—(CH₂)ₘaryl, C≡C—(CH₂)ₘ substituted aryl, C≡C—(CH₂)ₘ heteroaryl, or C≡C—(CH₂)ₘ substituted heteroaryl, wherein R⁵ is H or methyl;
R³ is hydrogen or fluoro;
R⁴ is (CO₂)ₙ(CH₂)ₘaryl, (CO₂)ₙ(CH₂)ₘ substituted aryl, (CO₂)ₙ(CH₂)ₘ heteroaryl, (CO₂)ₙ(CH₂)ₘ substituted heteroaryl, (CO₂)ₙ(CH₂)ₘ carbocycle, or (CO₂)ₙ(CH₂)ₘ substituted carbocycle, wherein:
n is 0 or 1, and
m is an integer of from 0 to 6.

Another invention embodiment is a compound of Formula VIII, or a pharmaceutically acceptable salt thereof, wherein:
R¹ is H or CH₃;
R² is C≡C—(CH₂)ₘaryl, C≡C—(CH₂)ₘ substituted aryl, C≡C—(CH₂)ₘ heteroaryl, or C≡C—(CH₂)ₘ substituted heteroaryl, wherein:
m is 1;
R₃ is hydrogen or fluoro; and
R₄ is (CO₂)ₙ(CH₂)ₘaryl, (CO₂)ₙ(CH₂)ₘ substituted aryl, (CO₂)ₙ(CH₂)ₘ heteroaryl, (CO₂)ₙ(CH₂)ₘ substituted heteroaryl, (CO₂)ₙ(CH₂)ₘcarbocycle, or (CO₂)ₙ(CH₂)ₘsubstituted carbocycle,
wherein n is 0 and m is 1.

Another invention embodiment is a compound of Formula VIII, or a pharmaceutically acceptable salt thereof, wherein:
R₁ is H or CH₃;
R₂ is C(═O)N(R⁵)—(CH₂)ₘaryl, C(═O)N(R⁵)—(CH₂)ₘsubstituted aryl, C(═O)N(R⁵)—(CH₂)ₘheteroaryl, or C(═O)N(R⁵)—(CH₂)ₘsubstituted heteroaryl,
wherein m is 1 and R⁵ is H or CH₃;
R³ is hydrogen or fluoro; and
R⁴ is (CO₂)ₙ(CH₂)ₘaryl, (CO₂)ₙ(CH₂)ₘsubstituted aryl, (CO₂)ₙ(CH₂)ₘheteroaryl, (CO₂)ₙ(CH₂)ₘsubstituted heteroaryl, (CO₂)ₙ(CH₂)ₘcarbocycle, or (CO₂)ₙ(CH₂)ₘsubstituted carbocycle,
wherein n is 0 and m is 1;

Another invention embodiment is a compound of Formula VIII, or a pharmaceutically acceptable salt thereof, selected from:

4-[8-Methyl-5,7-dioxo-2-(3-phenyl-prop-1-ynyl)-7H-thiazolo[3,2-c]pyrimidin-6-ylmethyl]-benzoic acid;

4-{2-[3-(4-Methoxy-phenyl)-prop-1-ynyl]-8-methyl-5,7-dioxo-7H-thiazolo[3,2-c]pyrimidin-6-ylmethyl}-benzoic acid;

4-{2-[3-(4-Fluoro-phenyl)-prop-1-ynyl]-8-methyl-5,7-dioxo-7H-thiazolo[3,2-c]pyrimidin-6-ylmethyl}-benzoic acid;

4-{2-[3-(3-Methoxy-phenyl)-prop-1-ynyl]-8-methyl-5,7-dioxo-7H-thiazolo[3,2-c]pyrimidin-6-ylmethyl}-benzoic acid;

4-{2-[3-(3,4-Difluoro-phenyl)-prop-1-ynyl]-8-methyl-5,7-dioxo-7H-thiazolo[3,2-c]pyrimidin-6-ylmethyl}-benzoic acid;

6-Benzyl-8-methyl-2-(3-pyridin4-yl-prop-1-ynyl)-thiazolo[3,2-c]pyrimidine-5,7-dione;

6-(3,4-Dichloro-benzyl)-8-methyl-2-(3-pyridin4-yl-prop-1-ynyl)-thiazolo[3,2-c]pyrimidine-5,7-dione;

6-(3,4-Dichloro-benzyl)-2-[3-(2-methoxy-pyridin-4-yl)-prop-1-ynyl]-8-methyl-thiazolo[3,2-c]pyrimidine-5,7-dione;

6-Benzyl-8-methyl-2-phenylethynyl-thiazolo[3,2-c]pyrimidine-5,7-dione;

6-(4-Bromo-benzyl)-2-[3-(3-methoxy-phenyl)-prop-1-ynyl]-8-methyl-thiazolo[3,2-c]pyrimidine-5,7-dione;

4-{2-[3-(3-Methoxy-phenyl)-prop-1-ynyl]-8-methyl-5,7-dioxo-7H-thiazolo[3,2-c]pyrimidin-6-ylmethyl}-benzenesulfonamide;

4-{2-[³-(³-Fluoro4-methoxy-phenyl)-prop-1-ynyl]-8-methyl-5,7-dioxo-7H-thiazolo[3,2-c]pyrimidin-6-ylmethyl}-benzoic acid;

6-(4-Fluoro-benzyl)-8-methyl-2-(3-phenyl-prop-1-ynyl)-thiazolo[3,2-c]pyrimidine-5,7-dione;

6-Benzyl-8-methyl-2-(3-phenyl-prop-1-ynyl)-thiazolo[3,2-c]pyrimidine-5,7-dione;

6-(3,4-Dichloro-benzyl)-2-[3-(3-methoxy-phenyl)-prop-1-ynyl]-8-methyl-thiazolo[3,2-c]pyrimidine-5,7-dione;

6-(4-Methanesulfonyl-benzyl)-8-methyl-2-(3-pyridin4-yl-prop-1-ynyl)-thiazolo[3,2-c]pyrimidine-5,7-dione;

4-{2-[3-(3-Methoxy-phenyl)-prop-1-ynyl]-8-methyl-5,7-dioxo-7H-thiazolo[3,2-c]pyrimidin-6 ylmethyl}-benzonitrile;

4-[8-Methyl-5,7-dioxo-2-(3-phenyl-prop-1-ynyl)-7H-thiazolo[3,2-c]pyrimidin-6-ylmethyl]-benzoic acid;

2-[3-(3-Methoxy-phenyl)-prop-1-ynyl]-8-methyl-6-[4-(2H-tetrazol-5-yl)-benzyl]-thiazolo[3,2-c]pyrimidine-5,7-dione;

6-Benzyl-2-[3-(3-methoxy-phenyl)-prop-1-ynyl]-8-methyl-thiazolo[3,2-c]pyrimidine-5,7-dione;

6-Benzyl-8-methyl-2-(3-phenyl-prop-1-ynyl)-thiazolo[3,2-c]pyrimidine-5,7-dione;

2-[3-(³-Methoxy-phenyl)-prop-1-ynyl]-8-methyl-6-[4-(morpholine-4-carbonyl)-benzyl]-thiazolo[3,2-c]pyrimidine-5,7-dione;

8-Methyl-6-[4-(morpholine-4-sulfonyl)-benzyl]-2-(3-pyridin-4-yl-prop-1-ynyl)-thiazolo[3,2-c]pyrimidine-5,7-dione;

2-[3-(4-Fluoro-phenyl)-prop-1-ynyl]-8-methyl-6-(2-oxo-2H-1-benzopyran-6-ylmethyl)-thiazolo[3,2-c]pyrimidine-5,7-dione;

2-[3-(3-Methoxy-phenyl)-prop-1-ynyl]-8-methyl-6-(2-oxo-2H-1-benzopyran-6-ylmethyl)-thiazolo[3,2-c]pyrimidine-5,7-dione;

4-[8-Methyl-5,7-dioxo-2-(4-phenyl-but-1-ynyl)-7H-thiazolo[3,2-c]pyrimidin-6-ylmethyl]-benzoic acid;

4-[8-Methyl-5,7-dioxo-2-(6-phenyl-hex-1-ynyl)-7H-thiazolo[3,2-c]pyrimidin-6-ylmethyl]-benzoic acid;

4-[8-Methyl-5,7-dioxo-2-(5-phenyl-pent-1-ynyl)-7H-thiazolo[3,2-c]pyrimidin-6-ylmethyl]-benzoic acid;

4-[8-Methyl-5,7-dioxo-2-(7-phenyl-hept-1-ynyl)-7H-thiazolo[3,2-c]pyrimidin-6-ylmethyl]-benzoic acid;

(4-{2-[3-(3,4-Difluoro-phenyl)-prop-1-ynyl]-8-methyl-5,7-dioxo-7H-thiazolo[3,2-c]pyrimidin-6-ylmethyl}-phenyl)-acetic acid;

6-(3-Fluoro-benzyl)-8-methyl-2-(3-pyridin-4-yl-prop-1-ynyl)-thiazolo[3,2-c]pyrimidine-5,7-dione;

6-(3,4-Difluoro-benzyl)-8-methyl-2-(3-pyridin-4-yl-prop-1-ynyl)-thiazolo[3,2-c]pyrimidine-5,7-dione;

6-(3-Fluoro-benzyl)-2-[3-(2-methoxy-pyridin-4-yl)-prop-1-ynyl]-8-methyl-thiazolo[3,2-c]pyrimidine-5,7-dione;

[3-(8-Methyl-5,7-dioxo-2-phenylethynyl-7H-thiazolo[3,2-c]pyrimidin-6-ylmethyl)-phenyl]-acetic acid;

6-(4-Bromo-benzyl)-2-[3-(4-fluoro-3-methoxy-phenyl)-prop-1-ynyl]-8-methyl-thiazolo[3,2-c]pyrimidine-5,7-dione;

4-{2-[3-(3-Methoxy-phenyl)-prop-1-ynyl]-8-methyl-5,7-dioxo-7H-methyl-thiazolo[3,2-c]pyrimidine-6-ylmethyl}-N,N-dimethyl-benzenesulfonamide;

4-{2-[3-(3-Fluoro-4-methoxy-phenyl)-prop-1-ynyl]-8-methyl-5,7-dioxo-7H-thiazolo[3,2-c]pyrimidin-6-ylmethyl}-cyclohexanecarboxylic acid;

6-(3,4-Difluoro-benzyl)-2-[3-(3,4-difluoro-phenyl)-prop-1-ynyl]-8-methyl-thiazolo[3,2-c]pyrimidine-5,7-dione;

4-[8-Methyl-5,7-dioxo-2-(3-phenyl-prop-1-ynyl)-7H-thiazolo[3,2-c]pyrimidin-6-ylmethyl]-cyclohexanecarboxylic acid;

2-Chloro-4-{2-[3-(3-methoxy-phenyl)-prop-1-ynyl]-8-methyl-5,7-dioxo-7H-thiazolo[3,2-c]pyrimidin-6-ylmethyl}-benzoic acid;

2-[3-(4-Fluoro-phenyl)-prop-1-ynyl]-6-(4-methanesulfonyl-benzyl)-8-methyl-thiazolo[3,2-c]pyrimidine-5,7-dione;

4-{2-[3-(4-Fluoro-3-methoxy-phenyl)-prop-1-ynyl]-8-methyl-5,7-dioxo-7H-thiazolo[3,2-c]pyrimidin-6-ylmethyl}-benzonitrile;

(3-{2-[3-(4-Fluoro-3-methoxy-phenyl)-prop-1-ynyl]-8-methyl-5,7-dioxo-7H-thiazolo[3,2-c]pyrimidin-6-ylmethyl}-phenyl)-acetic acid;

(4-{2-[3-(3-Methoxy-phenyl)-prop-1-ynyl]-8-methyl-5,7-dioxo-7H-thiazolo[3,2-c]pyrimidin-6-ylmethyl}-phenyl)-acetic acid;

6-(3,4-Difluoro-benzyl)-8-methyl-2-(3-phenyl-prop-1-ynyl)-thiazolo[3,2-c]pyrimidine-5,7-dione;

2-[3-(3-Methoxy-phenyl)-prop-1-ynyl]-8-methyl-6-[4-(thiomorpholine-4-carbonyl)-benzyl]-thiazolo[3,2-c]pyrimidine-5,7-dione;

8-Methyl-2-(3-pyridin-4-yl-prop-1-ynyl)-6-[4-(thiomorpholine-4-sulfonyl)-benzyl]-thiazolo[3,2-c]pyrimidine-5,7-dione;

2-[3-(4-Fluoro-3-methoxy-phenyl)-prop-1-ynyl]-8-methyl-6-(2-oxo-2H-1-benzopyran-6-ylmethyl)-thiazolo[3,2-c]pyrimidine-5,7-dione; and

2-[3-(3-Methoxy4-methyl-phenyl)-prop-1-ynyl]-8-methyl-6-(2-oxo-2H-1-benzopyran-6-ylmethyl)-thiazolo[3,2-c]pyrimidine-5,7-dione.

Another invention embodiment is a compound of Formula VIII, or a pharmaceutically acceptable salt thereof, selected from:

6-Benzyl-5,7-dioxo-6,7-dihydro-5H-thiazolo[3,2-c]pyrimidine-2-carboxylic acid benzylamide;

6-Benzyl-5,7-dioxo-6,7-dihydro-5H-thiazolo[3,2-c]pyrimidine-2-carboxylic acid biphenyl-4-ylamide;

6-Benzyl-5,7-dioxo-6,7-dihydro-5H-thiazolo[3,2-c]pyrimidine-2-carboxylic acid(pyridin-4-ylmethyl)-amide hydrochloride;

6-Benzyl-8-methyl-5,7-dioxo-6,7-dihydro-5H-thiazolo[3,2-c]pyrimidine-2-carboxylic acid 3-fluoro-benzylamide;

6-Benzoyl-5,7-dioxo-6,7-dihydro-5H-thiazolo[3,2-c]pyrimidine-2-carboxylic acid benzylamide;

6-(3,4-Dichloro-benzyl)-5,7-dioxo-6,7-dihydro-5H-thiazolo[3,2-c]pyrimidine-2-carboxylic acid benzylamide;

6-(4-Chloro-benzyl)-5,7-dioxo-6,7-dihydro-5H-thiazolo[3,2-c]pyrimidine-2-carboxylic acid benzylamide;

6-(4-Chloro-benzyl)-5,7-dioxo-6,7-dihydro-5H-thiazolo[3,2-c]pyrimidine-2-carboxylic acid 3,4-dichloro-benzylamide;

5,7-Dioxo-6-pyridin-4-ylmethyl-6,7-dihydro-5H-thiazolo[3,2-c]pyrimidine-2-carboxylic acid benzylamide hydrochloride;

6-Benzyl-8-methyl-5,7-dioxo-6,7-dihydro-5H-thiazolo[3,2-c]pyrimidine-2-carboxylic acid benzylamide;

6-Benzyl-8-methyl-5,7-dioxo-6,7-dihydro-5H-thiazolo[3,2-c]pyrimidine-2-carboxylic acid 4-methoxy-benzylamide;

6-Benzyl-8-methyl-5,7-dioxo-6,7-dihydro-5H-thiazolo[3,2-c]pyrimidine-2-carbozylic acid 3,4-dichlorobenzylamide;

6-Benzyl-5,7-dioxo-6,7-dihydro-5H-thiazolo[3,2-c]pyrimidine-2-carboxylic acid(pyridin-4-ylmethyl)-amide hydrochloride;

6-Benzyl-8-methyl-5,7-dioxo-6,7-dihydro-5H-thiazolo[3,2-c]pyrimidine-2-carboxylic acid 2,4-dichloro-benzylamide;

6-Benzyl-8-methyl-5,7-dioxo-6,7-dihydro-5H-thiazolo[3,2-c]pyrimidine-2-carboxylic acid 3-methyl-benzylamide;

6-Benzyl-8-methyl-5,7-dioxo-6,7-dihydro-5H-thiazolo[3,2-c]pyrimidine-2-carboxylic acid 4-fluoro-benzylamide;

6-Benzyl-8-formyl-5,7-dioxo-6,7-dihydro-5H-thiazolo[3,2-c]pyrimidine-2-carboxylic acid 4-methoxy-benzylamide;

6-Benzyl-8-methyl-5,7-dioxo-6,7-dihydro-5H-thiazolo[3,2-c]pyrimidine-2-carboxylic acid(1H-indol-5-ylmethyl)-amide;

6-Benzyl-8-methyl-5,7-dioxo-6,7-dihydro-5H-thiazolo[3,2-c]pyrimidine-2-carboxylic acid(thiazol-4-ylmethyl)-amide hydrochloride;

6-Benzyl-8-methyl-5,7-dioxo-6,7-dihydro-5H-thiazolo[3,2-c]pyrimidine-2-carboxylic acid(pyridin-4-ylmethyl)-amide hydrochloride;

6-Benzyl-8-methyl-5,7-dioxo-6,7-dihydro-5H-thiazolo[3,2-c]pyrimidine-2-carboxylic acid(6-methoxy-pyridin-3-ylmethyl)-amide;

6-Benzyl-8-methyl-5,7-dioxo-6,7-dihydro-5H-thiazolo[3,2-c]pyrimidine-2-carboxylic acid(6-methoxy-pyridin-3-ylmethyl)-amide hydrochloride;

6-Benzyl-8-methyl-5,7-dioxo-6,7-dihydro-5H-thiazolo[3,2-c]pyrimidine-2-carboxylic acid(imidazo[2,1-b]thiazol-6-ylmethyl)-amide;

6-Benzyl-8-methyl-5,7-dioxo-6,7-dihydro-5H-thiazolo[3,2-c]pyrimidine-2-carboxylic acid(1-methyl-1H-pyrazol-4-ylmethyl)-amide;

6-Benzyl-8-methyl-5,7-dioxo-6,7-dihydro-5H-thiazolo[3,2-c]pyrimidine-2-carboxylic acid(6-methyl-pyridin-2-ylmethyl)-amide;

6-Benzyl-8-methyl-5,7-dioxo-6,7-dihydro-5H-thiazolo[3,2-c]pyrimidine-2-carboxylic acid(2,1,3-benzothiadiazol-5-ylmethyl)-amide;

6-Benzyl-8-methyl-5,7-dioxo-6,7-dihydro-5H-thiazolo[3,2-c]pyrimidine-2-carboxylic acid 3,4-difluoro-benzylamide;

6-Benzyl-8-methyl-5,7-dioxo-6,7-dihydro-5H-thiazolo[3,2-c]pyrimidine-2-carboxylic acid(pyridin-3-ylmethyl)-amide;

6-Benzyl-8-methyl-5,7-dioxo-6,7-dihydro-5H-thiazolo[3,2-c]pyrimidine-2-carboxylic acid(pyridin-3-ylmethyl)-amide hydrochloride;

6-Benzyl-8-methyl-5,7-dioxo-6,7-dihydro-5H-thiazolo[3,2-c]pyrimidine-2-carboxylic acid 3-fluoro4-methoxy-benzylamide;

6-Benzyl-8-methyl-5,7-dioxo-6,7-dihydro-5H-thiazolo[3,2-c]pyrimidine-2-carboxylic acid(pyridin-2-ylmethyl)-amide hydrochloride;

6-Benzyl-8-methyl-5,7-dioxo-6,7-dihydro-5H-thiazolo[3,2-c]pyrimidine-2-carboxylic acid 4-methyl-benzylamide;

6-Benzyl-8-methyl-5,7-dioxo-6,7-dihydro-5H-thiazolo[3,2-c]pyrimidine-2-carboxylic acid 4-trifluoromethyl-benzylamide;

6-Benzyl-8-methyl-5,7-dioxo-6,7-dihydro-5H-thiazolo[3,2-c]pyrimidine-2-carboxylic acid 4-chloro-benzylamide;

6-Benzyl-8-methyl-5,7-dioxo-6,7-dihydro-5H-thiazolo[3,2-c]pyrimidine-2-carboxylic acid 4-trifluoromethoxy-benzylamide;

6-Benzyl-8-methyl-5,7-dioxo-6,7-dihydro-5H-thiazolo[3,2-c]pyrimidine-2-carboxylic acid(2-methyl-thiazol-4-ylmethyl)-amide hydrochloride;

4-[2-(4-Methoxy-benzylcarbamoyl)-8-methyl-5,7-dioxo-7H-thiazolo[3,2-c]pyrimidin-6-ylmethyl]-benzoic acid;

4-[2-(4-Methoxy-benzylcarbamoyl)-8-methyl-5,7-dioxo-7H-thiazolo[3,2-c]pyrimidin-6-ylmethyl]-benzoic acid Sodium salt;

4-[2-(4-Methoxy-benzylcarbamoyl)-8-methyl-5,7-dioxo-7H-thiazolo[3,2-c]pyrimidin-6-ylmethyl]-benzoic acid 2-dimethylamino-ethyl ester hydrochloride;

4-[2-(4-Fluoro-benzylcarbamoyl)-8-methyl-5,7-dioxo-7H-thiazolo[3,2-c]pyrimidin-6-ylmethyl]-benzoic acid;

4-[2-(4-Fluoro-benzylcarbamoyl)-8-methyl-5,7-dioxo-7H-thiazolo[3,2-c]pyrimidin-6-ylmethyl]-benzoic acid Sodium salt;

4-[2-(4-Fluoro-benzylcarbamoyl)-8-methyl-5,7-dioxo-7H-thiazolo[3,2-c]pyrimidin-6-ylmethyl]-benzoic acid 2-dimethylamino-ethyl ester;

4-[2-(4-Fluoro-benzylcarbamoyl)-8-methyl-5,7-dioxo-7H-thiazolo[3,2-c]pyrimidin-6-ylmethyl]-benzoic acid 2-dimethylamino-ethyl ester hydrochloride;

4-{8-Methyl-5,7-dioxo-2-[(pyridin-4-ylmethyl)-carbamoyl]-7H-thiazolo[3,2-c]pyrimidin-6-ylmethyl}-benzoic acid trifluoro-acetic acid salt;

4-{8-Methyl-5,7-dioxo-2-[(pyridin-4-ylmethyl)-carbamoyl]-7H-thiazolo[3,2-c]pyrimidin-6-ylmethyl}-benzoic acid 2-dimethylamino-ethyl ester dihydrochloride;

8-Methyl-6-(2-methyl-thiazol-4-ylmethyl)-5,7-dioxo-6,7-dihydro-5H-thiazolo[3,2-c]pyrimidine-2-carboxylic acid 4-fluoro-benzylamide;

2-Chloro4-[2-(4-fluoro-benzylcarbamoyl)-8-methyl-5,7-dioxo-7H-thiazolo[3,2-c]pyrimidin-6-ylmethyl]-benzoic acid methyl ester;

8-Methyl-5,7-dioxo-6-(2H-tetrazol-5-ylmethyl)-6,7-dihydro-5H-thiazolo[3,2-c]pyrimidine-2-carboxylic acid 4-fluoro-benzylamide;

8-Methyl-5,7-dioxo-6-thiazol-2-ylmethyl-6,7-dihydro-5H-thiazolo[3,2-c]pyrimidine-2-carboxylic acid 4-fluoro-benzylamide hydrochloride;

4-[2-(4-Fluoro-benzylcarbamoyl)-8-methyl-5,7-dioxo-7H-thiazolo[3,2-c]pyrimidin-6-ylmethyl]-2-methyl-benzoic acid methyl ester;

4-[2-(4-Fluoro-benzylcarbamoyl)-8-methyl-5,7-dioxo-7H-thiazolo[3,2-c]pyrimidin-6-ylmethyl]-2-methoxy-benzoic acid methyl ester;

6-(4-Fluoro-benzyl)-8-methyl-5,7-dioxo-6,7-dihydro-5H-thiazolo[3,2-c]pyrimidine-2-carboxylic acid(pyridin-4-ylmethyl)-amide hydrochloride;

6-(4-Bromo-benzyl)-8-methyl-5,7-dioxo-6,7-dihydro-5H-thiazolo[3,2-c]pyrimidine-2-carboxylic acid(pyridin-4-ylmethyl)-amide hydrochloride;

6-(4-Chloro-benzyl)-8-methyl-5,7-dioxo-6,7-dihydro-5H-thiazolo[3,2-c]pyrimidine-2-carboxylic acid(pyridin-4-ylmethyl)-amide hydrochloride;

8-Methyl-6-[4-(morpholine-4-carbonyl)-benzyl]-5,7-dioxo-6,7-dihydro-5H-thiazolo[3,2-c]pyrimidine-2-carboxylic acid(pyridin-4-ylmethyl)-amide hydrochloride;

{5-[2-(4-Fluoro-benzylcarbamoyl)-8-methyl-5,7-dioxo-7H-thiazolo[3,2-c]pyrimidin-6-ylmethyl]-isoxazol-3-yl}-carbamic acid methyl ester;

8-Methyl-5,7-dioxo-6-[4-(2H-tetrazol-5-yl)-benzyl]-6,7-dihydro-5H-thiazolo[3,2-c]pyrimidine-2-carboxylic acid 4-fluoro-benzylamide;

8-Methyl-6-[4-(morpholine-4-carbonyl)-benzyl]-5,7-dioxo-6,7-dihydro-5H-thiazolo[3,2-c]pyrimidine-2-carboxylic acid 4-fluoro-benzylamide;

6-(6-Fluoro-quinolin-2-ylmethyl)-8-methyl-5,7-dioxo-6,7-dihydro-5H-thiazolo[3,2-c]pyrimidine-2-carboxylic acid 4-fluoro-benzylamide;

2-[2-(4-Fluoro-benzylcarbamoyl)-8-methyl-5,7-dioxo-7H-thiazolo[3,2-c]pyrimidin-6-ylmethyl]-5-methoxy-pyrimidine4-carboxylic acid methyl ester;

6-But-2-ynyl-8-methyl-5,7-dioxo-6,7-dihydro-5H-thiazolo[3,2-c]pyrimidine-2-carboxylic acid 4-fluoro-benzylamide;

8-Methyl-5,7-dioxo-6-(2-oxo-2H-1-benzopyran-6-ylmethyl)-6,7-dihydro-5H-thiazolo[3,2-c]pyrimidine-2-carboxylic acid 4-fluoro-benzylamide;

6-(4-Methanesulfonyl-benzyl)-8-methyl-5,7-dioxo-6,7-dihydro-5H-thiazolo[3,2-c]pyrimidine-2-carboxylic acid(pyridin-4-ylmethyl)-amide hydrochloride;

6-(3-Cyano-benzyl)-8-methyl-5,7-dioxo-6,7-dihydro-5H-thiazolo[3,2-c]pyrimidine-2-carboxylic acid(pyridin-4-ylmethyl)-amide hydrochloride;

6-[2-(4-Chloro-benzenesulfonyl)-ethyl]-8-methyl-5,7-dioxo-6,7-dihydro-5H-thiazolo[3,2-c]pyrimidine-2-carboxylic acid(pyridin-4-ylmethyl)-amide hydrochloride;

8-Methyl-5,7-dioxo-6-(4-sulfamoyl-benzyl)-6,7-dihydro-5H-thiazolo[3,2-c]pyrimidine-2-carboxylic acid(pyridin-4-ylmethyl)-amide hydrochloride;

6-(4-Cyano-benzyl)-8-methyl-5,7-dioxo-6,7-dihydro-5H-thiazolo[3,2-c]pyrimidine-2-carboxylic acid(pyridin-4-ylmethyl)-amide hydrochloride;

8-Methyl-5,7-dioxo-6-(3-oxo-3-phenyl-propyl)-6,7-dihydro-5H-thiazolo[3,2-c]pyrimidine-2-carboxylic acid 4-fluoro-benzylamide;

8-Methyl-5,7-dioxo-6-(1-phenyl-ethyl)-6,7-dihydro-5H-thiazolo[3,2-c]pyrimidine-2-carboxylic acid 4-fluoro-benzylamide;

8-Methyl-5,7-dioxo-6-(2-phenylmethanesulfonyl-ethyl)-6,7-dihydro-5H-thiazolo[3,2-c]pyrimidine-2-carboxylic acid 4-fluoro-benzylamide;

6-(5-Cyano-pentyl)-8-methyl-5,7-dioxo-6,7-dihydro-5H-thiazolo[3,2-c]pyrimidine-2-carboxylic acid 4-fluoro-benzylamide;

6-(E)-But-2-enyl-8-methyl-5,7-dioxo-6,7-dihydro-5H-thiazolo[3,2-c]pyrimidine-2-carboxylic acid 4-fluoro-benzylamide;

8-Methyl-5,7-dioxo-6-(E)-pent-2-enyl-6,7-dihydro-5H-thiazolo[3,2-c]pyrimidine-2-carboxylic acid 4-fluoro-benzylamide;

6-sec-Butyl-8-methyl-5,7-dioxo-6,7-dihydro-5H-thiazolo[3,2-c]pyrimidine-2-carboxylic acid 4-fluoro-benzylamide;

6-(2-Benzenesulfonyl-ethyl)-8-methyl-5,7-dioxo-6,7-dihydro-5H-thiazolo[3,2-c]pyrimidine-2-carboxylic acid 4-fluoro-benzylamide;

6-(1-Ethyl-propyl)-8-methyl-5,7-dioxo-6,7-dihydro-5H-thiazolo[3,2-c]pyrimidine-2-carboxylic acid 4-fluoro-benzylamide;

8-Methyl-5,7-dioxo-6-pent-2-ynyl-6,7-dihydro-5H-thiazolo[3,2-c]pyrimidine-2-carboxylic acid 4-fluoro-benzylamide;

6-(2-Benzenesulfonyl-ethyl)-8-methyl-5,7-dioxo-6,7-dihydro-5H-thiazolo[3,2-c]pyrimidine-2-carboxylic acid 4-fluoro-benzylamide;

8-Methyl-6-(3-methyl-but-2-enyl)-5,7-dioxo-6,7-dihydro-5H-thiazolo[3,2-c]pyrimidine-2-carboxylic acid 4-fluoro-benzylamide;

6-[2-(4-Fluoro-benzenesulfonyl)-ethyl]-8-methyl-5,7-dioxo-6,7-dihydro-5H-thiazolo[3,2-c]pyrimidine-2-carboxylic acid 4-fluoro-benzylamide;

6-[3-(4-Fluoro-phenyl)-3-oxo-propyl]-8-methyl-5,7-dioxo-6,7-dihydro-5H-thiazolo[3,2-c]pyrimidine-2-carboxylic acid 4-fluoro-benzylamide;

6-(2-Benzoylamino-ethyl)-8-methyl-5,7-dioxo-6,7-dihydro-5H-thiazolo[3,2-c]pyrimidine-2-carboxylic acid 4-fluoro-benzylamide;

8-Methyl-5,7-dioxo-6-(2-phenoxy-ethyl)-6,7-dihydro-5H-thiazolo[3,2-c]pyrimidine-2-carboxylic acid 4-fluoro-benzylamide;

6-(3,4-Dichloro-benzyl)-5,7-dioxo-6,7-dihydro-5H-thiazolo[3,2-c]pyrimidine-2-carboxylic acid 4-methoxy-benzylamide;

6-(4-Cyano-benzyl)-8-methyl-5,7-dioxo-6,7-dihydro-5H-thiazolo[3,2-c]pyrimidine-2-carboxylic acid 4-fluoro-benzylamide;

6-Benzyl-8-methyl-5,7-dioxo-6,7-dihydro-5H-thiazolo[3,2-c]pyrimidine-2-carboxylic acid 3-methoxy-benzylamide;

6-Benzyl-8-methyl-5,7-dioxo-6,7-dihydro-5H-thiazolo[3,2-c]pyrimidine-2-carboxylic acid(tetrahydro-furan-2-ylmethyl)-amide;

6-Benzyl-8-methyl-5,7-dioxo-6,7-dihydro-5H-thiazolo[3,2-c]pyrimidine-2-carboxylic acid benzylamide;

6-Benzyl-8-methyl-5,7-dioxo-6,7-dihydro-5H-thiazolo[3,2-c]pyrimidine-2-carboxylic acid 3-fluoro-benzylamide;

6-(3-Fluoro-benzyl)-8-methyl-5,7-dioxo-6,7-dihydro-5H-thiazolo[3,2-c]pyrimidine-2-carboxylic acid 3-fluoro-benzylamide;

6-(3,4-Dichloro-benzyl)-8-methyl-5,7-dioxo-6,7-dihydro-5H-thiazolo[3,2-c]pyrimidine-2-carboxylic acid benzylamide;

6-(3,4-Dichloro-benzyl)-8-methyl-5,7-dioxo-6,7-dihydro-5H-thiazolo[3,2-c]pyrimidine-2-carboxylic acid 4-methyl-benzylamide;

8-Methyl-5,7-dioxo-6-pyridin-4-ylmethyl-6,7-dihydro-5H-thiazolo[3,2-c]pyrimidine-2-carboxylic acid benzylamide;

6-Benzyl-8-methyl-5,7-dioxo-6,7-dihydro-5H-thiazolo[3,2-c]pyrimidine-2-carboxylic acid(pyridin-4-ylmethyl)-amide;

6-Benzyl-8-methyl-5,7-dioxo-6,7-dihydro-5H-thiazolo[3,2-c]pyrimidine-2-carboxylic acid 4-methoxy-benzylamide;

6-(4-Methoxy-benzyl)-8-methyl-5,7-dioxo-6,7-dihydro-5H-thiazolo[3,2-c]pyrimidine-2-carboxylic acid 4-methoxy-benzylamide;

8-Methyl-5,7-dioxo-6-pyridin-4-ylmethyl-6,7-dihydro-5H-thiazolo[3,2-c]pyrimidine-2-carboxylic acid 4-methoxy-benzylamide;

6-Benzyl-8-methyl-5,7-dioxo-6,7-dihydro-5H-thiazolo[3,2-c]pyrimidine-2-carboxylic acid 3,4-dimethoxy-benzylamide;

6-(4-Methanesulfonyl-benzyl)-8-methyl-5,7-dioxo-6,7-dihydro-5H-thiazolo[3,2-c]pyrimidine-2-carboxylic acid 3,4-dimethoxy-benzylamide;

8-Methyl-5,7-dioxo-6-(4-sulfamoyl-benzyl)-6,7-dihydro-5H-thiazolo[3,2-c]pyrimidine-2-carboxylic acid 3,4-dimethoxy-benzylamide;

6-(4-Dimethylsulfamoyl-benzyl)-8-methyl-5,7-dioxo-6,7-dihydro-5H-thiazolo[3,2-c]pyrimidine-2-carboxylic acid 3,4-dimethoxy-benzylamide;

8-Methyl-5,7-dioxo-6-pyridin-3-ylmethyl-6,7-dihydro-5H-thiazolo[3,2-c]pyrimidine-2-carboxylic acid 3,4-dimethoxy-benzylamide;

8-Methyl-5,7-dioxo-6-pyridin-2-ylmethyl-6,7-dihydro-5H-thiazolo[3,2-c]pyrimidine-2-carboxylic acid 3,4-dimethoxy-benzylamide;

6-Benzyl-8-methyl-5,7-dioxo-6,7-dihydro-5H-thiazolo[3,2-c]pyrimidine-2-carboxylic acid 3-methoxy-benzylamide;

6-(3-Methoxy-benzyl)-8-methyl-5,7-dioxo-6,7-dihydro-5H-thiazolo[3,2-c]pyrimidine-2-carboxylic acid 3-methoxy-benzylamide;

6-(3-Methoxy-benzyl)-8-methyl-5,7-dioxo-6,7-dihydro-5H-thiazolo[3,2-c]pyrimidine-2-carboxylic acid(benzo[1,3]dioxol-5-ylmethyl)-amide;

6-Benzo[1,3]dioxol-5-ylmethyl-8-methyl-5,7-dioxo-6,7-dihydro-5H-thiazolo[3,2-c]pyrimidine-2-carboxylic acid(benzo[1,3]dioxol-5-ylmethyl)-amide;

6-Benzyl-8-methyl-5,7-dioxo-6,7-dihydro-5H-thiazolo[3,2-c]pyrimidine-2-carboxylic acid 4-methylsulfanyl-benzylamide;

6-Benzyl-8-methyl-5,7-dioxo-6,7-dihydro-5H-thiazolo[3,2-c]pyrimidine-2-carboxylic acid 3,4-dichlorobenzylamide;

6-Benzyl-8-methyl-5,7-dioxo-6,7-dihydro-5H-thiazolo[3,2-c]pyrimidine-2-carboxylic acid 4-methoxybenzylamide;

6-Benzyl-8-methyl-5,7-dioxo-6,7-dihydro-5H-thiazolo[3,2-c]pyrimidine-2-carboxylic acid benzylamide;

6-(4-Pyridylmethy)-5,7-dioxo-6,7-dihydro-5H-thiazolo[3,2-c]pyrimidine-2-carboxylic acid benzylamide hydrochloride;

6-(4-Chlorobenzyl)-5,7-dioxo-6,7-dihydro-5H-thiazolo[3,2-c]pyrimidine-2-carboxylic acid 3,4-dichlorobenzylamide;

6-(4-Chlorobenzyl)-5,7-dioxo-6,7-dihydro-5H-thiazolo[3,2-c]pyrimidine-2-carboxylic acid benzylamide;

6-(3,4-Dichlorobenzyl)-5,7-dioxo-6,7-dihydro-5H-thiazolo[3,2-c]pyrimidine-2-carboxylic acid benzylamide;

6-Benzoyl-5,7-dioxo-6,7-dihydro-5H-thiazolo[3,2-c]pyrimidine-2-carboxylic acid benzylamide;

6-Benzyl-8-methyl-5,7-dioxo-6,7-dihydro-5H-thiazolo[3,2-c]pyrimidine-2-carboxylic acid 3-fluoro-benzylamide;

6-Benzyl-5,7-dioxo-6,7-dihydro-5H-thiazolo[3,2-c]pyrimidine-2-carboxylic acid(pyridin-4-ylmethyl)-amide hydrochloride;

6-Benzyl-5,7-dioxo-6,7-dihydro-5H-thiazolo[3,2-c]pyrimidine-2-carboxylic acid biphenyl-4-ylamide;

6-Benzyl-8-formyl-5,7-dioxo-6,7-dihydro-5H-thiazolo[3,2-c]pyrimidine-2-carboxylic acid 4-fluoro-benzylamide; and

6-Benzyl-8-hydroxymethyl-5,7-dioxo-6,7-dihydro-5H-thiazolo[3,2-c]pyrimidine-2-carboxylic acid 4-fluoro-benzylamide.

Another invention embodiment is a compound of Formula VIII, or a pharmaceutically acceptable salt thereof, selected from:

4-[2-(4-Methoxy-benzylcarbamoyl)-8-methyl-5,7-dioxo-7H-thiazolo[3,2-c]pyrimidin-6-ylmethyl]-benzoic acid methyl ester;

4-[2-(3-Fluoro-benzylcarbamoyl)-8-methyl-5,7-dioxo-7H-thiazolo[3,2-c]pyrimidin-6-ylmethyl]-benzoic acid methyl ester;

4-[2-(4-Fluoro-benzylcarbamoyl)-8-methyl-5,7-dioxo-7H-thiazolo[3,2-c]pyrimidin-6-ylmethyl]-benzoic acid methyl ester;

6-Benzyl-5,7-dioxo-6,7-dihydro-5H-thiazolo[3,2-c]pyrimidine-2-carboxylic acid benzyl ester;

6-Benzyl-8-methyl-5,7-dioxo-6,7-dihydro-5H-thiazolo[3,2-c]pyrimidine-2-carboxylic acid benzyl ester;

6-Benzyl-3-methyl-5,7-dioxo-6,7-dihydro-5H-thiazolo[3,2-c]pyrimidine-2-carboxylic acid methyl ester;

6-Benzyl-8-methyl-5,7-dioxo-6,7-dihydro-5H-thiazolo[3,2-c]pyrimidine-2-carboxylic acid methyl ester;

6-Benzyl-8-methyl-5,7-dioxo-6,7-dihydro-5H-thiazolo[3,2-c]pyrimidine-2-carboxylic acid pyridin-4-ylmethyl ester;

6-Benzyl-5,7-dioxo-6,7-dihydro-5H-thiazolo[3,2-c]pyrimidine-2-carboxylic acid pyridin-4-ylmethyl ester;

8-Methyl-5,7-dioxo-6-pyridin-4-ylmethyl-6,7-dihydro-5H-thiazolo[3,2-c]pyrimidine-2-carboxylic acid 4-methoxy-benzyl ester; and

6-Benzyl-3-methyl-5,7-dioxo-6,7-dihydro-5H-thiazolo[3,2-c]pyrimidine-2-carboxylic acid benzyl ester.

Another invention embodiment is a compound of Formula I, or a pharmaceutically acceptable salt thereof, selected from:

6-Benzoyl-thiazolo[3,2-c]pyrimidine-5,7-dione;

6-(4-Chlorobenzyl)-thiazolo[3,2-c]pyrimidine-5,7-dione;

6-Pyridin-4-ylmethyl-thiazolo]3,2-c]pyrimidine-5,7-dione;

8-Methyl-thiazolo[3,2-c]pyrimidine-5,7-dione;

8-Methyl-5,7-dioxo-6,7-dihydro-5H-thiazolo[3,2-c]pyrimidine-2-carboxylic acid;

6-Benzyl-8-methyl-5,7-dioxo-6,7-dihydro-5H-thiazolo[3,2-c]pyrimidine-2-carboxylic acid;

4-(8-methyl-5,7-dioxo-7H-thiazolo[3,2-c]pyrimidin-6-yl-methyl)-benzoic acid tert-butyl ester; and

8-Methyl-6-[4-(Morpholine-4-sulfonyl)benzyl]-thiazolo[3,2-c]pyrimidine-5,7-dione.

Another invention embodiment is a compound of Formula III, or a pharmaceutically acceptable salt thereof, or a tautomer thereof, selected from:

6-Benzyl-8-methyl-5,7-dioxo-6,7-dihydro-5H-thiazolo[3,2-c]pyrimidine-2-carboxylic acid prop-2-ynylamide;

6-Benzyl-8-methyl-5,7-dioxo-6,7-dihydro-5H-thiazolo[3,2-c]pyrimidine-2-carboxylic acid(piperidin-4-ylmethyl)-amide hydrochloride;

6-(4-Bromo-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(4-Chloro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(3-Bromo4-fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(3-Bromo4-chloro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(3-Chloro4-fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(4-Chloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(3-Bromo4-fluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(3-Bromo4-chloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(3-Chloro-4-fluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(3-Bromo4-fluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(3-Bromo4-chloro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(3-Chloro4-fluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(4-Chloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(3-Bromo-4-fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(3-Bromo-4-chloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(3-Chloro4-fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(4-Chloro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(3-Bromo-4-fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(3-Bromo-4-chloro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(3-Chloro4-fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(4-Chloro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(4-Fluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(3-Bromo-4-fluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(3-Bromo-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(3-Chloro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(3-Fluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(3,4-Dichloro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(3,4-Difluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(3-Chloro4-fluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(4-Chloro-3-bromo-benzyl)-8-methyl-5,7-dioxo-6,7-dihydro-5H-thiazolo[3,2-c]pyrimidine-2-carboxylic acid(2-methylamino-pyridin-4-ylmethyl)-amide;

6-(4-Bromo-benzyl)-8-methyl-5,7-dioxo-6,7-dihydro-5H-thiazolo[3,2-c]pyrimidine-2-carboxylic acid(pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid(pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid(pyridin-3-ylmethyl)-amide;

6-(3-Bromo-4-fluoro-benzyl)-8-methyl-5,7-dioxo-6,7-dihydro-5H-thiazolo[3,2-c]pyrimidine-2-carboxylic acid(pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid(pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(pyridin-3-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid(pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid(pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(pyridin-3-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid(pyridin-3-ylmethyl)-amide;

6-(3-Bromo-4-chloro-benzyl)-8-methyl-5,7-dioxo-6,7-dihydro-5H-thiazolo[3,2-c]pyrimidine-2-carboxylic acid(pyridin-3-ylmethyl)-amide;

6-(3-Chloro4-fluoro-benzyl)-8-methyl-5,7-dioxo-6,7-dihydro-5H-thiazolo[3,2-c]pyrimidine-2-carboxylic acid(pyridin-3-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(3-Bromo-4-fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(3-Bromo-4-chloro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(3-Chloro-4-fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(3-Bromo-4-fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(3-Bromo-4-chloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(3-Chloro4-fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(3-Bromo-4-fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(3-Bromo-4-chloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(3-Chloro4-fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;

6-(4-Bromo-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(4-Chloro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(4-Fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(3-Bromo-4-fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(3-Bromo-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(4-Bromo-3-fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(3-Chloro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(3-Fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(3,4-Dichloro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(4-Bromo-3-chloro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(3,4-Difluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(3-Bromo-4-chloro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(3-Chloro-4-fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(4-Chloro-3-fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;

6-(4-Cyano-benzyl)-8-methyl-5,7-dioxo-6,7-dihydro-5H-thiazolo[3,2-c]pyrimidine-2-carboxylic acid(2-methoxy-pyridin-4-ylmethyl)-amide; and

6-(4-Isopropylsulfamoyl-benzyl)-8-methyl-5,7-dioxo-6,7-dihydro-5H-thiazolo[3,2-c]pyrimidine-2-carboxylic acid(2-methoxy-pyridin-4-ylmethyl)-amide.

Another invention embodiment is a compound of Formula I, selected from:

8-Methyl-5,7-dioxo-6-(3-oxo-3-phenyl-propyl)-6,7-dihydro-5H-thiazolo[3,2-c]pyrimidine-2-carboxylic acid 4-fluoro-benzylamide;

8-Methyl-6-(1-phenylethyl) 5,7-dioxo-6,7-dihydro-5H-thiazolo[3,2-c]pyrimidine-2-carboxylic acid 4-flouro-benzylamide;

8-Methyl-5,7-dioxo-6-(2-phenylmethanesulfonyl-ethyl)-6,7-dihydro-5H-thiazolo[3,2-c]pyrimidine-2-carboxylic acid 4-flouro-benzylamide;

6-(5-Cyano-pentyl)-8-Methyl-5,7-dioxo-6,7-dihydro-5H-thiazolo[3,2-c]pyrimidine-2-carboxylic acid 4-flouro-benzylamide;

6-(E)-But-2-enyl-8-Methyl-5,7-dioxo-6,7-dihydro-5H-thiazolo[3,2-c]pyrimidine-2-carboxylic acid 4-flouro-benzylamide;

8-Methyl-5,7-dioxo-6-(E)-pent-2-enyl-6,7-dihydro-5H-thiazolo[3,2-c]pyrimidine-2-carboxylic acid 4-flouro-benzylamide;

6-sec-Butyl-8-Methyl-5,7-dioxo-6,7-dihydro-5H-thiazolo[3,2-c]pyrimidine-2-carboxylic acid 4-flouro-benzylamide;

8-Methyl-6-(2-methyl-allyl)-5,7-dioxo-6,7-dihydro-5H-thiazolo[3,2-c]pyrimidine-2-carboxylic acid 4-flouro-benzylamide;

6-(1-Ethyl-propyl)-8-Methyl-5,7-dioxo-6,7-dihydro-5H-thiazolo[3,2-c]pyrimidine-2-carboxylic acid 4-flouro-benzylamide;

8-Methyl-5,7-dioxo-6-pent-2-ynyl-6,7-dihydro-5H-thiazolo[3,2-c]pyrimidine-2-carboxylic acid 4-flouro-benzylamide;

6-(2-Benzensulfonyl-ethyl)-8-Methyl-5,7-dioxo-6,7-dihydro-5H-thiazolo[3,2-c]pyrimidine-2-carboxylic acid 4-flouro-benzylamide;

8-Methyl-6-(3-methyl-but-2-enyl)-5,7-dioxo-6,7-dihydro-5H-thiazolo[3,2-c]pyrimidine-2-carboxylic acid 4-flouro-benzylamide;

6-[2-(4-Fluoro-benzensulfonyl)-ethyl]-8-Methyl-5,7-dioxo-6,7-dihydro-5H-thiazolo[3,2-c]pyrimidine-2-carboxylic acid 4-flouro-benzylamide;

6-[3-(4-Fluoro-phenyl)-3-oxo-propyl]-8-Methyl-5,7-dioxo-6,7-dihydro-5H-thiazolo[3,2-c]pyrimidine-2-carboxylic acid 4-flouro-benzylamide;

8-Methyl-5,7-dioxo-6-{2-[(1-phenyl-methanoyl)-amino]-ethyl}-6,7-dihydro-5H-thiazolo[3,2-c]pyrimidine-2-carboxylic acid 4-flouro-benzylamide;

8-Methyl-5,7-dioxo-6-(2-phenoxy-ethyl)-6,7-dihydro-5H-thiazolo[3,2-c]pyrimidine-2-carboxylic acid 4-flouro-benzylamide; and

{5-[2-(4-Fluoro-benzylcarbamoyl)-8-methyl-5,7-dioxo-7H-thiazolo[3,2-c]pyrimidine-6-ylmethyl]-isoxazol-3-yl]}-carbamic acid methyl ester.

A further embodiment of this invention is use of a compound of Formula I, or a pharmaceutically acceptable salt thereof, in the manufacture of a medicament for the treatment of a disease mediated by an MMP-13 enzyme.

Another invention embodiment is use of a compound of Formulas II, III, IV, V, VI, VII, or VIII, or a pharmaceutically acceptable salt thereof, in the manufacture of a medicament for the treatment of a disease mediated by an MMP-13 enzyme.

Another invention embodiment is use of a compound of Formula I, or a pharmaceutically acceptable salt thereof, in the manufacture of a medicament for the treatment of cancer.

Another invention embodiment is use of a compound of Formula I, or a pharmaceutically acceptable salt thereof, in the manufacture of a medicament for the treatment of rheumatoid arthritis.

Another invention embodiment is use of a compound of Formula I, or a pharmaceutically acceptable salt thereof, in the manufacture of a medicament for the treatment of osteoarthritis.

Another invention embodiment is use of a compound of Formula I, or a pharmaceutically acceptable salt thereof, in the manufacture of a medicament for the treatment of heart failure.

Another invention embodiment is use of a compound of Formula I, or a pharmaceutically acceptable salt thereof, in the manufacture of a medicament for the treatment of inflammation.

A further embodiment of this invention is a pharmaceutical composition, comprising a compound of Formula I, or a pharmaceutically acceptable salt thereof, admixed with a carrier, excipient, or diluent.

Another invention embodiment are compositions that comprise compounds of Formulas II, II, IV, V, VI, VII, or VIII, or a pharmaceutically acceptable salt thereof.

Another embodiment of this invention is a method for inhibiting MMP-13 in an animal, comprising administering to the animal an MMP-13 inhibiting amount of a compound of Formula I, or a pharmaceutically acceptable salt thereof.

A further embodiment is a method for treating a disease mediated by an MMP-13 enzyme, comprising administering to a patient suffering from such a disease an effective amount of a compound of Formula I, or a pharmaceutically acceptable salt thereof.

Another invention embodiment is a method of treating cancer, comprising administering to a patient suffering from such a disease an anticancer effective amount of a compound of Formula I, or a pharmaceutically acceptable salt thereof.

Another invention embodiment is a method of treating breast carcinoma, comprising administering to a patient suffering from such a disease an anticancer effective amount of a compound of Formula I, or a pharmaceutically acceptable salt thereof.

Another invention embodiment is a method of treating inflammation, comprising administering to a patient suffering from such a disease an effective amount of a compound of Formula I, or a pharmaceutically acceptable salt thereof.

Another invention embodiment is a method of treating heart failure, comprising administering to a patient suffering from such a disease an effective amount of a compound of Formula I, or a pharmaceutically acceptable salt thereof.

Another invention embodiment is a method of treating rheumatoid arthritis, comprising administering to a patient suffering from such a disease an effective amount of a compound of Formula I, or a pharmaceutically acceptable salt thereof.

Another invention embodiment is a method of treating osteoarthritis, comprising administering to a patient suffering from such a disease an effective amount of a compound of Formula I, or a pharmaceutically acceptable salt thereof.

Another embodiment of the present invention is a process for preparing a compound of Formula I 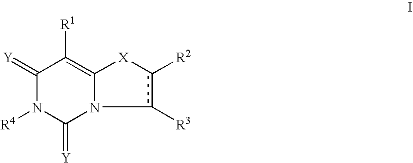
or a pharmaceutically acceptable salt thereof, wherein: [symbol]
is absent or is a bond;
X is O, S, SO, SO₂, CH₂, C═O, CHOH, NH, or NR⁵;
Y is O or S;
R¹ is H, (O)ₙC₁-C6 alkyl, (O)ₙsubstituted alkyl, NO₂, NR⁵R⁶, CHO, or halo;
R², R³, and R⁴ independently are hydrogen, halo, C₁-C₆ alkyl, substituted C₁-C₆ alkyl, C₂-C₆ alkenyl, substituted C₂-C₆ alkenyl, C₂-C₁₀ alkynyl, substituted C₂-C₁₀ alkynyl, (CH₂)ₘOH, (CH₂)ₘOR⁵, (CH₂)ₘ cycloalkyl, (CH₂)ₘ substituted cycloalkyl, CHOH(CH₂)ₘ aryl, CHOH(CH₂)ₘ substituted aryl, CHOH(CH₂)ₘ heteroaryl, CHOH(CH₂)ₘ substituted heteroaryl, (CO₂)ₙ(CH₂)ₘ aryl, (CO₂)ₙ(CH₂)ₘ substituted aryl, (CO₂)ₙ, (CH₂)ₘ heteroaryl, (CO₂)ₙ(CH₂)ₘ substituted heteroaryl, (CO₂)ₙ(CH₂)ₘ carbocycle, (CO₂)ₙ(CH₂)ₘ substituted heteroaryl, (CO₂)ₙ(CH₂)ₘ carbocycle, (CO₂)ₙ(CH₂)ₘ substituted carbocycle, (CO₂)ₙ(CH₂)ₘ heterocycle, (CO₂)ₙ(CH₂)ₘ substituted heterocycle, (CO₂)ₙ(CH₂)ₘNR⁵R⁶, (CH₂)ₘ—S(O)₀₋₂—(CH₂)ₙ-aryl, CH(C₁-C₆ alkyl)-aryl, (CH₂)ₘN(H)C(═O)aryl, (CH₂)ₘ—S(O)₀₋₂—(CH₂)ₙ-substituted aryl, CH(C₁-C₆ alkyl)-substituted aryl (CH₂)ₘN(H)C(═O) substituted aryl, C(═O)N(R⁵)—(CH₂)ₘ aryl, C(═O)N(R⁵)—(CH₂)ₘ substituted aryl, C(═O)N(R⁵)—(CH₂)ₘ heteroaryl, C(═O)N(R⁵)—(CH₂)ₘ substituted heteroaryl, C≡C—(CH₂)ₘ aryl, C≡C—(CH₂)ₘ substituted aryl, C≡C—(CH₂)ₘ-heteroaryl, C-C—(CH₂)ₘ substituted heteroaryl, C≡C—(CH₂)ₘ carbocycle, C≡C—(CH₂)ₘ substituted carbocycle, (CH₂)ₘ—O-aryl, (CH₂)ₘ—O-substituted aryl, (CH₂)ₘCOR⁵, (CH₂)ₘCONR⁵R⁶ 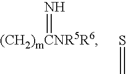
(CH₂)ₘCNR⁵R⁶, or (CH₂)ₘCO₂R⁵; m is an integer from 0 to 6;
R⁵ and R⁶ independently are hydrogen, C₁-C₆ alkyl, substituted C₁-C₆ alkyl, (CH₂)ₘ aryl, (CH₂)ₘ substituted aryl, (CH₂)ₘ heteroaryl or (CH₂)ₘ substituted heteroaryl, or R⁵ and R⁶ are taken together with the nitrogen atom to which they are attached complete a 3- to 7-membered ring containing carbon atoms, the nitrogen atom bearing R⁵ and R⁶, and optionally 1 or 2 heteroatoms independently selected form O, S, and NR², wherein R² is as defined above; and
n is 0 or 1; with the proviso that R² and R⁴ are not both selected from hydrogen and C₁-C₆ alkyl,
the process comprising the step of:
contacting a compound of Formula (A) 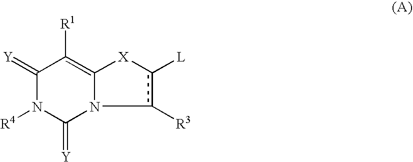
   wherein X, Y, R¹, R³ and R⁴ are as defined above; and

L is a group K or Q, wherein

K is halo, B(OH)₂, Sn(C₁-C₆ alkyl)₃, or OS(O)₂CF₃, and

Q is CO₂H, CO₂M, C(═O)-halo, C(═O)—OR⁷, C(═O)NR⁸R⁹, C(═O)—C(halo)₃, or C≡N,

wherein R⁷ is pentafluorophenyl, C(═O)R⁴, wherein R⁴ is as defined above, or S(O)₂R⁴, wherein R⁴ is as defined above;
R⁸ and R⁹ are taken together with the nitrogen atom to which they are attached to form imidazol-1-yl, phthalimid-1-yl, benzotriazol-1-yl, or tetrazol-1-yl; and M is an alkali earth metal cation or alkaline earth metal cation;
with a solvent and, when L is the group Q, a compound of Formula (B)
   D-R³ (B)
   wherein R³ is as defined above and D is HO, HN(R⁵), MO, or MN(R⁵);
wherein R⁵ and M are as defined above;
optionally in the presence of from 1 to 3 agents selected from:

a coupling agent, a tertiary organic amine, an acid catalyst, a base catalyst,

an acid halide, and an acid anhydride.

Another invention embodiment is the above process comprising the step of:

contacting a compound of Formula (A) as defined above with a solvent and, when L is the group K, a compound of Formula (C)
G-C≡C—R³ (C)
wherein R³ is as defined above and
G is hydrogen or halo;

optionally in the presence of a coupling catalyst.

Another invention embodiment is any invention process wherein Y is O and X is S.

Another invention embodiment is any invention process wherein Y is O, X is S, and R³ is C₂-C₁₀ alkynyl.

Another invention embodiment is any invention process wherein R³ is (CO₂)ₙ(CH₂)ₘaryl, (CO₂)ₙ(CH₂)ₘ substituted aryl, (CO₂)ₙ(CH₂)ₘ heteroaryl, or (CO₂)ₙ(CH₂)ₘ substituted heteroaryl, wherein n and m are as defined above, Y is O, and X is S.

Another invention embodiment is any invention process wherein R³ is C(═O)N(R⁵)(CH₂)ₘ aryl, C(═O)N(R⁵)(CH₂)ₘ substituted aryl, C(═O)N(R⁵)(CH₂)ₘ heteroaryl, or C(═O)N(R⁵)(CH₂)ₘ substituted heteroaryl, Y is O, and X is S.

Another invention embodiment is any invention process wherein L is CO₂H, CO₂M, or C(═O)-halo.

Another invention embodiment is any invention process wherein L is halo.

Another invention embodiment is any invention process wherein G is H.

### DETAILED DESCRIPTION OF THE INVENTION

The compounds provided by this invention are those defined by Formula I. In Formula I, R¹ to R⁴ include “C₁-C₆ alkyl” groups. These are straight and branched carbon chains having from 1 to 6 carbon atoms. Examples of such alkyl groups include methyl, ethyl, isopropyl, tert-butyl, neopentyl, and n-hexyl. The alkyl groups can be substituted if desired, for instance with groups such as hydroxy, amino, alkyl, and dialkylamino, halo, trifluoromethyl, carboxy, nitro, and cyano.

Examples of NR⁴R⁵ groups include amino, methylamino, di-isopropylamino, acetyl amino, propionyl amino, 3-aminopropyl amino, 3-ethylaminobutyl amino, 3-di-n-propylamino-propyl amino, 4-diethylaminobutyl amino, and 3-carboxypropionyl amino. R⁴ and R⁵ can be taken together with the nitrogen to which they are attached to form a ring having 3 to 7 carbon atoms and 1, 2, or 3 heteroatoms selected from the group consisting of nitrogen, substituted nitrogen, oxygen, and sulfur. Examples of such cyclic NR⁴R⁵ groups include pyrrolidinyl, piperazinyl, 4-methylpiperazinyl, 4-benzylpiperazinyl, pyridinyl, piperidinyl, pyrazinyl, morpholinyl, and the like.

“Halo” includes fluoro, chloro, bromo, and iodo. It should be appreciated that invention compounds do not include compounds containing an N-halo group.

“Alkenyl” means straight and branched hydrocarbon radicals having from 2 to 6 carbon atoms and one double bond and includes ethenyl, 3-buten-1-yl, 2-ethenylbutyl, 3-hexen-1-yl, and the like.

“Alkynyl” means straight and branched hydrocarbon radicals having from 2 to 10 carbon atoms and one triple bond and includes ethynyl, 3-butyn-1-yl, propynyl, 2-butyn-1-yl, 3-pentyn-1-yl, 1-hexyn-1-yl, 7,7-dimethyl-1-octyn-1-yl, and the like.

“Cycloalkyl” means a monocyclic or polycyclic hydrocarbyl group such as cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, and the like. Examples of substituted cycloalkyl include 4-carboxycyclohexyl, 4-oxo-cyclohexyl, 4-(carboxymethyl)-cyclobutyl, 3-methyl-cyclopentyl, and 3-(carboxymethyl)cyclopentyl. Also included are rings in which 1 to 3 heteroatoms replace carbons. Such groups are termed “heterocycle” or “heterocyclyl”, which mean a cycloalkyl group also bearing at least one heteroatom selected from O, S, or NR², examples being oxiranyl, pyrrolidinyl, 4-methylpiperazinyl, piperidyl, tetrahydropyranyl, and morpholinyl. The group R² here is as defined above for Formula I, except where R² contains the functional group “NR⁵R⁶”, the groups R⁵ and R⁶ are not taken together with the nitrogen atom to which they are attached to complete a 3- to 7-membered ring.

“Alkoxy” refers to the alkyl groups mentioned above bound through oxygen, examples of which include methoxy, ethoxy, isopropoxy, tert-butoxy, and the like. In addition, alkoxy refers to polyethers such as —O—(CH₂)₂—O—CH₃, and the like.

“Alkanoyl” groups are alkyl linked through a carbonyl, i.e., C₁-C₅—C(O)—. Such groups include formyl, acetyl, propionyl, butyryl, and isobutyryl.

“Acyl” means an alkyl or aryl (Ar) group bonded through a carbonyl group, i.e., R—C(O)—. For example, acyl includes a C₁-C₆ alkanoyl, including substituted alkanoyl, wherein the alkyl portion can be substituted by NR⁴R⁵ or a carboxylic or heterocyclic group. Typical acyl groups include acetyl, benzoyl, and the like.

The alkyl, alkenyl, alkoxy, and alkynyl groups described above are optionally substituted, preferably by 1 to 3 groups selected from NR⁴R⁵, phenyl, substituted phenyl, heteroaryl, substituted heteroaryl, heterocycle, thio C₁-C₆ alkyl, C₁-C₆ alkoxy, hydroxy, carboxy, C₁-C₆ alkoxycarbonyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₙPh where n is 1, 2, or 3. Perhalo and polyhalo substitution is also embraced.

Examples of substituted alkyl groups include 2-aminoethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, benzyl(Bₙ), 3-morpholinopropyl, piperazinylmethyl, pyridyl-4-methyl(Py-4-me), 3-(pyridyl-4-thio)propyl, and 2-(4-methylpiperazinyl)ethyl.

Examples of substituted alkynyl groups include 2-methoxyethynyl, 2-ethylsulfanyethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include dimethylaminomethyl, carboxymethyl, 4-dimethylamino-3-buten-1-yl, 5-ethylmethylamino-3-pentyn-1-yl, 3-(3-methoxyphenyl)-propyn-1-yl, 3-(3,4-difluorophenyl)-propyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The terms “Ar” and “aryl” refer to unsubstituted and substituted aromatic groups. Heteroaryl groups have from 4 to 10 ring atoms, from 1 to 4 of which are independently selected from the group consisting of O, S, and N. Preferred heteroaryl groups have 1 or 2 heteroatoms in a 5- or 6-membered aromatic ring. Mono- and bicyclic aromatic ring systems are included in the definition of aryl and heteroaryl. Typical aryl groups include phenyl and naphthyl. Typical substituted aryl groups include 3,4-difluorophenyl, 4-carboxyphenyl, 3,4-methylenedioxyphenyl, 4-carboxymethylphenyl, 3-methoxyphenyl, and 7-fluoro-1-naphthyl. Typical heteroaryl groups include pyridyl, thienyl, benzothienyl, indolyl, furanyl, thiazolyl, isothiazolyl, indazolyl, 2-oxo-2H-1-benzopyranyl, and imidazolyl. Typical substituted heteroaryl groups include 3-methoxy-isothiazolyl, 3-methoxypyridin-4-yl, 4-ethylbenzothienyl, 4-thiopyridyl, 2-methoxy-pyridin-4-yl, 1-methylpyrazol-4-yl, and 2-methyl-pyridin-3-yl.

Preferred Ar groups are phenyl and phenyl substituted by 1, 2, or 3 groups independently selected from alkyl, alkoxy, alkoxycarbonyl, thio, thioalkyl, (C₁-C₆ alkyl)sulfanyl, (C₁-C₆ alkyl)sulfonyl, halo, hydroxy, (CH₂)₀₋₆CO₂R⁷, trifluoromethyl, trifluoromethoxy, nitro, amino of the formula —NR⁴R⁵, C(═O)NR⁵R⁶, N(R⁴)C(═O)OR⁵, and T(CH₂)ₘQR⁴ or T(CH₂)ₘCO₂R⁴, wherein m is 1 to 6, T is O, S, NR⁴, N(O)R⁴, NR⁴R⁶Y, or CR⁴R⁵, Q is O, S, NR⁵, N(O)R⁵, or NR⁵R⁶Y, wherein R⁴-R⁶ are as described above, and R⁷ is hydrogen, alkyl, or substituted alkyl, for example, methyl, trichloroethyl, diphenylmethyl, and the like. The alkyl and alkoxy groups can be substituted as defined above. For example, typical groups are carboxyalkyl, alkoxycarbonylalkyl, hydroxyalkyl, hydroxyalkoxy, and alkoxyalkyl. Examples of substituted phenyl are 3-methoxyphenyl, 2,6-dichlorophenyl, 3-nitrophenyl, 4-dimethylaminophenyl, and biphenyl.

Preferred heteroaryl groups include thienyl, furanyl, pyrrolyl, isoxazolyl, isothiazolyl, imidazolyl, pyrazolyl, oxazolyl, thiazolyl, 1,2,4-oxadiazolyl, 1,2,4,-thiadiazolyl, 1,2,4-triazolyl, tetrazolyl, benzofuranyl, benzothienyl, indolyl, benzimidazolyl, benztriazolyl, benzoxazolyl, benzthiazolyl, pyridinyl, pyrimidinyl, quinolinyl, isoquinolinyl, and 2-oxo-2H-1-benzopyranyl.

Preferred heteroaryl groups may be substituted on a carbon atom as described above for substituted phenyl, and may further be substituted on a ring nitrogen atom (i.e, by replacing a hydrogen from a ring nitrogen atom, which is an NH group) with (C₁-C₆ alkyl) C(═O), C₁-C₆ alkyl, C₂-C₆ alkenyl, C₂-C₁₀ alkynyl, or benzyl.

The term “substituted”, unless otherwise defined, includes from 1 to 3 substituents selected from:
C₁-C₆ alkyl; C₂-C₆ alkenyl; C₂-C₆ alkynyl; C₁-C₆ alkoxy; phenyl;
(C₁-C₆ alkoxy)carbonyl; (C₁-C₆ alkyl)sulfanyl; (C₁-C₆ alkyl)carbonyl;
OH; NH₂; N(H)R⁴, wherein R⁴ is as defined above for Formula I;
NR⁴R⁵, wherein R⁴ and R⁵ are as defined above for Formula I, or R⁴ and R⁵ are taken together with the nitrogen atom to which they are attached to form a 3- to 7-membered saturated ring containing carbon atoms and optionally from 1 or 2 heteroatoms selected from O, S, S(O), S(O)₂, N(H), and N(C₁-C₆ alkyl), wherein the ring may be optionally substituted on a carbon atom with 1 oxo (i.e., ═O) group;
C(═O)NR⁴R⁵, wherein R⁴ and R⁵ are as defined immediately above, or R⁴ and R⁵ are taken together with the nitrogen atom to which they are attached to form a 3- to 7-membered saturated ring containing carbon atoms and optionally 1 or 2 heteroatoms selected from O, S, S(O), S(O)₂, N(H), and N(C₁-C₆ alkyl), wherein the ring may be optionally substituted on a carbon atom with 1 oxo (i.e., ═O) group;
CN; NO₂; CF₃; CO₂H; CHO; SH; (C₁-C₆alkyl) S(O);
(C₁-C₆ alkyl)sulfonyl; halo; S(O)₂NR⁴R⁵, wherein R⁴ and R⁵ are as defined above for Formula I, or R⁴ and R⁵ are taken together with the nitrogen atom to which they are attached to form a 3- to 7-membered saturated ring containing carbon atoms and optionally 1 or 2 heteroatoms selected from O, S, S(O), S(O)₂, N(H), and N(C₁-C₆ alkyl), wherein the ring may be optionally substituted on a carbon atom with 1 oxo (i.e., ═O) group;
OCF₃; and (CH₂)ₘCO₂H, wherein m is as defined above for Formula I.

The phrase “tertiary organic amine” means a trisubstituted nitrogen group wherein the 3 substituents are independently selected from C₁-C₁₂ alkyl, C₃-C₁₂ cycloalkyl, benzyl, or wherein two of the substituents are taken together with the nitrogen atom to which they are attached to form a 5- or 6-membered, monocyclic heterocycle containing one nitrogen atom and carbon atoms, and the third substituent is selected from C₁-C₁₂ alkyl and benzyl, or wherein the three substituents are taken together with the nitrogen atom to which they are attached to form a 7- to 12-membered bicyclic heterocycle containing 1 or 2 nitrogen atoms and carbon atoms, and optionally a C═N double bond when 2 nitrogen atoms are present. Illustrative examples of tertiary organic amine include triethylamine, diisopropylethylamine, benzyl diethylamino, dicyclohexylmethyl-amine, 1,8-diazabicycle[5.4.0]undec-7-ene (“DBU”), 1,4-diazabicyclo[2.2.2]octane (“TED”), and 1,5-diazabicycle[4.3.0]non-5-ene.

The term “coupling agent” includes any reagent, or any combination of two, three, or four reagents, conventionally used to promote coupling of a carboxylic acid, or a pharmaceutically acceptable salt thereof, with an alcohol or an amine to yield a carboxylic ester or carboxylic amide, respectively. The coupling agents are described in Reagents for Organic Synthesis, by Fieser and Fieser, John Wiley & Sons, Inc., New York, 2000; Comprehensive Organic Transformations, by Richard C. Larock, VCH Publishers, Inc., New York, 1989; the series Compendium of Organic Synthetic Methods (1989) by Wiley-Interscience; and the text Advanced Organic Chemistry, 5^{th} edition, by Jerry March, Wiley-Interscience, New York (2001). Illustrative examples of coupling agents include N,N′-carbonyldiimidazole (“CDI”), N,N′-dicyclohexylcarbodiimide (“DCC”), triphenylphosphine with diethylazodicarboxylate, bis(2-oxo-3-oxazolidinyl)phosphinic chloride (“BOP-C1”), POCl₃, Ti(Cl)₄, and 1-(3-dimethylaminopropyl)-3-ethylcarbodiimide hydrochloride (“EDAC”).

The phrase “acid catalyst” means any protic or Lewis acid that is conventionally used to catalyze coupling of a carboxylic acid, or a pharmaceutically acceptable salt thereof, a nitrile, carboxylic ester, carboxylic amide, carboxylic acid halide, or carboxylic acid anhydride with an alcohol or an amine to yield a carboxylic ester or carboxylic amide, respectively. The acid catalysts are described in Reagents for Organic Synthesis, by Fieser and Fieser, John Wiley & Sons, Inc., New York, 2000; Comprehensive Organic Transformations, by Richard C. Larock, VCH Publishers, Inc., New York, 1989; the series Compendium of Organic Synthetic Methods (1989) by Wiley-Interscience; and the text Advanced Organic Chemistry, 5^{th} edition, by Jerry March, Wiley-Interscience, New York (2001). Illustrative examples include anhydrous hydrogen chloride, hydrochloric acid, hydrogen bromide in acetic acid, zinc chloride, titanium tetrachloride, acetic acid, trifluoroacetic acid, phenol, sulfuric acid, methanesulfonic acid, magnesium sulfate, Amberlyst-15 resin, silica gel, and the like.

It should be appreciated that a nitrile may be contacted with an alcohol or an amine in the presence of an acid catalyst, and the resulting intermediate imidate or amidine, respectively, may be contacted with water to yield the carboxylic ester or carboxylic amide, respectively.

The phrase “base catalyst” means any base that is conventionally used to catalyze coupling of a carboxylic acid, or a pharmaceutically acceptable salt thereof, carboxylic ester, carboxylic amide, carboxylic acid halide, or carboxylic acid anhydride with an alcohol or an amine to yield a carboxylic ester or carboxylic amide, respectively. The base catalysts are described in Reagents for Organic Synthesis, by Fieser and Fieser, John Wiley & Sons, Inc., New York, 2000; Comprehensive Organic Transformations, by Richard C. Larock, VCH Publishers, Inc., New York, 1989; the series Compendium of Organic Synthetic Methods (1989) by Wiley-Interscience; and the text Advanced Organic Chemistry, 5^{th} edition, by Jerry March, Wiley-Interscience, New York (2001). Illustrative examples include sodium hydroxide, sodium hydride, potassium tert-butoxide, a tertiary organic amine, titanium tetraisopropoxide, sodium methoxide, sodium acetate, sodium bicarbonate, potassium carbonate, basic alumina, and the like.

The phrase “acid halide” means any carboxylic acid halide or sulfonic acid halide that is conventionally used to catalyze coupling of a carboxylic acid, or a pharmaceutically acceptable salt thereof, with an alcohol or an amine to yield a carboxylic ester or carboxylic amide, respectively. The acid halides are described in Reagents for Organic Synthesis, by Fieser and Fieser, John Wiley & Sons, Inc., New York, 2000; Comprehensive Organic Transformations, by Richard C. Larock, VCH Publishers, Inc., New York, 1989; the series Compendium of Organic Synthetic Methods (1989) by Wiley-Interscience; and the text Advanced Organic Chemistry, 5^{th} edition, by Jerry March, Wiley-Interscience, New York (2001). Illustrative examples include acetyl chloride, trifluoromethanesulfonyl chloride, 2,2-dimethylacetyl bromide, para-toluenesulfonyl chloride, pentafluoro-benzoyl chloride, and the like.

The phrase “acid anhydride” means any carboxylic acid anhydride or sulfonic acid anhydride that is conventionally used to catalyze coupling of a carboxylic acid, or a pharmaceutically acceptable salt thereof, with an alcohol or an amine to yield a carboxylic ester or carboxylic amide, respectively. The acid anhydrides are described in Reagents for Organic Synthesis, by Fieser and-Fieser, John Wiley & Sons, Inc., New York, 2000; Comprehensive Organic Transformations, by Richard C. Larock, VCH Publishers, Inc., New York, 1989; the series Compendium of Organic Synthetic Methods (1989) by Wiley-Interscience; and the text Advanced Organic Chemistry, 5^{th} edition, by Jerry March, Wiley-Interscience, New York (2001). Illustrative examples include acetic anhydride, trifluoroacetic anhydride, trifluoromethanesulfonic acid anhydride, pentafluoro-benzoic anhydride, mixed anhydrides like trifluoroacetyloxycarbonylmethyl, and the like.

The term “halide” includes fluoride, chloride, bromide, and iodide.

The phrase “coupling catalyst” means any metal catalyst, preferably a transition metal catalyst, that is conventionally used to catalyze coupling of an aryl halide, aryl trifluoromethanesulfonate, heteroaryl halide, or heteroaryl trifluoromethanesulfonate, or activated derivatives thereof, including arylboronic acids, heteroarylboronic acids, aryl stannanes, heteroarylstannanes, aryl magnesium halides, heteroaryl magnesium halides, aryl lithiums, or heteroaryl lithiums, with an terminal alkyne to yield an arylalkyne or heteroarylalkyne. The coupling catalysts are described in Reagents for Organic Synthesis, by Fieser and Fieser, John Wiley & Sons, Inc., New York, 2000; Comprehensive Organic Transformations, by Richard C. Larock, VCH Publishers, Inc., New York, 1989; the series Compendium of Organic Synthetic Methods (1989) by Wiley-Interscience; and the text Advanced Organic Chemistry, 5^{th} edition, by Jerry March, Wiley-Interscience, New York (2001). Illustrative examples of coupling catalysts include tetrakis(triphenylphosphine)palladium (0), palladium (II) chloride, palladium (II) acetate, iron (III) chloride, Heck reaction catalysts, Suzuki reaction catalysts, Stille reaction catalysts, and the like.

The term “patient” means a mammal. Preferred patients include humans, cats, dogs, cows, horses, pigs, and sheep.

The term “animal” means a mammal. Preferred animals are include humans, rats, mice, guinea pigs, rabbits, monkeys, cats, dogs, cows, horses, pigs, and sheep.

The phrases “therapeutically effective amount” and “effective amount” are synonymous unless otherwise indicated, and mean an amount of a compound of the present invention that is sufficient to improve the condition, disease, or disorder being treated. Determination of a therapeutically effective amount, as well as other factors related to effective administration of a compound of the present invention to a patient in need of treatment, including dosage forms, routes of administration, and frequency of dosing, may depend upon the particulars of the condition that is encountered, including the patient and condition being treated, the severity of the condition in a particular patient, the particular compound being employed, the particular route of administration being employed, the frequency of dosing, and the particular formulation being employed. Determination of a therapeutically effective treatment regimen for a patient is within the level of ordinary skill in the medical or veterinarian arts.

The phrase “admixed” or “in admixture” means the ingredients so mixed comprise either a heterogeneous or homogeneous mixture. Preferred is a homogeneous mixture.

The phrases “pharmaceutical preparation” and “preparation” are synonymous unless otherwise indicated, and include the formulation of the active compound with encapsulating material as a carrier providing a capsule in which the active component, with or without other carriers, is surrounded by a carrier, which is thus in association with it. Similarly, cachets and lozenges are included. Pharmaceutical preparations are fully described below.

The phrase “anticancer effective amount” means an amount of invention compound, or a pharmaceutically acceptable salt thereof, sufficient to inhibit, halt, or cause regression of the cancer being treated in a particular patient or patient population. For example in humans or other mammals, an anticancer effective amount can be determined experimentally in a laboratory or clinical setting, or may be the amount required by the guidelines of the United States Food and Drug Administration, or equivalent foreign agency, for the particular cancer and patient being treated.

The phrase “anti-arthritic effective amount” means an amount of invention compound, or a pharmaceutically acceptable salt thereof, sufficient to inhibit, halt, or cause regression of the arthritis being treated in a particular patient or patient population. For example in humans or other mammals, an anti-arthritic effective amount can be determined experimentally in a laboratory or clinical setting, or may be the amount required by the guidelines of the United States Food and Drug Administration, or equivalent foreign agency, for the particular arthritis and patient being treated.

The phrase “MMP-13 inhibiting amount” means an amount of invention compound, or a pharmaceutically acceptable salt thereof, sufficient to inhibit an enzyme matrix metalloproteinase-13, including a truncated form thereof, including a catalytic domain thereof, in a particular animal or animal population. For example in a human or other mammal, an MMP-13 inhibiting amount can be determined experimentally in a laboratory or clinical setting, or may be the amount required by the guidelines of the United States Food and Drug Administration, or equivalent foreign agency, for the particular MMP-13 enzyme and patient being treated.

It should be appreciated that the matrix metalloproteinases include the following enzymes:

MMP-1, also known as interstitial collagenase, collagenase-1, or fibroblast-type collagenase;

MMP-2, also known as gelatinase A or 72 kDa Type IV collagenase;

MMP-3, also known as stromelysin or stromelysin-1;

MMP-7, also known as matrilysin or PUMP-1;

MMP-8, also known as collagenase-2, neutrophil collagenase or polymorphonuclear-type (“PMN-type”) collagenase;

MMP-9, also known as gelatinase B or 92 kDa Type IV coliagenase;

MMP-10, also known as stromelysin-2;

MMP-11, also known as stromelysin-3;

MMP-12, also known as metalloelastase;

MMP-13, also known as collagenase-3;

MMP-14, also known as membrane-type (“MT”) 1-MMP or MT1-MMP;

MMP-15, also known as MT2-MMP;

MMP-16, also known as MT3-MMP;

MMP-17, also known as MT4-MMP;

MMP-18; and

MMP-19.

Other known MMPs include MMP-26 (Matrilysin-2).

One aspect of the present invention is compounds that are selective inhibitors of the enzyme MMP-13. A selective inhibitor of MMP-13, as used in the present invention, is a compound that is ≧5 times more potent in vitro versus MMP-13, or a truncated form thereof, than versus at least one other matrix metalloproteinase enzyme such as, for example, MMP-1, MMP-2, MMP-3, MMP-7, MMP-8, MMP-9, or MMP-14, or versus tumor necrosis factor alpha convertase (“TACE”). A preferred aspect of the present invention is compounds that are selective inhibitors of MMP-13 versus MMP-1.

Other aspects of the present invention are compounds of Formula I, or a pharmaceutically acceptable salt thereof, that are ≧10, ≧20, ≧50, ≧100, or ≧1000 times more potent versus MMP-13 than versus at least one of any other MMP enzyme or TACE. Still other aspects of the present invention are compounds of Formula I, or a pharmaceutically acceptable salt thereof, that are selective inhibitors of MMP-13 versus 2, 3, 4, 5, 6, or 7 other MMP enzymes, or versus TACE and 1, 2, 3, 4, 5, 6, or 7 other MMP enzymes.

It should be appreciated that the invention compounds of Formula I such as thiazolo[3,2-c]pyrimidines and oxazolo[3,2-c]pyrimidines that are only substituted with hydrogen or unsubstituted C₁-C₆ alkyl groups would not be potent or selective inhibitors of MMP-13, and are thus excluded from the compounds of the invention.

It should be appreciated that determination of proper dosage forms, dosage amounts, and routes of administration, is within the level of ordinary skill in the pharmaceutical and medical arts, and is described below.

The term “IC₅₀” means the concentration of test compound required to inhibit activity of a biological target, such as a receptor or enzyme, by 50%.

The phrase “catalytic domain” means the domain containing a catalytic zinc cation of the MMP enzyme, wherein the MMP enzyme contains two or more domains. A catalytic domain includes truncated forms thereof that retain at least some of the catalytic activity of MMP-13 or MMP-13CD. For example, the collagenases, of which MMP-13 is a member, have been reported to contain a signal peptide domain, a propeptide domain, a catalytic domain, and a hemopexin-like domain (Ye Qi-Zhuang, Hupe D., Johnson L., Current Medicinal Chemistry, 1996;3:407-418).

The phrase “a method for inhibiting MMP-13” includes methods of inhibiting full length MMP-13, truncated forms thereof that retain catalytic activity, including forms that contain the catalytic domain of MMP-13, as well as the catalytic domain of MMP-13 alone, and truncated forms of the catalytic domain of MMP-13 that retain at least some catalytic activity.

It should be appreciated that it has been shown previously (Ye Qi-Zhuang, et al., 1996, supra) that inhibitor activity against a catalytic domain of an MMP is predictive of the inhibitor activity against the respective full-length enzyme.

The compounds to be used in the present invention can exist in unsolvated forms as well as solvated forms, including hydrated forms. In general, the solvated forms, including hydrated forms, are equivalent to unsolvated forms and are intended to be encompassed within the scope of the present invention. Some of the invention compounds may have one or more chiral centers, and as such can exist as individual enantiomers and mixtures. This invention contemplates all racemic mixtures, pure enantiomers, as well as geometric and positional isomers.

The compounds of Formulas I to VIII are capable of further forming both pharmaceutically acceptable formulations comprising salts, including but not limited to acid addition and/or base salts, solvents, and N-oxides of a compound of Formulas I to VIII. This invention also provides pharmaceutical formulations comprising a compound of Formulas I to VIII together with a pharmaceutically acceptable carrier, diluent, or excipient therefor. All of these forms can be used in the method of the present invention.

Pharmaceutically acceptable acid addition salts of the compounds of Formulas I to VIII include nontoxic salts derived form inorganic acids such as hydrochloric, nitric, phosphoric, sulfuric, hydrobromic, hydroiodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are the salts of amino acids such as arginate, gluconate, galacturonate, and the like; see, for example, Berge et al., “Pharmaceutical Salts,” J. of Pharmaceutical Science, 1977;66:1-19.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metal hydroxides, or of organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine; see, for example, Berge et al., supra.

The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

The compounds of the present invention can be formulated and administered in a wide variety of oral and parenteral dosage forms, including transdermal and rectal administration. All that is required is that an MMP inhibitor be administered to a mammal suffering from a disease in an effective amount, which is that amount required to cause an improvement in the disease and/or the symptoms associated with such disease. It will be recognized to those skilled in the art that the following dosage forms may comprise as the active component, a compound of Formula I or a corresponding pharmaceutically acceptable salt or solvate of a compound of Formula I.

A compound of Formula I, or a pharmaceutically acceptable salt thereof, may be prepared by one of ordinary skill in the art of organic chemistry by procedures found in the chemical literature such as, for example, Reagents for Organic Synthesis, by Fieser and Fieser, John Wiley & Sons, Inc., New York, 2000; Comprehensive Organic Transformations, by Richard C. Larock, VCH Publishers, Inc., New York, 1989; the series Compendium of Organic Synthetic Methods (1989) by Wiley-Interscience; the text Advanced Organic Chemistry, 5^{th} edition, by Jerry March, Wiley-Interscience, New York (2001); or the Handbook of Heterocyclic Chemistry, by Alan R. Katritzky, Pergamon Press Ltd., London, (1985), to name a few. Alternatively, a skilled artisan may find methods useful for preparing the invention compounds in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the compounds of the present invention may use starting materials, reagents, solvents, and catalysts that may be purchased from commercial sources or they may be readily prepared by adapting procedures in the references or resources cited above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A.G., Switzerland, or Lancaster Synthesis Ltd., United Kingdom.

Reagents for Organic Synthesis, by Fieser and Fieser, John Wiley & Sons, Inc., New York, 2000; Comprehensive Organic Transformations, by Richard C. Larock, VCH Publishers, Inc., New York, 1989; the series Compendium of Organic Synthetic Methods (1989) by Wiley-Interscience; the text Advanced Organic Chemistry, 5^{th} edition, by Jerry March, Wiley-Interscience, New York (2001); and the Handbook of Heterocyclic Chemistry, by Alan R. Katritzky, Pergamon Press Ltd., London, (1985) are hereby incorporated by reference.

The invention compounds are prepared by methods well-known to those skilled in the art of organic chemistry. The compounds of Formula I are prepared utilizing commercially available starting materials, or reactants that are readily prepared by standard organic synthetic techniques. A typical synthesis of the invention compounds of Formula I is shown in Scheme 1 below. The first step in Scheme 1 comprises reacting a substituted (R⁴) urea or thiourea (1) with a substituted or unsubstituted (R¹) malonic acid or ester (2) in the presence of acetic anhydride (with malonic acids) or alkali alkoxide (with malonic esters), respectively, to give a pyrimidinetrione (3). Reaction of the pyrimidinetrione (3) with phosphorus oxychloride at reflux for 1 to 2 hours gives the chloropyrimidinedione (4). Reaction of the chloropyrimidinedione (4) with excess sodium hydrosulfide in dimethylformamide at 40° C. to 45° C., followed by reaction with bromoacetaldehyde dimethylacetal at 40° C. to 50° C. gives a thio substituted pyrimidinedione (5). Cyclization of the thio substituted pyrimidinedione (5) in the presence of catalytic para-toluenesulfonic acid in refluxing xylenes with azeotropic removal of methanol gives a thiazolopyrimidine of Formula I (6) (where R² and R³ both are H). Esters of structure (7) (Formula I where R² is (CH₂)ₘCO₂R⁵) are prepared by deprotonation of (6) with lithiumhexamethyldisilazane at −70° C. to −80° C. and reaction with chloroformates. Amides or thioamides (Formula I where R² is 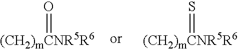
are obtained by reaction of the lithiothiazolopyrimidines with isocyanates and isothiocyanates, respectively.

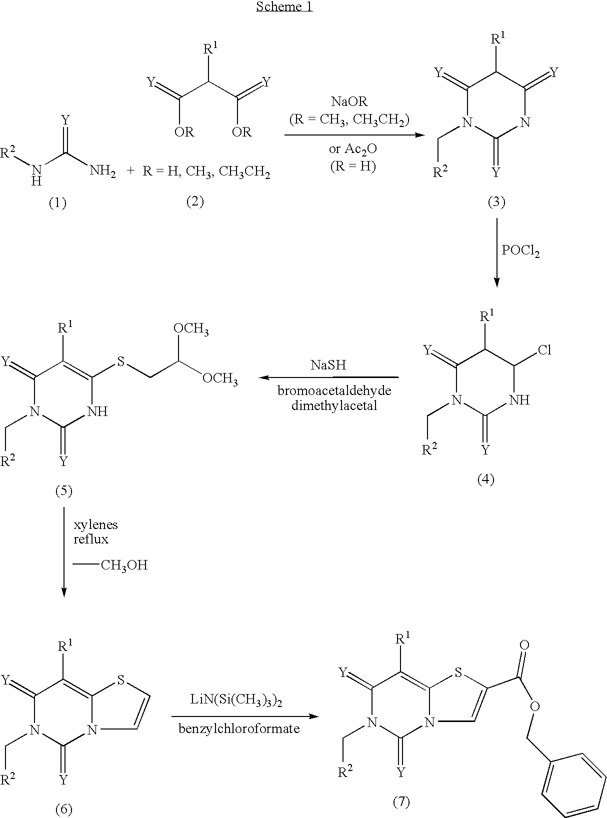

Invention compounds of Formula I, wherein X is O or NH, can be prepared according to essentially the same methodology described above for those compounds wherein X is S. The general process is illustrated in Scheme 1a. The chloropyrimidinedione (4) is reacted with a 2-hydroxyacetaldehyde dimethylacetal (for invention compounds where X is O), or with a 2-aminoacetaldehyde dimethylacetal (for invention compounds where X is NH). These reactions are carried out in an organic solvent such as dimethylformamide, and in the presence of a base such as sodium hydride or triethylamine to give the corresponding ether acetal (X═O) or amine acetal (X═NH), (Compound 5a). The ether acetals and amine acetals readily undergo cyclization by reaction with para-toluenesulfonic acid in refluxing xylenes (with azeotropic removal of methanol) to give the corresponding oxazolopyrimidine of Formula I (X═O, Compound 6a) or the imidazopyrimidine (where X═NH in Compound 6a).

These compounds can then be derivatized at the 2 and 3 positions as described above in Scheme 1, for example as illustrated in Scheme 1b. A cyclized compound (6a) wherein X═O can be reacted with benzylchloroformate in the presence of a base to cause acylation at the 2 position. The 1-amino group can be further derivatized, for instance alkylated or acylated, by standard methods, to give invention compounds such as 6c.

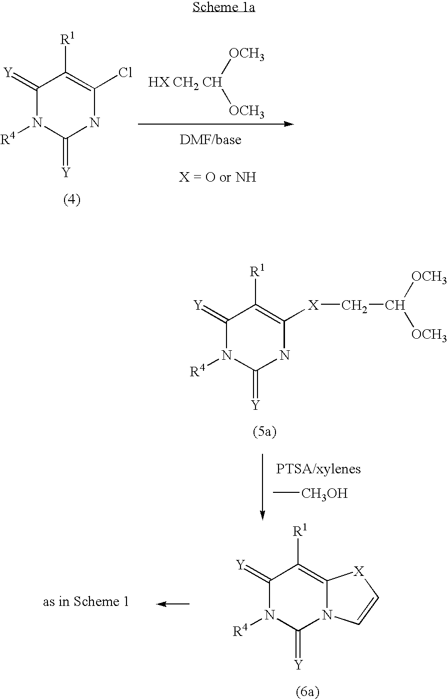

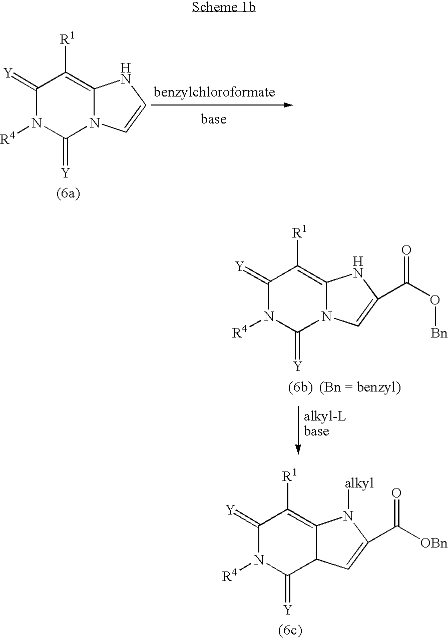

An alternative method for making invention compounds of Formula I is illustrated in Scheme 2. 6-Chloropyrimidine-2,4-dione (1) is reacted with sodium hydrogen sulfide and bromoacetaldehyde dimethyl acetal, and then treated with 1-(trimethylsilyl)iodide (TMSI), to afford a key intermediate, namely 5,7-dioxo-6,7-dihydro-5H-thiazolo[3,2-c]pyrimidine (2). This unsubstituted thiazolopyrimidine readily reacts with alkylating agents such as alkylhalides, arylalkyl halides, and heteroarylalkyl halides, (where L is a good leaving group such as halo) generally in the presence of a base such as triethylamine or cesium carbonate, to effect alkylation at the 6-position to give 6-alkyl, 6-arylalkyl, and 6-heteroarylalkyl (R⁴) thiazolopyrimidines of Formula 1 (3, wherein R¹, R², and R³ are hydrogen). These compounds are especially useful as intermediates to 2-substituted thiazolopyrimidines (where R² in Formula I is other than hydrogen). For example, the compounds (3) are readily converted to invention esters and amides by the general method described above in Scheme 1.

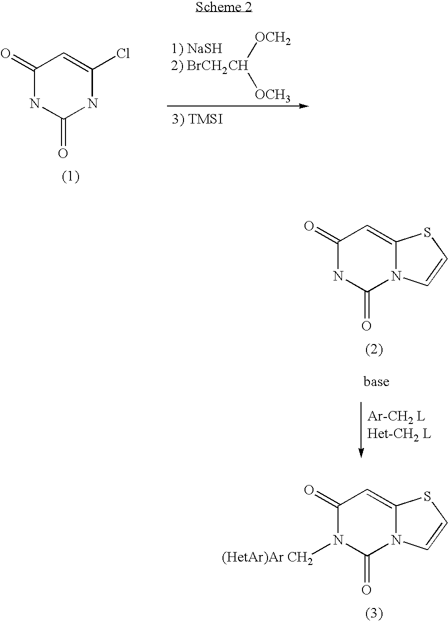

A method of preparing an intermediate that may be used to prepare a variety of compounds of Formula I is shown below in Scheme 3.

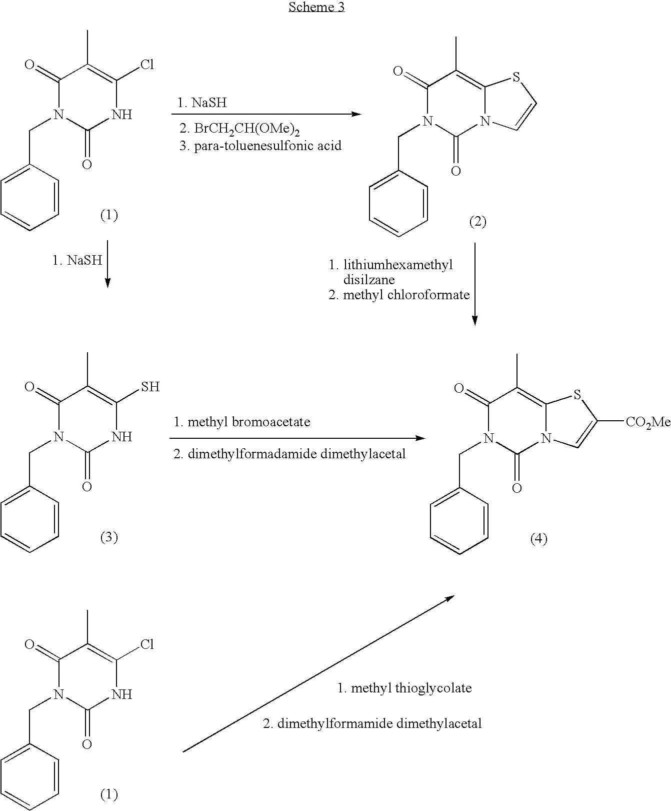

Scheme 3, a compound of formula (1) is allowed to react with NaSH to give an intermediate thiol derivative, which is allowed to react with 2-bromoacetaldehyde dimethyl acetal, followed by acid-catalyzed ring closure of the resulting thioether, to give a compound of formula (2). Deprotonation of the compound of formula (2) with lithium hexamethyldisilazane (“LiHMDS”), and quenching of the resulting anion with methylchloroformate gives a compound of formula (4).

Alternately in Scheme 3, the intermediate thiol derivative described above may be allowed to react with methyl bromoacetate, and the resulting thioether allowed to condense and cyclize with dimethylformamide dimethylacetal to give a compound of formula (4).

Still alternatively in Scheme 3, the compound of formula (1) may be allowed to react with methyl thioglycolate to give the same intermediate as that obtained by reaction of the thiol derivative described above with methyl bromoacetate. This intermediate so formed may again be allowed to react with dimethylformamide as described above to give a compound of formula (4).

The compound of formula (4) is a compound of Formula I wherein R² is CO₂CH₃, R⁴ is benzyl, R¹ is methyl, and R³ is H. The compound of formula (4) may be converted by conventional means well known to an artisan of ordinary skill in organic chemistry to compounds of Formula I independently containing esters, amides, or alkynes, to name a few, at R², arylmethyl, substituted arylmethyl, heteroarylmethyl, or substituted heteroarylmethyl, to name a few, at R⁴, and CHO or CH₂OH at R¹. For example, the compound of formula (4) may be converted to a compound of Formula I wherein R² is an amide as shown below in Method 2 of Scheme 4.

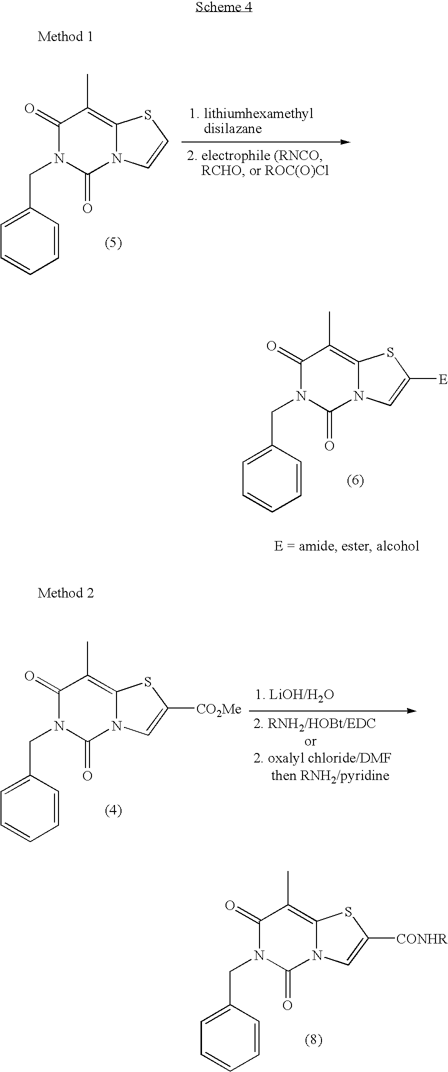

In Method 2 of Scheme 4, the compound of formula (4) is saponified with lithium hydroxide in water, and the resulting carboxylic acid derivative is allowed to couple with an amine of formula RNH₂, wherein R may be defined as R⁵ or R² (provided R² is not halo), to give an amide of formula (8). The coupling may be accomplished a number of different ways. Two such ways are using a coupling agent such as 1-(3-dimethylaminopropyl)-3-ethylcarbodiimide hydrochloride (“EDAC”) and 1-hydroxybenzotriazole hydrate (“HOBT”) together with the two reactants, or by first allowing the intermediate carboxylic acid derivative reactant to react with oxalyl chloride in the presence of a catalytic amount of N,N-dimethylformamide (“DMF”), followed by reaction of the resulting acid chloride with RNH₂ in the presence of pyridine.

Alternatively, in Scheme 4, compounds of Formula I wherein R² is an amide, ester, or alcohol may be prepared as shown by Method 1. In Method 1 of Scheme 4, a compound of formula (5) may be deprotonated with LiHMDS, and the resulting intermediate anion reacted with an electrophile such as an isocyanate (RNCO), an aldehyde (RCHO), or a chloroformate (ROC(O)Cl), wherein R is as defined above, to give an amide, alcohol, or ester, respectively of formula (6).

Alternatively the compound of formula (4) prepared according to the procedure illustrated in Scheme 3 may be converted to a compound of Formula I wherein R² is an amide and R⁴ is arylmethyl, substituted arylmethyl, heteroarylmethyl, substituted heteroarylmethyl, and further, alkenylmethyl, substituted alkenylmethyl, alkynyl methyl, or substituted alkynylmethyl, as illustrated below in Scheme 5.

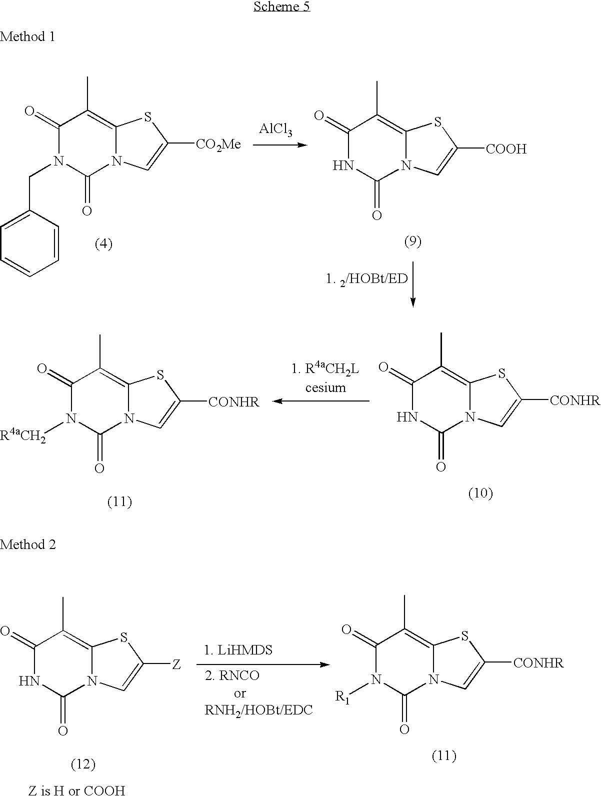

In Scheme 5, Method 1, the compound of formula (4) may be debenzylated and demethylated by reacting it with aluminum chloride to give a compound of formula (9). The compound of formula (9) can be allowed to react with an amine of formula RNH₂, wherein R is as defined above, following the EDAC procedure described above to give a compound of formula (10). The compound of formula (10) may be allowed to react with a compound of formula R^{4a}CH₂L, wherein L is a leaving group such as chloro, bromo, iodo, acetoxy, trifluoromethyl sulfonyl, or trifluoroacetyl, and R^{4a}CH₂ is a subset of the set of groups defined above for R⁴ wherein the carbon atom in R⁴ bonded to the pyrimidine ring nitrogen atom N-6 is a CH₂ group, in the presence of a base such as cesium carbonate to give a compound of formula (11).

Alternatively in Scheme 5, Method 2, the order of functionalization of the pyrimidine ring substituent groups R² and R⁴ may be reversed from that described above for Method 1 in Scheme 5.

In Scheme 5, Method 2, a compound of formula (12) may be allowed to react with the compound of formula R^{4a}CH₂L, wherein R^{4a} and LX are as defined above, first to give an intermediate containing the group R^{4a}CH₂ for R⁴, followed by conversion to an amide at R² according to the procedure described for Scheme 4, Method 1, when Z is H or conversion to an amide at R² according to the procedure described above for Method 1 of Scheme 5 (for the conversion of a compound of formula (9) to a compound of formula (10)), when Z is COOH.

During the synthesis of some of the invention compounds, it may be desirable to protect reactive functional groups such as hydroxy, amino, and carboxylic groups, so as to avoid unwanted side reactions. The use of protecting groups in synthetic organic chemistry is well-established and is fully described by Greene and Wuts in “Protecting Groups in Organic Synthesis” (John Wiley & Son Press, 3^{rd} ed). Examples of common amino protecting groups include acyl groups such as formyl and acetyl, and arylalkyl groups such as benzyl. Typical hydroxy protecting groups include ether forming groups such as methyl and ethyl, and acyl groups such as acetyl and tert-butoxycarbonyl (tBOC). Carboxylic acids generally are protected as esters, for example 2,2,2-trichloroethyl, tert-butyl, or benzyl. These protecting groups are readily cleaved by standard methods where desired.

Sulfoxides and sulfones of Formula I, wherein X is SO or SO₂, may be prepared by oxidation of the corresponding sulfides with one or two equivalents of an oxidizing agent such as peracetic acid or meta-chloroperbenzoic acid.

The following detailed examples further illustrate the synthesis of typical invention compounds of Formula I. The examples are representative only and are not to be construed as limiting the invention in any respect. All references cited herein are incorporated by reference.

### EXAMPLE 1

### 6-Benzyl-5,7-dioxo-6,7-dihydro-5H-thiazolo[3,2-c]pyrimidine-2-carboxylic acid benzyl ester

### 1-Benzyl-pyrimidine-2,4,6-trione

Step A: Freshly cut sodium metal (15.9 g, 690 mmol) was dissolved in 100% ethanol, diethylmalonate (53 mL, 349 mmol), and benzylurea (50.33 g, 335 mmol) were added, and the mixture was heated to reflux. The heat was reduced just below reflux, and ethanol (100 mL) was added. The reaction mixture was stirred 3 days at just below ethanol reflux and was then allowed to cool. Water (300 mL) and then 2N HCl (500 mL) were added, and the entire mixture was cooled to 0° C. The resulting solid was collected by filtration, washed with water, and air-dried. Two crops totaling 64.52 g (88%) were obtained.

Calcd for C₁₁H₁₀N₂O₃:
C, 60.55; H, 4.62; N, 12.84.

Found: C, 60.65; H, 4.61; N, 12.60.

### 3-Benzyl-6-chloro-1H-pyrimidine-2,4-dione

Step B: Phosphorus oxychloride (240 mL) was added in small portions over about 0.75 hour to a mixture of 1-benzyl-pyrimidine-2,4,6-trione (47.48 g, 217 mmol) and water (10 mL). Upon completing the addition, the reaction mixture was heated to reflux for 1 hour, then allowed to cool somewhat, then the phosphorus oxychloride was removed on the rotary evaporator, the resulting brown oil was added to ice, and the ice was allowed to slowly melt. The resulting precipitate was collected by filtration, washed with water, slurried in hexanes, collected by filtration, taken up in tetrahydrofuran, dried (magnesium sulfate) filtered, concentrated, and the resulting solid collected by filtration. The product was obtained in 2 portions; total 38.61 g (75.2%).

Calcd for C₁₁H₉ClN₂O₂:
C, 55.83; H, 3.83; N, 11.84.

Found: C, 55,76; H, 3.78; N, 11.62.

### 3-Benzyl-6-(2,2-dimethoxy-ethylsulfanyl)-1H-pyrimidine-2,4-dione

Step C: Ground sodium hydrosulfide hydrate (4.72 g, 84 mmol) was added to 3-benzyl-6-chloro-1H-pyrimidine-2,4-dione (4.72 g, 20 mmol) in dimethylformamide (20 mL), and the mixture was warmed to 45° C. for about 0.25 hour, and then bromacetaldehyde dimethylacetal (11 mL, 93 mmol) was added in portions over about 0.5 hour. The reaction mixture was stirred 3 days at 45° C. and was then partitioned between ethyl acetate (400 mL) and sodium bicarbonate solution (200 mL). The layers were separated, and the organic layer washed with water (200 mL) and brine (100 mL) and dried over magnesium sulfate. The solution was filtered and concentrated and triturated with hexanes/ethyl acetate and the solid collected by filtration. The solid was dissolved in methylene chloride, concentrated and triturated (1:1, hexanes/ethyl acetate), filtered and the solid dissolved in methylene chloride, concentrated and triturated (1:1, hexanes/ethyl acetate), filtered again to give 1.128 g of product. An additional 1.76 g was obtained by chromatography of the mother liquors on silica gel using hexanes/ethyl acetate as eluant. Total yield 44.8%.

Calcd for C₁₅H₁₈N₂O₄S:
C, 55.89; H, 5.63; N, 8.69.

Found: C, 55,79; H, 5.32; N, 8.63.

### 6-Benzyl-thiazolo[3,2-c]pyrimidine-5,7-dione

Step D: To a solution of 3-benzyl-6-(2,2-dimethyloxy-ethylsulfanyl)-1H-pyrimidine-2,4-dione (1.34 g, 3.83 mmol) in xylene was added 100 mg of para-toluenesulfonic acid. The resulting solution was refluxed for 5 hours while removing methanol using a Dean-Stark trap. The reaction was then cooled to room temperature and purified using flash chromatography to give the desired product as a white solid (1.01 g, 100%). R_{f}=0.26 (1:1 hexane/EtOAc); ¹H NMR (CDCl₃) δ 7.20-7.55 (m, 5H), 6.47 (d, 1H, d=4.6 Hz), 6.00 (s, 1H), 5.18 (s, 2H); MS (ACPI), m/z 259.1 (M⁺+1).

### 6-Benzyl-5,7-dioxo-6,7-dihydro-5H-thiazolo[3,2-c]pyrimidine-2-carboxylic acid benzyl ester

Step E: To a solution of diisopropylamine in THF (5 mL) at 0° C. was added n-BuLi (1.6 M, 0.15 mL, 0.24 mmol), and the resulting solution was stirred at 0° C. for 10 minutes and cooled to −78° C. A solution of 6-benzyl-thiazolo[3,2-c]pyrimidine-5,7-dione (52 mg, 0.2 mmol) in THF (5 mL) was added, and the resulting solution was stirred at −78° C. for 30 minutes. Neat benzylchloroformate (0.041 g, 0.24 mmol) was added dropwise, and the reaction was quenched by addition of NH₄Cl after 30 minutes at −78° C. After extraction with EtOAc, the organic layers were combined and washed with brine, dried, filtered and concentrated in vacuo. The residue was purified using flash chromatography to give the desired product as a yellowish solid (became white after trituration with 1:1 hexane/EtOAc, 0.014 g, 18%).

R_{f}=0.54 (1:1 hexane/EtOAc); ¹H NMR (CDCl₃) δ 7.84 (s, 1H), 6.92-7.18 (m, 10H), 5.64 (s, 1H), 5.00 (S, 2H), 4.82 (s, 2H); MS (ACPI), m/z 392.0 (M⁺+1).

### EXAMPLE 2

### 6-Benzyl-8-methyl-5,7-dioxo-6,7-dihydro-5H-thiazolo[3,2-c]pyrimidine-2-carboxylic acid benzyl ester

### 1-Benzyl-5-methyl-pyrimidine-2,4,6-trione

Step A: Sodium metal (7.68 g, 334 mmol) was dissolved in 100% ethanol (500 mL), benzylurea (25.12 g, 168 mmol) and diethylmethyl malonate (29 mL, 169 mmol) were added, and the mixture was heated at just below ethanol reflux overnight. The reaction mixture was concentrated to remove ethanol, water (200 mL), and 1 N hydrochloric acid(350 mL) were added and an oil separated. The oil would not crystallize and could not be purified by chromatography. The oil was treated with ethanol/sodium ethoxide, (400 mL/7.4 g, 322 mmol) overnight at just below ethanol reflux and was worked up as before to give an oil that would not crystallize. This material was used directly in the next step.

### 3-Benzyl-6-chloro-5-methyl-1H-pyrimidine-2,4-dione

Step B: The crude pyrimidinedione from above was taken up in tetrahydrofuran (about 10 mL), water (5 mL) was added, concentrated to remove tetrahydrofuran, and phosphorous oxychloride (110 mL) was added in portions over about 45 minutes. Then the mixture was heated at reflux for 2 hours, stirred at room temperature overnight, then the phosphorous oxychloride was removed on the rotary evaporator. Crushed ice (about 300 g) was added, and the mixture was allowed to slowly warm to room temperature and the resulting dark oil solidified on standing. The solid was collected by filtration, washed with water, taken up in tetrahydrofuran, dried over magnesium sulfate, filtered and concentrated to a brown solid. The solid was triturated with hexanes/ethyl acetate, 1:1, v/v, collected by filtration, and washed with hexanes. The product was obtained in 4 portions; total 14 g.(33.2% for the 2 steps).

### 3-Benzyl-6-(2,2-dimethoxy-ethylsulfanyl)-5-methyl-H-pyrimidine-2,4-dione

Step C: The procedure for Example 1, Step C, was used starting with 3-benzyl-6-chloro-5-methyl-1H-pyrimidine-2,4-dione (5.0 g, 20 mmol) from Step B, sodium hydrosulfide hydrate (5.06 g, 90.4 mmol), and bromoacetaldehyde dimethylacetal (13 mL, 110 mmol) to give 3-benzyl-6-(2,2-dimethoxy-ethylsulfanyl)-5-methyl-H-pyrimidine-2,4-dione in two portions; total 2.57 g. (38%).

Calcd for C₁₆H₂₀N₂O₄S:
C, 57.13; H, 5.49; N, 8.33.

Found: C, 57.30; H, 5.50; N, 8.78.

### 6-Benzyl-8-methyl-thiazolo[3,2-c]pyrimidine-5,7-dione

Step D: The thioether acetal, 3-benzyl-6-(2,2-dimethoxy-ethylsulfanyl)-5-methyl-H-pyrimidine-2,4-dione, (0.95 g, 2.8 mmol), was treated according to the procedure for Example 1, Step D to give the product, 6-benzyl-8-methyl-thiazolo[3,2-c]pyrimidine-5,7-dione (0.622 g) as a light tan solid (80.8%).

Calcd for C₁₄H₁₂N₂O₂S:
C, 61.75; H, 4.44; N, 10.29.

Found: C,61.63;H,4.51;N,10.19.

### 6-Benzyl-8-methyl-5,7-dioxo-6,7-dihydro-5H-thiazolo[3,2-c]pyrimidine-2-carboxylic acid benzyl ester

Step E: 6-Benzyl-8-methyl-thiazolo[3,2-c]pyrimidine-5,7-dione (0.262 g, 0.96 mmol) from Step D was taken up in tetrahydrofuran (25 mL), and lithium hexamethyldisilazane (1.3 mL, 1 M in tetrahydrofuran, 1.3 mmol) was added at −78° C., and the reaction was allowed to proceed for 3 minutes, then benzyl chloroformate (0.5 mL, 3.5 mmol) was added, and the reaction was stirred for 10 minutes at −78° C., ammonium chloride solution (4 mL) was added, and the reaction mixture was allowed to warm until the ice in the flask melted. The reaction mixture was partitioned between ethyl acetate (200 mL) and brine (100 mL). The layers were separated, the organic layer was dried over magnesium sulfate, filtered, and concentrated. The residue was chromatographed on silica gel using hexanes/ethyl acetate, 6:4, v/v as eluant to give the product in 2 portions, 0.158 g (40.5%)

Calcd for C₂₂H₁₈N₂O₄S:
C, 64.92; H, 4.31; N, 6.63.

Found: C, 65.01; H, 4.46; N, 6.89.

### EXAMPLE 3

### 6-Benzyl-5,7-dioxo-6,7-dihydro-5H-thiazolo[3,2-c]pyrimidine-2-carboxylic acid pyridin-4-ylmethyl ester hydrochloride

### 6-Benzyl-5,7-dioxo-6,7-dihydro-5H-thiazolo[3,2-c]pyrimidine-2-carboxylic acid methyl ester

Step A: The product from Example 1, Step D (0.518 g, 2.0 mmol), was reacted according to the procedure of Example 2, Step E, using methyl chloroformate (3.0 mL, 39 mmol) in the place of benzyl chloroformate to give the product, 6-benzyl-5,7-dioxo-6,7-dihydro-5H-thiazolo[3,2-c]pyrimidine-2-carboxylic acid methyl ester (0.084 g). An additional 0.26 g of impure product was also obtained (Total yield 54.2%).

Calcd for C₁₅H₁₂N₂O₄S:
C, 56.95; H, 3.82; N, 8.86.

Found: C, 56.87; H, 3.75; N, 8.61.

6-Benzyl-5,7-dioxo-6,7-dihydro-5H-thiazolo[3,2-c]pyrimidine-2-carboxylic acid methyl ester may also be obtained according to the following procedure.

Step A-1: The product from Example 1, Step B, namely 3-benzyl-6-chloro-1H-pyrimidine-2,4-dione, (23.7 g, 100 mmol), methyl thioglycolate (11 mL, 130 mmol), and dimethylformamide (100 mL) was heated at 55° C. for 1 hour, then triethylamine (15 mL, 108 mmol) was added and the mixture heated at 70° C. for 2 hours then stirred 3 days at room temperature. More methyl thioglycolate (4.3 mL, 50 mmol) and triethylamine (4.5 mL, 32 mmol) were added, and the mixture was heated at 70° C. for 1 hour. The volatiles were removed on the rotary evaporator at 80° C. The residue was partitioned between ethyl acetate (400 mL) and water (400 mL), and the layers were separated. The organic layer was washed with 10% citric acid solution (100 mL), and brine (100, mL), dried over magnesium sulfate, and filtered and concentrated. The aqueous washes were back-extracted with ethyl acetate (400 mL), the organic layer washed with brine (100 mL), dried, and the organics combined and concentrated to a yellow solid. Trituration with hexanes/ethyl acetate and collection by filtration gave 3-(1-benzyl-2,6-dioxo-1,2,3,6-tetrahydro-pyrimidin-4-ylsulfanyl)-acetic acid methyl ester, 18.6 g, (61%); MS (APCI+), m/z (%): 307(100), 275(40).

Step A-2: To a solution of 3-(1-benzyl-2,6-dioxo-1,2,3,6-tetrahydro-pyrimidin-4-ylsulfanyl)-acetic acid methyl ester (6.12 g, 20 mmol) and tetrahydrofuran (250 mL) at 50° C. was added dropwise dimethylformamide dimethylacetal (6 mL, 45 mmol), and the mixture was stirred at 50° C. for 0.5 hour and then was heated on the rotary evaporator (no vacuum) at 70° C. until all the solvent boiled off. Tetrahydrofuran (200 mL) was added, and the mixture stirred overnight at room temperature. Dioxane (50 mL) was added and then the reaction mixture concentrated to a viscous oil. The oil was filtered through silica gel (70-230 mesh) using hexanes/ethyl acetate, 1:1, v/v to give the product, 6-benzyl-5,7-dioxo-6,7-dihydro-5H-thiazolo[3,2-c]pyrimidine-2-carboxylic acid methyl ester, 2.94 g (46%). MS (APCI+), m/z (%): 317(100), 259(10).

### 6-Benzyl-5,7-dioxo-6,7-dihydro-5H-thiazolo[3,2-c]pyrimidine-2-carboxylic acid

Step B: The product from Example 3, Step A, namely 6-benzyl-5,7-dioxo-6,7-dihydro-5H-thiazolo[3,2-c]pyrimidine-2-carboxylic acid methyl ester, (0.226 g, 0.71 mmol) was taken up in methanol (5 mL), and tetrahydrofuran (5 mL) and 1 M sodium hydroxide solution (0.8 mL, 0.8 mmol) was added at room temperature, and the solution turned orange. Water was added until the volume reached about 25 mL, and no cloudiness occurred. The reaction mixture was allowed to stand about 10 minutes and was then poured into a separatory funnel containing ethyl acetate (200 mL), brine (100 mL), and 1 N HCl solution (3 mL). The layers were separated, dried over magnesium sulfate and concentrated to a yellow solid. The solid was triturated with hexanes/ethyl acetate and the insoluble portion collected by filtration (0.093 g) (44%). This was used directly in the next step.

### 6-Benzyl-5,7-dioxo-6,7-dihydro-5H-thiazolo[3,2-c]pyrimidine-2-carboxylic acid pyridin-4-ylmethyl ester hydrochloride

Step C: The product from Example 3, Step B, namely 6-benzyl-5,7-dioxo-6,7-dihydro-5H-thiazolo[3,2-c]pyrimidine-2-carboxylic acid(0.084 g, 0.28 mmol), 4-pyrimidinemethanol (0.082 g, 0.75 mmol), 4-dimethylaminopyridine (0.014 g, 0.11 mmol), and dichloromethane (5 mL) were stirred at room temperature and dicyclohexylcarbodiimide (0.059 g, 0.29 mmol) was added all at once, and the reaction mixture was cooled to 0° C. The reaction mixture was allowed to slowly warm to room temperature and was stirred overnight. The reaction mixture was concentrated to dryness, chromatographed on silica gel using ethyl acetate as eluant, the product-containing fractions combined and concentrated, and triturated. Dicyclohexylurea was present. The solid was taken up in tetrahydrofuran (about 3 mL), and HCl gas in ether (1 M, 1 mL, 1 mmol) was added and a precipitate formed. The mixture was concentrated to dryness, tetrahydrofuran (about 7 mL) was added and the insoluble portion collected by filtration and washed with tetrahydrofuran and air dried. The product, 6-benzyl-5,7-dioxo-6,7-dihydro-5H-thiazolo[3,2-c]pyrimidine-2-carboxylic acid pyridin-4-ylmethyl ester hydrochloride was obtained as a light yellow solid (0.0396 g), (33%).

Calcd for C₂₀H₁₅N₃O₄S HCl:
C, 55.88; H, 3.75; N, 9.77.

Found: C, 55.49; H, 3.92; N, 9.60.

### EXAMPLE 4

### 6-Benzyl-5,7-dioxo-6,7-dihydro-5H-thiazolo[3,2-c]pyrimidine-2-carboxylic acid benzylamide

To a solution of the product from Example 1, Step D, namely 6-benzyl-thiazolo[3,2-c]pyrimidine-5,7-dione, (550 mg, 2.13 mmol) in THF (5 mL) was added LiN(TMS)₂ (3.0 mL, 1.0 M, 3.0 mmol), and the resulting solution was stirred at −78° C. for 30 minutes. Neat benzylisocyanate (0.34 mL, 2.77 mmol) was added dropwise, and the reaction was stirred at −78° C. for 30 minutes and quenched by addition of NH4Cl solution. After extraction with EtOAc, the organic layers were combined and washed with brine, dried, filtered and concentrated in vacuo. The residue was purified using flash chromatography to give the desired product as a yellowish solid (became white after trituration with 1:1 hexane/EtOAc, 0.123 g, 15%). R_{f}=0.35 (1:1 hexane/EtOAc); ¹H NMR (d₈-THF) δ 8.16 (s, 1H), 7.99 (S, 1H), 7.06-7.32 (m, 10H), 5.88 (S, 1H), 4.96 (S, 2H), 4.38 (d, 2H, J=5.6 Hz); MS (ACPI), m/z 392.4 (M⁺+1).

Calcd for C₂₁H₁₇N₃O₃S₁:
C, 64.44; H, 4.38; N, 10.73.

Found: C, 63.95; H, 4.46; N, 10.72.

### EXAMPLE 5

### 6-Benzyl-2-(1-hydroxy-3-phenyl-propyl)-thiazolo[3,2-c]pyrimidine-5,7-dione

To a solution of the product from Example 1, Step D, namely 6-benzyl-thiazolo[3,2-c]pyrimidine-5,7-dione (720 mg, 2.79 mmol) in THF (5 mL) was added lithiumhexamethyldisilazane (3.91 mL, 1.0 M, 3.91 mmol), and the resulting solution was stirred at −78° C. for 30 minutes. Neat hydrocinnamaldehyde (0.5 mL, 2.77 mmol) was added dropwise, and the reaction was stirred at −78° C. for 30 minutes and quenched by addition of NH₄Cl solution. After extraction with EtOAc, the organic layers were combined and washed with brine, dried, filtered and concentrated in vacuo. The residue was purified using flash chromatography to give the desired product as a yellowish oil (450 mg, 45%). R_{f}=0.60 (1:1 hexane/EtOAc); ¹H NMR (d₈-THF) −7.47 (d, 11H), 5.92 (S, 1H), 5.14 (S, 2H), 4.64 (t, 1H), 2.70 (m, 2H), 2.01 (m, 2H); MS (ACPI), m/z 393.0 (M⁺+1).

### EXAMPLE 6

### 6-Benzyl-5,7-dioxo-6,7-dihydro-5H-thiazolo[3,2-c]pyrimidine-2-carboxylic acid biphenyl-4-ylamide

To a solution of the product from Example 1, Step D, namely 6-benzyl-thiazolo[3,2-c]pyrimidine-5,7-dione (380 mg, 1.47 mmol) in THF (5 mL) was added lithiumhexamethyldisilazane (2.2 mL, 1.0 M, 2.2 mmol), and the resulting solution was stirred at −78° C. for 30 minutes. Neat 4-biphenylisocyanate (507 mg, 2.06 mmol) was added dropwise, and the reaction was stirred at −78° C. for 30 minutes and quenched by addition of NH₄Cl solution. After extraction with EtOAc, the organic layers were combined and washed with brine, dried, filtered and concentrated in vacuo. The residue was purified using flash chromatography to give the desired product as a white solid, 0.400 g (60%). R_{f}=0.80 (1:1 hexane/EtOAc); ¹H NMR (DMSO) δ 10.61 (s, 1H), 8.90 (S, 1H), 7.21-7.90 (m, 10H), 6.26 (S, 1H), 5.13 (S, 2H); MS (ACPI+), m/z 454.2 (M⁺+1).

### EXAMPLE 7

### 6-Benzyl-2-(hydroxy-phenyl-methyl)-thiazolo[3,2-c]pyrimidine-5,7-dione

To a solution of the product from Example 1, Step D, namely 6-benzyl-thiazolo[3,2-c]pyrimidine-5,7-dione (490 mg, 1.90 mmol) in THF (5 mL) was added lithiumhexamethyldisilazane (2.66 mL, 1.0 M, 2.66 mmol), and the resulting solution was stirred at −78° C. for 30 minutes. Benzaldehyde (0.39 mL, 3.80 mmol) was added dropwise, and the reaction was stirred at −78° C. for 30 minutes and quenched by addition of NH₄Cl solution. After extraction with EtOAc, the organic layers were combined and washed with brine, dried, filtered and concentrated in vacuo. The residue was purified using flash chromatography to give the desired product as a yellowish oil (200 mg, 29%). R_{f}=0.31 (1:1 hexane/EtOAc); ¹H NMR (d₈-THF) δ 7.47 (d, 10H), 5.92 (S, 1H), 5.14 (S, 2H); MS (ACPI), m/z 365.0 (M⁺+1).

### EXAMPLE 8

### 6-Benzyl-2-(3-phenyl-propionyl)-thiazolo[3,2-c]pyrimidine-5,7-dione

To a solution of 6-benzyl-2-(1-hydroxy-3-phenyl-propyl)-thiazolo[3,2-c]pyrimidine-5,7-dione (230 mg, 0.59 mmol) in toluene was added activated MnO₂ (10 equivalents). The reaction was refluxed for 30 minutes while removing water using a Dean-Stark trap. The reaction was then cooled to room temperature and purified using flash chromatography to give the desired product as a yellow solid (190 mg, 83%). R_{f}=0.30 (2:1 hexane/EtOAc); ¹H NMR (CDCl₃) δ 8.24 (s, 1H), 7.20-7.55 (m, 10H), 5.96 (s, 1H), 5.13 (s, 2H), 2.98 (m, 4H); MS (ACPI+), m/z 381.2 (M⁺+1).

### EXAMPLE 9

### 6-Benzyl-5,7-dioxo-6,7-dihydro-5H-thiazolo[3,2-c]pyrimidine-2-carboxylic acid(pyridin-4-ylmethyl)-amide hydrochloride

The product from Example 3, Step B, namely 6-benzyl-5,7-dioxo-6,7-dihydro-5H-thiazolo[3,2-c]pyrimidine-2-carboxylic acid, (0.280 g, 0.93 mmol), 4-aminomethylpyridine (0.149 g, 1.38 mmol), 1-hydroxybenzotriazole hydrate (0.130 g, 0.96 mmol), dichloromethane (40 mL) and dimethylformamide (about 3 mL) were stirred at 0° C., and dicyclohexylcarbodiimide (0.204 g, 0.99 mmol) was added all at once, and the reaction mixture was stirred overnight at room temperature. The reaction mixture was concentrated to dryness with minimal heating, partitioned between ethyl acetate (about 400 mL) and sodium bicarbonate solution. The layers were separated, and the organic layer was washed with brine, dried over magnesium sulfate, filtered, and concentrated. The residue was filtered through silica gel (70-230 mesh) using tetrahydrofuran as eluant. The product-enriched fractions were taken up in tetrahydrofuran and treated with excess hydrogen chloride gas in diethyl ether. The mixture was concentrated to dryness, and diethyl ether was added and the insoluble material collected by filtration. The solid was washed with tetrahydrofuran and dried in vacuo to give the product, 0.055 g.

Calcd for C₂₀H₁₆N₄O₃S₁HCl H₂O:
C, 54.50; H, 4.33; N, 12.46.

Found: C, 54.66; H, 4.19; N, 12.08.

### EXAMPLE 10

### 6-Benzyl-8-methyl-5,7-dioxo-6,7-dihydro-5H-thiazolo[3,2-c]pyrimidine-2-carboxylic acid 3-fluoro-benzylamide

The product from Example 2, Step D, namely benzyl-8-methyl-thiazolo[3,2-c]pyrimidine-5,7-dione, (0.276 g (crude), 1.01 mmol) was taken up in tetrahydrofuran (40 mL) and the solution cooled to −70° C. A solution of 1 M lithiumhexamethyldisilazane in tetrahydrofuran (1.5 mL, 1.5 mmol) was added. The reaction mixture was stirred for 3 minutes at −69° C., and then neat 3-fluorobenzyl isocyanate (0.3 mL, 2.3 mmol) was added all at once. The mixture was stirred 12 minutes at −70° C. The reaction was quenched with ammonium chloride solution and partitioned between ethyl acetate (200 mL) and sodium bicarbonate solution. The layers were separated, the organic layer washed with brine, dried (magnesium sulfate) and concentrated to an orange oil. The oil was chromatographed on silica gel (70-230 mesh) using 7:3, then 2:1, hexanes/ethyl acetate then ethyl acetate as eluant. The product enriched fractions were concentrated and chromatographed again using 7:3, hexanes/ethyl acetate as eluant. The product was obtained upon trituration with diethyl ether/hexanes, 0.0085 g. 1H-NMR (CDCl₃) δ 7.95 (s, 1H), 7.37 (d, 2H), 7.2-7.4 (m, 5H), 7.26 (d 1H), 7.01 (bd, 1H), 6.47 (bt, 1H), 5.14 (s, 2H), 4.54 (d, 2H), 1.98 (s, 3H).

### EXAMPLE 11

### 6-Benzoyl-5,7-dioxo-6,7-dihydro-5H-thiazolo[3,2-c]pyrimidine-2-carboxylic acid benzylamide

### (2,6-dioxo-1,2,3,6-tetrahydropyrimidine-4-yl-sulfanyl)acetaldehyde dimethylacetal

Step A: A suspension of 6-chloro-1H-pyrimidine-2,4-dione (10.0 g, 68.3 mmol) was suspended in dimethylformamide (80 mL) at 40° C. The heat source was removed, and ground sodium hydrogen sulfide (17.3 g, 308 mmol) was added in portions. The temperature was maintained at 40° C. for 30 minutes, then bromoacetaldehyde dimethylacetal (36 mL, 308 mmol) was added. The suspension was stirred and heated at 40° C. for 18 hours. At the end of the reaction time, the dimethylformamide was removed by vacuum distillation. The residue was triturated with ethyl acetate (100 mL) for 1 hour. The resulting solid was isolated by filtration. The solid was triturated with water (100 mL), filtered, and rinsed with water. The solid was dried in a vacuum at 50° C. for 18 hours to give 5.90 g (37%) of (2,6-dioxo-1,2,3,6-tetrahydropyrimidine4-yl-sulfanyl)-acetaldehyde dimethyl acetal. ¹H-NMR (DMSO-d₆) δ 11.22 (s, 1H), 10.92 (s, 1H), 5.43 (s, 1H), 4.53 (t, 1H), 3.31 (d, 3H), 3,27 (d,3H), 3.18 (d, 2H); MS (APCI−) m/z 231, 199, 143.

### Thiazolo[3,2-c]pyrimidine-5,7-dione

Step B: To a suspension of (2,6-dioxo-1,2,3,6-tetrahydropyrimidine4-yl-sulfanyl)acetaldehyde dimethyl acetal (5.90 g, 25.4 mmol) in acetonitrile (400 mL) was added trimethylsilyl iodide (7.2 mL, 50.6 mmol). The mixture was refluxed for 4 hours. The mixture was cooled (ice bath) and isolated by filtration. The solid was rinsed twice with cold acetonitrile, then vacuum dried at 40° C. to give 4.08 g (96%) of thiazolo[3,2-c]pyrimidine-5,7-dione.

¹H-NMR (DMSO-d₆) δ 11.48 (s, 1H), 7.55 (d, 1H), 6.94 (d, 1H), 5.97 (s, 1H); MS (APCI+) m/z 169.

### 6-Benzoyl-thiazolo[3,2-c]pyrimidine-5,7-dione

Step C: To a suspension of thiazolo[3,2-c]pyrimidine-5,7-dione (0.506 g, 3.01 mmol) in tetrahydrofuran (20 mL) was added diisopropylethylamine (0.78 mL, 4.5 mmol) followed by benzoyl chloride (0.52 mL, 4.5 mmol). The mixture was stirred at room temperature for 22 hours. The reaction was filtered, and the isolated solid rinsed with ethyl acetate. The combined filtrate was washed with aqueous sodium bicarbonate, dried (Na₂SO₄), and evaporated to an oil. The oil was triturated (hexane:ethyl acetate, 1:1). The resulting solid was purified by flash chromatography (silica gel, dichloromethane:ethyl acetate, 17:3) to give 336 mg (41%) of product. TLC R_{f}=0.42 (CH₂Cl₂:EtOAc, 9:2); ¹H-NMR (DMSO-d₆) δ 8.05 (d, 2H), 7.76 (q, 1H), 7.65 (d, 1H), 7.55-7.64 (m, 2H), 7.08 (t, 1H), 6.24 (s, 1H); MS (APCI+) m/z 273, 189, 169.

Calcd for C₁₃H₈N₂O₃S:
C, 57.35; H, 2.96; N, 10.29.

Found: C, 57.39; H, 2.62; N, 10.09.

### 6-Benzoyl-5,7-dioxo-6,7-dihydro-5H-thiazolo[3,2-c]pyrimidine-2-carboxylic acid benzylamide

Step D: Lithium hexamethyldisilazane (1.7 mL, 1 M in THF, 1.7 mmol) was added to a solution of 6-benzoyl-thiazolo[3,2-c]pyrimidine-5,7-dione (0.319 g, 1. 14 mmol) in tetrahydrofuran (25 mL), under nitrogen at −72° C. After 3 minutes, benzyl isocyanate (0.49 mL, 4.0 mmol) was added. The reaction was stirred 15 minutes, then aqueous ammonium chloride was added and the reaction allowed to warm to room temperature. The reaction was partitioned between EtOAc and water. The organic layer was washed with brine, dried (Mg SO₄), and evaporated to a glass. The residue was triturated with hexane:EtOAc, 1:1, and the resulting solid was chromatographed on silica gel eluting with hexanes:THF, 1:1. The isolated product was triturated with diethyl ether to give 81 mg (18%) of 6-benzoyl-5,7-dioxo-6,7-dihydro-5H-thiazolo[3,2-c]pyrimidine-2-carboxylic acid benzylamide: mp 204-207° C. (dec.); TLC R_{f}=0.34 (CH₂Cl₂:EtOAc 9:2);

¹H-NMR ([DMSO-d₆) δ 9.37 (t, 1H), 8.53 (s, 1H), 8.08 (d, 2H), 7.58 (t, 2H), 7.35-7.23 (m, 5H), 6.28 (s, 1H), 4.42 (d, 2H); MS (AP-) m/z 404, 323, 300, 271, 257, 231.

Calcd for C₂₀H₁₅N₃O₄S:
C, 62.21; H, 3.73; N, 10.36.

Found: C, 62.09; H, 3.82; N, 9.93.

### EXAMPLE 12

### 6-(3,4-Dichlorobenzyl)-5,7-dioxo-6,7-dihydro-5H-thiazolo[3,2-c]pyrimidine-2-carboxylic acid benzylamide

### 6-(3,4-Dichlorobenzyl)-thiazolo[3,2-c]pyrimidine-5,7-dione

Step A: To a solution of thiazolo[3,2-c]pyrimidine-5,7-dione (1.0 g, 5.95 mmol) in dimethylformamide (20 mL) was added cesium carbonate (2.9 g, 9.1 mmol). The mixture was stirred at room temperature for 15 minutes. To the mixture was added 3,4-dichlorobenzyl chloride (1.2 mL, 8.9 mL) and the reaction stirred at room temperature for 20 hours. The dimethylformamide was removed by vacuum distillation at 60° C. The residue was triturated with EtOAc. The filtrate was evaporated, and the resulting solid was purified by flash chromatography on silica gel eluting with CH₂Cl₂:EtOAc, 19:1 to give 1.252 g (64%) of 6-(3,4-Dichlorobenzyl)-thiazolo[3,2-c]pyrimidine-5,7-dione. TLC R_{f}=0.30 (CH₂Cl₂:EtOAc 19:1); ¹H-NMR (DMSO-d₆) δ 7.61 (d, 1H), 7.56 (s, 1H), 7.54 (d, 1H), 7.28 (d, 1H), 7.00 (d, 1H), 6.19 (s, 1H), 4.99 (s, 2H); MS (APCI+) m/z 331, 329, 327.

### 6-(3,4-Dichlorobenzyl)-5,7-dioxo-6,7-dihydro-5H-thiazolo[3,2-c]pyrimidine-2-carboxylic acid benzylamide

Step B: Lithium hexamethyldisilazane (0.7 mL, 1 M in THF, 0.7 mmol) was added to a solution of 6-(3,4-dichlorobenzyl)-thiazolo[3,2-c]pyrimidine-5,7-dione (0.233 g, 0.68 mmol) in tetrahydrofuran (10 mL), under nitrogen at −72° C. After 3 minutes benzyl isocyanate (0.25 mL, 2.0 mmol) was added. The reaction was stirred 15 minutes, then aqueous ammonium chloride was added and the reaction allowed to warm to room temperature. To the reaction was added EtOAc (50 mL). The water layer was removed, and the organic layer was dried (Na₂SO₄), and evaporated to an oil. The residue was triturated with hexane:EtOAc, 1:1. The resulting filtrate was evaporated to foam. This was chromatographed on silica gel eluting with hexane:EtOAc, 1:1. The isolated product was triturated with diethyl ether and dried in vacuum at 45° C. to give 18 mg (5.6%) of 6-(3,4-dichlorobenzyl)-5,7-dioxo-6,7-dihydro-5H-thiazolo[3,2-c]pyrimidine-2-carboxylic acid benzylamide: mp 216-217° C.; TLC R_{f}=0.23 (hexane:EtOAc, 1:1); ¹H-NMR (DMSO-d₆) δ 9.34 (t, 1H), 8.50 (s, 1H), 7.56 (s, 1H), 7.54 (d, 1H), 7.35-7.25 (m, 6H), 6.22 (s, 1H), 4.99 (s, 2H), 4.41 (d, 2H); MS (APCI+) m/z 463, 462, 460, 329, 327, 233.

Calcd for C₂₁H₁₅Cl₂N₃O₃S:
C, 54.79; H, 3,28; N, 9.13.

Found: C, 54.71; H, 3.06; N, 8.93.

### EXAMPLE 13

### 6-(4-Chlorobenzyl)-5,7-dioxo-6,7-dihydro-5H-thiazolo[3,2-c]pyrimidine-2-carboxylic acid benzylamide

### 6-(4-Chlorobenzyl)-thiazolo[3,2-c]pyrimidine-5,7-dione

Step A: To a solution of thiazolo[3,2-c]pyrimidine-5,7-dione (0.505 g, 3.00 mmol) in dimethylformamide (10 mL) was added cesium carbonate (1.47 g, 4.5 mmol). The mixture was stirred at room temperature for 20 minutes. To the mixture was added 4-chlorobenzyl chloride (0.725 g, 4.5 mL) in dimethylformamide (2 mL), and the reaction was stirred at room temperature for 23 hours. The dimethylformamide was removed by vacuum distillation at 60° C. The residue was triturated with EtOAc. The filtrate was evaporated, and the resulting solid was purified by flash chromatography on silica gel eluting with CH₂Cl₂:EtOAc, 9:1 to give 437 mg (50%) of 6-(4-chlorobenzyl)-thiazolo[3,2-c]pyrimidine-5,7-dione: mp 152-153.5; TLC R_{f}=0.51 (CH₂Cl₂:EtOAc 17:3); ¹H-NMR (DMSO-d₆) δ 7.60 (d, 1H), 7.36-7.7.30 (m, 4H), 7.00 (d, 1H), 6.19 (s, 1H), 4.99 (s, 2H); MS (APCI+) m/z 296, 295, 294, 293.

Calcd for C₁₃H₉ClN₂O₂S:
C, 53.34; H, 3.10; N, 9.57.

Found: C, 53,22; H, 3.31; N, 9.31.

### 6-(4-Chlorobenzyl)-5,7-dioxo-6,7-dihydro-5H-thiazolo[3,2-c]pyrimidine-2-carboxylic acid benzylamide

Step B: Lithium hexamethyldisilazane (0.96 mL, 1 M in THF, 0.96 mmol) was added to a solution of 6-(3,4-dichlorobenzyl)-thiazolo[3,2-c]pyrimidine-5,7-dione (0.188 g, 0.64 mmol) in tetrahydrofuran (20 mL) under nitrogen at −72° C. After 3 minutes benzyl isocyanate (0.28 mL, 2.2 mmol) was added. The reaction was stirred 20 minutes, then aqueous ammonium chloride was added, and the reaction allowed to warm to room temperature. To the reaction was added EtOAc (50 mL). The water layer was removed, and the organic layer was dried (Na₂SO₄) and evaporated to foam. This was chromatographed on silica gel eluting with hexane:EtOAc, 1:1. The isolated product was triturated twice with diethyl ether and dried in vacuum to give 108 mg (39%) of 6-(4-chlorobenzyl)-5,7-dioxo-6,7-dihydro-5H-thiazolo[3,2-c]pyrimidine-2-carboxylic acid benzylamide: mp>220° C.; TLC R_{f}=0.18 (hexane:EtOAc, 1:1); ¹H-NMR (DMSO-d₆) δ 9.34 (t, 1H), 8.50 (s, 1H), 7.35-7.23 (m, 9H), 6.22 (s, 1H), 4.99 (s, 2H), 4.40 (d, 2H); MS (APCI+) m/z 428, 426, 295, 293, 233.

Calcd for C₂₁H₁₆ClN₃O₃S:
C, 59.22; H, 3.79; N, 9.87.

Found: C, 59.18; H, 3.37; N, 9.34.

### EXAMPLE 14

### 6-(4-Chlorobenzyl)-5,7-dioxo-6,7-dihydro-5H-thiazolo[3,2-c]pyrimidine-2-carboxylic acid 3,4-dichlorobenzylamide

Lithium hexamethyldisilazane (0.96 mL, 1 M in THF, 0.96 mmol) was added to a solution of 6-(4-chlorobenzyl)-thiazolo[3,2-c]pyrimidine-5,7-dione (0.188 g, 0.64 mmol) in tetrahydrofuran (20 mL), under nitrogen at −72° C. After 3 minutes 3,4-dichlorobenzyl isocyanate (0.33 mL, 2.2 mmol) was added. The reaction was stirred 15 minutes, then aqueous ammonium chloride was added and the reaction allowed to warm to room temperature. To the reaction was added EtOAc (50 mL). The water layer was removed and the organic layer,was dried (Na₂SO₄) and evaporated to an oil. The residue was triturated with hexane:EtOAc, 1:1. The resulting filtrate was evaporated to foam. This was chromatographed on silica gel eluting with hexane:EtOAc, 1:1. The isolated product was triturated with diethyl ether and dried in vacuum at 45° C. to give 77 mg (24%) of 6-(4-chlorobenzyl)-5,7-dioxo-6,7-dihydro-5H-thiazolo[3,2-c]pyrimidine-2-carboxylic acid 3,4-dichloro-benzylamide:

mp 218-219° C.; TLC R_{f}=0.26 (CH₂Cl₂:EtOAc 17:3); ¹H-NMR (DMSO-d₆) δ 9.37 (t, 1H), 8.48 (s, 1H), 7.60 (d, 1H), 7.55 (s,1H), 7.36-7.27 (m, 5H), 6.23 (s, 1H), 4.99 (s, 2H), 4.40 (d, 2H); MS (APCI+) m/z 497, 495, 493, 303, 301, 295, 293.

Calcd for C₂₁H₁₄C₁₃N₃O₃S:
C, 50.98; H, 2.85; N, 8.49.

Found: C, 51.29; H, 2.86; N, 8.35.

### EXAMPLE 15

### 6-(4-Pyridylmethyl)-5,7-dioxo-6,7-dihydro-5H-thiazolo[3,2-c]pyrimidine-2-carboxylic acid benzylamide hydrochloride

### 6-(4-Pyridylmethyl)-thiazolo[3,2-c]pyrimidine-5,7,dione

Step A: To a solution of thiazolo[3,2-c]pyrimidine-5,7-dione (0.505 g, 3.00 mmol) in dimethylformamide (20 mL) was added sodium hydride (0.39 g, 9.7 mmol, 60% oil dispersion) in small portions over 20 minutes. Over a 1-hour period, 4-bromomethylpyridine hydrobromide (0.92 g, 3.6 mmol) was added. The reaction was stirred at room temperature for 90 minutes. The dimethylformamide was removed by vacuum distillation at 60° C. The residue was triturated with tetrahydrofuran (50 mL) for 16 hours. The mixture was filtered. The filtrate was evaporated, and the resulting solid purified by chromatography on silica gel eluting with CH₂Cl₂:THF, 2:1. There was recovered 277 mg (33%) of desired product: mp 174-176° C.; TLC R_{f}=0.24 (CH₂Cl₂:THF 2:1); ¹H-NMR (DMSO-d₆) δ 8.45 (d, 2H), 7.61 (d, 1H), 7.24 (d, 2H), 7.01 (d, 1H), 6.21 (s, 1H), 2H); MS (AP+) m/z 260.

Calcd for C₁₂H₉N₃O₂S.0.2 H₂O:
C, 54.83; H, 3.60; N, 15.98.

Found: C, 55.10; H, 3.60; N, 15.77.

### 6-(4-Pyridylmethyl)-5,7-dioxo-6,7-dihydro-5H-thiazolo[3,2-c]pyrimidine-2-carboxylic acid benzylamide

Step B: Lithium hexamethyldisilazane (0.59 mL, 1 M in THF, 0.59 mmol) was added to a solution of 6-(4-pyridylmethyl)-thiazolo[3,2-c]pyrimidine-5,7-dione (0.100 g, 0.39 mmol) in tetrahydrofuran (15 mL), under nitrogen at −72° C. After 3 minutes benzyl isocyanate (0.12 mL, 0.98 mmol) was added. The reaction was stirred 15 minutes, then aqueous ammonium chloride was added and the reaction allowed to warm to room temperature. To the reaction was added EtOAc (50 mL). The water layer was removed and the organic layer was dried (Na₂SO₄) and evaporated to an oil. This was chromatographed on silica gel eluting with CH₂Cl₂:THF, 2:1 to give 75 mg (49%) of product. This material was combined with other lots and chromatographed in the same manner prior to conversion to the hydrochloride salt. 1H-NMR (DMSO-d₆) δ 9.34 (t, 1H), 8.51 (s, 1H), 8.46 (d, 2H), 7.35-7.23 (m, 7H), 6.25 (s, 1H), 5.03 (s, 2H), 4.41 (d, 2H).

### 6-(4-Pyridylmethy)-5,7-dioxo-6,7-dihydro-5H-thiazolo[3,2-c]pyrimidine-2-carboxylic acid benzylamide hydrochloride

Step C: The product from Step B (0.115 g, 0.29 mmol) in tetrahydrofuran (30 mL), under nitrogen, was mixed with anhydrous hydrogen chloride in diethyl ether (0.5 mL, 1 M). The suspension was stirred at room temperature for 16 hours. The resulting solid was isolated by filtration and triturated with water (0.5 mL) for 30 minutes. The solid was isolated and dried in vacuum at room temperature for 22 hours to give 95.2 mg (77%) of the hydrochloride monohydrate: mp>210° C.,

¹H-NMR (DMSO-d₆) δ 9.41 (t, 1H), 8.72 (d, 2H), 8.55 (s, 1H), 7.76 (d, 2H), 7.35-7.25 (m, 5H), 5.20 (s, 1H), 4.41 (d, 1H); MS (APCI+) m/z 394, 393, 260.

Calcd for C₂₀H₁₆N₄O₃S.HCl.H₂O.
C, 53.75; H, 4.29; N, 12.54.

Found: C, 54.06; H, 4.24; N, 12.51.

### EXAMPLE 16

### 6-Benzyl-8-methyl-5,7-dioxo-6,7-dihydro-5H-thiazolo[3,2-c]pyrimidine-2-carboxylic acid benzylamide

Lithium hexamethyldisilazane (0.67 mL, 1 M in THF, 0.67 mmol) was added to a solution of 6-benzyl-8-methyl-thiazolo[3,2-c]pyrimidine-5,7-dione (0.122 g, 0.45 mmol) in tetrahydrofuran (10 mL), under nitrogen at −70° C. After 3 minutes benzyl isocyanate (0.20 mL, 0.67 mmol) was added. The reaction was stirred 20 minutes, then aqueous ammonium chloride was added and the reaction allowed to warm to room temperature. Water was added and the mixture stirred overnight. To the reaction was added EtOAc (50 mL). The layers were separated and the organic layer washed with brine, dried (Na₂SO₄), and evaporated to an oil. This material was chromatographed on silica gel eluting with CH₂Cl₂:EtOAc, 9:1. The isolated product was triturated with diethyl ether and dried in vacuum at room temperature for 16 hours to give 62 mg (34%) of 6-benzyl-8-methyl-5,7-dioxo-6,7-dihydro-5H-thiazolo[3,2-c]pyrimidine-2-carboxylic acid benzylamide. mp 176-178° C.; TLC R_{f}=0.33 (CH₂Cl₂:EtOAc 9:1); ¹H-NMR (CDCl₃) δ 7.90 (s, 1H), 7.46 (d, 2H), 7.39-7.27 (m, 8H), 6.10 (t, 1H), 5.16 (s, 4,57 (d, 2H), 1.99 (s, 3H); MS (APCI+) m/z 406, 273.

Calcd for C₂₂H₁₉N₃O₃S:
C, 65.17; H, 4.72; N, 10.36.

Found: C, 65.16; H, 4.76; N, 10.15.

### EXAMPLE 17

### 6-Benzyl-8-methyl-5,7-dioxo-6,7-dihydro-5H-thiazolo[3,2-c]pyrimidine-2-carboxylic acid 4-methoxybenzylamide

Lithium hexamethyldisilazane (0.83 mL, 1 M in THF, 0.83 mmol) was added to a solution of 6-benzyl-8-methyl-thiazolo[3,2-c]pyrimidine-5,7-dione (0.150 g, 0.55 mmol) in tetrahydrofuran (15 mL), under nitrogen at −73° C. After 3 minutes, 4-methoxybenzyl isocyanate (0.27 mL, 1.9 mmol) was added. The reaction was stirred 20 minutes, then aqueous ammonium chloride was added and the reaction allowed to warm to room temperature. To the reaction was added EtOAc (50 mL). The water layer was removed and the organic layer was, dried (Na₂SO₄) and evaporated to an oil. This material was chromatographed on silica gel eluting with hexane:EtOAc, 2:1. The isolated product was triturated with diethyl ether and dried in vacuum at room temperature for 16 hours to give 108 mg (45%) of 6-benzyl-8-methyl-5,7-dioxo-6,7-dihydro-5H-thiazolo[3,2-c]pyrimidine-2-carboxylic acid 4-methoxybenzylamide: mp 160-162° C.; TLC R_{f}=0.15 (hexane:EtOAc, 2:1); ¹H-NMR (DMSO-d₆) δ 9.23 (t,1H), 8.52 (s, 1H), 7.29-7.20 (m, 7H), 6.88 (d, 2H), 5.03 (s, 2H), 4.33 (d, 2H), 3.71 (s, 3H), 1.87 (s, 3H); MS (APCI+) m/z 436, 273, 121.

Calcd for C₂₃H₂₁N₃O₄S:
C, 63.43; H, 4.86; N, 9.65.

Found: C, 63.35; H, 4.87; N, 9.51.

### EXAMPLE 18

### 6-Benzyl-8-methyl-5,7-dioxo-6,7-dihydro-5H-thiazolo[3,2-c]pyrimidine-2-carboxylic acid 3,4-dichlorobenzylamide

Borane-tetrahydrofuran complex (0.65 mL, 1 M in THF, 0.65 mmol) was added to a solution of 6-benzyl-8-methyl-thiazolo[3,2-c]pyrimidine-5,7-dione (0.178 g, 0.65 mmol) in tetrahydrofuran (15 mL), under nitrogen at −73° C. To this solution was added lithium hexamethyldisilazane (0.72 mL, 1 M in THF, 0.72 mmol) was added. After 3 minutes 3,4-dichlorobenzyl isocyanate (0.30 mL, 0.2.04 mmol) was added. The reaction was stirred 40 minutes, then 5% AcOH in EtOH was added and the reaction allowed to warm to room temperature. The solvent was evaporated in vacuum and the residue partitioned between EtOAc (50 mL) and water. The water layer was removed and the organic layer was, dried (Na₂SO₄) and evaporated to a solid. The residue was triturated with diethyl ether overnight. The resulting solid was chromatographed on silica gel eluting with hexane:EtOAc, 1:1. The isolated product was triturated with diethyl ether and dried in vacuum at room temperature for 3.5 hours to give 94 mg (30%) of 6-benzyl-8-methyl-5,7-dioxo-6,7-dihydro-5H-thiazolo[3,2-c]pyrimidine-2-carboxylic acid 3,4-dichlorobenzylamide: mp 180-182° C.; TLC R_{f}=0.33 (hexane:EtOAc 1:1); ¹H-NMR (DMSO-d₆) δ 9.35 (t, 1H), 8.52 (d, 1H), 7.59 (s, 1H), 7.55 (m, 6H), 7.28 (s, 1H), 5.04 (s, 2H), 4.41 (d, 2H), 1.87 (s, 3H);

MS (APCI+) m/z 474, 273. Calcd for C₂₂H₁₇Cl₂N₃03S:
C, 55,70; H, 3.61; N, 8.86.

Found: C, 55.61; H, 3.79; N, 8.56.

### EXAMPLE 19

### 6-Benzyl-3-methyl-5,7-dioxo-6,7-dihydro-5H-thiazolo[3,2-c]pyrimidine-2-carboxylic acid benzyl ester

### 3-Benzyl-6-(2-oxopropylsulfanyl)-1H-pyrimidine-2,4-dione

Step A: Ground sodium hydrosulfide hydrate (2.36 g, 42 mmol) was added to 3-benzyl-6-chloro-1H-pyrimidine-2,4-dione (2.36g, 10 mmol) in dimethylformamide (12 mL), and the mixture was warmed to 45° C. for about 10 minutes and then chloroacetone (4.0 mL, 50 mmol) was added. The reaction mixture was stirred overnight at room temperature and was then partitioned between ethyl acetate (200 mL) and water (200 mL). The layers were separated, and the organic layer washed with water (2×100 mL) and dried over magnesium sulfate. The solution was filtered and concentrated to a yellow oil. The oil was chromatographed on silica gel (70-230 mesh) using hexanes/ethyl acetate, 1:1, v/v, as eluant. The product was obtained in several portions as a mixture of the ring-opened and aminal forms. The mixture was used directly in the next step.

### 6-Benzyl-3-methyl-thiazolo[3,2-c]pyrimidine-5,7-dione

Step B: A mixture of the ring-opened and aminal forms of 3-benzyl-6-(2-oxopropylsulfanyl)-1H-pyrimidine-2,4-dione (0.746 g, 2.6 mmol), xylenes (35 mL), and a catalytic amount of p-toluenesulfonic acid was refluxed with removal of water using a Dean-Stark trap. The reaction mixture was refluxed overnight, concentrated to dryness, and partitioned between ethyl acetate (150 mL) and sodium bicarbonate solution. The layers were separated, the organic layer dried over magnesium sulfate, filtered and concentrated to a light brown solid. The solid was triturated with hexanes/ethyl acetate to give the product as a tan solid, 0.220 g. An additional 0.258 g was obtained by silica gel filtration of the mother liquors.

Calcd for C₁₄H₁₂N₂O₂S:
C, 61.75; H, 4.44; N, 10.29.

Found: C, 61.59; H, 4.43; N, 10.11.

### 6-Benzyl-3-methyl-5,7-dioxo-6,7-dihydro-5H-thiazolo[3,2-c]pyrimidine-2-carboxylic acid benzyl ester

Step C: The product from Step B, 6-benzyl-3-methyl-thiazolo[3,2-c]pyrimidine-5,7-dione, (0.220 g, 0.81 mmol) was reacted according to the procedure from Example 2, Step E, to give the product, 0.209g (63.7%) in 2 portions.

Calcd for C₂₂H₁₈N₂O₄S:
C, 65.01; H, 4.46; N, 6.89.

Found: C, 65.01; H, 4.47; N, 6.78.

### EXAMPLE 20

### 6-Benzyl-5,7-dioxo-2,3,6,7-tetrahydro-5H-thiazolo[3,2-c]pyrimidine-3-carboxylic acid benzyl ester

### 2,3-Dihydroxypropionic acid benzyl ester

Step A: To a solution of benzylacrylate (10 g, 61.7 mmol) in acetone (20 mL) and water (7 mL) was added morpholine N-oxide (8.6 g, 73.4 mmol). Osmium tetroxide (3 mL of a 2.5% solution in tertiary butanol) was added and the exothermic reaction moderated by cooling with an ice bath. The reaction was complete in 1 hour. A second portion of benzylacrylate (10 g, 61.7 mmol) and morpholine N-oxide (8.6 g, 73.4 mmol) was added and the reaction mixture stirred at room temperature. When the reaction was complete by TLC, sodium sulfite (10 g, 120 mmol) was added, and the mixture was stirred 0.5 hour. The reaction mixture was extracted with ethyl acetate and washed with 1N hydrochloric acid solution, brine, and dried over magnesium sulfate. The solution was filtered, concentrated, and distilled (150° C. at 0.1 mm Hg) to give 18.09 g of the product as a colorless oil. This was used directly in the next step.

### 2,2-Dioxo-2l⁶-[1,2]oxathiolane-4-carboxylic acid benzyl ester

Step B: The product from Step A, namely 2,3-dihydroxypropionic acid benzyl ester (1.00 g, 5,7 mmol), in carbon tetrachloride (20 mL) was treated with thionyl chloride (0.39 mL, 5.36 mmol). Nitrogen gas was bubbled through the solution while refluxing. The reaction was complete in about 0.5 hour. Acetonitrile (10 mL), ruthenium chloride trihydrate (10 mg), sodium metaperiodate (1.64 g), and water (10 mL) were added and the mixture stirred 0.5 hour. The reaction mixture was diluted with water and ether, and the ether layer dried over magnesium sulfate. The product was obtained as a solid upon filtration through a pad of silica gel; 1.163 g.

Calcd for C₁₀H₁₀O₆S:
C, 47.04; H, 3.96.

Found: C,46.51;H,3.90.

### 6-Benzyl-5,7-dioxo-2,3,6,7-tetrahydro-5H-thiazolo[3,2-c]pyrimidine-3-carboxylic acid benzyl ester

Step C: 3-Benzyl-6-chloro-1H-pyrimidine-2,4-dione (1.083 g, 4.59 mmol) in tetrahydrofuran (10 mL) was treated with potassium t-butoxide (1 M in tetrahydrofuran, 5.6 mL, 5.6 mmol) for 5 minutes. Then 2,2-dioxo-2l⁶-[1,2]oxathiolane-4-carboxylic acid benzyl ester (1.895 g, 7.34 mmol) was added in 1 portion. The reaction mixture was stirred for 2 hours at room temperature and then concentrated in vacuo. Drying under high vacuum afforded a foam. The foam was dissolved in dimethylformamide and sodium hydrosulfide (1.29 g, 23 mmol) was added causing an exotherm. The reaction mixture was stirred 2 hours and then treated with 1 N HCl (20 mL) and extracted into ethyl acetate, washed with brine, dried over sodium sulfate, filtered and evaporated. The residue was chromatographed on silica gel using 40% ethyl acetate in hexanes then 70% ethyl acetate in hexanes to give the product; 0.656 g.

Calcd for C₂₁H₁₈N₂O₄S:
C, 63.95; H, 4.60; N, 7.10.

Found: C, 63.70; H, 4.70; N, 6.82.

### EXAMPLE 21

### 6-Benzyl-5,7-dioxo-2,3,6,7-tetrahydro-5H-thiazolo[3,2-c]pyrimidine-2-carboxylic acid pyridin-4-ylmethyl ester hydrochloride

### 6-Benzyl-5,7-dioxo-2,3,6,7-tetrahydro-5H-thiazolo[3,2-c]pyrimidine-2-carboxylic acid

Step A: To a solution of 6-benzyl-5,7-dioxo-2,3,6,7-tetrahydro-5H-thiazolo[3,2-c]pyrimidine-2-carboxylic acid benzyl ester (1.68 g, 4.25 mmol) in a mixture of tetrahydrofuran (48 mL), methanol (14 mL), and water (14 mL) was added at room temperature, lithium hydroxide hydrate (0.37 g, 8.8 mmol). The reaction mixture was stirred 2 hours at room temperature and was partitioned between 1 N hydrochloric acid solution (100 mL) and ethyl acetate (200 mL). The layers were separated, the organic layer washed with sodium chloride solution, dried over magnesium sulfate, filtered, and concentrated to an oil that crystallized from dichloromethane/ethyl ether. The entire mixture was concentrated to dryness and triturated with ethyl ether and the solid collected by filtration; 0.833 g. An additional 0.632 g was obtained from the mother liquors.

Calcd for C₁₄H₁₂N₂O₄S:
C, 55.26; H, 3.97; N, 9.21.

Found: C, 55.16; H, 4.09; N, 8.75.

### 6-Benzyl-5,7-dioxo-2,3,6,7-tetrahydro-5H-thiazolo[3,2-c]pyrimidine-2-carboxylic acid pyridin-4-ylmethyl ester hydrochloride

Step B: To a solution of 6-benzyl-5,7-dioxo-2,3,6,7-tetrahydro-5H-thiazolo[3,2-c]pyrimidine-2-carboxylic acid(.305 g, 1 mmol), 4-hydroxymethylpyridine (0.160 g, 1.47 mmol), 4-dimethylamino pyridine (0.043 g, 0.35 mmol), and tetrahydrofuran (20 mL) at 0° C. was added dicyclohexylcarbodiimide (0.224 g, 1.08 mmol), and the mixture was stirred 5 days at room temperature. The reaction mixture was concentrated to dryness, partitioned between ethyl acetate (200 mL) and water (100 mL), the layers separated, dried over magnesium sulfate, filtered, and concentrated. Some material was insoluble in both layers and was collected by filtration. This solid was treated with HCl gas in diethyl ether (1 M) and the insoluble material collected by filtration; 0.129 g.

Calcd for C₂₀H₁₇N₃O₄S.HCl.0.5H₂O:
C, 54.48; H, 4.34; N, 9.53.

Found: C, 54.26; H, 4.31; N, 9.35.

### EXAMPLE 22

### 6-Benzyl-5,7-dioxo-2,3,6,7-tetrahydro-5H-thiazolo[3,2-c]pyrimidine-2-carboxylic acid(pyridin-4-ylmethyl)-amide

To a solution of 6-benzyl-5,7-dioxo-2,3,6,7-tetrahydro-5H-thiazolo[3,2-c]pyrimidine-2-carboxylic acid(.304 g, 1 mmol), 4-aminomethylpyridine (0.115 g, 1.06 mmol), 1-hydroxybenzotriazole hydrate (0.140 g, 1.04 mmol), tetrahydrofuran (20 mL) and dimethylformamide (8 mL) at room temperature was added 1-[3-dimethylamino)propyl)-3-ethylcarbodiimide hydrochloride (0.201 g, 1.05 mmol) and the mixture was stirred 5 days at room temperature. The reaction mixture was concentrated to dryness, partitioned between ethyl acetate (200 mL) and water (100 mL), the layers separated, dried over magnesium sulfate, filtered, and concentrated to a yellow oil. The oil was triturated with hexanes/ethyl acetate and the resulting solid collected by filtration; 0.198 g.

Calcd for C₂₀H₁₈N₄O₃S.0.5H₂O:
C, 59.54; H, 4.75; N, 13.89.

Found: C, 59.85; H, 4.71; N, 13.97.

### EXAMPLE 23

### 6-Benzyl-1,5,7-trioxo-1,2,3,5,6,7-hexahydro-114-thiazolo[3,2-c]pyrimidine-3-carboxylic acid benzyl ester

A mixture of 6-benzyl-5,7-dioxo-6,7-dihydro-5H-thiazolo[3,2-c]pyrimidine-2-carboxylic acid benzyl ester (2.55 g, 6.46 mmol), 4 Å molecular sieves (3 g) in dichloromethane 150 mL was stirred at 0° C. m-Chloroperbenzoic acid(2.53 g, 15.2 mmol) was added in 3 portions at 0, 2, and 14 hours. After 20 hours the reaction was filtered, concentrated, and taken up in ethyl acetate. The organic layer was washed with sodium bicarbonate solution (2×200 mL), brine, and dried over magnesium sulfate. The mixture was filtered, concentrated, and chromatographed on silica gel eluting with 5% acetone in dichloromethane to afford the title compound (1.69 g, 64%).

Calcd for C₂₁H₁₈N₂O₅S:
C, 61.45; H, 4.42; N, 6.83.

Found: C, 61.31; H, 4.44; N, 6.69.

### EXAMPLE 24

### 6-Benzyl-8-methyl-5,7-dioxo-6,7-dihydro-5H-thiazolo[3,2-c]pyrimidine-2-carbothioic acid benzylamide

Lithium hexamethyldisilazane (0.8 mL, 1 M in THF, 0.8 mmol) was added to a solution of 6-benzyl-8-methyl-thiazolo[3,2-c]pyrimidine-5,7-dione (0.136 g, 0.50 mmol) in tetrahydrofuran (20 mL), under nitrogen at −68° C. After 3 minutes benzyl isothiocyanate (0.20 mL, 1.5 mmol) was added. The reaction was stirred 14 minutes, then aqueous ammonium chloride was added and the reaction allowed to warm to room temperature. To the reaction was added EtOAc (200 mL). The layers were separated and the organic layer washed with brine, dried over magnesium sulfate, and evaporated to an orange oil. This material was chromatographed 2 times on silica gel eluting with hexanes:EtOAc, 7:3, to give the product as a yellow solid;. 0.045 g.

Calcd for C₂₂H₁₉N₃O₂S₂:
C, 62.69; H, 4.54; N, 9.97.

Found: C, 62.38; H, 4.48; N, 9.51.

### EXAMPLE 25

### 6-Benzyl-3-ethoxy-2,3-dihydro-oxazolo[3,2-c]pyrimidine-5,7-dione 3-Benzyl-6-(2,2-diethoxy-ethoxy)-1H-pyrimidine-2,4-dione

Step A: A solution of the sodium alkoxide of 2-hydroxyacetaldehyde diethyl acetal was prepared from sodium hydride (1.74 g (60% in mineral oil, 43.5 mmol)) and 2-hydroxyacetaldehyde diethyl acetal (5.4 g, 40.3 mmol) in dimethylformamide (40 mL) at room temperature. The alkoxide solution was warmed to 50° C. and then 3-benzyl-6-chloro-1H-pyrimidine-2,4-dione (4.70 g, 20 mmol) was added, and the mixture was heated to 80° C. overnight and then was heated to 110° C. overnight. A second portion of alkoxide (from 2.70 g alcohol and 0.97 g sodium hydride (60% in mineral oil)) in dimethylformamide (10 mL) was added, and the reaction mixture was stirred overnight at 110° C. The reaction mixture was cooled, concentrated, and partitioned between ethyl acetate (400 mL) and sodium bicarbonate solution (400 mL). The layers were separated, the organic layer washed with water (2×100 mL), brine (100 mL), and dried over magnesium sulfate, filtered, and concentrated to an oil. The oil was filtered through silica gel (70-230 mesh) using tetrahydrofuran as eluant and was then filtered through silica gel (70-230 mesh) again using ethyl acetate as eluant. Several portions of product were obtained. ¹H-NMR (CDCl₃) δ 8.93 (bs, 1H), 7.22-7.44 (m, 5H), 5.10 (s, 1H), 5.05 (s, 2H), 4.76 (t, 1H), 3.98 (d, 2H), 3.69-3.78 (m, 2H), 3.55-3.63 (m, 2H), 1.22 (m, 6H).

This material was used directly in the next step.

### 6-Benzyl-3-ethoxy-2,3-dihydro-oxazolo[3,2-c]pyrimidine-5,7-dione

Step B: A mixture of 3-benzyl-6-(2,2-diethoxy-ethoxy)-1H-pyrimidine-2,4-dione (3.14 g, 9.4 mmol), xylenes (70 mL), and a catalytic amount of p-toluenesulfonic acid hydrate was heated to reflux employing a Dean-Stark trap. After 4 hours, no starting material remained. The reaction mixture was concentrated to dryness and the oil/gum taken up in ethyl acetate (200 mL) and was washed with sodium bicarbonate solution, dried over magnesium sulfate, filtered and concentrated to a brown oil/gum. Upon addition of ethyl acetate a small amount of precipitated formed and was collected; 0.036 g. ¹H-NMR (DMSO-d₆) δ 8.93 (bs, 1H), 7.1-7.3 (m, 5H), 5.96 (d, 1H), 4.83 (dd, 2H), 4.6-4.8 (m, 2H), 3.67-3.85 (m, 2H), 1.09 (t, 3H).

### EXAMPLE 26

### 6-Benzyl-3-methyl-5,7-dioxo-6,7-dihydro-5H-thiazolo[3,2-c]pyrimidine-2-carboxylic acid methyl ester

Step A: Ground sodium hydrosulfide hydrate (4.35g, 78 mmol) was added to 3-benzyl-6-chloro-1H-pyrimidine-2,4-dione (4.72 g, 20 mmol) in dimethylformamide (15 mL), and the mixture was warmed to 44° C. and then neat methyl-2-chloroacetoacetate (10 mL, 82 mmol) was added in portions over about 10 minutes. The reaction mixture was stirred 0.5 hour at 50° C. and was then partitioned between ethyl acetate (450 mL) and sodium bicarbonate solution (100 mL) and water (200 mL). The layers were separated and the organic layer washed with water (2×200 mL) and brine (100 mL) and dried over magnesium sulfate. The solution was filtered and concentrated and triturated with hexanes/ethyl acetate, 1:1, v/v, and the solid collected by filtration, 2.17 g. A second crop was obtained from the mother liquors, 0.68 g, (41%). MS (APCI+) m/z (%): 349.1(100), 317.1(50).

Step B: The product from Example 26, Step A, (0.837 g, 2.4 mmol) was heated to reflux in toluene (50 mL) in the presence of a catalytic amount of para-toluenesulfonic acid hydrate employing a Dean-Stark trap for the azeotropic removal of methanol. The reaction mixture was refluxed 9 hours then concentrated to an oil. The oil was filtered through silica gel (70-230 mesh) using hexanes/ethyl acetate, 12:1, v/v as eluant. The product, 6-benzyl-3-methyl-5,7-dioxo-6,7-dihydro-5H-thiazolo[3,2-c]pyrimidine-2-carboxylic acid methyl ester, was obtained as a white solid, 0.142 g (18%). MS (APCI+), m/z (%): 331.1(100).

### EXAMPLE 27

### 6-Benzyl-8-methyl-5,7-dioxo-6,7-dihydro-5H-thiazolo[3,2-c]pyrimidine-2-carboxylic acid 2,4-dichloro-benzylamide

Employing the procedure of Example 2, Step E, 6-benzyl-8-methyl-thiazolo[3,2-c]pyrimidine-5,7-dione (0.272 g, 1.0 mmol) was taken up in tetrahydrofuran (10 mL) and lithium hexamethyldisilazane (1.5 mL, 1 M in tetrahydrofuran, 1.5 mmol) was added at −78° C., and the reaction was allowed to proceed for 3 minutes; then 2,4-dichlorobenzyl isocyanate (0.5 mL, 3.4 mmol) was added, and the reaction was stirred for 15 minutes at −78° C., ammonium chloride solution was added, and the reaction mixture was partitioned between ethyl acetate and brine. The layers were separated, the organic layer was dried over magnesium sulfate, filtered, and concentrated. The residue was chromatographed on silica gel using hexanes/ethyl acetate, 65:35, v/v as eluant to give the product, 6-benzyl-8-methyl-5,7-dioxo-6,7-dihydro-5H-thiazolo[3,2-c]pyrimidine-2-carboxylic acid 2,4-dichloro-benzylamide, as a white solid upon trituration with hexanes/ethyl acetate, 100 mg (21%). MS (APCI+), m/z (%): 476.1(60), 474.1 (80) 273.1 (100).

### EXAMPLE 28

### 6-Benzyl-8-methyl-5,7-dioxo-6,7-dihydro-5H-thiazolo[3,2-c]pyrimidine-2-carboxylic acid 3-methyl-benzylamide

Employing the procedure of Example 2, Step E, 6-benzyl-8-methyl-thiazolo[3,2-c]pyrimidine-5,7-dione (0.272 g, 1.0 mmol) was taken up in tetrahydrofuran (10 mL), and lithium hexamethyldisilazane (1.5 mL, 1 M in tetrahydrofuran, 1.5 mmol) was added at −78° C., and the reaction was allowed to proceed for 3 minutes; then 3-methylbenzyl isocyanate (0.35 mL, 2.7mmol) was added, and the reaction was stirred for 15 minutes at −78° C., ammonium chloride solution was added, and the reaction mixture was partitioned between ethyl acetate and brine. The layers were separated, the organic layer was dried over magnesium sulfate, filtered, and concentrated. The residue was chromatographed on silica gel using hexanes/ethyl acetate, 65:35, v/v as eluant to give the product, 6-benzyl-8-methyl-5,7-dioxo-6,7-dihydro-5H-thiazolo[3,2-c]pyrimidine-2-carboxylic acid 3-methyl-benzylamide, as a white solid upon trituration with hexanes/ethyl acetate, 120 mg (29%). MS (APCI+), m/z (%): 420.1(100), 273.1 (75).

### EXAMPLE 29

### 6-Benzyl-2-(1-hydroxy-3-phenyl-allyl)-8-methyl-thiazolo[3,2-c]pyrimidine-5,7-dione

Employing the procedure of Example 2, Step E, 6-benzyl-8-methyl-thiazolo[3,2-c]pyrimidine-5,7-dione (0.407 g, 1.5 mmol) was taken up in tetrahydrofuran (12 mL) and lithium hexamethyldisilazane (3,2 mL, 1 M in tetrahydrofuran, 3,2 mmol) was added at −72° C., and the reaction was allowed to proceed for 3 minutes, then trans-cinnamaldehyde (0.5 mL, 4.0 mmol) was added, and the reaction was stirred for 15 minutes at −72° C., ammonium chloride solution (3 mL) was added, and the reaction mixture was allowed to slowly warm to room temperature and was then partitioned between ethyl acetate (150 mL) and brine (50 mL). The layers were separated, the organic layer was dried over magnesium sulfate, filtered, and concentrated. The residue was chromatographed on silica gel using hexanes/ethyl acetate, 70:30, v/v as eluant to give the product, 6-benzyl-2-(1-hydroxy-3-phenyl-allyl)-8-methyl-thiazolo[3,2-c]pyrimidine-5,7-dione, 24.8 mg (4.1%). MS (APCI+), m/z (%): 405.1(100).

### EXAMPLE 30

### 6-Benzyl-8-methyl-5,7-dioxo-6,7-dihydro-5H-thiazolo[3,2-c]pyrimidine-2-carboxylic acid 4-fluoro-benzylamide

Procedure 1:

Employing the procedure of Example 2, Step E, 6-benzyl-8-methyl-thiazolo[3,2-c]pyrimidine-5,7-dione (0.407 g, 1.5 mmol) was taken up in tetrahydrofuran (40 mL) and lithium hexamethyldisilazane (1.8 mL, 1 M in tetrahydrofuran, 1.8 mmol) was added at −72° C., and the reaction was allowed to proceed for 3 minutes; then 4-fluorobenzyl isocyanate (0.336 g, 2.2 mmol) in tetrahydrofuran (0.5 mL) was added, and the reaction was stirred for 14 minutes at −72° C., ammonium chloride solution (2 mL) was added, and the reaction mixture was allowed to slowly warm until the ice in the reaction flask melted. The reaction mixture was diluted with ethyl acetate (50 mL) and washed with brine (50 mL). The layers were separated, the organic layer was dried over magnesium sulfate, filtered, and concentrated. The residue was chromatographed on silica gel using hexanes/ethyl acetate, 70:30, v/v as eluant, then chromatographed a second time using hexanes/ethyl acetate, 1:1, v/v as eluant to give the product, 6-benzyl-8-methyl-5,7-dioxo-6,7-dihydro-5H-thiazolo[3,2-c]pyrimidine-2-carboxylic acid 4-fluoro-benzylamide, 50 mg in 2 portions (7.9%). MS (APCI+), m/z (%): 424.1(100), 273.1 (50).

### 6-Benzyl-8-methyl-5,7-dioxo-6,7-dihydro-5H-thiazolo[3,2-c]pyrimidine-2-carboxylic acid 4-fluoro-benzylamide (prepared by carbodiimide coupling)

Procedure 2:

The product of Example 33, Step B (see below), namely 6-benzyl-8-methyl-5,7-dioxo-6,7-dihydro-5H-thiazolo[3,2-c]pydine-2-carboxylic acid (158 mg, 0.50 mmol) (see below) was dissolved in dimethylformamide (4.5 mL). Added were 4-fluorobenzyl amine (84 mg, 67 mmol), 1-hydroxybenzotriazole hydrate (69 mg, 0.50 mmol) and 1-[3-(dimethylamino)propyl]-3-ethylcarbodiimide hydrochloride (104 mg, 0.54 mmol). The reaction mixture was stirred overnight at room temperature. The reaction mixture was partitioned between ethyl acetate (200 mL) and water (100 mL), and the layers were separated. The organic layer was washed with saturated sodium bicarbonate solution (50 mL) and brine (50 mL). The layers were separated and the organic layer dried over magnesium sulfate, filtered, and evaporated in vacuo. The resulting was triturated with ethyl ether and collected by filtration, 0.182 g (86%).

### EXAMPLE 31

### 6-Benzyl-2-(1-hydroxy-3-phenyl-prop-2-ynyl)-8-methyl-thiazolo[3,2-c]pyrimidine-5,7-dione

Employing the procedure of Example 2, Step E, 6-benzyl-8-methyl-thiazolo[3,2-c]pyrimidine-5,7-dione (1.08 g, 4.0 mmol) was taken up in tetrahydrofuran (30 mL) and lithium hexamethyldisilazane (6.0 mL, 1 M in tetrahydrofuran, 6.0 mmol) was added over 2 minutes at −70° C., and the reaction was allowed to proceed for 2 minutes, then phenylpropargyl aldehyde (0.822 g, 6.3 mmol) was added, and the reaction was stirred for 13 minutes at −70° C., ammonium chloride solution was added, and the reaction mixture was allowed to slowly warm until the ice in the reaction flask melted and was then partitioned between ethyl acetate (200 mL) and brine (50 mL). The layers were separated, the organic layer was dried over magnesium sulfate, filtered, and concentrated. The residue was filtered through silica gel using methylene chloride, then hexanes/ethyl acetate, 70:30, v/v as eluant to give the product, 6-benzyl-2-(1-hydroxy-3-phenyl-prop-2-ynyl)-8-methyl-thiazolo[3,2-c]pyrimidine-5,7-dione, 419 mg, in 2 portions (26%); MS (APCI+), m/z (%): 403.2(100), 387.2 (25).

### EXAMPLE 32

### 6-Benzyl-8-formyl-5,7-dioxo-6,7-dihydro-5H-thiazolo[3,2-c]pyrimidine-2-carboxylic acid 4-methoxy-benzylamide

Step A: Employing the procedure of Example 2, Step E, the product of Example 1, Step D, namely 6-benzyl-thiazolo[3,2-c]pyrimidine-5,7-dione (3.56 g, 13.8 mmol) was taken up in tetrahydrofuran (150 mL), and lithium hexamethyldisilazane (21 mL, 1 M in tetrahydrofuran, 6.0 mmol) was added over 7 minutes at −73° C. to −70° C., and the reaction was allowed to proceed for 1 minute; then 4-methoxybenzyl isocyanate (5.06 g, 31 mmol) in tetrahydrofuran (2 mL) was added as rapidly as possible, keeping the temperature below −60° C., and the reaction was stirred for 16 minutes at −70° C.; ammonium chloride solution was added, and the reaction mixture was then partitioned between ethyl acetate (200 mL) and brine (50 mL). The layers were separated, the organic layer was dried over magnesium sulfate, filtered, and concentrated. The residue was filtered through silica gel using hexanes/ethyl acetate, 70:30, v/v as eluant to give the product as a mixture, 1.044 g, in 2 portions (18%). The product could not be purified by trituration with ethyl ether, and was used directly in the next step. MS (APCI+), m/z (%): 422.1(100), 259.1 (65).

Step B: The product from Step A, (0.523 g, 1.24 mmol) was taken up in dimethylformamide (7.5 mL) and phosphorus oxychloride (1 mL) was added and the mixture stirred overnight at room temperature. The reaction mixture was then heated on the rotary evaporator (no vacuum) at 90° C. for 3 hours and was then concentrated. The resulting dark oil was partitioned between ethyl acetate (200 mL) and aqueous sodium bicarbonate/sodium chloride (100 mL), the layers separated, the organic layer dried over magnesium sulfate, filtered, and concentrated. The residue was filtered through silica gel (70-230 mesh) using hexanes/ethyl acetate, 1: 1, v/v to give the product, 6-benzyl-8-formyl-5,7-dioxo-6,7-dihydro-5H-thiazolo[3,2-c]pyrimidine-2-carboxylic acid 4-methoxy-benzylamide, 0.083 g (14.9%). MS (APCI+), m/z (%): 450.1(100), 287.1 (80).

### EXAMPLES 33 and 33a

### 6-Benzyl-8-methyl-5,7-dioxo-6,7-dihydro-5H-thiazolo[3,2-c]pyrimidine-2-carboxylic acid(Example 33)

### 6-Benzyl-8-methyl-5,7-dioxo-6,7-dihydro-5H-thiazolo[3,2-c]pyrimidine-2-carboxylic acid methyl ester (Example 33a)

Step A: Employing the procedure of Example 3, Step E, 6-Benzyl-8-methyl-thiazolo[3,2-c]pyrimidine-5,7-dione (554 mg, 2.03 mmol) in tetrahydrofuran (40 mL) under N₂ at −73° C. was treated with lithium hexamethyldisilazane (3.05 mL 1 M in THF, 3.05 mmol). After 3 minutes, methyl chloroformate was added. After 20 minutes, saturated NH₄Cl was added and the reaction allowed to warm to room temperature. The water layer was removed, the organic layer was dried (Na₂SO₄), decanted and evaporated in vacuo to an oil. Chromatography on silica gel eluting with Hexane:EtOAc, 3:1 gave 6-benzyl-8-methyl-5,7-dioxo-6,7-dihydro-5H-thiazolo[3,2-c]pyrimidine-2-carboxylic acid methyl ester (Example 33a) (53%); MS (APCI+), m/z (%): 331(100), 273(20). 6-Benzyl-8-methyl-5,7-dioxo-6,7-dihydro-5H-thiazolo[3,2-c]pyrimidine-2-carboxylic acid methyl ester also may be prepared according to the procedure of Step A-I below.

Step A-1: The product from Example 2, Step B, namely 3-benzyl-6-chloro-5-methyl-1H-pyrimidine-2,4-dione, (5.02, 20 mmol) was reacted according to the procedure for Example 3, Step A-1, using cesium carbonate in place of triethylamine to give (1-benzyl-5-methyl-2,6-dioxo-1,2,3,6-tetrahydro-pyrimidin-4-ylsulfanyl)-acetic acid methyl ester, 3.165 g (49%). MS (APCI+), m/z (%): 321(100), 289(90).

Step A-2: 1-Benzyl-5-methyl-2,6-dioxo-1,2,3,6-tetrahydro-pyrimidin-4-ylsulfanyl)-acetic acid methyl ester from Step A-1 above, 0.320 g, 1 mmol) was reacted according to the procedure of Example 3, Step A-2 to give benzyl-8-methyl-5,7-dioxo-6,7-dihydro-5H-thiazolo[3,2-c]pyrimidine-2-carboxylic acid methyl ester (Example 33a), 0.203 g (61%). MS (APCI+), m/z (%): 331(100).

### 6-Benzyl-8-methyl-5,7-dioxo-6,7-dihydro-5H-thiazolo[3,2-c]pyrimidine-2-carboxylic acid

Step B: The product from Step A (350 mg, 1.06 mmol) was dissolved in tetrahydrofuran (20 mL). To the solution was added methanol (10 mL) and water (10 mL). To the solution was added lithium hydroxide hydrate (134 mg, 3.2 mmol) in water (10 mL). After 10 minutes at room temperature, the reaction was poured into a separatory funnel of EtOAc and water. Hydrochloric acid (10 mL of 1 M, 10 mmol) was added. The layers were separated, and the aqueous layer was extracted with EtOAc. The combined organic layer was washed with brine, dried (Na₂SO₄), and evaporated in vacuo to an oil. The material was triturated with diethyl ether to give 6-benzyl-8-methyl-5,7-dioxo-6,7-dihydro-5H-thiazolo[3,2-c]pyrimidine-2-carboxylic acid (Example 33) (86%); MS (APCI+), m/z (%): 317(50), 273(100).

### EXAMPLE 34

### 6-Benzyl-8-methyl-5,7-dioxo-6,7-dihydro-5H-thiazolo[3,2-c]pyrimidine-2-carboxylic acid(1H-indol-5-ylmethyl)-amide

6-Benzyl-8-methyl-5,7-dioxo-6,7-dihydro-5H-thiazolo[3,2-c]pyrimidine-2-carboxylic acid(80 mg, 0.25 mmol) was dissolved in tetrahydrofuran (10 mL). Added in order were 5-aminomethylindole (43 mg, 0.29 mmol), 1-hydroxybenzotriazole hydrate (55 mg, 0.41 mmol) and 1-[3-(dimethylamino)propyl]-3-ethylcarbodiimide hydrochloride (57 mg, 0.30 mmol). The mixture was stirred for 2.5 hours at room temperature. The tetrahydrofuran was evaporated in vacuo and the residue partitioned between EtOAc and water. The organic layer was washed twice with 1 M HCl, dried (Na₂SO₄), and evaporated in vacuo. The residue was chromatographed on silica gel eluting with CH₂Cl₂:EtOAc, 9:1. The resulting solid was triturated with diethyl ether and dried in a vacuum to give 6-benzyl-8-methyl-5,7-dioxo-6,7-dihydro-5H-thiazolo[3,2-c]pyrimidine-2-carboxylic acid(1H-indol-5-ylmethyl)-amide (46%); mp 189-191; MS (APCI+), m/z (%): 445(20), 273(20), 130(100).

### EXAMPLE 35

### 6-Benzyl-8-methyl-5,7-dioxo-6,7-dihydro-5H-thiazolo[3,2-c]pyrimidine-2-carboxylic acid(thiazol-4-ylmethyl)-amide hydrochloride

### C-Thiazol-4-yl-methylamine

Step A: C-Thiazol-4-yl-methylamine was made from 4-chloromethyl-thiazole in 2 steps using the procedure of Culbertson, T. P., Domagala, J. M., Peterson, P., Bongers, S., Nichols, J. B.; J. Heterocyclic Chemistry 1987;24: 1509.

### 6-Benzyl-8-methyl-5,7-dioxo-6,7-dihydro-5H-thiazolo[3,2-c]pyrimidine-2-carboxylic acid(thiazol-4-ylmethyl)-amide hydrochloride

Step B: 6-Benzyl-8-methyl-5,7-dioxo-6,7-dihydro-5H-thiazolo[3,2-c]pyrimidine-2-carboxylic acid(333 mg, 1.05 mmol) was dissolved in dimethylformamide (10 mL). Added in order were 4-aminomethylthiazole (138 mg, 1.21 mmol), 1-hydroxybenzotriazole hydrate (148 mg, 1.10 mmol) and 1-[3-(dimethylamino)propyl]-3-ethylcarbodiimide hydrochloride (210 mg, 1.10 mmol). The mixture was stirred for 16 hours at room temperature. The dimethylformamide was evaporated in vacuo and the residue partitioned between EtOAc and water. The organic layer was washed twice with water, twice with 10% sodium carbonate, then brine. The organic layer was dried (Na₂SO₄) and evaporated in vacuo. The residue was crystallized from ethyl acetate to give 6-benzyl-8-methyl-5,7-dioxo-6,7-dihydro-5H-thiazolo[3,2-c]pyrimidine-2-carboxylic acid(thiazol-4-ylmethyl)-amide (60% yield). The material was dissolved in tetrahydrofuran (10 mL) and treated with 1 M HCl in diethyl ether (1 mL, 1 mmol). Filtration gave 6-benzyl-8-methyl-5,7-dioxo-6,7-dihydro-5H-thiazolo[3,2-c]pyrimidine-2-carboxylic acid(thiazol-4-ylmethyl)-amide hydrochloride (91%). MS (APCI+), m/z (%): 413(100), 273(10).

### EXAMPLE 36

### 6-Benzyl-8-methyl-5,7-dioxo-6,7-dihydro-5H-thiazolo[3,2-c]pyrimidine-2-carboxylic acid(pyridin-4-ylmethyl)-amide hydrochloride

The product of Example 33, Step B, namely 6-benzyl-8-methyl-5,7-dioxo-6,7-dihydro-5H-thiazolo[3,2-c]pyrimidine-2-carboxylic acid, (0.32 mmol, based on amount of starting ester from the previous step) was dissolved in tetrahydrofuran (15 mL), and 1-hydroxybenzotriazole hydrate (47 mg, 0.35 mmol), 4-aminomethylpyridine (54 mg, 0.5 mmol) were added. The reaction mixture was cooled to zero degrees, and dicyclohexylcarbodiimide (77 mg, 0.37 mmol) was added. The reaction mixture was allowed to slowly warm to room temperature and was then stirred overnight at room temperature. The reaction mixture was diluted to 100 mL with ethyl acetate and was washed with saturated sodium bicarbonate solution, dried over magnesium sulfate, filtered and concentrated. The residue was chromatographed on silica gel (70-230 mesh) using ethyl acetate as eluant. The product-containing fractions were concentrated and treated with HCl gas in ether to give the product as a white solid in 2 portions, 0.074 g (52%). MS (APCI+), m/z (%): 407.1(100), 273.1 (30).

### EXAMPLES 37 and 37a

### 6-Benzyl-8-methyl-5,7-dioxo-6,7-dihydro-5H-thiiazolo[3,2-c]pyrimidine-2-carboxylic acid(6-methoxy-pyridin-3-ylmethyl)-amide (Example 37); and 6-Benzyl-8-methyl-5,7-dioxo-6,7-dihydro-5H-thiazolo[3,2-c]pyrimidine-2-carboxylic acid(6-methoxy-pyridin-3-ylmethyl)-amide hydrochloride (Example 37a)

The product of Example 33, Step B, namely 6-benzyl-8-methyl-5,7-dioxo-6,7-dihydro-5H-thiazolo[3,2-c]pyrimidine-2-carboxylic acid(158 mg, 0.5 mmol) was dissolved in dimethylformamide (4.5 mL). Added were 2-methoxy-4-aminomethylpyridine (73 mg, 0.54 mmol), 1-hydroxybenzotriazole hydrate (68 mg, 0.5 mmol) and 1-[3-(dimethylamino)propyl]-3-ethylcarbodiimide hydrochloride (102 mg, 0.53 mmol). The mixture was stirred for 3 days at room temperature. The reaction mixture was partitioned between ethyl acetate (200 mL) and water (100 mL). The organic layer was washed with water (100 mL), saturated sodium bicarbonate solution (50 mL), and brine (50 mL). The layers were separated, and the organic layer was dried over magnesium sulfate, filtered, and evaporated in vacuo. The residue was chromatographed on silica gel (70-230 mesh) using ethyl acetate as eluant. The product-containing fractions were concentrated and the residue triturated with ethyl ether/ethyl acetate to give 6-benzyl-8-methyl-5,7-dioxo-6,7-dihydro-5H-thiazolo[3,2-c]pyrimidine-2-carboxylic acid(6-,methoxy-pyridin-3-ylmethyl)-amide, 41.7 mg, (19%) (Example 37). Treatment of the mother liquors with HCl gas in ethyl ether gave 6-benzyl-8-methyl-5,7-dioxo-6,7-dihydro-5H-thiazolo[3,2-c]pyrimidine-2-carboxylic acid(6-methoxy-pyridin-3-ylmethyl)-amide hydrochloride (Example 37a), 45 mg (19%). MS (APCI+), m/z (%): 437.2(100), 273.1 (70).

### EXAMPLE 38

### 6-Benzyl-8-methyl-5,7-dioxo-6,7-dihydro-5H-thiazolo[3,2-c]pyrimidine-2-carboxylic acid(imidazo[2,1-b]thiazol-6-ylmethyl)-amide

The product of Example 33, Step B, namely 6-benzyl-8-methyl-5,7-dioxo-6,7-dihydro-5H-thiazolo[3,2-c]pyrimidine-2-carboxylic acid(158 mg, 0.5 mmol) was dissolved in dimethylformamide (4 mL). Added were C-imidazo[2,1,b]thiazol-6-yl-methylamine(122 mg, 65 mmol), 1-hydroxybenzotriazole hydrate (72 mg, 0.53 mmol) and 1-[3-(dimethylamino)propyl]-3-ethylcarbodiimide hydrochloride (96 mg, 0.50 mmol). The mixture was stirred for 3 days at room temperature. The reaction mixture was concentrated at 60° C. on the rotary evaporator. The residue was partitioned between ethyl acetate/tetrahydrofuran, 1:1, v/v, (200 mL) and water (250 mL). The layers were separated, and the organic layer was washed with saturated sodium bicarbonate solution, the layers were separated and the organic layer dried over magnesium sulfate, filtered, and evaporated in vacuo. The residue was chromatographed on silica gel (70-230 mesh) using ethyl acetate as eluant. The product-containing fractions were concentrated and the residue triturated with ethyl ether/ethyl acetate/tetrahydrofuran to give 6-benzyl-8-methyl-5,7-dioxo-6,7-dihydro-5H-thiazolo[3,2-c]pyrimidine-2-carboxylic acid (imidazo[2,1-b]thiazol-6-ylmethyl)-amide, 143 mg (63%). MS (APCI+) m/z (%): 452.1(100), 273.1 (70).

### EXAMPLE 39

### 6-Benzyl-8-methyl-5,7-dioxo-6,7-dihydro-5H-thiazolo[3,2-c]pyrimidine-2-carboxylic acid(1-methyl-1H-pyrazol-4-ylmethyl)-amide

The product of Example 33, Step B, namely 6-benzyl-8-methyl-5,7-dioxo-6,7-dihydro-5H-thiazolo[3,2-c]pyrimidine-2-carboxylic acid (158 mg, 0.5 mmol) was dissolved in dimethylformamide (4.5 mL). Added were C-(1-methyl-1H-pyrazol4-yl)methylamine (56 mg, 51 mmol), 1-hydroxybenzotriazole hydrate (71 mg, 0.53 mmol) and 1-[3-(dimethylamino)propyl]-3-ethylcarbodiimide hydrochloride (96 mg, 0.50 mmol). The mixture was stirred for 3 days at room temperature. The reaction mixture was concentrated at 58° C. on the rotary evaporator. The residue was partitioned between ethyl acetate (200 mL) and water (200 mL). The layers were separated, and the organic layer was washed with saturated sodium bicarbonate solution (100 ML), the layers were separated and the organic layer dried over magnesium sulfate, filtered, and evaporated in vacuo. The residue was chromatographed on silica gel (70-230 mesh) using ethyl acetate as eluant. The product-containing fractions were concentrated and the residue triturated with ethyl ether to give 6-benzyl-8-methyl-5,7-dioxo-6,7-dihydro-5H-thiazolo[3,2-c]pyrimidine-2-carboxylic acid(1-methyl-1H-pyrazol-4-ylmethyl)-amide, 133 mg (65%). MS (APCI+), m/z (%): 410.2(100), 273.1 (80).

### EXAMPLE 40

### 6-Benzyl-8-methyl-5,7-dioxo-6,7-dihydro-5H-thiazolo[3,2-c]pyrimidine-2-carboxylic acid prop-2-ynylamide

The product of Example 33, Step B, namely 6-benzyl-8-methyl-5,7-dioxo-6,7-dihydro-5H-thiazolo[3,2-c]pyrimidine-2-carboxylic acid (157 mg, 0.5 mmol) was dissolved in dimethylformamide (5 mL). Added were propargyl amine (46 mg, 83 mmol), 1-hydroxybenzotriazole hydrate (71 mg, 0.53 mmol) and 1-[3-(dimethylamino)propyl]-3-ethylcarbodiimide hydrochloride (98 mg, 0.51 mmol). The mixture was stirred for 3 days at room temperature. The reaction mixture was concentrated on the rotary evaporator. The reaction mixture was partitioned between ethyl acetate (200 mL) and saturated sodium bicarbonate solution (100 mL), and the layers were separated. The organic layer was washed with 10% citric acid solution (50 mL) and brine (50 mL) the layers were separated and the organic layer dried over magnesium sulfate, filtered, and evaporated in vacuo. The residue was triturated with ethyl ether/ethyl acetate to give 6-benzyl-8-methyl-5,7-dioxo-6,7-dihydro-5H-thiazolo[3,2-c]pyrimidine-2-carboxylic acid prop-2-ynylamide, 49 mg (28%). MS (APCI+), m/z (%): 354.2(15), 273.2 (20).

### EXAMPLE 41

### 6-Benzyl-8-methyl-5,7-dioxo-6,7-dihydro-5H-thiazolo[3,2-c]pyrimidine-2-carboxylic acid(6-methyl-pyridin-2-ylmethyl)-amide

The product of Example 33, Step B, namely 6-benzyl-8-methyl-5,7-dioxo-6,7-dihydro-5H-thiazolo[3,2-c]pyrimidine-2-carboxylic acid (134 mg, 0.42 mmol) was dissolved in dimethylformamide (6 mL). Added were 2-methyl-6-aminomethyl pyridine (55 mg, 45 mmol), 1-hydroxybenzotriazole hydrate (57 mg, 0.42 mmol) and 1-[3-(dimethylamino)propyl]-3-ethylcarbodiimide hydrochloride (84 mg, 0.44 mmol). The mixture was stirred overnight at room temperature. The reaction mixture was concentrated on the rotary evaporator at 58° C. The residue was partitioned between ethyl acetate (200 mL) and water (100 mL), and the layers were separated. The organic layer was washed with saturated sodium bicarbonate solution (100 mL) and brine (50 mL), the layers were separated, and the organic layer dried over magnesium sulfate, filtered, and evaporated in vacuo. The residue was triturated with hexanes/ethyl acetate to give 6-benzyl-8-methyl-5,7-dioxo-6,7-dihydro-5H-thiazolo[3,2-c]pyrimidine-2-carboxylic acid(6-methyl-pyridin-2-ylmethyl)-amide, 119 mg (67%). MS (APCI+), m/z (%): 421.2(100).

### EXAMPLE 42

### 6-Benzyl-8-methyl-5,7-dioxo-6,7-dihydro-5H-thiazolo[3,2-c]pyrimidine-2-carboxylic acid(2,1,3-benzothiadiazol-5-ylmethyl)-amide

The product of Example 33, Step B, namely 6-benzyl-8-methyl-5,7-dioxo-6,7-dihydro-5H-thiazolo[3,2-c]pyrimidine-2-carboxylic acid (164 mg, 0.52 mmol) was dissolved in dimethylformamide (6 mL). Added were C-benzo[1,2,5]thiadiazol-5-yl-methyl amine hydrochloride (104 mg, 52 mmol), 1-hydroxybenzotriazole hydrate (71 mg, 0.53 mmol) and 1-[3-(dimethylamino)propyl]-3-ethylcarbodiimide hydrochloride (104 mg, 0.52 mmol). The mixture was stirred overnight at room temperature. The reaction mixture was concentrated on the rotary evaporator at 58° C. The residue was partitioned between ethyl acetate (200 mL) and water (100 mL), and the layers were separated. The organic layer was washed with saturated sodium bicarbonate solution (100 mL) and brine (50 mL), the layers were separated, and the organic layer dried over magnesium sulfate, filtered, and evaporated in vacuo. The residue was triturated with ethyl ether/ethyl acetate to give 6-benzyl-8-methyl-5,7-dioxo-6,7-dihydro-5H-thiazolo[3,2-c]pyrimidine-2-carboxylic acid(2,1,3-benzothiadiazol-5-ylmethyl)-amide, 169 mg (70%). MS (APCI+), m/z (%): 464.2(100).

### EXAMPLE 43

### 6-Benzyl-8-methyl-5,7-dioxo-6,7-dihydro-5H-thiazolo[3,2-c]pyrimidine-2-carboxylic acid 3,4-difluoro-benzylamide

The product of Example 33, Step B, namely 6-benzyl-8-methyl-5,7-dioxo-6,7-dihydro-5H-thiazolo[3,2-c]pyrimidine-2-carboxylic acid (105 mg, 0.33 mmol) was dissolved in dimethylformamide (3 mL). Added were 3,4-difluorobenzyl amine (50 mg, 35 mmol), 1-hydroxybenzotriazole hydrate (45 mg, 0.33 mmol) and 1-[3-(dimethylamino)propyl]-3-ethylcarbodiimide hydrochloride (66 mg, 0.35 mmol). The mixture was stirred overnight at room temperature. The reaction mixture was concentrated on the rotary evaporator. The residue was partitioned between ethyl acetate (200 mL) and water (100 mL), and the layers were separated. The organic layer was washed with saturated sodium bicarbonate solution (50 mL) and brine (50 mL), the layers were separated and the organic layer dried over magnesium sulfate, filtered, and evaporated in vacuo. The residue was triturated with hexanes/ethyl acetate to give 6-benzyl-8-methyl-5,7-dioxo-6,7-dihydro-5H-thiazolo[3,2-c]pyrimidine-2-carboxylic acid 3,4-difluoro-benzylamide, 105 mg (72%). MS (APCI+), m/z (%): 442.1(100), 273.1 (100).

### EXAMPLES 44 and 44a

### 6-benzyl-8-methyl-5,7-dioxo-6,7-dihydro-5H-thiazolo[3,2-c]pyrimidine-2-carboxylic acid(pyridin-3-ylmethyl)-amide (Example 44)

### 6-Benzyl-8-methyl-5,7-dioxo-6,7-dihydro-5H-thiazolo[3,2-c]pyrimidine-2-carboxylic acid(pyridin-3-ylmethyl)-amide hydrochloride (Example 44a)

The product of Example 33, Step B, namely 6-benzyl-8-methyl-5,7-dioxo-6,7-dihydro-5H-thiazolo[3,2-c]pyrimidine-2-carboxylic acid (156 mg, 0.49 mmol) was dissolved in dimethylformamide (4 mL). Added were 3-aminomethylpyridine (74 mg, 69 mmol), 1-hydroxybenzotriazole hydrate (71 mg, 0.53 mmol), and 1-[3-(dimethylamino)propyl]-3-ethylcarbodiimide hydrochloride (99 mg, 0.52 mmol). The reaction mixture was stirred overnight at room temperature. The reaction mixture was partitioned between ethyl acetate (300 mL) and water (100 mL), and the layers were separated. The organic layer was washed with saturated sodium bicarbonate solution, the layers were separated and the organic layer dried over magnesium sulfate, filtered, and evaporated in vacuo. The resulting oil began to crystallize on standing. The oil/solid was taken up in ethyl acetate, filtered, concentrated to dryness, and triturated with hexanes/ethyl acetate. The resulting solid, 6-benzyl-8-methyl-5,7-dioxo-6,7-dihydro-5H-thiazolo[3,2-c]pyrimidine-2-carboxylic acid(pyridin-3-ylmethyl)-amide (Example 44) was collected by filtration, 140 mg (70%). The oil from the concentrated mother liquors was taken up in tetrahydrofuran and treated with HCl gas in ethyl ether to give the hydrochloride, 35 mg (Example 44a) (7.9%). MS (APCI+), m/z (%): 407.2(100), 273.1 (50).

### EXAMPLE 45

### 6-Benzyl-8-methyl-5,7-dioxo-6,7-dihydro-5H-thiazolo[3,2-c]pyrimidine-2-carboxylic acid(piperidin-4-ylmethyl)-amide hydrochloride

Step A: The product of Example 33, Step B, namely 6-benzyl-8-methyl-5,7-dioxo-6,7-dihydro-5H-thiazolo[3,2-c]pyrimidine-2-carboxylic acid (157 mg, 0.50 mmol) was dissolved in dimethylformamide (3 mL). Added were 4-aminomethyl-N-tert-butyloxycarbonylpiperidine (113 mg, 53 mmol), 1-hydroxybenzotriazole hydrate (71 mg, 0.53 mmol) and 1-[3-(dimethylamino)propyl]-3-ethylcarbodiimide hydrochloride (105 mg, 0.55 mmol). The reaction mixture was stirred overnight at room temperature. The reaction mixture was partitioned between ethyl acetate (200 mL) and water (100 mL), and the layers were separated. The organic layer was washed with saturated sodium bicarbonate solution (100 mL), the layers were separated, and the organic layer dried over magnesium sulfate, filtered, and evaporated in vacuo. The resulting oil slowly crystallized on standing. This was used directly in the next step. MS (APCI+), m/z (%): 513.3(1), 457.3 (15), 413.3 (40).

Step B: The product of Example 45, Step A, 230 mg, 45 mmol, was taken up in dichloromethane (20 mL), and HCl gas was bubbled in for about 2 minutes, and the flask was stoppered and allowed to stand overnight at room temperature. The reaction mixture was concentrated to a foam, and ethyl ether was added. The resulting solid was collected by filtration. The solid was very hygroscopic and turned to a gum that solidified on standing, 6-benzyl-8-methyl-5,7-dioxo-6,7-dihydro-5H-thiazolo[3,2-c]pyrimidine-2-carboxylic acid(piperidin-4-ylmethyl)-amide hydrochloride, 116 mg (56%); MS (APCI+), m/z (%): 413.3 (100).

### EXAMPLE 46

### 6-Benzyl-8-methyl-5,7-dioxo-6,7-dihydro-5H-thiazolo[3,2-c]pyrimidine-2-carboxylic acid 3-fluoro-4-methoxy-benzylamide

The product of Example 33, Step B, namely 6-benzyl-8-methyl-5,7-dioxo-6,7-dihydro-5H-thiazolo[3,2-c]pyrimidine-2-carboxylic acid (158 mg, 0.50 mmol) was dissolved in dimethylformamide (4.5 mL). Added were 3-fluoro-4-methoxybenzyl amine (80 mg, 52 mmol), 1-hydroxybenzotriazole hydrate (71 mg, 0.53 mmol) and 1-[3-(dimethylamino)propyl]-3-ethylcarbodiimide hydrochloride (102 mg, 0.53 mmol). The reaction mixture was stirred 3 days at room temperature. The reaction mixture was partitioned between ethyl acetate (200 mL) and water (100 mL), and the layers were separated. The organic layer was washed with saturated sodium bicarbonate solution (100 mL), the layers were separated, and the organic layer dried over magnesium sulfate, filtered, and evaporated in vacuo. The residue was filtered through silica gel (70-230 mesh) using hexanes/ethyl acetate, 7:3, v/v, as eluant. The product-containing fractions were concentrated and the residue triturated with ethyl ether. The resulting solid, 6-benzyl-8-methyl-5,7-dioxo-6,7-dihydro-5H-thiazolo[3,2-c]pyrimidine-2-carboxylic acid 3-fluoro4-methoxy-benzylamide, was collected by filtration, 53 mg (23%). MS (APCI+), n/z (%): 454.2 (80), 273.2 (100).

### EXAMPLE 47

### 6-Benzyl-8-methyl-5,7-dioxo-6,7-dihydro-5H-thiazolo[3,2-c]pyrimidine-2-carboxylic acid(pyridin-2-ylmethyl)-amide hydrochloride

Step A: The product of Example 33, Step B, namely 6-benzyl-8-methyl-5,7-dioxo-6,7-dihydro-5H-thiazolo[3,2-c]pyrimidine-2-carboxylic acid (183 mg, 0.58 mmol) was dissolved in tetrahydrofuran (30 mL). The reaction mixture was cooled to 0° C., 2 drops of dimethylformamide were added, then oxalyl chloride (0.2 mL, 2.29 mmol) was added, and the mixture was stirred at 0° C. under an atmosphere of nitrogen gas for 10 minutes. Then the reaction mixture was allowed to warm to room temperature, stirred ten minutes, and then concentrated to an oil/solid without heating. This material was used directly in the next step.

Step B: To the product from Example 47, Step A, (146 mg, 0.44 mmol), was added under a nitrogen atmosphere, 2-aminomethyl pyridine (68 mg, 0.63 mmol) in pyridine (3 mL). The reaction mixture was stirred 30 minutes, and then water was added, and the resulting mixture was extracted with ethyl acetate. The ethyl acetate solution was washed with water and brine and dried over magnesium sulfate, filtered, and concentrated to an oil. Water was added to the oil and decanted. The oil was taken up in tetrahydrofuran and dried over magnesium sulfate. The process of washing with water and drying was repeated twice more to remove pyridine. The resulting brown solid was chromatographed on silica gel (70-230 mesh) using ethyl acetate as eluant. The product-containing fractions were concentrated, the residue triturated with hexanes/ethyl acetate to give an off-white solid. The solid was taken up in tetrahydrofuran and treated with HCl gas in ethyl ether. The resulting solid was collected by filtration, 6-benzyl-8-methyl-5,7-dioxo-6,7-dihydro-5H-thiazolo[3,2-c]pyrimidine-2-carboxylic acid(pyridin-2-ylmethyl)-amide hydrochloride, 73 mg (44%). MS (APCI+), m/z (%): 407.1 (100), 331.1 (50).

### EXAMPLE 48

### 6-Benzyl-8-methyl-5,7-dioxo-6,7-dihydro-5H-thiazolo[3,2-c]pyrimidine-2-carboxylic acid 4-methyl-benzylamide

The product of Example 33, Step B, namely 6-benzyl-8-methyl-5,7-dioxo-6,7-dihydro-5H-thiazolo[3,2-c]pyrimidine-2-carboxylic acid (158 mg, 0.50 mmol) was dissolved in dimethylformamide (4 mL). Added were 4-methylbenzyl amine (61 mg, 50 mmol), 1-hydroxybenzotriazole hydrate (68 mg, 0.50 mmol) and 1-[3-(dimethylamino)propyl]-3-ethylcarbodiimide hydrochloride (100 mg, 0.52 mmol). The mixture was stirred overnight at room temperature. The reaction mixture was concentrated on the rotary evaporator. The residue was partitioned between ethyl acetate and water. The layers were separated, and the organic layer was washed with saturated sodium bicarbonate solution, the layers were separated, and the organic layer dried over magnesium sulfate, filtered, and evaporated in vacuo. The residue was triturated with hexanes/ethyl acetate to give the product, 6-benzyl-8-methyl-5,7-dioxo-6,7-dihydro-5H-thiazolo[3,2-c]pyrimidine-2-carboxylic acid 4-methyl-benzylamide, 141 mg (67%). MS (APCI+), m/z (%): 420.2 (100).

### EXAMPLE 49

### 6-Benzyl-8-methyl-5,7-dioxo-6,7-dihydro-5H-thiazolo[3,2-c]pyrimidine-2-carboxylic acid 4-trifluoromethyl-benzylamide

The product of Example 33, Step B, namely 6-benzyl-8-methyl-5,7-dioxo-6,7-dihydro-5H-thiazolo[3,2-c]pyrimidine-2-carboxylic acid (158 mg, 0.50 mmol) was dissolved in dimethylformamide (4 mL). Added were 4-trifluoromethylbenzyl amine (88 mg, 50 mmol), 1-hydroxybenzotriazole hydrate (68 mg, 0.50 mmol) and 1-[3-(dimethylamino)propyl]-3-ethylcarbodiimide hydrochloride (100 mg, 0.52 mmol). The mixture was stirred overnight at room temperature. The reaction mixture was concentrated on the rotary evaporator. The residue was partitioned between ethyl acetate and water. The layers were separated and the organic layer was washed with saturated sodium bicarbonate solution, the layers were separated, and the organic layer dried over magnesium sulfate, filtered, and evaporated in vacuo. The residue was triturated with hexanes/ethyl acetate to give the product, 6-benzyl-8-methyl -5,7-dioxo-6,7-dihydro-5H-thiazolo[3,2-c]pyrimidine-2-carboxylic acid 4-trifluoromethyl-benzylamide, 154 mg (65%). MS (APCI+), m/z (%): 474.2 (100), 371.3 (30).

### EXAMPLE 50

### 6-Benzyl-8-methyl-5,7-dioxo-6,7-dihydro-5H-thiazolo[3,2-c]pyrimidine-2-carboxylic acid 4-chloro-benzylamide

The product of Example 33, Step B, namely 6-benzyl-8-methyl-5,7-dioxo-6,7-dihydro-5H-thiazolo[3,2-c]pyrimidine-2-carboxylic acid (158 mg, 0.50 mmol) was dissolved in dimethylformamide (4 mL). Added were 4-chlorobenzyl amine (71 mg, 50 mmol), 1-hydroxybenzotriazole hydrate (68 mg, 0.50 mmol) and 1-[3-(dimethylamino)propyl]-3-ethylcarbodiimide hydrochloride (100 mg, 0.52 mmol). The mixture was stirred overnight at room temperature. The reaction mixture was concentrated on the rotary evaporator. The residue was partitioned between ethyl acetate and water. The layers were separated, and the organic layer was washed with saturated sodium bicarbonate solution; the layers were separated and the organic layer dried over magnesium sulfate, filtered, and evaporated in vacuo. The residue was triturated with hexanes/ethyl acetate to give the product, 6-benzyl-8-methyl-5,7-dioxo-6,7-dihydro-5H-thiazolo[3,2-c]pyrimidine-2-carboxylic acid 4-chloro-benzylamide; 170 mg (77%). MS (APCI+), m/z (%): 440.1 (100), 442.1 (50).

### EXAMPLE 51

### 6-Benzyl-8-methyl-5,7-dioxo-6,7-dihydro-5H-thiazolo[3,2-c]pyrimidine-2-carboxylic acid 4-trifluoromethoxy-benzylamide

The product of Example 33, Step B, namely 6-benzyl-8-methyl-5,7-dioxo-6,7-dihydro-5H-thiazolo[3,2-c]pyrimidine-2-carboxylic acid (158 mg, 0.50 mmol) was dissolved in dimethylformamide (4 mL). Added were 4-trifluoromethylbenzyl amine (96 mg, 50 mmol), 1-hydroxybenzotriazole hydrate (68 mg, 0.50 mmol) and 1-[3-(dimethylamino)propyl]-3-ethylcarbodiimide hydrochloride (100 mg, 0.52 mmol). The mixture was stirred overnight at room temperature. The reaction mixture was concentrated on the rotary evaporator. The residue was partitioned between ethyl acetate and water. The layers were separated, and the organic layer was washed with saturated sodium bicarbonate solution. The layers were separated and the organic layer dried over magnesium sulfate, filtered, and evaporated in vacuo. The residue was triturated with hexanes/ethyl acetate to give the product, 6-benzyl-8-methyl-5,7-dioxo-6,7-dihydro-5H-thiazolo[3,2-c]pyrimidine-2-carboxylic acid 4-trifluoromethoxy-benzylamide; 191 mg (80%). MS (APCI+), m/z (%): 490.2 (100), 273.2 (50).

### EXAMPLE 52

### 6-Benzyl-8-methyl-5,7-dioxo-6,7-dihydro-5H-thiazolo[3,2-c]pyrimidine-2-carboxylic acid(2-methyl-thiazol-4-ylmethyl)-amide hydrochloride

### C-(2-Methyl-thiazol-4-yl)-methylamine

Step A: C-(2-Methyl-thiazol4-yl)-methylamine was made from 4-chloromethyl-2-methyl-thiazole in 2 steps using the procedure of Culbertson T P, Domagala J M, Peterson P, Bongers S, Nichols J B; J. Heterocyclic Chemistry 1987, 24, 1509.

6-Benzyl-8-methyl-5,7-dioxo-6,7-dihydro-5H-thiazolo[3,2-c]pyrimidine-2-carboxylic acid(2-methyl-thiazol-4-ylmethyl)-amide hydrochloride

Step B: 6-Benzyl-8-methyl-5,7-dioxo-6,7-dihydro-5H-thiazolo[3,2-c]pyrimidine-2-carboxylic acid was treated as in Example 35, Step B with C-(2-Methyl-thiazol4-yl)-methylamine. The free base crystallized from ethyl acetate. The HCl salt made as in Example 35, Step B gave 6-benzyl-8-methyl-5,7-dioxo-6,7-dihydro-5H-thiazolo[3,2-c]pyrimidine-2-carboxylic acid(2-methyl-thiazol4-ylmethyl)-amide hydrochloride (45%); MS (APCI+), m/z (%): 427(100), 169(25).

### EXAMPLE 53

### 8-Methyl-thiazolo[3,2-c]pyrimidine-5,7-dione

The product of Example 2, Step D, namely 6-benzyl-8-methyl-thiazolo[3,2-c]pyrimidine-5,7-dione (1.00 g, 3.67 mmol) was dissolved in benzene (25 mL). Aluminum chloride (2.00 g, 15 mmol) was added, and the mixture heated at reflux for 16 hours. The warm mixture was poured over ice and stirred until the ice melted. The resulting yellow solid was filtered and rinsed with water. The solid was triturated with diethyl ether then dried in vacuo at 50° C. for 16 hours to give 8-methyl-thiazolo[3,2-c]pyrimidine-5,7-dione (83%). mp>230° C. MS (APCI+), m/z (%): 183(100), 140(20).

### EXAMPLE 54

### 8-Methyl-5,7-dioxo-6,7-dihydro-5H-thiazolo[3,2-c]pyrimidine-2-carboxylic acid

The product from Example 3, Step A, namely 6-benzyl-8-methyl-5,7-dioxo-6,7-dihydro-5H-thiazolo[3,2-c]pyrimidine-2-carboxylic acid methyl ester (4.0 g, 12 mmol) was dissolved in benzene (115 mL). Aluminum chloride (6.4 g, 48 mmol) was added and the suspension heated at reflux for 21 hours. The warm mixture was poured over ice and stirred until the ice melted. The solid was filtered. The solid was suspended in water and 1 M sodium hydroxide (1.1 eq.) was added. The mixture was stirred for 90 minutes, then filtered. The filtrate was treated with 1 M hydrochloric acid (1.1 eq.). The resulting solid was isolated and dried in vacuo at 50° C. to give 8-methyl-5,7-dioxo-6,7-dihydro-5H-thiazolo[3,2-c]pyrimidine-2-carboxylic acid (99%). mp>205° C. MS (APCI+), m/z (%): 227(25), 183(100), 139(20).

### EXAMPLE 55

### 4-[2-(4-Methoxy-benzylcarbamoyl)-8-methyl-5,7-dioxo-7H-thiazolo[3,2-c]pyrimidin-6-ylmethyl]-benzoic acid

### 8-Methyl-5,7-dioxo-6,7-dihydro-5H-thiazolo[3,2-c]pyrimidine-2-carboxylic acid 4-methoxy-benzylamide

Step A: The product of Example 54 (10.0 g, 41 mmol) was dissolved in dimethylformamide (300 mL). To the solution was added 1-hydroxybenzotriazole hydrate (6.08 g, 45 mmol) and 1-[3-(dimethylamino)propyl]-3-ethylcarbodiimide hydrochloride (10.2 g, 53 mmol), then 4-methoxybenzylamine (5.9 mL, 45 mmol). The mixture was stirred for 22 hours at room temperature. The dimethylformamide was removed in vacuum at 60° C. The residue was stirred in water for 30 minutes then filtered. The resulting solid was stirred with 10% aqueous sodium carbonate for 30 minutes. The mixture was filtered and rinsed with water, then vacuum dried at 45° C. for 16 hours to give 8-methyl-5,7-dioxo-6,7-dihydro-5H-thiazolo[3,2-c]pyrimidine-2-carboxylic acid 4-methoxy-benzylamide (77%). MS (APCI+), m/z (%): 346(100), 303(30), 277(45).

### 4-Methylbenzoic acid tert-butyl ester

Step B: To a solution of pyridine (125 mL) and tert-butanol (125 mL, 1.31 mole) was added 4-methylbenzoyl chloride (171 mL, 1.29 mole). The reaction was stirred at room temperature for 88 hours, then poured into water (325 mL) and EtOAc (325 mL). The layers were separated. The EtOAc layer was washed with 0.5 M HCl (3×200 mL), water (200 mL), aqueous sodium bicarbonate, and brine. The solvent was evaporated in vacuo to give the crude ester. The material was dissolved in hexanes (250 mL) and passed through silica gel eluting with additional hexanes. The solvent was evaporated in vacuo to give 4-methylbenzoic acid tert-butyl ester (96%). ¹H-NMR (CDCl₃) δ 7.87 (d, 2H), 7.20(d, 2H), 2.39(s, 3H), 1.58(s, 9H).

### 4-Bromomethylbenzoic acid tert-butyl ester

Step C: The product from Example 55, Step B (50.0 g, 0.26 mole) was dissolved in carbon tetrachloride (250 mL). N-Bromosuccinimide (46.3 g, 0.26 mole) was added followed by benzoyl peroxide (0.6 g, 0.0026 mole). The mixture was heated at reflux for 4 hours. The cooled reaction was filtered, rinsing the solid with hexanes. The combined filtrate was washed with aqueous sodium bisulfite, and 0.5 M sodium hydroxide. The organic layer was dried (Na₂SO₄) and passed through silica gel eluting with hexanes. The solvent was removed in vacuo to give 4-bromomethylbenzoic acid tert-butyl ester (72%). The material could be crystallized from methanol; mp 46-48; ¹H-NMR (CDCl₃) δ 7.95(d, 2H), 7.41(d, 2H), 4.50(s, 2H), 1.59(s, 9H).

### 4-[2-(4-Methoxy-benzylcarbamoyl)-8-methyl-5,7-dioxo-7H-thiazolo[3,2-c]pyrimidin-6-ylmethyl]-benzoic acid tert-butyl ester

### Step D: The product from Example 55, Step A (10.0 g, 29.0 mmol) was suspended in dimethylformamide (300 mL). Cesium carbonate (9.55 g, 29.3 mmol) was added followed by the product of Example 55, Step C, namely 4-Bromomethylbenzoic acid tert-butyl ester (7.86 g, 29.0 mmol). After 17 hours, the dimethylformamide was removed in a vacuum at 70° C. The residue was mixed with tetrahydrofuran and filtered through a pad of Celite over silica gel eluting with additional tetrahydrofuran. The filtrate was evaporated in vacuo to an oil. The material was purified by chromatography on silica gel, eluting with CH₂Cl₂:tetrahydrofuran (19:1) to give 4-[2-(4-methoxy-benzylcarbamoyl)-8-methyl-5,7-dioxo-7H-thiazolo[3,2-c]pyrimidin-6-ylmethyl]-benzoic acid tert-butyl ester (80%). MS (APCI+), m/z (%): 536(35), 480(100), 317(80).

### 4-[2-(4-Methoxy-benzylcarbamoyl)-8-methyl-5,7-dioxo-7H-thiazolo[3,2-c]pyrimidin-6-ylmethyl]-benzoic acid

Step E: The product from Example 55, Step D (12.2 g, 22.8 mmol) was dissolved in trifluoroacetic acid (100 mL) and stirred at room temperature for 1.5 hours. The solvent was removed in vacuo at 40° C. The resulting oil crystallized in tetrahydrofuran. The tetrahydrofuran was evaporated in vacuo. The solid was triturated with diethyl ether, then vacuum dried at 45° C. to give 4-[2-(4-methoxy-benzylcarbamoyl)-8-methyl-5,7-dioxo-7H-thiazolo[3,2-c]pyrimidin-6-ylmethyl]-benzoic acid (80%); mp>210° C.; MS (APCI+), m/z (%): 480(10), 317(100).

### EXAMPLE 56

### 4-[2-(4-Methoxy-benzylcarbamoyl)-8-methyl-5,7-dioxo-7H-thiazolo[3,2-c]pyrimidin-6-ylmethyl]-benzoic acid sodium salt

To the product of Example 55, Step E (1.05 g, 2.19 mmol), suspended in ethanol (120 mL) was added 1 M sodium hydroxide (2.23 mL, 2.23 mmol). After 20 minutes, water (2 mL) was added to complete the solution. The solution was filtered and the filtrate evaporated to a white solid. The material was triturated with ethanol (10 mL) and rinsed twice with diethyl ether. The solid was vacuum dried at 55° C. for 18 hours to give 4-[2-(4-methoxy-benzylcarbamoyl)-8-methyl-5,7-dioxo-7H-thiazolo[3,2-c]pyrimidin-6-ylmethyl]-benzoic acid sodium salt (95%); MS (APCI+), m/z (%): 480(20), 317(100).

### EXAMPLE 57

### 4-[2-(4-Methoxy-benzylcarbamoyl)-8-methyl-5,7-dioxo-7H-thiazolo[3,2c]pyrimidin-6-ylmethyl]-benzoic acid 2-dimethylamine-ethyl ester hydrochloride

The product of Example 55, Step E was treated as in the procedure of Example 55, Step A using N,N-dimethylaminoethanol. The crude product was dissolved in ethyl acetate/tetrahydrofuran and washed with water, 10% aqueous sodium carbonate and brine, dried (Na_{S}SO₄) and evaporated to the free base. The material was dissolved in tetrahydrofuran and treated with 1 M HCl in diethyl ether (1.2 equivalents). The resulting solid was filtered and rinsed with diethyl ether, then vacuum dried to give 4-[2-(4-methoxy-benzylcarbamoyl)-8-methyl-5,7-dioxo-7H-thiazolo[3,2-]pyrimidin-6-ylmethyl]-benzoic acid 2-dimethylamino-ethyl ester hydrochloride (67%); MS (APCI+), m/z (%): 551(100), 317 (30).

### EXAMPLE 58

### 4-[2-(4-Fluoro-benzylcarbamoyl)-8-methyl-5,7-dioxo-7H-thiazolo[3,2-c]pyrimidin-6-ylmethyl]-benzoic acid

### 8-Methyl-5,7-dioxo-6,7-dihydro-5H-thiazolo[3,2-c]pyrimidine-2-carboxylic acid 4-fluoro-benzylamide

Step A: 8-Methyl-5,7-dioxo-6,7-dihydro-5H-thiazolo[3,2-c]pyrimidine-2-carboxylic acid was treated as in Example 55, Step A using 4-fluoro-benzylamine to give 8-methyl-5,7-dioxo-6,7-dihydro-5H-thiazolo[3,2-c]pyrimidine-2-carboxylic acid 4-fluoro-benzylamide (99%); MS (APCI+), m/z (%): 334(100), 291(50), 265(95).

### 4-[2-(4-Fluoro-benzylcarbamoyl)-8-methyl-5,7-dioxo-7H-thiazolo[3,2-c]pyrimidin-6-ylmethyl]-benzoic acid tert-butyl ester

Step B: The product of Step A was treated as in Example 55, Step D to give 4-[2-(4-fluoro-benzylcarbamoyl)-8-methyl-5,7-dioxo-7H-thiazolo[3,2-c]pyrimidin-6-ylmethyl]-benzoic acid tert-butyl ester (63%); MS (APCI+), m/z (%): 524(35), 468(100), 317(55).

### 4-[2-(4-Fluoro-benzylcarbamoyl)-8-methyl-5,7-dioxo-7H-thiazolo[3,2-c]pyrimidin-6-ylmethyl]-benzoic acid

Step C: The product from Step B was treated as in Example 55, Step E to give 4-[2-(4-fluoro-benzylcarbamoyl)-8-methyl-5,7-dioxo-7H-thiazolo[3,2-c]pyrimidin-6-ylmethyl]-benzoic acid (93%); MS (APCI+), m/z (%): 468(100), 317(50).

### EXAMPLE 59

### 4-[2-(4-Fluoro-benzylcarbamoyl)-8-methyl-5,7-dioxo-7H-thiazolo[3,2-c]pyrimidin-6-ylmethyl]-benzoic acid Sodium Salt

4-[2-(4-Fluoro-benzylcarbamoyl)-8-methyl-5,7-dioxo-7H-thiazolo[3,2-c]pyrimidin-6-ylmethyl]-benzoic acid was treated as in Example 56 to give 4-[2-(4-fluoro-benzylcarbamoyl)-8-methyl-5,7-dioxo-7H-thiazolo[3,2-c]pyrimidin-6-ylmethyl]-benzoic acid sodium salt (85%); MS (APCI+), m/z (%): 468(30), 317 (100).

### EXAMPLE 60

### 4-[2-(4-Fluoro-benzylcarbamoyl)-8-methyl-5,7-dioxo-7H-thiazolo[3,2-c]pyrimidin-6-ylmethyl]-benzoic acid 2-dimethylamino-ethyl ester

4-[2-(4-Fluoro-benzylcarbamoyl)-8-methyl-5,7-dioxo-7H-thiazolo[3,2-c]pyrimidin-6-ylmethyl]-benzoic acid was treated with N,N-dimethylaminoethanol as in Example 57. The crude compound was dissolved in ethyl acetate and washed with water and 10% aqueous sodium carbonate, dried (Na₂SO₄) and evaporated. The residue was chromatographed on silica gel eluting with CH₂Cl₂:MeOH 9:1 to give 4-[2-(4-fluoro-benzylcarbamoyl)-8-methyl-5,7-dioxo-7H-thiazolo[3,2-c]pyrimidin-6-ylmethyl]-benzoic acid 2-dimethylamino-ethyl ester (30%); MS (APCI+), m/z (%): 539(100), 388(15), 317(20).

### EXAMPLE 61

### 4-[2-(4-Fluoro-benzylcarbamoyl)-8-methyl-5,7-dioxo-7H-thiazolo[3,2-c]pyrimidin-6-ylmethyl]-benzoic acid 2-dimethylamino-ethyl ester hydrochloride

The product of Example 60 was dissolved in tetrahydrofuran and treated with 1 M HCl in diethyl ether (1.2 equivalents). The resulting solid was filtered and rinsed with diethyl ether, then vacuum dried to give 4-[2-(4-fluoro-benzylcarbamoyl)-8-methyl-5,7-dioxo-7H-thiazolo[3,2-c]pyrimidin-6-ylmethyl]-benzoic acid 2-dimethylamino-ethyl ester hydrochloride (41%); MS (APCI+), m/z (%): 539(100), 388(20), 317(40).

### EXAMPLE 62

### 4-{8-Methyl-5,7-dioxo-2-[(pyridin-4-ylmethyl)-carbamoyl]-7H-thiazolo[3,2-c]pyrimidin-6-ylmethyl}-benzoic acid trifluoro-acetate

### 8-Methyl-5,7-dioxo-6,7-dihydro-5H-thiazolo[3,2-c]pyrimidine-2-carboxylic acid(pyridin-4-ylmethyl)-amide

Step A: 8-Methyl-5,7-dioxo-6,7-dihydro-5H-thiazolo[3,2-c]pyrimidine-2-carboxylic acid was treated as in Example 55, Step A using C-pyridin-4-yl-methylamine to give 8-methyl-5,7-dioxo-6,7-dihydro-5H-thiazolo[3,2-c]pyrimidine-2-carboxylic acid(pyridin-4-ylmethyl)-amide (82%); MS (APCI+), m/z (%): 317(100), 274(50), 248(95).

### 4-{8-Methyl-5,7-dioxo-2-[(pyridin-4-ylmethyl)-carbamoyl]-7H-thiazolo[3,2-c]pyrimidin-6-ylmethyl}-benzoic acid tert-butyl ester

Step B: The product of Step A was treated as in Example 55, Step D to give 4-{8-methyl-5,7-dioxo-2-[(pyridin-4-ylmethyl)-carbamoyl]-7H-thiazolo[3,2-c]pyridin-6-ylmethyl}-benzoic acid tert-butyl ester (47%); MS (AP+) m/z (%): 507(100), 451(35), 317(35), 147(40).

### 4-{8-Methyl-5,7-dioxo-2-[(pyridin-4-ylmethyl)-carbamoyl]-7H-thiazolo[3,2-c]pyrimidin-6-ylmethyl}-benzoic acid trifluoro-acetate

Step C: The product from Step B was treated as in Example 55, Step E. Trituration with diethyl ether, ethyl acetate and again with diethyl ether gave 4-{8-methyl-5,7-dioxo-2-[(pyridin-4-ylmethyl)-carbamoyl]-7H-thiazolo[3,2-c]pyrimidin-6-ylmethyl}-benzoic acid trifluoro-acetate (93%); MS (APCI+), m/z (%): 451(40), 317(100), 135(30).

### EXAMPLE 63

### 4-{8-Methyl-5,7-dioxo-2-[(pyridin-4-ylmethyl)-carbamoyl]-7H-thiazolo[3,2-c]pyrimidin-6-ylmethyl}-benzoic acid 2-dimethylamino-ethyl ester dihydrochloride

4-{8-Methyl-5,7-dioxo-2-[(pyridin-4-ylmethyl)-carbamoyl]-7H-thiazolo[3,2-c]pyrimidin-6-ylmethyl}-benzoic acid trifluoro-acetate was treated as in Example 57. The crude product was dissolved in water and sodium bicarbonate was added until pH 8. The aqueous layer was extracted with ethyl acetate and ethyl acetate/tetrahydrofuran. The combined organic layers were washed with water, 10% sodium carbonate, and brine then evaporated to a foam. The material was chromatographed on silica gel eluting with tetrahydrofuran to give the free base. The foam was dissolved in tetrahydrofuran and treated with 1 M HCl in diethyl ether (2.06 eq.) to give 4-{8-methyl-5,7-dioxo-2-[(pyridin-4-ylmethyl)-carbamoyl]-7H-thiazolo[3,2-c]pyrimidin-6-ylmethyl}-benzoic acid 2-dimethylamino-ethyl ester dihydrochloride (93%); MS (APCI+), m/z (%): 522(100), 388(20), 317(40), 135(20).

### EXAMPLE 64

### 8-Methyl-6-(2-methyl-thiazol-4-ylmethyl)-5,7-dioxo-6,7-dihydro-5H-thiazolo[3,2-c]pyrimidine-2-carboxylic acid 4-fluoro-benzylamide

8-Methyl-5,7-dioxo-6,7-dihydro-5H-thiazolo[3,2-c]pyrimidine-2-carboxylic acid 4-fluoro-benzylamide was treated as in Example 55, Step D with 2-methyl-4-bromomethyl-thiazole. The crude product was chromatographed with silica gel eluting with CH₂Cl₂:tetrahydrofuran 9:1 to give 8-methyl-6-(2-methyl-thiazol-4-ylmethyl)-5,7-dioxo-6,7-dihydro-5H-thiazolo[3,2-c]pyrimidine-2-carboxylic acid 4-fluoro-benzylamide (37%); MS (APCI+), m/z (%): 445(100), 294(80).

### EXAMPLE 65

### 2-Chloro-4-[2-(4-fluoro-benzylcarbamoyl)-8-methyl-5,7-dioxo-7H-thiazolo[3,2-c]pyrimidin-6-ylmethyl]-benzoic acid methyl ester

8-Methyl-5,7-dioxo-6,7-dihydro-5H-thiazolo[3,2-c]pyrimidine-2-carboxylic acid 4-fluoro-benzylamide was treated as in Example 55, Step D with 4-bromomethyl-2-chloro-benzoic acid methyl ester. The crude product was chromatographed on silica gel eluting with CH₂Cl₂:tetrahydrofuran 9:1, then triturated with diethyl ether to give 2-chloro-4-[2-(4-fluoro-benzylcarbamoyl)-8-methyl-5,7-dioxo-7H-thiazolo[3,2-c]pyrimidin-6-ylmethyl]-benzoic acid methyl ester (86%); mp 176-178° C.; MS (APCI+), m/z (%): 518(40), 516(100), 367(20), 365(50).

### EXAMPLE 66

### 8-Methyl-5,7-dioxo-6-(2H-tetrazol-5-ylmethyl)-6,7-dihydro-5H-thiazolo[3,2-c]pyrimidine-2-carboxylic acid 4-fluoro-benzylamide

### 8-Methyl-5,7-dioxo-6-[(2-triphenylmethyl)-2H-tetrazol-5-ylmethyl]-6,7-dihydro-5H-thiazolo[3,2-c]pyrimidine-2-carboxylic acid 4-fluoro-benzylamide

Step A: 8-Methyl-5,7-dioxo-6,7-dihydro-5H-thiazolo[3,2-c]pyrimidine-2-carboxylic acid 4-fluoro-benzylamide was treated as in Example 55, Step D with 5-chloromethyl-2-(triphenylmethyl)-2H-tetrazole. The crude product was chromatographed on silica gel eluting with CH₂Cl₂:tetrahydrofuran, 19:1 to give 8-methyl-5,7-dioxo-6-[(2-triphenylmethyl)-2H-tetrazol-5-ylmethyl]-6,7-dihydro-5H-thiazolo[3,2-c]pyrimidine-2-carboxylic acid 4-fluoro-benzylamide (50%);

¹H-NMR (DMSO-d₆) δ 9.32(t, 1H), 7.36(m, 11H), 7.16(t,2H), 6.95(m, 6H), 5.34(s, 2H), 4.39(s, 2H), 1.88(s, 3H).

### 8-Methyl-5,7-dioxo-6-(2H-tetrazol-5-ylmethyl)-6,7-dihydro-5H-thiazolo[3,2-c]pyrimidine-2-carboxylic acid 4-fluoro-benzylamide

Step B: The product from Example 66, Step A (295 mg, 0.45 mmol) was suspended in trifluoroacetic acid (5.0 mL). Triethylsilane was added dropwise until the mixture became colorless. The solvent was evopoarated in vacuo, and the residue triturated twice with diethyl ether. The material was vacuum dried at room temperature to give 8-methyl-5,7-dioxo-6-(2H-tetrazol-5-ylmethyl)-6,7-dihydro-5H-thiazolo[3,2-c]pyrimidine-2-carboxylic acid 4-fluoro-benzylamide (53%); MS (APCI+), m/z (%): 416(35), 373(25), 306(30), 265(95), 222(100), 209(20).

### EXAMPLE 67

### 8-Methyl-5,7-dioxo-6-thiazol-2-ylmethyl-6,7-dihydro-5H-thiazolo[3,2-c]pyrimidine-2-carboxylic acid 4-fluoro-benzylamide hydrochloride

8-Methyl-5,7-dioxo-6,7-dihydro-5H-thiazolo[3,2-c]pyrimidine-2-carboxylic acid 4-fluoro-benzylamide was treated as in Example 55, Step D with 4-chloromethyl-thiazole. The crude product was chromatographed on silica gel eluting with CH₂Cl₂:tetrahydrofuran, 5:1. The resulting free base was dissolved in tetrahydrofuran and treated with 1M HCl in diethyl ether (1.2 eq.) and stirred 30 minutes. The solid was isolated by filtration and dried in vacuo at room temperature to give 8-methyl-5,7-dioxo-6-thiazol-2-ylmethyl-6,7-dihydro-5H-thiazolo[3,2-c]pyrimidine-2-carboxylic acid 4-fluoro-benzylamide hydrochloride (44%); mp 183-185° C.; MS (APCI+), m/z (%): 431(100), 280(20).

### EXAMPLE 68

### 4-[2-(4-Fluoro-benzylcarbamoyl)-8-methyl-5,7-dioxo-7H-thiazolo[3,2-c]pyrimidin-6-ylmethyl]-2-methyl-benzoic acid methyl ester

8-Methyl-5,7-dioxo-6,7-dihydro-5H-thiazolo[3,2-c]pyrimidine-2-carboxylic acid 4-fluoro-benzylamide was treated as in Example 55, Step D with 4-bromomethyl-2-methyl-benzoic acid methyl ester. The crude product was chromatographed on silica gel eluting with CH₂Cl₂:tetrahydrofuran 9:1, then triturated with diethyl ether to give 4-[2-(4-fluoro-benzylcarbamoyl)-8-methyl-5,7-dioxo-7H-thiazolo[3,2-c]pyrimidin-6-ylmethyl]-2-methyl-benzoic acid methyl ester (75%); mp 179-180° C.; MS (APCI+), mlz (%): 496(100), 345(15).

### EXAMPLE 69

### 4-[2-(4-Fluoro-benzylcarbamoyl)-8-methyl-5,7-dioxo-7H-thiazolo[3,2-c]pyrimidin-6-ylmethyl]-2-methoxy-benzoic acid methyl ester

8-Methyl-5,7-dioxo-6,7-dihydro-5H-thiazolo[3,2-c]pyrimidine-2-carboxylic acid 4-fluoro-benzylamide was treated as in Example 55, Step D with 4-bromomethyl-2-methoxy-benzoic acid methyl ester. The crude product was chromatographed on silica gel eluting with CH₂Cl₂:tetrahydrofuran 19:1, then crystallized from diethyl ether and vacuum dried at room temperature to give 4-[2-(4-fluoro-benzylcarbamoyl)-8-methyl-5,7-dioxo-7H-thiazolo[3,2-c]pyrimidin-6-ylmethyl]-2-methoxy-benzoic acid methyl ester (75%); mp 191-193° C.; MS (APCI+), m/z (%): 512(100), 361(10).

### EXAMPLE 70

### 6-(4-Fluoro-benzyl)-8-methyl-5,7-dioxo-6,7-dihydro-5H-thiazolo[3,2-c]pyrimidine-2-carboxylic acid(pyridin-4-ylmethyl)-amide hydrochloride

8-Methyl-5,7-dioxo-6,7-dihydro-5H-thiazolo[3,2-c]pyrimidine-2-carboxylic acid(pyridin-4-ylmethyl)-amide was treated as in Example 55, Step D with 4-fluorobenzyl bromide. The crude product was chromatographed on silica gel eluting with CH₂Cl₂:tetrahydrofuran 2:1. The isolated free base was dissolved in tetrahydrofuran and treated with 1M HCl in diethyl ether to give 6-(4-fluoro-benzyl)-8-methyl-5,7-dioxo-6,7-dihydro-5H-thiazolo[3,2-c]pyrimidine-2-carboxylic acid(pyridin-4-ylmethyl)-amide hydrochloride (48%); MS (APCI+), m/z (%): 425(100), 291(40), 257(20).

### EXAMPLE 71

### 6-(4-Bromo-benzyl)-8-methyl-5,7-dioxo-6,7-dihydro-5H-thiazolo[3,2-c]pyrimidine-2-carboxylic acid(pyridin-4-ylmethyl)-amide hydrochloride

8-Methyl-5,7-dioxo-6,7-dihydro-5H-thiazolo[3,2-c]pyrimidine-2-carboxylic acid(pyridin-4-ylmethyl)-amide was treated as in Example 55, Step D with 4-bromobenzyl bromide. The crude product was chromatographed on silica gel eluting with CH₂Cl₂:tetrahydrofuran, 2:1. The isolated free base was dissolved in tetrahydrofuran and treated with 1M HCl in diethyl ether to give 6-(4-bromo-benzyl)-8-methyl-5,7-dioxo-6,7-dihydro-5H-thiazolo[3,2-c]pyrimidine-2-carboxylic acid(pyridin-4-ylmethyl)-amide hydrochloride (48%); MS (APCI+), m/z (%): 487(100), 485(100).

### EXAMPLE 72

### 6-(4-Chloro-benzyl)-8-methyl-5,7-dioxo-6,7-dihydro-5H-thiazolo[3,2-c]pyrimidine-2-carboxylic acid(pyridin-4-ylmethyl)-amide

8-Methyl-5,7-dioxo-6,7-dihydro-5H-thiazolo[3,2-c]pyrimidine-2-carboxylic acid(pyridin-4-ylmethyl)-amide was treated as in Example 55, Step D with 4-chlorobenzyl bromide. The crude product was chromatographed on silica gel eluting with CH₂Cl₂:tetrahydrofuran, 2:1. The isolated free base was dissolved in tetrahydrofuran and treated with 1M HCl in diethyl ether to give 6-(4-chloro-benzyl)-8-methyl-5,7-dioxo-6,7-dihydro-5H-thiazolo[3,2-c]pyrimidine-2-carboxylic acid(pyridin-4-ylmethyl)-amide hydrochloride (65%); MS (APCI+), m/z (%): 441(100), 307(20).

### EXAMPLE 73

### 8-Methyl-6-[4-(morpholine-4-carbonyl)-benzyl]-5,7-dioxo-6,7-dihydro-5H-thiazolo[3,2-c]pyrimidine-2-carboxylic acid(pyridin-4-ylmethyl)-amide hydrochloride

8-Methyl-5,7-dioxo-6,7-dihydro-5H-thiazolo[3,2-c]pyrimidine-2-carboxylic acid(pyridin-4-ylmethyl)-amide was treated as in Example 55, Step D with 1-(4-bromomethylphenyl)-1-morpholin-4-ylmethanone. The crude product was triturated with ethyl acetate to give the free base. The isolated free base was dissolved in tetrahydrofuran and treated with 1M HCl in diethyl ether to give 8-methyl-6-[4-(morpholine-4-carbonyl)-benzyl]-5,7-dioxo-6,7-dihydro-5H-thiazolo[3,2-c]pyrimidine-2-carboxylic acid(pyridin-4-ylmethyl)-amide hydrochloride (56%); MS (APCI+), m/z (%): 520(80), 392(100), 336(70), 292(80).

### EXAMPLE 74

### {5-[2-(4-Fluoro-benzylcarbamoyl)-8-methyl-5,7-dioxo-7H-thiazolo[3,2-c]pyrimidin-6-ylmethyl]-isoxazol-3-yl}-carbamic acid methyl ester

8-Methyl-5,7-dioxo-6,7-dihydro-5H-thiazolo[3,2-c]pyrimidine-2-carboxylic acid 4-fluoro-benzylamide (100 mg, 0.3 mmol) was dissolved in dimethylformamide (3 mL), and cesium carbonate (98 mg, 0.3 mmol) was added, then (5-bromomethyl-isoxazol-3-yl)-carbamic acid methyl ester (71 mg, 0.30 mmol) was added and the mixture stirred overnight at room temperature. The reaction mixture was partitioned between ethyl acetate and 10% citric acid solution, the layers separated, the organic layer washed with brine, dried over magnesium sulfate, filtered and concentrated. The residue was chromatographed on silica gel (70-230 mesh) using hexanes/ethyl acetate, 1:1, v/v, then 1/2, v/v as eluant to give the product, {5-[2-(4-fluoro-benzylcarbamoyl)-8-methyl-5,7-dioxo-7H-thiazolo[3,2-c]pyrimidin-6-ylmethyl]-isoxazol-3-yl}-carbamic acid methyl ester, 58 mg (40%). MS (APCI+), m/z (%): 488.1(100), 265.2(98).

### EXAMPLE 75

### 8-Methyl-5,7-dioxo-6-[4-(2H-tetrazol-5-yl)-benzyl]-6,7-dihydro-5H-thiazolo[3,2-c]pyrimidine-2-carboxylic acid 4-fluoro-benzylamide

Step A: 8-Methyl-5,7-dioxo-6,7-dihydro-5H-thiazolo[3,2-c]pyrimidine-2-carboxylic acid 4-fluoro-benzylamide (200 mg, 0.6 mmol) was dissolved in dimethylformamide (5 mL), and cesium carbonate (196 mg, 0.6 mmol) was added; then (4-bromomethyl-phenyl)-trityl-2H -tetrazole (289 mg, 0.6 mmol) was added and the mixture stirred over three days at room temperature. The reaction mixture was partitioned between ethyl acetate and 10% citric acid solution, the aqueous layer back-extracted, the organic layers combined, the organic layer washed with 10% citric acid and three times with brine, dried over magnesium sulfate, filtered and concentrated. The residue was chromatographed on silica gel (70-230 mesh) using hexanes/ethyl acetate, 2:1, v/v, then 1:1, v/v as eluant to give the product, which was used directly in the next step, 186 mg (42%). MS (APCI+), m/z (%): 678.3(10), 243.2(100).

Step B: The product from Step A, (186 mg, 0.25 mmol) was taken up in trifluoroacetic acid (6 mL) at room temperature and stirred for 3 hours. The reaction mixture was concentrated to dryness, and water was added and a white precipitate formed and was collected by filtration and washed with ethyl ether and hexanes to give the product, 8-methyl-5,7-dioxo-6-[4-(2H-tetrazol-5-yl)-benzyl]-6,7-dihydro-5H-thiazolo[3,2-c]pyrimidine-2-carboxylic acid 4-fluoro-benzylamide, 80 mg (65%). MS (APCI+), m/z (%): 492.2(60).

### EXAMPLE 76

### 8-Methyl-6-[4-(morpholine-4-carbonyl)-benzyl]-5,7-dioxo-6,7-dihydro-5H-thiazolo[3,2-c]pyrimidine-2-carboxylic acid 4-fluoro-benzylamide

8-Methyl-5,7-dioxo-6,7-dihydro-5H-thiazolo[3,2-c]pyrimidine-2-carboxylic acid 4-fluoro-benzylamide (150 mg, 0.45 mmol) was dissolved in dimethylformamide (4 mL), and cesium carbonate (148 mg, 0.45 mmol) was added and then 1-(4-bromomethyl-phenyl)-1-morpholino4-ylmethanone (128 mg, 0.45 mmol) was added and the mixture stirred four days at room temperature. The dimethylformamide was removed in vacuo. The residue was partitioned between ethyl acetate and 10% citric acid solution, the layers separated, the organic layer washed with brine, dried over magnesium sulfate, filtered and concentrated. The residue was triturated with hexanes/ethyl acetate to give the product, 8-methyl-6-[4-(morpholine-4-carbonyl)-benzyl]-5,7-dioxo-6,7-dihydro-5H-thiazolo[3,2-c]pyrimidine-2-carboxylic acid 4-fluoro-benzylamide), 143 mg (59%). MS (APCI+), m/z (%): 537.2(50), 386.2 (100).

### EXAMPLE 77

### 6-(6-Fluoro-quinolin-2-ylmethyl)-8-methyl-5,7-dioxo-6,7-dihydro-5H-thiazolo[3,2-c]pyrimidine-2-carboxylic acid 4-fluoro-benzylamide

8-Methyl-5,7-dioxo-6,7-dihydro-5H-thiazolo[3,2-c]pyrimidine-2-carboxylic acid 4-fluoro-benzylamide (150 mg, 0.45 mmol) was dissolved in dimethylformamide (4 mL) and cesium carbonate (148 mg, 0.45 mmol) was added and then 2-(chloromethyl)-6-fluoro-quinoline (88 mg, 0.45 mmol) was added and the mixture stirred 4 days at room temperature. The dimethylformamide was removed in vacuo. The residue was partitioned between ethyl acetate and water, the layers separated, the organic layer washed with brine, dried over magnesium sulfate, filtered and concentrated. The residue was triturated with hexanes/ethyl acetate to give the product, 6-(6-fluoro-quinolin-2-ylmethyl)-8-methyl-5,7-dioxo-6,7-dihydro-5H-thiazolo[3,2-c]pyrimidine-2-carboxylic acid 4-fluoro-benzylamide, 143 mg (64%). MS (APCI+), m/z (%): 493.2(50), 342.2 (100).

### EXAMPLE 78

### 2-[2-(4-Fluoro-benzylcarbamoyl)-8-methyl-5,7-dioxo-7H-thiazolo[3,2-c]pyrimidin-6-ylmethyl]-5-methoxy-pyrimidine-4-carboxylic acid methyl ester

8-Methyl-5,7-dioxo-6,7-dihydro-5H-thiazolo[3,2-c]pyrimidine-2-carboxylic acid 4-fluoro-benzylamide (150 mg, 0.45 mmol) was dissolved in dimethylformamide (4 mL) and cesium carbonate (148 mg, 0.45 mmol) was added and then 2-chloromethyl-5-methoxy-pyrimidine-4-carboxylic acid methyl ester (117 mg, 0.45 mmol) was added, and the mixture stirred 4 days at room temperature. The dimethylformamide was removed in vacuo. The residue was partitioned between ethyl acetate and water, the layers separated, the organic layer washed with brine, dried over magnesium sulfate, filtered and concentrated. The residue was triturated with hexanes/ethyl acetate to give the product, 2-[2-(4-fluoro-benzylcarbamoyl)-8-methyl-5,7-dioxo-7H-thiazolo[3,2-c]pyrimidin-6-ylmethyl]-5-methoxy-pyrimidine-4-carboxylic acid methyl ester, 30 mg (13%). MS (APCI+), m/z (%): 514.2(100), 363.2 (50).

### EXAMPLE 79

### 6-But-2-ynyl-8-methyl-5,7-dioxo-6,7-dihydro-5H-thiazolo[3,2-c]pyrimidine-2-carboxylic acid 4-fluoro-benzylamide

8-Methyl-5,7-dioxo-6,7-dihydro-5H-thiazolo[3,2-c]pyrimidine-2-carboxylic acid 4-fluoro-benzylamide (150 mg, 0.45 mmol) was dissolved in dimethylformamide (4 mL) and cesium carbonate (148 mg, 0.45 mmol) was added, and then 1-bromo-2-butyne (60 mg, 0.45 mmol) was added and the mixture stirred 5 days at room temperature. The dimethylformamide was removed in vacuo. The residue was partitioned between ethyl acetate and 10% citric acid solution, the layers separated, the organic layer washed with brine, dried over magnesium sulfate, filtered and concentrated. The residue was triturated with hexanes/ethyl acetate to give the product, 6-but-2-ynyl-8-methyl-5,7-dioxo-6,7-dihydro-5H-thiazolo[3,2-c]pyrimidine-2-carboxylic acid 4-fluoro-benzylamide, 125 mg (72%). MS (APCI+), m/z (%): 386.2(100).

### EXAMPLE 80

### 8-Methyl-5,7-dioxo-6-(2-oxo-2H-1-benzopyran-6-ylmethyl)-6,7-dihydro-5H-thiazolo[3,2-c]pyrimidine-2-carboxylic acid 4-fluoro-benzylamide

8-Methyl-5,7-dioxo-6,7-dihydro-5H-thiazolo[3,2-c]pyrimidine-2-carboxylic acid 4-fluoro-benzylamide (377 mg, 1.1 mmol) was dissolved in dimethylformamide (7 mL) and cesium carbonate (365 mg, 1.1 mmol) was added, and then 6-bromomethyl-chromen-2-one (270 mg, 1.1 mmol) was added and the mixture stirred 13 days at room temperature. The dimethylformamide was removed in vacuo. The residue was partitioned between ethyl acetate and 10% citric acid solution, the layers separated, the organic layer washed with 10% citric acid and brine, dried over magnesium sulfate, filtered and concentrated. The residue was triturated with hexanes/ethyl acetate and then chromatographed on a short pad of silica gel (70-230 mesh) followed by column chromatography using hexanes/ethyl acetate to give the product, 8-methyl-5,7-dioxo-6-(2-oxo-2H-1-benzopyran-6-ylmethyl)-6,7-dihydro-5H-thiazolo[3,2-c]pyrimidine-2-carboxylic acid 4-fluoro-benzylamide, 78 mg (14%). MS (APCI+), m/z (%): 492.3(100), 341.1 (80).

### EXAMPLE 81

### 6-(4-Methanesulfonyl-benzyl)-8-methyl-5,7-dioxo-6,7-dihydro-5H-thiazolo[3,2-c]pyrimidine-2-carboxylic acid(pyridin-4-ylmethyl)-amide hydrochloride

The product from Example 62, Step A, namely 8-methyl-5,7-dioxo-6,7-dihydro-5H-thiazolo[3,2-c]pyrimidine-2-carboxylic acid(pyridin-4-ylmethyl)-amide (158 mg, 0.5 mmol) was dissolved in dimethylformamide (5 mL), and cesium carbonate (163 mg, 0.5 mmol) was added followed by 4-methylsulfonylbenzyl chloride (102 mg, 0.5 mmol), and the mixture stirred overnight at room temperature. The dimethylformamide was removed in vacuo. The residue was partitioned between ethyl acetate and water, the layers separated, the organic layer washed with brine, dried over magnesium sulfate, filtered and concentrated. No product was in the ethyl acetate layer. The product was insoluble in both phases. The insoluble material was collected by filtration and dried in vacuo. The solid was stirred in ethereal HCl to give the product, 6-(4-methanesulfonyl-benzyl)-8-methyl-5,7-dioxo-6,7-dihydro-5H-thiazolo[3,2-c]pyrimidine-2-carboxylic acid(pyridin-4-ylmethyl)-amide hydrochloride, 0.082 g (32%). MS (APCI+), m/z (%): 485.1(100), 351.0 (50).

### EXAMPLE 82

### 6-(3-Cyano-benzyl)-8-methyl-5,7-dioxo-6,7-dihydro-5H-thiazolo[3,2-c]pyrimidine-2-carboxylic acid(pyridin-4-ylmethyl)-amide hydrochloride

The product from Example 62, Step A, namely 8-methyl-5,7-dioxo-6,7-dihydro-5H-thiazolo[3,2-c]pyrimidine-2-carboxylic acid(pyridin-4-ylmethyl)-amide (158 mg, 0.5 mmol) was dissolved in dimethylformamide (5 mL) and cesium carbonate (163 mg, 0.5 mmol) was added and then 3-cyanobenzyl bromide (98 mg, 0.5 mmol) was added, and the mixture stirred overnight at room temperature. The dimethylformamide was removed in vacuo. The residue was partitioned between ethyl acetate and water, the layers separated, the organic layer washed with brine, dried over magnesium sulfate, filtered and concentrated. The residue was triturated with hexanes/ethyl acetate to give the product, 151 mg (70%). A portion of the solid was stirred in ethereal HCl to give the product, 6-(3-cyano-benzyl)-8-methyl-5,7-dioxo-6,7-dihydro-5H-thiazolo[3,2-c]pyrimidine-2-carboxylic acid(pyridin-4-ylmethyl)-amide hydrochloride, 0.082 g. MS (APCI+), m/z (%): 432.1(100), 298.1 (50).

### EXAMPLE 83

### 6-[2-(4-Chloro-benzenesulfonyl)-ethyl]-8-methyl-5,7-dioxo-6,7-dihydro-5H-thiazolo[3,2-c]pyrimidine-2-carboxylic acid(pyridin-4-ylmethyl)-amide hydrochloride

The product from Example 62, Step A, namely 8-methyl-5,7-dioxo-6,7-dihydro-5H-thiazolo[3,2-c]pyrimidine-2-carboxylic acid(pyridin-4-ylmethyl)-amide (158 mg, 0.5 mmol) was dissolved in dimethylformamide (5 mL), and cesium carbonate (163 mg, 0.5 mmol) was added; then 2-chloroethyl-4-chlorophenyl sulfone (120 mg, 0.5 mmol) was added, and the mixture stirred overnight at room temperature. The dimethylformamide was removed in vacuo. The residue was partitioned between ethyl acetate and water, the layers separated, the organic layer washed with brine, dried over magnesium sulfate, filtered and concentrated. The solid was stirred 2 hours in ethereal HCl to give the product, 6-[2-(4-chloro-benzenesulfonyl)-ethyl)-8-methyl-5,7-dioxo-6,7-dihydro-5H-thiazolo[3,2-c]pyrimidine-2-carboxylic acid(pyridin-4-ylmethyl)-amide hydrochloride, 156 mg (57%). MS (APCI+), m/z (%): 519.1(40), 521.1 (20).

### EXAMPLE 84

### 8-Methyl-5,7-dioxo-6-(4-sulfamoyl-benzyl)-6,7-dihydro-5H-thiazolo[3,2-c]pyrimidine-2-carboxylic acid(pyridin-4-ylmethyl)-amide hydrochloride

The product from Example 62, Step A, namely 8-methyl-5,7-dioxo-6,7-dihydro-5H-thiazolo[3,2-c]pyrimidine-2-carboxylic acid(pyridin-4-ylmethyl)-amide (158 mg, 0.5 mmol) was dissolved in dimethylformamide (5 mL), and cesium carbonate (163 mg, 0.5 mmol) was added; then 4-bromomethylbenzene sulfonamide (125 mg, 0.5 mmol) was added and the mixture stirred overnight at room temperature. The dimethylformamide was removed in vacuo. The residue was partitioned between ethyl acetate and water, the layers separated, the organic layer washed with brine, dried over magnesium sulfate, filtered and concentrated. The solid was stirred 1 hour in ethereal HCl to give the product, 8-methyl-5,7-dioxo-6-(4-sulfamoyl-benzyl)-6,7-dihydro-5H-thiazolo[3,2-c]pyrimidine-2-carboxylic acid(pyridin-4-ylmethyl)-amide hydrochloride, 145 mg (56%). MS (APCI+), m/z (%): 486.1(100), 352.0 (90).

### EXAMPLE 85

### 6-(4-Cyano-benzyl)-8-methyl-5,7-dioxo-6,7-dihydro-5H-thiazolo[3,2-c]pyrimidine-2-carboxylic acid(pyridin-4-ylmethyl)-amide hydrochloride

The product from Example 62, Step A, namely 8-methyl-5,7-dioxo-6,7-dihydro-5H-thiazolo[3,2-c]pyrimidine-2-carboxylic acid(pyridin-4-ylmethyl)-amide (158 mg, 0.5 mmol) was dissolved in dimethylformamide (5 mL), and cesium carbonate (163 mg, 0.5 mmol) was added; then 4-cyanobenzyl bromide (98 mg, 0.5 mmol) was added and the mixture stirred overnight at room temperature. The dimethylformamide was removed in vacuo. The residue was partitioned between ethyl acetate and water, the layers separated, the organic layer washed with brine, dried over magnesium sulfate, filtered and concentrated. The residue was triturated with hexanes/ethyl acetate to give a pale brown solid. The solid was stirred in ethereal HCl for 1 hour to give the product, 6-(4-cyano-benzyl)-8-methyl-5,7-dioxo-6,7-dihydro-5H-thiazolo[3,2-c]pyrimidine-2-carboxylic acid(pyridin-4-ylmethyl)-amide hydrochloride, 0.102 g. MS (AP+) m/z (%): 432.1(100), 298.0 (30).

### EXAMPLE 86

### 8-Methyl-5,7-dioxo-6-(3-oxo-3-phenyl-propyl)-6,7-dihydro-5H-thiazolo[3,2-c]pyrimidine-2-carboxylic acid 4-fluoro-benzylamide

In an 8 mL screw cap vial was added a solution of 8-methyl-5,7-dioxo-6,7-dihydro-5H-thiazolo[3,2-c]pyrimidine-2-carboxylic acid 4-fluoro-benzylamide, (0.033 g, 0.1 mmol) in dimethylformamide (1 mL), a solution of 3-chloro-1-phenyl-propan-1-one (0.039 g, 0.23 mmol) in dimethylformamide (0.575 mL) and anhydrous cesium carbonate (0.075 g, 0.023 mmol). The vial was capped and the reaction mixture was shaken for 24 hours at room temperature. The reaction mixture was filtered and the solvent was removed under vacuum. Purification was carried out via reverse-phase HPLC (3% n-propanol in acetonitrile and 3% n-propanol in water as the eluent; C-18 column). 0.028 g (60%). MS (APCI), m/z ([M+H]⁺) 466.5.

### EXAMPLE 87

### 8-Methyl-5,7-dioxo-6-(1-phenyl-ethyl)-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 438.4.

### EXAMPLE 88

### 8-Methyl-5,7-dioxo-6-(2-phenylmethanesulfonyl-ethyl)-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 516.5.

### EXAMPLE 89

### 6-(5-Cyano-pentyl)-8-methyl-5,7-dioxo-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 429.4.

### EXAMPLE 90

### 6-(E)-But-2-enyl-8-methyl-5,7-dioxo-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 388.4.

### EXAMPLE 91

### 8-Methyl-5,7-dioxo-6-(E)-pent-2-enyl-6,7-dihydro-5H-thiazolo[3,2-c]pyrimidine-2-carboxylic acid 4-fluoro-benzylamide

The reaction was run according to the procedure of Example 86.

MS (APCI), mn/z ([M+H]⁺) 402.4.

### EXAMPLE 92

### 6-sec-Butyl-8-methyl-5,7-dioxo-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 390.4.

### EXAMPLE 93

### 8-Methyl-6-(2-methyl-allyl)-5,7-dioxo-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 388.4.

### EXAMPLE 94

### 6-(1-Ethyl-propyl)-8-methyl-5,7-dioxo-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 404.4.

### EXAMPLE 95

### 8-Methyl-5,7-dioxo-6-pent-2-ynyl-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 400.4.

### EXAMPLE 96

### 6-(2-Benzenesulfonyl-ethyl)-8-methyl-5,7-dioxo-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 502.5.

### EXAMPLE 97

### 8-Methyl-6-(3-methyl-but-2-enyl)-5,7-dioxo-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 402.4.

### EXAMPLE 98

### 6-[2-(4-Fluoro-benzenesulfonyl)-ethyl]-8-methyl-5,7-dioxo-6,7dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 520.5.

### EXAMPLE 99

### 6-[3-(4-Fluoro-phenyl)-3-oxo-propyl]-8-methyl-5,7-dioxo-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 484.4.

### EXAMPLE 100

### 6-(2-Benzoylamino-ethyl)-8-methyl-5,7-dioxo-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 481.5.

### EXAMPLE 101

### 8-Methyl-5,7-dioxo-6-(2-phenoxy-ethyl)-6,7-dihydro-5H-thiazolo[3,2-c]pyrimidine-2-carboxylic acid 4-fluoro-benzylamide

The reaction was run according to the procedure of Example 86.

MS (APCI), m/z ([M+H]⁺) 454.4.

### EXAMPLE 102

### 6-(3,4-Dichloro-benzyl)-5,7-dioxo-6,7-dihydro-5H-thiazolo[3,2-c]pyrimidine-2-carboxylic acid 4-methoxy-benzylamide

Lithium hexamethyldisilazane (0.9 mL, 1 M in THF, 0.9 mmol) was added to a solution of 6-(3,4-dichlorobenzyl)-thiazolo[3,2-c]pyrimidine-5,7-dione (Example 12, Step A, 0.200 g, 0.61 mmol) in tetrahydrofuran (10 mL), under nitrogen at −72° C. After 3 minutes, 1-isocyanatomethyl-4-methoxy-benzene (0.22 mL, 1.5 mmol) was added. The reaction was stirred 15 minutes, then aqueous ammonium chloride was added, and the reaction allowed to warm to room temperature. EtOAc (50 mL) was added to the reaction, water layer was removed, and the organic layer was, dried (Na₂SO₄) and evaporated. The residue was chromatographed on silica gel eluting with CH₂Cl₂:EtOAc, 9:1. The isolated product was triturated with diethyl ether and dried in vacuum to give 45.2 mg (15%) of 6-(3,4-dichlorobenzyl)-5,7-dioxo-6,7-dihydro-5H-thiazolo[3,2-c]pyrimidine-2-carboxylic acid 4-methoxy-benzylamide: mp 206-207° C.; MS (APCI+), m/z (%): 493(15), 492(80), 490(100), 329(40), 326(55), 263(121(30).

### EXAMPLE 103

### 4-[2-(4-Methoxy-benzylcarbamoyl)-8-methyl-5,7-dioxo-7H-thiazolo[3,2-c]pyrimidin-6-ylmethyl]-benzoic acid methyl ester

### 8-methyl-5,7-dioxo-7H-thiazolo[3,2-c]pyrimidin-6-ylmethyl]-benzoic acid methyl ester

Step A: 8-Methyl-thiazolo[3,2-c]pyrimidine-5,7-dione (4.55 g, 25.0 mmol) was suspended in dimethylformamide (100 mL) under a nitrogen atmosphere. Cesium carbonate (8.96 g, 27.5 mmol) was added followed by 4-bromomethyl-benzoic acid methyl ester (5.96 g, 26.0 mmol). The reaction was stirred at 50° C. for 4 hours. The solvent was evaporated in vacuo. The resulting solid was triturated with diethyl ether to give 8-methyl-5,7-dioxo-7H-thiazolo[3,2-c]pyrimidin-6-ylmethyl]-benzoic acid methyl ester (91%); MS (APCI+), m/z (%): 331(100), 661(15).

### 4-[2-(4-Methoxy-benzylcarbamoyl)-8-methyl-5,7-dioxo-7H-thiazolo[3,2-c]pyrimidin-6-ylmethyl]-benzoic acid methyl ester

Step B: The reaction was run as in Example 102 to give 4-[2-(4-methoxy-benzylcarbamoyl)-8-methyl-5,7-dioxo-7H-thiazolo[3,2-c]pyrimidin-6-ylmethyl]-benzoic acid methyl ester (23%): mp 163-165° C. MS (APCI+), m/z (%): 494(100), 331(50), 121(40).

### EXAMPLE 104

### 4-(8-Methyl-5,7-dioxo-7H-thiazolo[3,2-c]pyrimidin-6-ylmethyl)-benzoic acid tert-butyl ester

The reaction was run as in Example 103, Step A, using 4-bromomethyl-benzoic acid tert-butyl ester. 4-(8-Methyl-5,7-dioxo-7H-thiazolo[3,2-c]pyrimidin-6-ylmethyl)-benzoic acid tert-butyl ester was isolated (90%): mp 163-165° C. MS (APCI+), m/z (%): 373(90), 317(100).

### EXAMPLE 105

### 4-[2-(3-Fluoro-benzylcarbamoyl)-8-methyl-5,7-dioxo-7H-thiazolo[3,2-c]pyrimidin-6-ylmethyl]-benzoic acid methyl ester

8-Methyl-5,7-dioxo-7H-thiazolo[3,2-c]pyrimidin-6-ylmethyl]-benzoic acid methyl ester was treated as in Example 102 using 1-fluoro-3-isocyanatomethyl-benzene. The resulting solid was purified by flash chromatography on silica gel eluting with CH₂Cl₂:THF, 19:1 to give 4-[2-(3-fluoro-benzylcarbamoyl)-8-methyl-5,7-dioxo-7H-thiazolo[3,2-c]pyrimidin-6-ylmethyl]-benzoic acid methyl ester (36%): mp 209-211° C.; MS (APCI+), m/z (%): 482(100), 331(75).

### EXAMPLE 106

### 4-[2-(4-Fluoro-benzylcarbamoyl)-8-methyl-5,7-dioxo-7H-thiazolo[3,2-c]pyrimidin-6-ylmethyl]-benzoic acid methyl ester

8-Methyl-5,7-dioxo-7H-thiazolo[3,2-c]pyrimidin-6-ylmethyl]-benzoic acid methyl ester was treated as in Example 102 using 1-fluoro4-isocyanatomethyl-benzene. The resulting solid was purified by flash chromatography on silica gel eluting with CH₂Cl₂:tetrahydrofuran, 24:1 to give 4-[2-(4-fluoro-benzylcarbamoyl)-8-methyl-5,7-dioxo-7H-thiazolo[3,2-c]pyrimidin-6-ylmethyl]-benzoic acid methyl ester (30%): mp >215° C.; MS (APCI+), m/z (%): 482(100), 331(70).

### EXAMPLE 107

### 6-(4-Cyano-benzyl)-8-methyl-5,7-dioxo-6,7-dihydro-5H-thiazolo[3,2-c]pyrimidine-2-carboxylic acid 4-fluoro-benzylamide

### 6-(4-Cyano-benzyl)-8-methyl-5,7-dioxo-6,7-dihydro-5H-thiazolo[3,2-c]pyrimidine-2-carboxylic acid

Step A: The product of Example 53, namely 8-methyl-thiazolo[3,2-c]pyrimidine-5,7-dione-2-carboxylic acid was reacted with 4-bromomethyl-benzonitrile according to the procedure of Example 103, Step A, to give 6-(4-cyano-benzyl)-8-methyl-5,7-dioxo-6,7-dihydro-5H-thiazolo[3,2-c]pyrimidine-2-carboxylic acid (36%); MS (APCI+), m/z (%): 298(100).

### 6-(4-Cyano-benzyl)-8-methyl-5,7-dioxo-6,7-dihydro-5H-thiazolo[3,2-c]pyrimidine-2-carboxylic acid 4-fluoro-benzylamide

Step B: 6-(4-Cyano-benzyl)-8-methyl-5,7-dioxo-6,7-dihydro-5H-thiazolo[3,2-c]pyrimidine-2-carboxylic acid was treated as in Example 102 using 1-fluoro4-isocyanatomethyl-benzene. The crude product was chromatographed on silica gel eluting with CH₂Cl₂:tetrahydrofuran, 19:1. The product was then triturated with diethyl ether and vacuum dried at room temperature to give 6-(4-cyano-benzyl)-8-methyl-5,7-dioxo-6,7-dihydro-5H-thiazolo[3,2-c]pyrimidine-2-carboxylic acid 4-fluoro-benzylamide (25%): mp >220° C.; MS (APCI+), m/z (%): 449(100), 298(85).

### EXAMPLE 108

### 8-Methyl-6-[4-(morpholine-4-sulfonyl)-benzyl]-thiazolo[3,2-c]pyrimidine-5,7-dione

The product from Example 53, namely 8-methyl-thiazolo[3,2-c]pyrimidine-5,7-dione (586 mg, 3.2 mmol), 4-morpholinosulfonylbenzyl bromide, (1.031 g, 3.2 mmol), cesium carbonate (1.105 g, 3.4 mmol) and dimethylformamide (9 mL) were stirred at room temperature overnight. The dimethylformamide was removed on the rotary evaporator at 60° C. The residue was partitioned between ethyl acetate (200 mL) and water (100 mL), the layers separated, the organic layer washed with brine (50 mL) and dried over magnesium sulfate, filtered and concentrated to a tan solid. Trituration with hexanes/ethyl acetate and filtration gave the product, 8-methyl-6-[4-(morpholine-4-sulfonyl)-benzyl]-thiazolo[3,2-c]pyrimidine-5,7-dione, as a tan solid, 1.195 g (88%). MS (APCI+), m/z (%): 422.1(100), 183.1 (25).

### EXAMPLE 109

### 6-Benzyl-8-methyl-5,7-dioxo-6,7-dihydro-5H-thiazolo[3,2-c]pyrimidine-2-carboxylic acid 3-methoxy-benzylamide

The product of Example 33, Step B, namely 6-benzyl-8-methyl-5,7-dioxo-6,7-dihydro-5H-thiazolo[3,2-c]pyrimidine-2-carboxylic acid (158 mg, 0.50 mmol) was dissolved in dimethylformamide (4 mL). Added were 3-methoxybenzyl amine (64 mg, 50 mmol), 1-hydroxybenzotriazole hydrate (68 mg, 0.50 mmol) and 1-[3-(dimethylamino)propyl]-3-ethylcarbodiimide hydrochloride (100 mg, 0.52 mmol). The mixture was stirred overnight at room temperature. The reaction mixture was concentrated on the rotary evaporator. The residue was partitioned between ethyl acetate and water. The layers were separated, and the organic layer was washed with water, saturated sodium bicarbonate solution, and brine. The layers were separated, and the organic layer dried over magnesium sulfate, filtered, and evaporated in vacuo. The residue was triturated with diethyl ether/hexanes/ethyl acetate to give the product, 6-benzyl-8-methyl-5,7-dioxo-6,7-dihydro-5H-thiazolo[3,2-c]pyrimidine-2-carboxylic acid 3-methoxy-benzylamide, 164 mg (75%). MS (APCI+), m/z (%): 436(100), 273(90).

### EXAMPLE 110

### 6-Benzyl-8-methyl-5,7-dioxo-6,7-dihydro-5H-thiazolo[3,2-c]pyrimidine-2-carboxylic acid(tetrahydro-furan-2-ylmethyl)-amide

The product of Example 33, Step B, namely 6-benzyl-8-methyl-5,7-dioxo-6,7-dihydro-5H-thiazolo[3,2-c]pyrimidine-2-carboxylic acid (158 mg, 0.50 mmol) was dissolved in dimethylformamide (4 mL). Added were tetrahydrofurfuryl amine (51 mg, 50 mmol), 1-hydroxybenzotriazole hydrate (68 mg, 0.50 mmol) and 1-[3-(dimethylamino)propyl]-3-ethylcarbodiimide hydrochloride (100 mg, 0.52 mmol). The mixture was stirred 13 days at room temperature. The reaction mixture was concentrated on the rotary evaporator. The residue was partitioned between ethyl acetate and water. The layers were separated, and the organic layer was washed with water, saturated sodium bicarbonate solution, and brine. The layers were separated and the organic layer dried over magnesium sulfate, filtered, and evaporated in vacuo to give the product, 6-benzyl-8-methyl-5,7-dioxo-6,7-dihydro-5H-thiazolo[3,2-c]pyrimidine-2-carboxylic acid(tetrahydro-furan-2-ylmethyl)-amide, 147 mg (73%). MS (APCI+), m/z (%): 400(100), 273(50).

### EXAMPLE 111

### {5-[2-(4-Fluoro-benzylcarbamoyl)-8-methyl-5,7-dioxo-7H-thiazolo[3,2-c]pyrimidine-6-ylmethyl]-isoxazol-3-yl}-carbamic acid methyl ester

MS-APCI (M+1): 489.5

### EXAMPLE 112

### 6-Benzyl-8-formyl-5,7-dioxo-6,7-dihydro-5H-thiazolo[3,2-c]pyrimidine-2-carboxylic acid 4-fluoro-benzylamide

### (1-Benzyl-2,6-dioxo-1,2,3,6-tetrahydro-pyrimidin-4-ylsulfanyl)-acetic acid methyl ester

Step A: 3-Benzyl-6-chloro-1H-pyrimidine-2,4-dione (23.7 g, 100 mmol), methyl thioglycolate (11 mL, 123 mmol), and dimethylformamide (100 mL) were heated at 55° C. for about one hour. Then triethylamine (15 mL, 108 mmol) was added, and the mixture heated at 70° C. for 2 hours. The mixture was then stirred for 3 days at room temperature at which time more methyl thioglycolate (4.3 mL, 44.6 mmol) and triethylamine (94.5 mL, 30 mmol) were added, and the mixture was heated at 70° C. for 1 hour. The volatiles were removed on a rotary evaporator (80° C.), and the residue was partitioned between ethyl acetate (400 mL) and water (400 mL). The layers were separated, and the organic layer was washed with 10% aqueous citric acid solution (100 mL) and brine (100 mL), and dried over magnesium sulfate. The organic layer was filtered and concentrated. The aqueous washes were extracted again with ethyl acetate (400 mL) and the organic layer washed with brine (100 mL) and dried over magnesium sulfate, filtered and combined with the first ethyl acetate wash. The solution was concentrated to a yellow solid. The solid was triturated with ethyl acetate/hexanes (1:1), and the resulting solid collected by filtration; yield 18.6 g. MS (APCI+), m/z (%): 307.1(100), 275.0 (40).

### 6-Benzyl-5,7-dioxo-6,7-dihydro-5H-thiazolo[3,2,c]pyrimidine-2-carboxylic acid methyl ester

Step B: Neat dimethylformamide dimethylacetal (6 mL, 45 mmol) was added dropwise to a rapidly stirred solution of (1-benzyl-2,6-dioxo-1,2,3,6-tetrahydro-pyrimidin-4-ylsulfanyl)-acetic acid methyl ester (6.12 g, 20 mmol) in tetrahydrofuran (250 mL) at 50° C. The solution was stirred at 50° C. for 0.5 hour, and then was heated at 70° C. on the rotary evaporator (no vacuum) until the solvent boiled off. More tetrahydrofuran (200 mL) was added, and the mixture was stirred overnight at room temperature. Dioxane (50 mL) was added, and the reaction mixture was concentrated to a brown oil. The oil was purified by filtration through silica gel (70-230 mesh) using ethyl acetate/hexanes (1:1) as eluant; yield 2.94 g (46%). MS (APCI+), m/z (%): 317.1(100), 259.0 (10).

### 6-Benzyl-5,7-dioxo-6,7-dihydro-5H-thiazolo[3,2,c]pyrimidine-2-carboxylic acid

Step C: 6-Benzyl-5,7-dioxo-6,7-dihydro-5H-thiazolo[3,2,c]pyrimidine-2-carboxylic acid methyl ester (3.87 g, 12.2 mmol) was taken up in tetrahydrofuran (80 mL), and the solution was cooled to 0° C. A solution of lithium hydroxide hydrate (0.49 g, 11.9 mmol) in water (10 mL) was added dropwise over 3 minutes. The solution was stirred 12 minutes at 0° C., and was then poured into a separatory funnel containing ethyl acetate (350 mL) and 10% citric acid solution (25 mL). The layers were separated, the organic layer washed with brine (50 mL), dried over magnesium sulfate, and filtered. The organic layer was concentrated on the rotary without heating. The residue was triturated with diethyl ether, and the solid collected by filtration; yield 2.996 g (81%). MS (APCI+), m/z (%): 303.0 (20), 259.0 (100).

### 6-Benzyl-5,7-dioxo-6,7-dihydro-5H-thiazolo[3,2,c]pyrimidine-2-carboxylic acid 4-fluoro-benzylamide

Step D: 6-Benzyl-5,7-dioxo-6,7-dihydro-5H-thiazolo[3,2,c]pyrimidine-2-carboxylic acid (2.25 g, 7.45 mmol) was mixed with 1-hydroxybenzotriazole (1.02 g, 7.55 mmol), 4-fluorobenzyl amine (0.95 g, 7.6 mmol), and dimethylformamide (12 mL), and 1-[3-(dimethylamino)propyl]-3-ethylcarbodiimide hydrochloride (1.45 g, 7.6 mmol) was added. The mixture was stirred for 3 days at room temperature. The volatiles were removed on the rotary evaporator with a bath temperature at 65° C., and the resulting residue was partitioned between ethyl acetate (300 mL) and water. The organic layer was washed with water (100 mL), sodium bicarbonate solution (2×50 mL), and brine (50 mL). The organic layer was dried over magnesium sulfate, filtered, and concentrated to a solid, which was triturated with hexanes/diethyl ether. The insoluble portion was collected by filtration, washed with diethyl ether, and dried; yield 2.599 g (85%). MS (APCI+), m/z (%): 410.0 (100), 259.0 (20).

### 6-Benzyl-8-formyl-5,7-dioxo-6,7-dihydro-5H-thiazolo[3,2,c]pyrimidine-2-carboxylic acid 4-fluoro-benzylamide

Step E: 6-Benzyl-5,7-dioxo-6,7-dihydro-5H-thiazolo[3,2,c]pyrimidine-2-carboxylic acid 4-fluoro-benzylamide (2.09 g, 5.84 mmol) was taken up in dimethylformamide (7.5 mL), and phosphorus oxychloride (1.2 mL, 12.9 mmol) was added dropwise over 5 minutes. The reaction mixture was stirred for 0.5 hour at room temperature, and then for 1.5 hours at 75° C. No starting material remained by mass spectrum analysis. The reaction mixture was cooled to room temperature, and was added dropwise to rapidly stirred water (150 mL). A yellow solid precipitated and was collected by filtration. The solid was taken up in ethyl acetate/tetrahydrofuran, and the solution was dried over magnesium sulfate, filtered, and concentrated to a solid. The solid was triturated with hexanes/ethyl acetate and collected. The solid was slurried in ethyl acetate and collected; yield 0.665 g of 6-benzyl-8-formyl-5,7-dioxo-6,7-dihydro-5H-thiazolo[3,2,c]pyrimidine-2-carboxylic acid 4-fluoro-benzylamide. An additional 1.6 g of material was obtained in several portions by filtration through silica gel using dichloromethane/tetrahydrofuran, 95/5 as eluant. MS (APCI+), m/z (%): 438.0 (100), 287.0 (25).

### EXAMPLE 113

### 6-Benzyl-8-hydroxymethyl-5,7-dioxo-6,7-dihydro-5H-thiazolo[3,2,c]pyrimidine-2-carboxylic acid 4-fluoro-benzylamide

A portion of the aldehyde prepared in Example 112, Step E, namely 6-benzyl-8-formyl-5,7-dioxo-6,7-dihydro-5H-thiazolo[3,2,c]pyrimidine-2-carboxylic acid 4-fluoro-benzylamide, (0.31 g, 0.71 mmol) was taken up in tetrahydrofuran (30 mL) and water (20 mL) containing sodium borohydride (0.21 g, 5.5 mmol), and the mixture was stirred at room temperature. After 0.5 hour, no starting material remained. The reaction was quenched by addition of 10% aqueous citric acid solution (5 mL). Brine (50 mL) was added, and the mixture was extracted with ethyl acetate (100 mL). The organic layer was washed with brine and dried over magnesium sulfate, filtered, and concentrated to a yellow oil. The oil was chromatographed on silica gel using ethyl acetate, then tetrahydrofuran as eluant; yield 0.024 g (6.5%) of 6-benzyl-8-hydroxymethyl-5,7-dioxo-6,7-dihydro-5H-thiazolo[3,2,c]pyrimidine-2-carboxylic acid 4-fluoro-benzylamide. MS (APCI−), m/z (%): 438.0 (50), 287.0 (100).

### EXAMPLE 114

### 6-(3,4-Fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide hydrochloride

### Step A: 8-Methyl-5,7-dioxo-6,7-dihydro-5H-thiazolo[3,2-c]pyrimidine-2-carboxylic acid(2-methoxy-pyridin-4-ylmethyl)-amide

8-Methyl-5,7-dioxo-6,7-dihydro-5H-thiazolo[3,2-c]pyrimidine-2-carboxylic acid (3.61 g) was dissolved in DMF (20 mL). To the solution was added 1-hydroxybenzotriazole hydrate (2.15 g) and 2-methoxy4-aminomethylpyridine, followed by 1-[3-(dimethylamino)propyl]-3-ethylcarbodiimide hydrochloride (3.05 g). The mixture was stirred at room temperature overnight. The DMF was removed in vacuum at 60° C. The residue was stirred in water, and the water was decanted. The residue was again stirred with water, and the resulting solid filtered. The solid was stirred with aqueous sodium bicarbonate, filtered, and air dried to give 8-methyl-5,7-dioxo-6,7-dihydro-5H-thiazolo[3,2-c]pyrimidine-2-carboxylic acid(2-methoxy-pyridin4-ylmethyl)-amide (59% yield); MS (APCI+), m/z (%): 347(20), 174(100), 135(20), 128(65).

### Step B: 6-(3,4-Fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide hydrochloride

The product of Step (A), namely 8-methyl-5,7-dioxo-6,7-dihydro-5H-thiazolo[3,2-c]pyrimidine-2-carboxylic acid(2-methoxy-pyridin4-ylmethyl)-amide (200 mg, 0.58 mmol), was suspended in DMF (10 mL), and cesium carbonate (204 mg, 0.63 mmol) was added, followed by 3,4-difluorobenzyl bromide (0.08 mL, 0.63 mmol). After 17 hours, the DMF was removed in a vacuum at 70° C. The residue was mixed with THF and filtered through a pad of Celite over silica gel eluting with additional THF. The filtrate was evaporated in vacuo to an oil. The material was purified by chromatography on silica gel, eluting with CH₂Cl₂:THF (9:1) to give 6-(3,4-fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide (30% yield). The residue was dissolved in THF (2 mL) and treated with HCl/diethyl ether (1 M, 0.2 mL). The resulting solid was isolated by filtration and dried in a vacuum at 55° C. to give 6-(3,4-fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide hydrochloride; MS (APCI+), m/z (%): 473(100), 309(30).

The invention compounds of Formula I have been evaluated in standard assays for their ability to inhibit the catalytic activity of various MMP enzymes. The assays used to evaluate the biological activity of the invention compounds are well-known and routinely used by those skilled in the study of MMP inhibitors and their use to treat clinical conditions.

The assays measure the amount by which a test compound reduces the hydrolysis of a thiopeptolide substrate catalyzed by a matrix metalloproteinase enzyme. Such assays are described in detail by Ye et al., in Biochemistry, 1992;31(45):11231-11235, which is incorporated herein by reference.

Thiopeptolide substrates show virtually no decomposition or hydrolysis at or below neutral pH in the absence of a matrix metalloproteinase enzyme. A typical thiopeptolide substrate commonly utilized for assays is Ac-Pro-Leu-Gly-thioester-Leu-Leu-Gly-OEt. A 100 μL assay mixture will contain 50 mM of N-2-hydroxyethylpiperazine-N′-2-ethanesulfonic acid buffer (−HEPES,= pH 7.0), 10 mM CaCl₂, 100 μM thiopeptolide substrate, and 1 mM 5,5′-dithio-bis-(2-nitro-benzoic acid) (DTNB). The thiopeptolide substrate concentration may be varied, for example from 10 to 800 μM to obtain Kₘ and K_{cat} values. The change in absorbance at 405 nm is monitored on a Thermo Max microplate reader (molecular Devices, Menlo Park, Calif.) at room temperature (22° C.). The calculation of the amount of hydrolysis of the thiopeptolide substrate is based on E₄₁₂=13600 M⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Several representative compounds have been evaluated for their ability to inhibit various matrix metalloproteinase enzymes. Table 1 below presents inhibitory activity for compounds from various classes. In Table 1, MMP-1FL refers to full length interstitial collagenase; MMP-2FL refers to full length Gelatinase A; MMP-3CD refers to the catalytic domain of stromelysin-1; MMP-7FL refers to full length matrilysin; MMP-9FL refers to full length Gelatinase B; MMP-13CD refers to the catalytic domain of collagenase 3; and MMP-14CD refers to the catalytic domain of MMP-14. Test compounds were evaluated at various concentrations in order to determine their respective IC₅₀ values, the micromolar concentration of compound required to cause a 50% inhibition of catalytic activity of the respective enzyme.

It should be appreciated that the assay buffer used with MMP-3CD was 50 mM N-morpholinoethane sulfonate (−MES=) at pH 6.0 rather than the HEPES buffer at pH 7.0 described above.

TABLE 1
IC₅₀ (μM) Versus Certain MMPs

| Example | | MMP-13 | MMP-01 | MMP-02 | MMP-03 | MMP-07 | MMP-09 | MMP-14 |
|---|---|---|---|---|---|---|---|---|
| No. | Structure | CD | FL | FL | CD | FL | FL | CD |
| 1 | 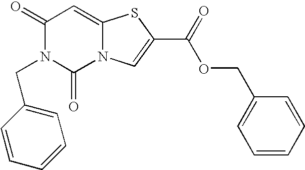 | 0.0285 | >100 | >100 | 23 | 96 | >100 | >30 |
| 2 | 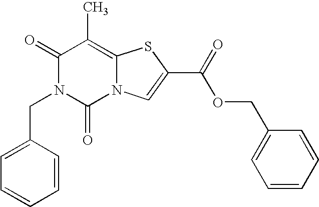 | 0.0056 | >100 | >100 | >30 | >100 | >100 | >100 |
| 3 | 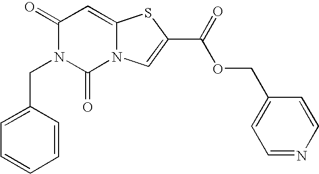 | 0.0054 | >100 | >100 | 11 | >100 | >100 | >100 |
| 4 | 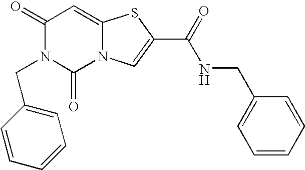 | 0.5725 | 81 | >100 | 27 | 65 | 83.5 | 47.5 |
| 5 | 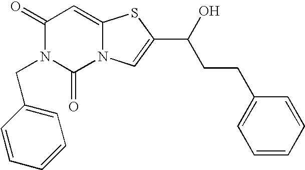 | 14.3333 | >100 | >100 | >100 | >100 | >100 | >100 |
| 6 | 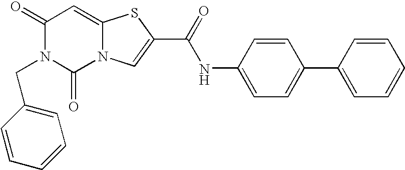 | 23.0000 | >30 | >30 | 3.5 | 9.2 | >30 | 7.3 |
| 7 | 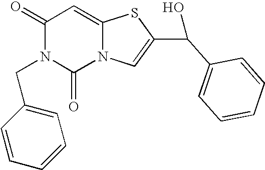 | 32.0000 | | | | | | |
| 8 | 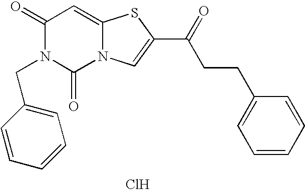 | 6.6 | >100 | >100 | 3.2 | >30 | >100 | 8.8 |
| 9 | 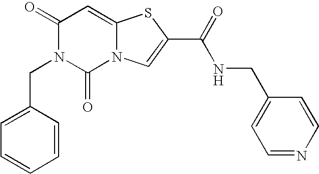 | 0.4900 | >100 | >100 | 68 | >100 | >100 | >100 |
| 10 | 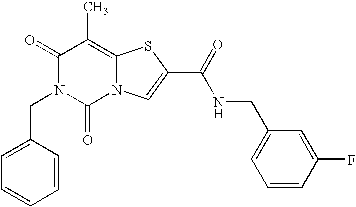 | 0.1200 | >30 | >100 | >30 | 21 | >30 | >30 |
| 11 | 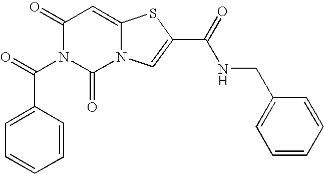 | 6.7000 | >30 | >30 | >30 | >30 | >30 | >30 |
| | 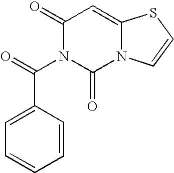 | >100 | | | | | | |
| 12 | 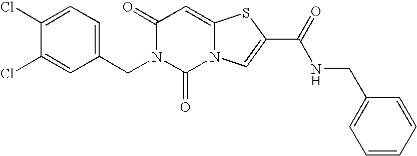 | 0.2933 | >30 | >100 | >30 | 20 | >30 | >30 |
| 13 | 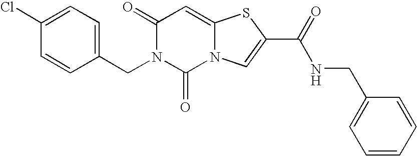 | 0.4150 | >30 | >100 | 43 | >100 | >100 | >100 |
| | 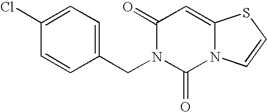 | >100 | | | | | | |
| 14 | 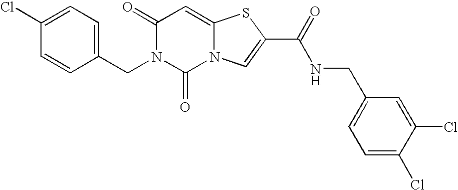 | 4.3000 | >30 | >30 | >100 | 25 | >30 | >100 |
| 15 | 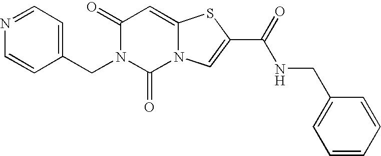 | 1.8000 | >100 | >100 | >100 | >100 | >100 | >100 |
| | 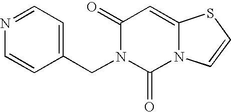 | >100 | | | | | | |
| 16 | 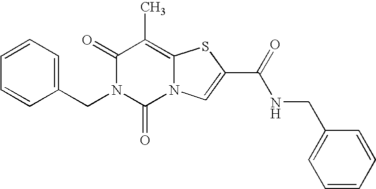 | 0.0940 | >100 | >100 | 51 | >30 | >100 | >30 |
| 17 | 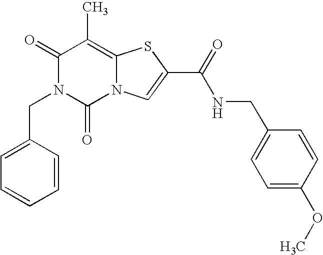 | 0.0217 | >30 | >30 | 16 | >30 | >30 | >30 |
| 18 | 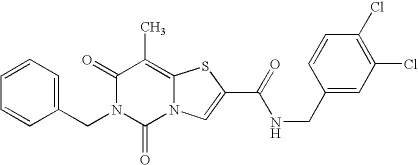 | 0.2650 | >30 | >30 | 20 | >30 | >30 | >30 |
| 19 | 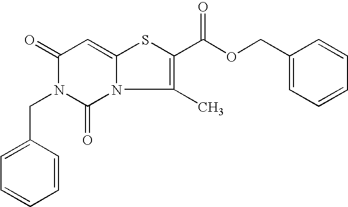 | 21.0000 | >100 | >100 | >100 | 8.8 | >30 | >30 |
| 20 | 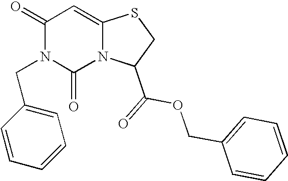 | none | | | | | | |
| 21 | 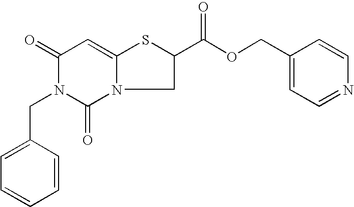 | 6.5000 | >100 | >100 | >100 | >100 | >100 | >100 |
| 22 | 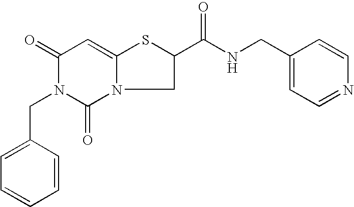 | 0.5900 | >100 | >100 | >100 | >100 | >100 | >100 |
| 23 | 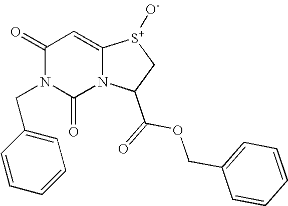 | 0.0720 | >100 | >100 | 23 | >100 | >100 | >100 |
| 24 | 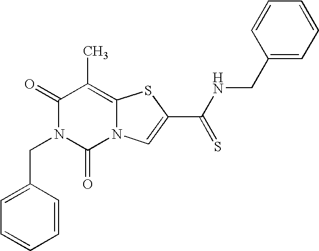 | >30 | | | | | | |
| 35^{a} | | >100 | | | | | | |
| 26 | 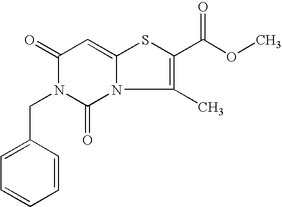 | >100 | | | | | | |
| 27 | 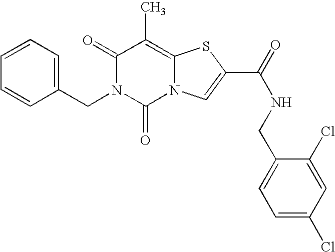 | >30 | | | | | | |
| 28 | 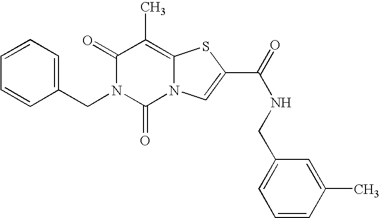 | 0.0250 | >100 | >30 | 19 | 71 | >30 | >100 |
| 29 | 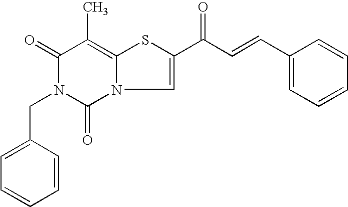 | 25 | | | | | | |
| 30 | 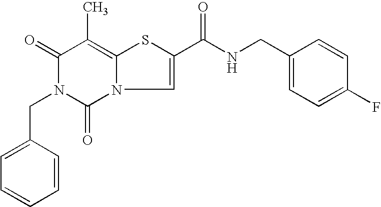 | 0.0245 | >30 | >30 | >100 | >30 | >30 | >30 |
| 31 | 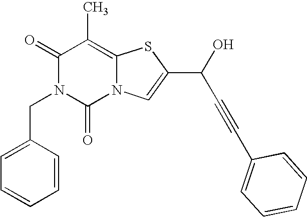 | >30 | | | | | | |
| 32 | 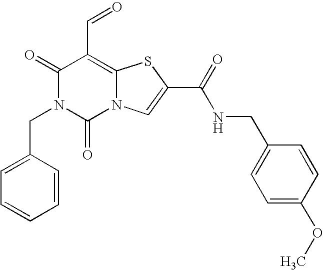 | 0.0224 | >100 | >100 | 11 | >30 | >100 | >30 |
| 33 | 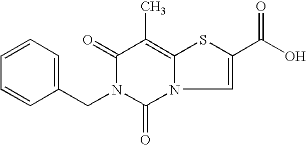 | >100 | | | | | | |
| 33a | 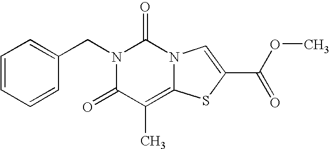 | none | | | | | | |
| 34 | 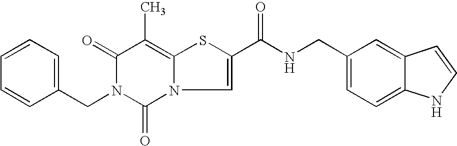 | 23 | | | | | | |
| 36 | 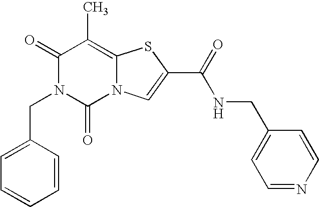 | 0.0487 | >30 | >100 | >30 | >100 | >30 | >100 |
| 37 | 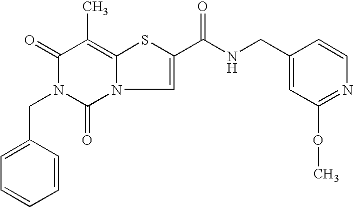 | 0.0175 | >30 | >30 | 10 | >30 | >30 | >30 |
| 37a | 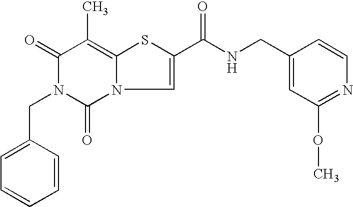 | 0.0208 | >30 | >30 | 20.5 | 26 | >30 | >30 |
| 38 | 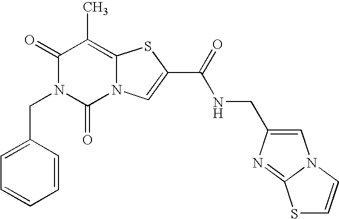 | 25 | | | | | | |
| 39 | 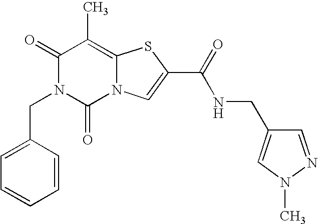 | 2.2999 | | | | | | |
| 40 | 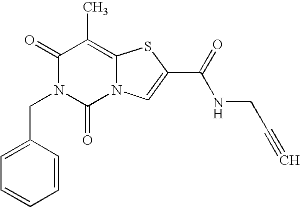 | >100 | | | | | | |
| 41 | 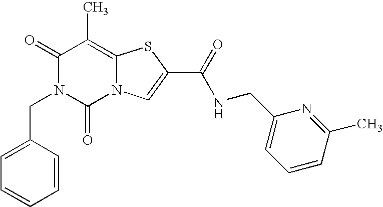 | 6.2000 | | | | | | |
| 42 | 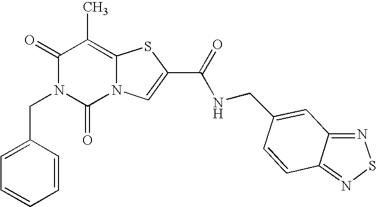 | 0.0046 | >30 | >30 | 10 | >30 | >30 | >100 |
| 43 | 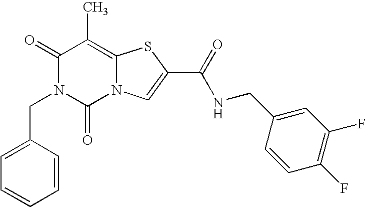 | 0.0290 | >100 | >30 | >30 | >30 | >30 | >100 |
| 44 | 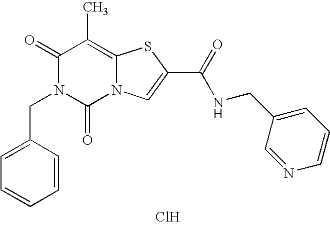 | 0.2250 | >100 | >100 | >30 | >100 | >30 | >100 |
| 44a | 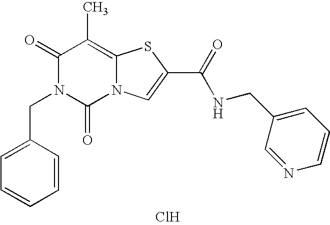 | 0.2600 | >30 | >100 | >30 | >100 | >30 | >100 |
| 45 | 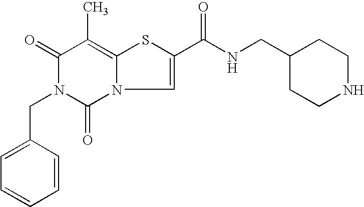 | >100 | | | | | | |
| 46 | 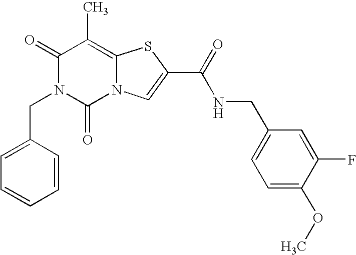 | 0.0250 | >100 | >100 | 12 | >30 | >100 | >100 |
| 47 | 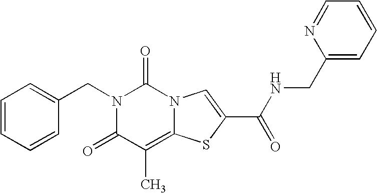 | 9.9000 | | | | | | |
| 48 | 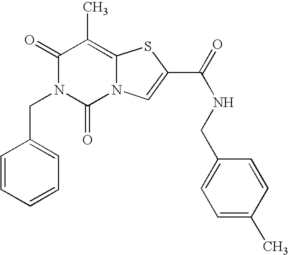 | 0.2250 | >30 | >30 | 19 | 28 | >100 | >100 |
| 49 | 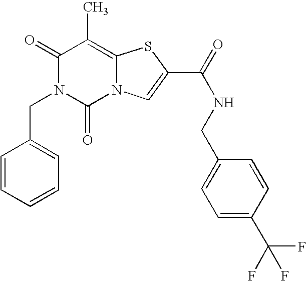 | 2.21 | >30 | >30 | >100 | >100 | >100 | >100 |
| 50 | 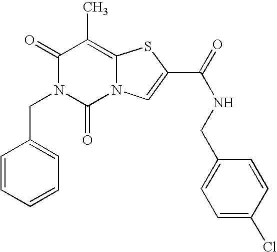 | 0.0869 | >30 | >30 | 23 | 31 | >100 | >100 |
| 51 | 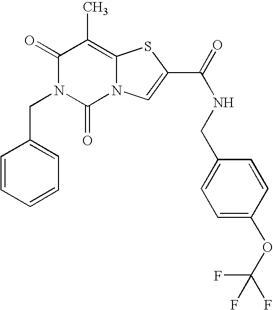 | 0.8150 | >30 | >30 | >100 | >30 | >100 | >100 |
| 52 | 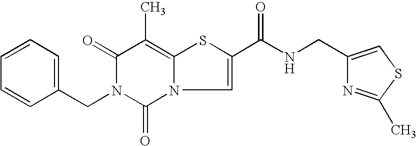 | >100 | | | | | | |
| 53 | 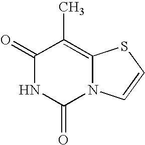 | >100 | | | | | | |
| 55 | 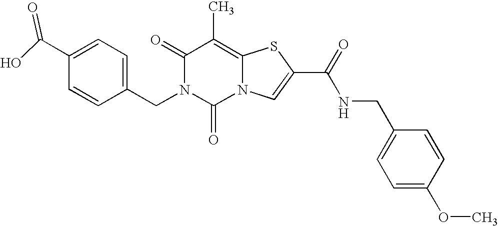 | 0.0015 | >100 | >100 | 29 | >100 | >100 | >100 |
| 56 | 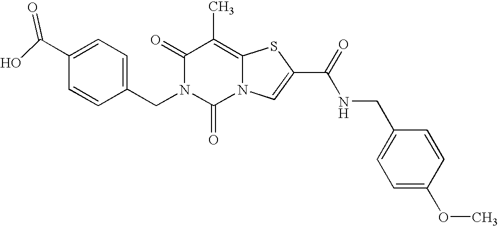 | 0.00175 | >100 | >100 | 36 | >100 | >100 | >100 |
| 57 | 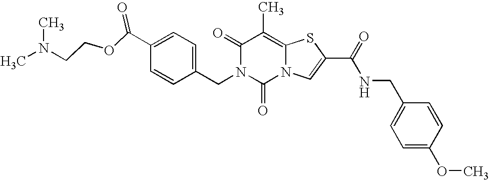 | 0.0455 | >30 | >30 | >100 | >30 | >30 | >30 |
| 58 | 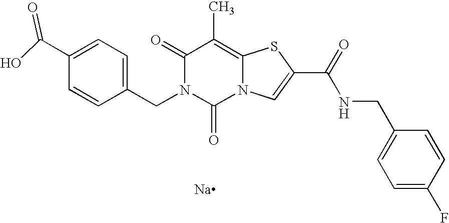 | 0.002225 | >100 | >100 | 68 | >100 | >100 | >100 |
| 59 | 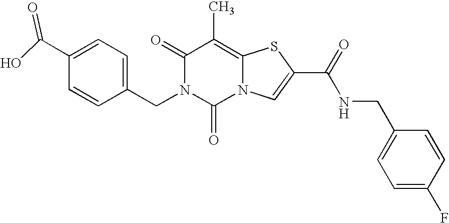 | 0.0020 | >100 | >100 | 55.5 | >100 | >100 | >100 |
| 60 | 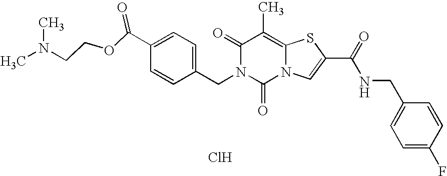 | 0.094 | >100 | >100 | >30 | >100 | >100 | >100 |
| 61 | 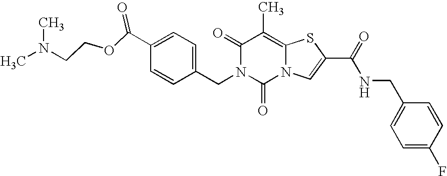 | 0.0710 | >30 | >100 | >100 | >100 | >100 | >100 |
| 62 | 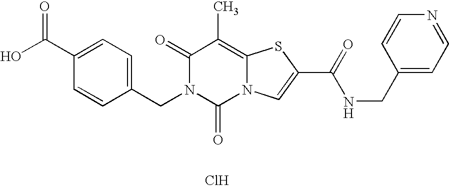 | 0.0057 | >100 | >100 | >100 | >100 | >100 | >100 |
| 63 | 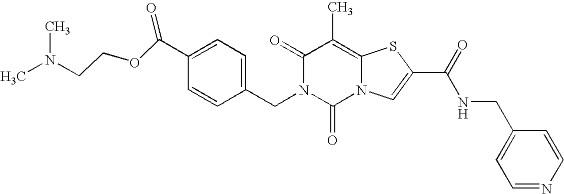 | 0.2350 | >100 | >100 | >100 | >100 | >100 | >100 |
| 64 | 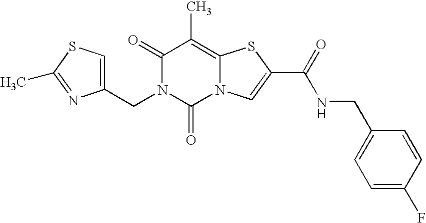 | 0.7700 | >100 | >100 | >30 | >100 | >30 | >100 |
| 65 | 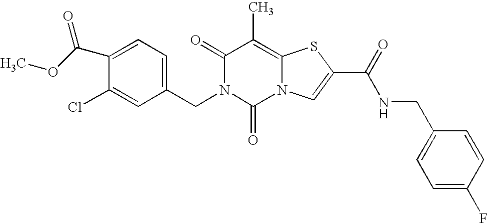 | 0.2400 | >30 | >30 | >30 | 26 | >30 | >30 |
| 66 | 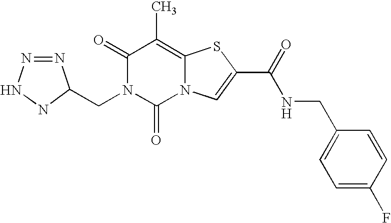 | 14 | | | | | | |
| 67 | 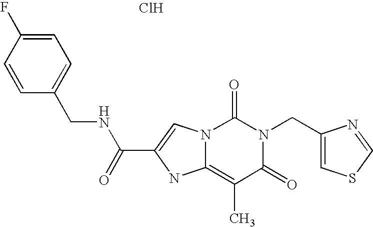 | 0.5300 | >100 | >100 | >30 | >100 | >100 | >100 |
| 68 | 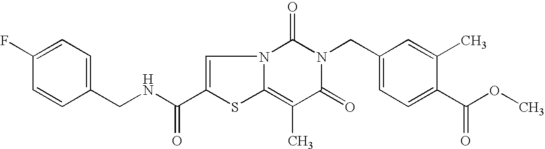 | 0.0180 | >100 | >100 | 18 | >100 | >100 | >100 |
| 69 | 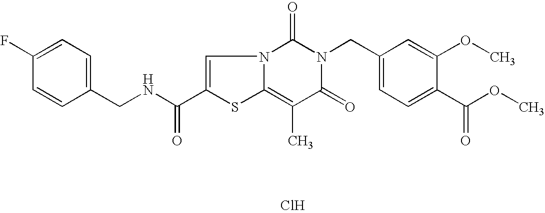 | 0.0990 | >30 | >30 | 10 | >30 | >30 | >30 |
| 70 | 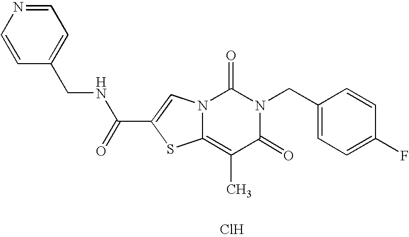 | 0.0605 | >30 | >30 | >30 | >30 | >30 | >30 |
| 71 | 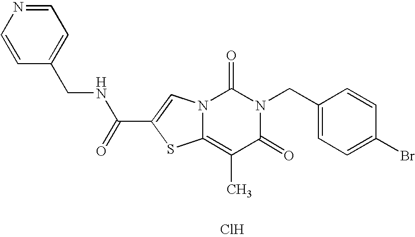 | 0.0365 | >30 | >30 | >30 | >30 | >30 | >30 |
| 72 | 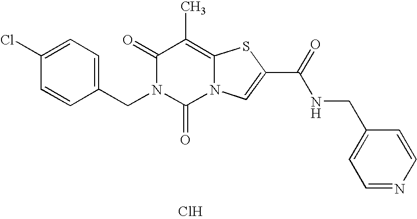 | 0.0530 | >30 | >30 | >30 | >30 | >30 | >30 |
| 73 | 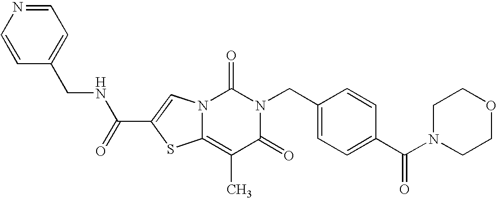 | 0.0370 | >100 | >100 | >100 | >100 | >100 | >30 |
| 74 | 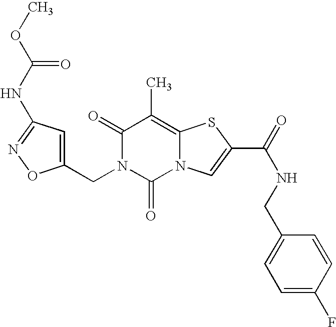 | 0.2050 | >30 | >30 | >30 | 87 | >30 | >30 |
| 75 | 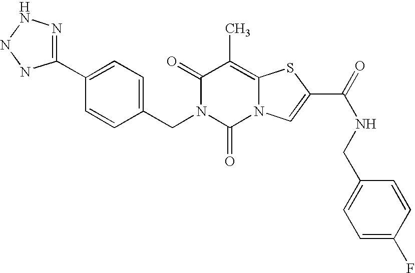 | 0.0009 | >100 | >100 | 16 | >100 | >100 | >100 |
| 76 | 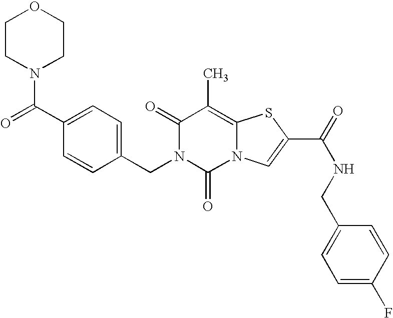 | 0.0110 | >30 | >30 | >30 | >30 | >30 | >30 |
| 77 | 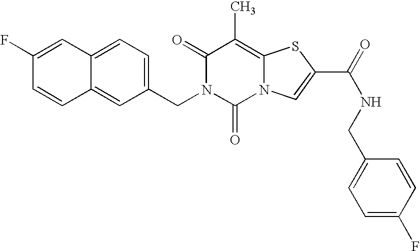 | 0.0860 | >30 | >30 | 18 | >30 | >30 | >30 |
| 78 | 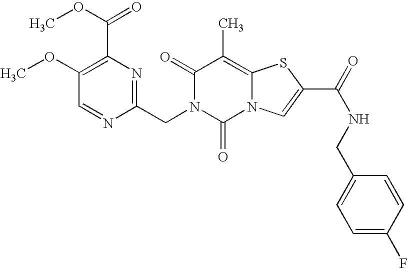 | 1.8500 | >30 | >30 | >30 | >30 | >30 | >30 |
| 79 | 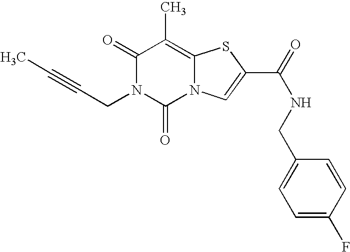 | 0.315 | >100 | >100 | >30 | >100 | >100 | >100 |
| 80 | 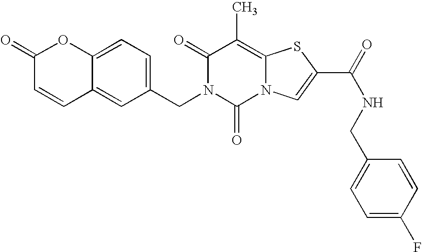 | 0.012 | >30 | >30 | >30 | >30 | >30 | >30 |
| 81 | 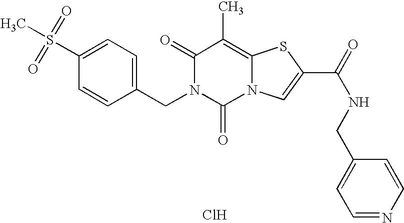 | 0.0235 | >100 | >100 | >100 | >100 | >100 | >100 |
| 82 | 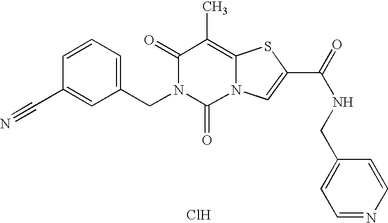 | 0.1733 | >30 | >30 | >30 | >30 | >30 | >30 |
| 83 | 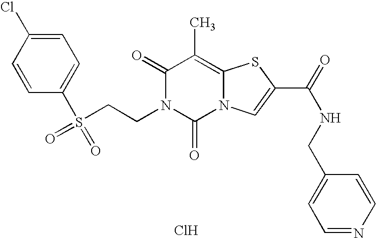 | 2.6000 | | | | | | |
| 84 | 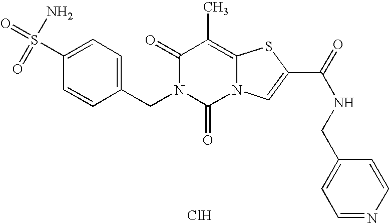 | 0.0463 | >30 | >100 | >100 | >100 | >100 | >100 |
| 85 | 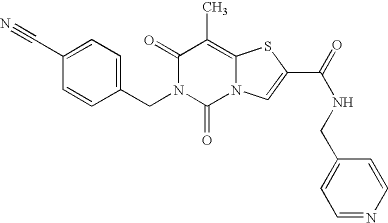 | 0.054667 | >30 | >30 | >30 | >30 | >30 | >30 |
| 86 | 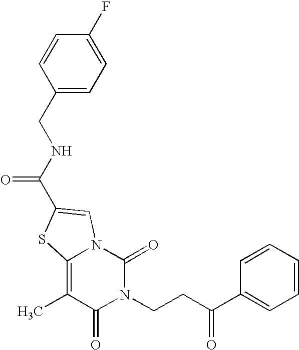 | 0.3500 | | | | | | |
| 87 | 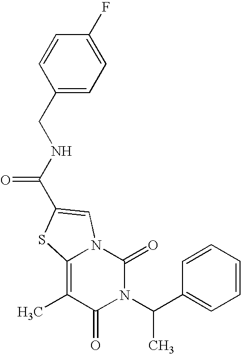 | 2.1000 | | | | | | |
| 88 | 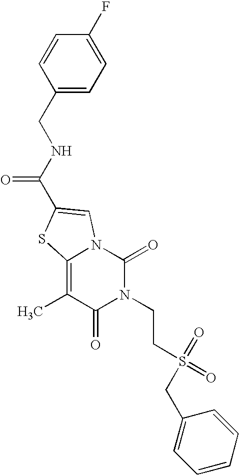 | 0.5600 | | | | | | |
| 89 | 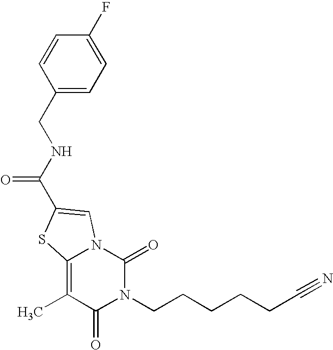 | 1.0850 | | | | | | |
| 90 | 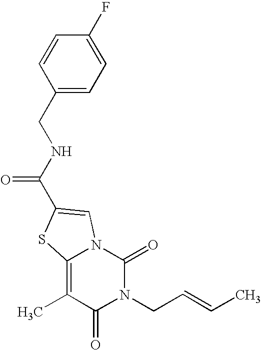 | 0.5300 | | | | | | |
| 91 | 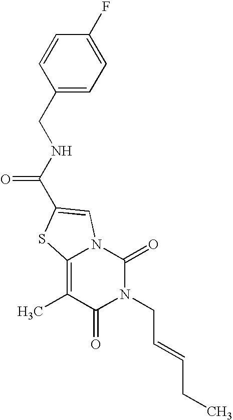 | 0.1600 | | | | | | |
| 92 | 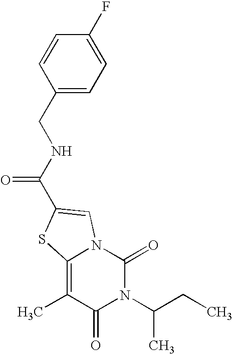 | 0.4000 | | | | | | |
| 93 | 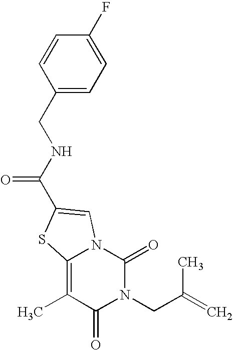 | 1.5500 | | | | | | |
| 94 | 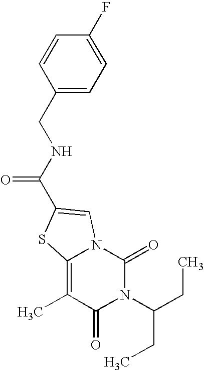 | >30 | | | | | | |
| 95 | 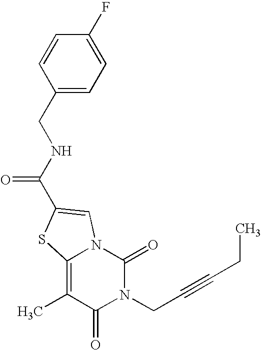 | 0.0920 | | | | | | |
| 96 | 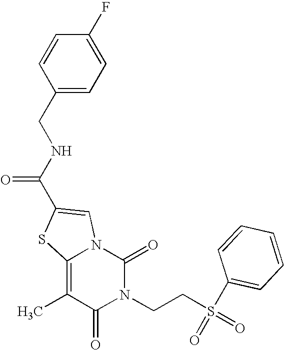 | 1.2000 | | | | | | |
| 97 | 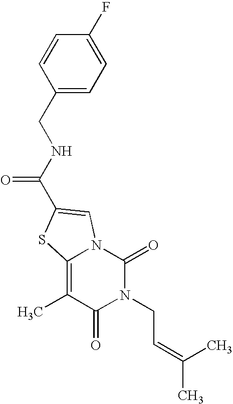 | 0.8200 | | | | | | |
| 98 | 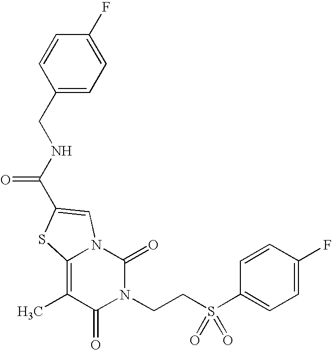 | 0.6450 | | | | | | |
| 99 | 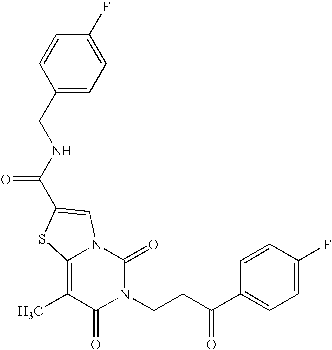 | 0.5100 | | | | | | |
| 100 | 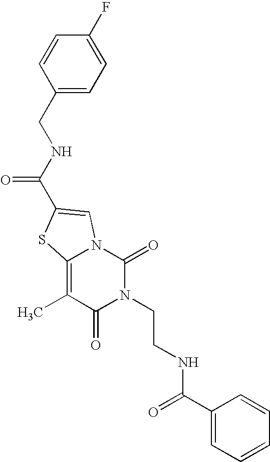 | 10.0 | | | | | | |
| 101 | 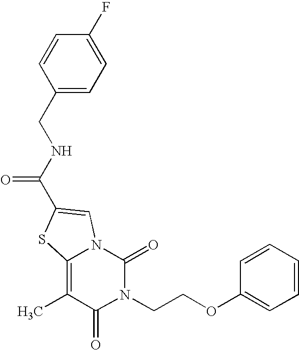 | 0.0580 | | | | | | |
| 102 | 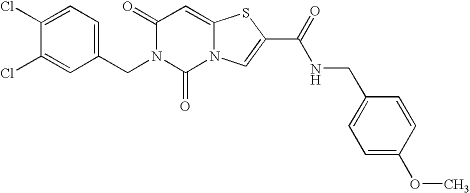 | 0.0840 | >100 | >100 | >100 | >100 | >100 | >100 |
| 103 | 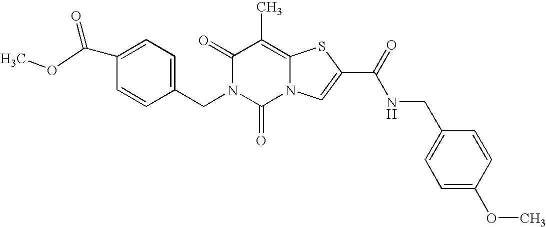 | 0.01065 | >100 | 65 | 65 | >100 | >100 | >100 |
| 104 | 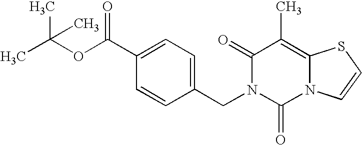 | ×30 | | | | | | |
| 105 | 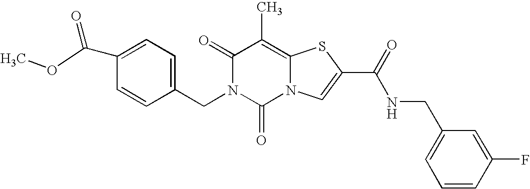 | 0.0715 | >30 | >30 | 19 | 27 | >30 | >30 |
| 106 | 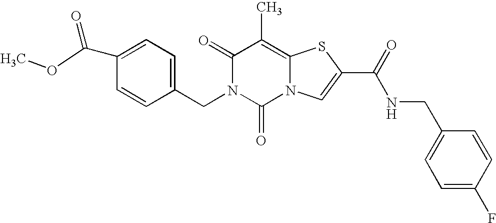 | 0.0180 | >100 | >30 | 17 | >30 | >100 | >100 |
| 107 | 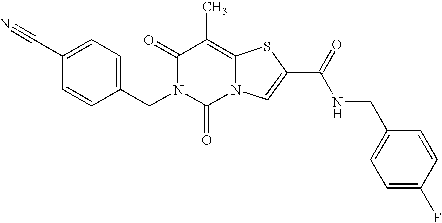 | 0.023 | >100 | >30 | 25 | >30 | >30 | >30 |
| 108 | 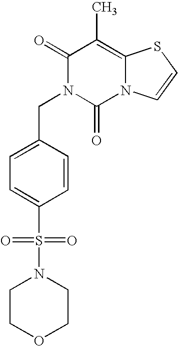 | >100 | | | | | | |
| 109 | 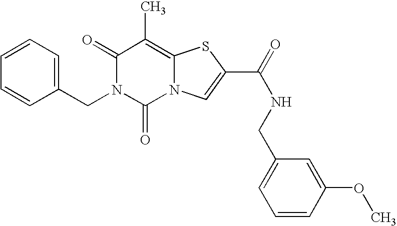 | 0.0060 | >30 | >30 | 22 | >30 | >30 | >30 |
| 110 | 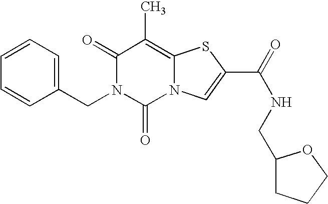 | 54 | | | | | | |

(a) This entry is for Example 35, not Example 25.

Further, IC₅₀ inhibition of MMP-13CD by the compounds of Examples 111 and 114 is 0.17 micromolar and 0.0155 micromolar, respectively.

The foregoing data establish that the invention compounds of Formula I are potent inhibitors of MMP enzymes, and are especially useful due to their selective inhibition of MMP-13. Because of this potent and selective inhibitory activity, the invention compounds are especially useful to treat diseases mediated by the MMP enzymes, and particularly those mediated by MMP-13.

Administration of a compound of Formula I, or a pharmaceutically acceptable salt thereof, to a mammal to treat the diseases mediated by MMP enzymes is preferably, although not necessarily, accomplished by administering the compound, or the salt thereof, in a pharmaceutical dosage form.

The compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds of the present invention can be administered by inhalation, for example, intranasally. Additionally, the compounds of the present invention can be administered transdermally. It will be obvious to those skilled in the art that the following dosage forms may comprise as the active component, either a compound of Formula I or a corresponding pharmaceutically acceptable salt of a compound of Formula I. The active compound generally is present in a concentration of about 5% to about 95% by weight of the formulation.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from 5% or 10% to about 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term −preparation= is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 1 to 1000 mg, preferably 10 to 100 mg according to the particular application and the potency of the active component. The composition can, if desired, also contain other compatible therapeutic agents.

In therapeutic use as agents to inhibit a matrix metalloproteinase enzyme for the treatment of atherosclerotic plaque rupture, aortic aneurism, heart failure, restenosis, periodontal disease, corneal ulceration, cancer metastasis, tumor angiogenesis, arthritis, or other autoimmune or inflammatory disorders dependent upon breakdown of connective tissue, the compounds utilized in the pharmaceutical method of this invention are administered at a dose that is effective to inhibit the hydrolytic activity of one or more matrix metalloproteinase enzymes. The initial dosage of about 1 mg/kg to about 100 mg/kg daily will be effective. A daily dose range of about 25 mg/kg to about 75 mg/kg is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstance is reached. For convenience, the total daily dosage may be divided and administered in portions during the day if desired. Typical dosages will be from about 0.1 mg/kg to about 500 mg/kg, and ideally about 25 mg/kg to about 250 mg/kg, such that it will be an amount which is effective to treat the particular disease being prevented or controlled.

The following examples illustrate typical formulations provided by the invention.

### FORMULATION EXAMPLE 1

Tablet Formulation

| Ingredient | Amount (mg) |
|---|---|
| Bicyclic pyrimidine of Example 3 | 25 |
| Lactose | 50 |
| Corn starch (for mix) | 10 |
| Corn starch (paste) | 10 |
| Magnesium stearate (1%) | 5 |
| Total | 100 |

The bicyclic pyrimidine of Example 3, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of atherosclerosis and arthritis.

### FORMULATION EXAMPLE 2

Preparation for Oral Solution

| Ingredient | Amount | |
|---|---|---|
| Bicyclic pyrimidine of Example 4 | 400 | mg |
| Sorbitol solution (70% N.F.) | 40 | mL |
| Sodium benzoate | 20 | mg |
| Saccharin | 5 | mg |
| Red dye | 10 | mg |
| Cherry flavor | 20 | mg |
| Distilled water q.s. | 100 | mL |

1-3. (canceled)

4. A compound, or a pharmaceutically acceptable salt thereof, selected from the group consisting of:
6-Benzyl-8-methyl-5,7-dioxo-6,7-dihydro-5H-thiazolo[3,2]pyrimidine-2-carbothioic acid benzylamide;
6-Benzyl-8-methyl-5,7-dioxo-6,7-dihydro5H-thiazolo[3,2-c]pyrimidine-2-carbothioic acid 4-methoxy-benzylamide.
6-Benzyl-2-(3-phenyl-propionyl)thiazolo[3,2-c]pyrimidine-5,7-dione;
6-Benzyl-8-methyl-5,7-dioxo-6,7-dihydro-5H-thiazolo[3,2-c]pyrimidine-2-carboxylic acid prop-2-ynylamide;
6-Benzyl-8-methyl-5,7-dioxo-6,7-dihydro-5H-thiazolo[3,2-c]pyrimidine2-carboxylic acid(piperidin-4-ylmethyl)-amide hydrochloride;
6-Benzyl-2-(1-hydroxy-3-phenyl-allyl)-8-methyl-thiazolo[3,2-c]pyrimidine-5,7-dione;
6-Benzyl-2-(1-hydroxy-3-phenyl-prop-2-ynyl)-8-methyl-thiazolo[3,2-c]pyrimidine-5,7-dione;
6-Benzyl-2-(hydroxy-phenyl-methyl)-thiazolo[3,2-c]pyrimidine-5,7-dione; and
6-Benzyl-2-(1-hydroxy-3-phenyl-propyl)-thiazolo[3,2-c]pyrimidine-5,7-dione.

5-9. (canceled)

10. A compound, or a pharmaceutically acceptable salt thereof, selected from the group consisting of:
6-Benzyl-5,7-dioxo-2,3,6,7-tetrahydro-5H-thiazolo[3,2-c]pyrimidine-2-carboxylic acid benzyl ester 2,3-Dihydroxypropionic acid benzyl ester,
6-Benzyl-5,7-dioxo-2,3,6,7-tetrahydro-5H-thiazolo[3,2-c]pyrimidine-2-carboxylic acid pyridin-4-ylmethyl ester hydrochloride; and
6-Benzyl-1,5,7-trioxo-1,2,3,5,6,7-hexahydro-14-thiazolo[3,2-c]pyrimidine-3-carboxylic acid benzyl ester.

11-14. (canceled)

15. A compound or a pharmaceutically acceptable salt thereof, selected from the group consisting of:
4-[8-Methyl-5,7-dioxo-2-(3-phenyl-prop-1-ynyl)-7H-thiazolo[3,2-c]pyrimidin-6-ylmethyl]-benzoic acid;
4-{2-[3-(4-Methoxy-phenyl)-prop-1-ynyl]-8-methyl-5,7-dioxo-7H-thiazolo[3,2-c]pyrimidin-6-ylmethyl}-benzoic acid;
4-{2-[3-(4-Fluoro-phenyl)-prop-1-ynyl]-8-methyl-5,7dioxo-7H-thiazolo[3,2c]pyrimidin-6-ylmethyl}-benzoic acid;
4-{2-[3-(3-Methoxy-phenyl)-prop-1-ynyl]-8-methyl-5,7-dioxo-7H-thiazolo[3,2-pyrimidin-6-ylmethyl}-benzoic acid,
4-{2-[3-3,4-Difluoro-phenyl)-prop-1-ynyl]-8-methyl-5,7-dioxo-7H-thiazolo[3,2-c]pyrimidin-6-ylmethyl}-benzoic acid;
6-Benzyl-8-methyl-2-(3-pyridin-4-yl-prop-1-ynyl)thiazolo[3,2-c]pyrimidine-5,7-dione;
6-(3,4-Dichloro-benzyl)-8-methyl-2-(3-pyridin-4yl-prop-1-ynyl)-thiazolo[3,2-c]pyrimidine-5,7-dione;
6-(3,4-Dichloro-benzyl)-2-[3-(2-methoxy-pyridin-4-yl)prop-1-ynyl]-8-methyl-thiazolo[3,2-c]pyrimidine-5,7-dione;
6-Benzyl-8-methyl-2-phenylethynyl-thiazolo[3,2-c]pyrimidine-5,7-dione;
6-(4Bromo-benzyl)-2-[3-3-methoxy-phenyl)-prop-1-ynyl]-8-methyl-thiazolo[3,2-c]pyrimidine-5,7-dione;
4-{2-[3-(3-Methoxy-phenyl)-prop-1-ynyl]-8-methyl-5,7-oxo-7H-thiazolo[3,2-c]pyrimidin-6-ylmethyl}-benzenesulfonamide;
4-{2-[3-(3-Fluoro-4-methoxy-phenyl)-prop-1-ynyl]-8-methyl-5,7-dioxo-7H-thiazolo[3,2-c]pyrimidin-6-ylmethyl}-benzoic acid;
6-(4-Fluoro-benzyl)-8-methyl-2-(3-phenyl-prop-1-ynyl)-thiazolo[3,2-c]pyrimidine-5,7-dione;
6-Benzyl-8-methyl-2-(3-phenyl-prop-1-ynyl)-thiazolo[3,2-c]pyrimidine-5,7-dione;
6-(3,4-Dichloro-benzyl)-2-[3-(3-methoxy-phenyl)prop-1-ynyl]-8-methyl-thiazolo[3,2-c]pyrimidine-5,7-dione;
6-(4-Methanesulfonyl-benzyl)-8-methyl-2-(3-pyridin-4-yl-prop-1-ynyl)-thiazolo[3,2-c]pyrimidine-5,7-dione;
4-{2-[3-(3-Methoxy-phenyl)-prop-1-ynyl]-8-methyl-5,7-dioxo-7H-thiazolo[3,2c]pyrimidin-ylmethyl}-benzonitrile;
4-[8-Methyl-5,7-dioxo-2-(3-phenyl-prop-1-ynyl)-7H-thiazolo[3,2-c]pyrimidin-6-ylmethyl]-benzoic acid;
2-[3-(3-Methoxy-phenyl)-prop-1-ynyl]-8-methyl-6-[4-(2H-tetrazol-5-yl)-benzyl]-thiazolo[3,2c]pyrimidine-5,7-dione;
6-Benzyl-2-[3-(3-methoxy-phenyl)-prop-1-ynyl)-8-methyl-thiazolo[3,2-c]pyrimidine-5,7dione;
6-Benzyl-8-methyl-2-(3-phenyl-prop-1-ynyl)-thiazolo[3,2-c]pyrimidine-5,7-dione;
2-[3-(3-Methoxy-phenyl)-prop-1-ynyl)-8-methyl-6-[4-(morpholine-4-carbonyl)-benzyl]-thiazolo[3,2-c]pyrimidine-5,7-dione;
8-Methyl-6-[4-(morpholine-4-sulfonyl)-benzyl-2-(3-pyridin-4-yl-prop-1-ynyl)-thiazolo[3,2-c]pyrimidine-5,7-dione;
2-[3-(4-Fluoro-phenyl)-prop-1-ynyl]-8-methyl-6-(2-oxo-2H-1-benzopyran-6-ylmethyl)-thiazolo[3,2c]pyrimidine-5,7-dione;
2-[3-(3-Methoxy-phenyl)-prop-1-ynyl)-8-methyl-6-(2-oxo-2H-1-benzopyran-6-ylmethyl)-thiazolo[3,2-c]pyrimidine-5,7-dione;
4-[8-Methyl-5,7-dioxo-2-(4-phenyl-but-1-ynyl)-7H-thiazolo[3,2-c]pyrimidin-6-ylmethyl]-benzoic acid;
4-[8-Methyl-5,7-dioxo-2-6-phenyl-hex-1-ynyl)-7H-thiazolo[3,2-c]pyrimidin-6-ylmethyl]-benzoic acid;
4-[8-Methyl-5,7-dioxo-2-(5-phenyl-pent-1-ynyl)-7H-thiazolo[3,2-c]pyrimidin-6-ylmethyl)-benzoic acid;
4-[8-Methyl-5,7-dioxo-2-(7-phenyl-hept-1-ynyl)-7H-thiazolo[3,2-c]pyrimidin-6-ylmethyl]-benzoic acid;
(4{2-[3-(3,4-Difluoro-phenyl)prop-1-ynyl]-8-methyl-5,7-dioxo-7H-thiazolo[3,2-c]pyrimidin-6-ylmethyl)-phenyl)-acetic acid;
6-(3-Fluoro-benzyl)-8-methyl-2-(3-pyridin-4-yl-prop-1-ynyl)-thiazolo[3,2-c]pyrimidine-5,7-dione;
6-(3,4Difluoro-benzyl)-8-methyl-2-(3-pyridin-4-yl-prop-1-ynyl)-thiazolo[3,2-c]pyrimidine-5,7-dione;
6-(3-Fluoro-benzyl)-2-[3-(2-methoxy-pyridin-4-yl)-prop-1-ynyl]-8-methyl-thiazolo[3,2-c]pyrimidine-5,7-dione;
[3-(8-Methyl-5,7-dioxo-2-phenylethynyl-7H-thiazolo[3,2-c]pyrimidin-6-ylmethyl)-phenyl]-acetic acid;
6-(4Bromo-benzyl)-2-[3-(4fluoro-3-methoxy-phenyl)-prop-1-ynyl]-8-methyl-thiazolo[3,2-c]pyrimidine-5,7-dione;
4-{2-[3-(3-Methoxy-phenyl)-prop-1-ynyl]-8-methyl-5,7-dioxo-7H-thiazolo[3,2-c]pyrimidin-6-ylmethyl}-N,N-dimethyl-benzenesulfonamide;
4-{2-[3-(3-Fluoro-4-methoxy-phenyl)-prop-1-ynyl]-8-methyl-5,7-dioxo-7H-thiazolo[3,2-c]pyrimidin-6-ylmethyl}-cyclohexanecarboxylic acid;
6-(3,4-Difluoro-benzyl)-2-[3-(3,4-difluoro-phenyl)-prop-1-ynyl]-8-methyl-thiazolo[3,2-c]pyrimidine-5,7-dione;
4-[8-Methyl-5,7 dioxo-2-(3-phenyl-prop-1-ynyl)-7H-thiazolo[3,2-c]pyrimidin-6-ylmethyl]cyclohexanecarboxylic acid;
2-Chloro-4-{2-[3-(3-methoxy-phenyl)-prop-1-ynyl]-8-methyl-5,7-dioxo-7H-thiazolo[3,2-c]pyrimidin-6-ylmethyl}-benzoic acid;
2-[3-(4-Fluoro-phenyl)-prop-1-ynyl]-6-4-methanesulfonyl-benzyl)-8-methyl-thiazolo[3,2-c]pyrimidine-5,7-dione;
4-{2-[3-(4-Fluoro-3-methoxy-phenyl)-prop-1-ynyl]-8-methyl-5,7-dioxo-7H-thiazolo[3,2-c]pyrimidin-6-ylmethyl}-benzonitrile;
(3-{2-(3-(4-Fluoro-3-methoxy-phenyl)-prop-1-ynyl]-8-methyl-5,7-dioxo-7H-thiazolo[3,2-c]pyrimidin-6-ylmethyl}-phenyl)-acetic acid;
(4-{2-[3-3-Methoxy-phenyl)-prop-1-ynyl]-8-methyl-5,7-dioxo-7H-thiazolo[3,2-c]pyrimidin-6-ylmethyl}-phenyl)-acetic acid;
6-(3,4-Difluoro-benzyl)-8-methyl-2-(3-phenyl-prop-1-ynyl)-thiazolo[3,2-c]pyrimidine-5,7-dione;
2-[3-(3-Methoxy-phenyl)-prop-1-ynyl]-8-methyl-6-[4-(thiomorpholine carbonyl)-benzyl]-thiazolo[3,2-c]pyrimidine-5,7-dione;
8-Methyl-2-(3-pyridin-prop-1-ynyl)-6-[4-(thiomorpholine-4-sulfonyl)-benzyl]-thiazolo[3,2-c]pyrimidine-5,7-dione;
2-[3-(4-Fluoro-3-methoxy-phenyl)-prop-1-ynyl]-8-methyl-6-(2-oxo-2H-1-benzopyran-6-ylmethyl)-thiazolo[3,2-c]pyrimidine-5,7-dione; and
2-[3-(3Methoxy-4-methyl-phenyl)-prop-1-ynyl)-8-methyl-6-(2-oxo-2H-1-benzopyran-6-ylmethyl)-thiazolo[3,2c]pyrimidine-5,7-dione.

16. A compound or a pharmaceutically acceptable salt thereof, selected from the group consisting of:
6-Benzyl-5,7-dioxo-6,7-dihydro-5H-thiazolo[3,2-c]pyrimidine-2-carboxylic acid benzylamide;
6-Benzyl-5,7-dioxo-6,7-dihydro-5H-thiazolo[3,2-c]pyrimidine-2-carboxylic acid biphenylylamide;
6-Benzyl-5,7-dioxo-6,7-dihydro-5H-thiazolo[3,2-c]pyrimidine-2-carboxylic acid(pyridin-4-ylmethyl)-amide hydrochloride;
6-Benzyl-8-methyl-5,7-dioxo-6,7-dihydro-5H-thiazolo[3,2-c]pyrimidine-2-carboxylic acid 3-fluoro-benzylamide;
6-Benzoyl-5,7-dioxo-6,7-dihydro-5H-thiazolo[3,2-c]pyrimidine-2-carboxylic acid benzylamide;
6-(3,4-Dichloro-benzyl)-5,7-dioxo-6,7-dihydro-5H-thiazolo[3,2-c]pyrimidine-2-carboxylic acid benzylamide;
6-(4-Chloro-benzyl)-5,7-dioxo-6,7-dihydro-5H-thiazolo[3,2-c]pyrimidine-2-carboxylic acid benzylamide;
6-(4-Chloro-benzyl)-5,7-dioxo-6,7-dihydro-5H-thiazolo[3,2-c]pyrimidine-2-carboxylic acid 3,4-dichloro-benzylamide;
5,7-Dioxo-6-pyridin-4-ylmethyl-6,7-dihydro-5H-thiazolo[3,2-c]pyrimidine-2-carboxylic acid benzylamide hydrochloride;
6-Benzyl-8-methyl-5,7-dioxo-6,7-dihydro-5H-thiazolo[3,2-c]pyrimidine-2-carboxylic acid benzylamide;
6-Benzyl-8-methyl-5,7-dioxo-6,7-dihydro-5H-thiazolo[3,2-c]pyrimidine-2-carboxylic acid 4-methoxy-benzylamide;
6-Benzyl-8-methyl-5,7-dioxo -5,7-dihydro-5H-thiazolo[3,2-c]pyrimidine-2-carboxylic acid 3,4-dichlorobenzylamide,
6Benzyl-5,7-dioxo-6,7-dihydro-5H-thiazolo[3,2-c]pyrimidine-2-carboxylic acid(pyridin-4-ylmethyl)-amide hydrochloride;
6-Benzyl-8-methyl-5,7-dioxo-6,7-dihydro-5H-thiazolo[3,2-c]pyrimidine-2-carboxylic acid 2,4-dichloro-benzylamide;
6-Benzyl-8-methyl-5,7-dioxo-6,7-dihydro-5H-thiazolo[3,2-c]pyrimidine-2-carboxylic acid 3-methyl-benzylamide;
6-Benzyl-8-methyl-5,7-dioxo-6,7-dihydro-5H-thiazolo[3,2-c]pyrimidine-2-carboxylic acid 4-fluoro-benzylamide;
6-Benzyl-8-formyl-5,7-dioxo-6,7-dihydro-5H-thiazolo[3,2-c]pyrimidine-2-carboxylic acid 4-methoxy-benzylamide;
6-Benzyl-8-methyl-5,7-dioxo-6,7-dihydro-5H-thiazolo[3,2-c]pyrimidine-2-carboxylic acid(1H-indol-5-ylmethyl)-amide;
6-Benzyl-8-methyl-5,7-dioxo-6,7-dihydro-5H-thiazolo[3,2-c]pyrimidine-2-carboxylic acid(thiazol-4-ylmethyl)-amide hydrochloride;
6-Benzyl-8-methyl-5,7-dioxo-6,7-dihydro-5H-thiazolo[3,2-c]pyrimidine-2-carboxylic acid(pyridin-4-methyl)-amide hydrochloride;
6-Benzyl-8-methyl-5,7-dioxo-6,7-dihydro-5H-thiazolo[3,2-c]pyrimidine-2-carboxylic acid(6-methoxy-pyridin-3-ylmethyl)-amide;
6-Benzyl-8-methyl-5,7-dioxo-6,7-dihydro-5H-thiazolo[3,2-c]pyrimidine-2-carboxylic acid(6-methoxy-pyridin-3-ylmethyl)-amide hydrochloride;
6-Benzyl-8-methyl-5,7-dioxo-6,7-dihydro-5H-thiazolo[3,2-c]pyrimidine-2-carboxylic acid(imidazo[2,1-b]thiazol-6-ylmethyl)-amide;
6-Benzyl-8-methyl-5,7-dioxo-6,7-dihydro-5H-thiazolo[3,2-c]pyrimidine-2-carboxylic acid(1-methyl-1H-pyrazol-4-ylmethyl)-amide;
6-Benzyl-8-methyl-5,7-dioxo-6,7-dihydro-5H-thiazolo[3,2-c]pyrimidine-2-carboxylic acid(6-methyl-pyridin-2-ylmethyl)-amide;
6-Benzyl-8-methyl-5,7-dioxo-6,7-dihydro-5H-thiazolo[3,2-c]pyrimidine-2-carboxylic acid(2,1,3-benzothiadiazol-5-ylmethyl)-amide;
6-Benzyl-8-methyl-5,7-dioxo-6,7-dihydro-5H-thiazolo[3,2-c]pyrimidine-2-carboxylic acid 3,4-difluoro-benzylamide;
6-Benzyl-8-methyl-5,7-dioxo-6,7-dihydro-5H-thiazolo[3,2-c]pyrimidine-2-carboxylic acid(pyridin-3-ylmethyl)-amide;
6-Benzyl-8-methyl-5,7-dioxo-6,7-dihydro-5H-thiazolo[3,2-c]pyrimidine-2-carboxylic acid(pyridin-3-ylmethyl)-amide hydrochloride;
6-Benzyl-8-methyl-5,7-dioxo-6,7-dihydro-5H-thiazolo[3,2-c]pyrimidine-2-carboxylic acid 3-fluoro-4-methoxy-benzylamide;
6-Benzyl-8-methyl-5,7-dioxo-6,7-dihydro-5H-thiazolo[3,2-c]pyrimidine-2-carboxylic acid(pyridin-2-ylmethyl)-amide hydrochloride;
6-Benzyl-8-methyl-5,7-dioxo-6,7-dihydro-5H-thiazolo[3,2-c]pyrimidine-2-carboxylic acid 4-methyl-benzylamide;
6-Benzyl-8-methyl-5,7-dioxo-6,7-dihydro-5H-thiazolo[3,2-c]pyrimidine-2-carboxylic acid 4-trifluoromethyl-benzylamide;
6-Benzyl-8-methyl-5,7-dioxo-6,7-dihydro-5H-thiazolo[3,2-c]pyrimidine-2-carboxylic acid 4-chloro-benzylamide;
6-Benzyl-8-methyl-5,7-dioxo-6,7-dihydro-5H-thiazolo[3,2-c]pyrimidine-2-carboxylic acid 4-trifluoromethoxy-benzylamide;
6-Benzyl-8-methyl-5,7-dioxo-6,7-dihydro-5H-thiazolo[3,2-c]pyrimidine-2-carboxylic acid(2-methyl-thiazol-4-ylmethyl)-amide hydrochloride;
4-2-(4-Methoxy-benzylcarbamoyl)-8-methyl-5,7-dioxo-7H-thiazolo[3,2-c]pyrimidin-6-ylmethyl]-benzoic acid;
4-[2-(4-Methoxy-benzylcarbamoyl)-8-methyl-5,7-dioxo-7H-thiazolo[3,2-c]pyrimidin-6-ylmethyl]-benzoic acid Sodium salt;
4-[2-(4-Methoxy-benzylcarbamoyl)-8-methyl-5,7-dioxo-7H-thiazolo[3,2-c]pyrimidin-6-ylmethyl]-benzoic acid 2-dimethylamino-ethyl ester hydrochloride;
4-[2-(4-Fluoro-benzylcarbamoyl)-8-methyl-5,7-dioxo-7H-thiazolo[3,2-c]pyrimidin-6-ylmethyl]-benzoic acid;
4-[2-(4-Fluoro-benzylcarbamoyl)-8-methyl-5,7-dioxo-7H-thiazolo[3,2-c]pyrimidin-6-ylmethyl]-benzoic acid Sodium salt;
4-[2-(4-Fluoro-benzylcarbamoyl)-8-methyl-5,7-dioxo-7H-thiazolo[3,2-c]pyrimidin-6-ylmethyl]-benzoic acid 2-dimethylamino-ethyl ester,
4-[2-(4-Fluoro-benzylcarbamoyl)-8-methyl-5,7-dioxo-7H-thiazolo[3,2-c]pyrimidin-6-ylmethyl]-benzoic acid 2-dimethylamino-ethyl ester hydrochloride;
4-{8-Methyl-5,7-dioxo-2-[(pyridin-4-ylmethyl)-carbamoyl]-7H-thiazolo[3,2-c]pyrimidin-6-ylmethyl}-benzoic acid trifluoro-acetic acid salt;
4{8-Methyl-5,7-dioxo-2-[(pyridin-4-ylmethyl)-carbamoyl]-7H-thiazolo[3,2-c]pyrimidin-4-ylmethyl}-benzoic acid 2-dimethylamino-ethyl ester dihydrochloride;
8-Methyl-6-(2-methyl-thiazol-4-ylmethyl)-5,7-dioxo-6,7-dihydro-5H-thiazolo[3,2-c]pyrimidine-2-carboxylic acid 4-fluoro-benzylamide;
2-Chloro-4-fluoro-benzylcarbamoyl)-8-methyl-5,7-dioxo-7H-thiazolo[3,2-c]pyrimidin-6-ylmethyl]-benzoic acid methyl ester;
8-Methyl-5,7-dioxo-6-(2H-tetrazol-5-ylmethyl)-6,7-dihydro-5H-thiazolo[3,2-c]pyrimidine-2-carboxylic acid 4-fluoro-benzylamide;
8-Methyl-5,7-dioxo-6-thiazol-2-ylmethyl-6,7-dihydro-5H-thiazolo[3,2-c]pyrimidine-2-carboxylic acid 4-fluoro-benzylamide hydrochloride;
4-[2-(4-Fluoro-benzylcarbamoyl)-8-methyl-5,7-dioxo-7H-thiazolo[3,2-c]pyrimidin-6-ylmethyl]-2-methyl-benzoic acid methyl ester;
4-[2-(4-Fluoro-benzylcarbamoyl)-8-methyl-5,7-dioxo-7H-thiazolo[3,2-c]pyrimidin-6-ylmethyl]-2-methoxy-benzoic acid methyl ester,
6-(4-Fluoro-benzyl)-8-methyl-5,7-dioxo-6,7-dihydro-5H-thiazolo[3,2-c]pyrimidine-2-carboxylic acid(pyridin-4-ylmethyl)-amide hydrochloride;
6-(4-Bromo-benzyl)-8-methyl-5,7-dioxo-6,7-dihydro-5H-thiazolo[3,2-c]pyrimidine-2-carboxylic acid(pyridin-4-ylmethyl)-amide hydrochloride;
6-(4-Chloro-benzyl)-8-methyl-5,7-dioxo-6,7-dihydro-5H-thiazolo[3,2-c]pyrimidine-2-carboxylic acid(pyridin-4-ylmethyl)-amide hydrochloride;
8-Methyl-6-[4-(morpholine-4-carbonyl)benzyl]-5,7-dioxo-6,7-dihydro-5H-thiazolo[3,2-c]pyrimidine-2-carboxylic acid(pyridin-4-ylmethyl)-amide hydrochloride;
{5-[2-(4-Fluoro-benzylcarbamoyl)-8-methyl-5,7-dioxo-7H-thiazolo[3,2-c]pyrimidin-6-ylmethyl]isoxazol-3-yl}-carbamic acid methyl ester;
8-Methyl-5,7-dioxo-6-[4-(2H-tetrazol-5-yl)-benzyl]-6,7-dihydro-5H-thiazolo[3,2-c]pyrimidine-2-carboxylic acid 4-fluoro-benzylamide;
8-Methyl-6-[4-(morpholine-4-carbonyl)benzyl]-5,7-dioxo-6,7-dihydro-5H-thiazolo[3,2-c]pyrimidine-2-carboxylic acid 4-fluoro-benzylamide;
6-(6-Fluoro-quinolin-2-ylmethyl-8-methyl-5,7-dioxo-6,7-dihydro-5H-thiazolo[3,2-c]pyrimidine-2-carboxylic acid 4-fluoro-benzylamide;
2-[2-(4-Fluoro-benzylcarbamoyl)-8-methyl-5,7-dioxo-7H-thiazolo[3,2-c]pyrimidin-6-ylmethyl-5-methoxy-pyrimidine-4-carboxylic acid methyl ester;
6-But-2-ynyl-8-methyl-5,7-dioxo-6,7-dihydro-5H-thiazolo[3,2-c]pyrimidine-2-carboxylic acid 4-fluoro-benzylamide;
8-Methyl-5,7-dioxo-6-(2-oxo-2H-1-benzopyran-6-ylmethyl)-6,7-dihydro-5H-thiazolo[3,2-c]pyrimidine-2-carboxylic acid 4-fluoro-benzylamide;
6-(4-Methanesulfonyl-benzyl)-8-methyl-5,7-dioxo-6,7-dihydro-5H-thiazolo[3,2-c]pyrimidine-2-carboxylic acid(pyridin-4-ylmethyl)-amide hydrochloride;
6-(3-Cyano-benzyl)-8-methyl-5,7-dioxo-6,7-dihydro-5H-thiazolo[3,2-c]pyrimidine-2-carboxylic acid(pyridin-4-ylmethyl)-amide hydrochloride;
6-[2-(4-Chloro-benzenesulfonyl)-ethyl]-8-methyl-5,7-dioxo-6,7-dihydro-5H-thiazolo[3,2-c]pyrimidine-2-carboxylic acid(pyridin-4-ylmethyl)-amide hydrochloride;
8-Methyl-5,7-dioxo-6-(4-sulfamoyl-benzyl)-6,7-dihydro-5H-thiazolo[3,2-c]pyrimidine-2-carboxylic acid(pyridin-4-ylmethyl)-amide hydrochloride;
6-(4Cyano-benzyl)-8-methyl-5,7-dioxo-6,7-dihydro-5H-thiazolo[3,2-c]pyrimidine-2-carboxylic acid(pyridin-4-ylmethyl)-amide hydrochloride;
8-Methyl-5,7-dioxo-6-(3-oxo-3-phenyl-propyl)-6,7-dihydro-5H-thiazolo[3,2-c]pyrimidine-2-carboxylic acid 4-fluoro-benzylamide;
8-Methyl-5,7-dioxo-6-(1-phenyl-ethyl)-6,7-dihydro-5H-thiazolo[3,2-c]pyrimidine-2-carboxylic acid 4-fluoro-benzylamide;
8-Methyl-5,7-dioxo-6-(2-phenylmethanesulfonyl-ethyl)-6,7-dihydro-5H-thiazolo[3,2-c]pyrimidine-2-carboxylic acid 4-fluoro-benzylamide;
6-(5-Cyano-pentyl-8-methyl-5,7-dioxo-6,7-dihydro-5H-thiazolo[3,2-c]pyrimidine-2-carboxylic acid 4-fluoro-benzylamide;
6-(E)-But-2-enyl-8-methyl-5,7-dioxo-6,7-dihydro-5H-thiazolo[3,2-c]pyrimidine-2-carboxylic acid 4-fluoro-benzylamide;
8-Methyl-5,7-dioxo-6-(E)-pent-2-enyl-6,7-dihydro-5H-thiazolo[3,2-c]pyrimidine-2-carboxylic acid 4-fluoro-benzylamide;
6-sec-Butyl-8-methyl-5,7-dioxo-6,7-dihydro-5H-thiazolo[3,2-c]pyrimidine-2-carboxylic acid 4-fluoro-benzylamide;
6-(2-Benzenesulfonyl-ethyl)-8-methyl-5,7-dioxo-6,7-dihydro-5H-thiazolo[3,2-c]pyrimidine-2-carboxylic acid 4-fluoro-benzylamide;
6-(1-Ethyl-propyl)-8-methyl-5,7-dioxo-6,7-dihydro-5H-thiazolo[3,2-c]pyrimidine-2-carboxylic acid 4-fluoro-benzylamide;
8-Methyl-5,7-dioxo-6-pent-2-ynyl-6,7dihydro-5H-thiazolo[3,2-c]pyrimidine-2-carboxylic acid 4-fluoro-benzylamide;
6-(2-Benzenesulfonyl-ethyl)-8-methyl-5,7-dioxo-6,7-dihydro-5H-thiazolo[3,2-c]pyrimidine-2-carboxylic acid 4-fluoro-benzylamide;
8-Methyl-6-(3-methyl-but-2-enyl)-5,7-dioxo-6,7-dihydro-5H-thiazolo[3,2-c]pyrimidine-2-carboxylic acid 4-fluoro-benzylamide;
6-[2-(4-Fluoro-benzenesulfonyl)-ethyl]-8-methyl-5,7-dioxo-6,7-dihydro-5H-thiazolo[3,2-c]pyrimidine-2-carboxylic acid 4-fluoro-benzylamide;
6-[3-(4-Fluoro-phenyl)-3-oxo-propyl]-8-methyl-5,7-dioxo-6,7-dihydro-5H-thiazolo[3,2-c]pyrimidine-2-carboxylic acid 4-fluoro-benzylamide;
6-(2-Benzoylamino-ethyl)-8-methyl-5,7-dioxo-6,7-dihydro-5H-thiazolo[3,2-c]pyrimidine-2-carboxylic acid 4-fluoro-benzylamide;
8-Methyl-5,7-dioxo-6-(2-phenoxy-ethyl)-6,7-dihydro-5H-thiazolo[3,2-c]pyrimidine-2-carboxylic acid 4-fluoro-benzylamide;
6-(3,4-Dichloro-benzyl)-5,7-dioxo-6,7-dihydro-5H-thiazolo[3,2-c]pyrimidine-2-carboxylic acid 4-methoxy-benzylamide;
6-(4-Cyano-benzyl)-8-methyl-5,7-dioxo-6,7-dihydro-5H-thiazolo[3,2-c]pyrimidine-2-carboxylic acid 4-fluoro-benzylamide;
6-Benzyl-8-methyl-5,7-dioxo-6,7-dihydro-5H-thiazolo[3,2-c]pyrimidine-2-carboxylic acid 3-methoxy-benzylamide; and
6-Benzyl-8-methyl-5,7-dioxo-6,7-dihydro-5H-thiazolo[3,2-c]pyrimidine-2-carboxylic acid(tetrahydro-furan-2-ylmethyl-amide;
6-Benzyl-8-methyl-5,7-dioxo-6,7-dihydro-5H-thiazolo[3,2-c]pyrimidine-2-carboxylic acid benzylamide;
6-Benzyl-8-methyl-5,7-dioxo-6,7-dihydro-5H-thiazolo[3,2-c]pyrimidine-2-carboxylic acid 3-fluoro-benzylamide;
6-(3-Fluoro-benzyl)-8-methyl-5,7-dioxo-6,7-dihydro-5H-thiazolo[3,2-c]pyrimidine-2-carboxylic acid 3-fluoro-benzylamide;
6-(3,4-Dichloro-benzyl)-8-methyl-5,7-dioxo-6,7-dihydro-5H-thiazolo[3,2-c]pyrimidine-2-carboxylic acid benzylamide;
6-(3,4-Dichloro-benzyl)-8-methyl-5,7-dioxo-6,7-dihydro-5H-thiazolo[3,2-c]pyrimidine-2-carboxylic acid 4-methyl-benzylamide;
8-Methyl-5,7-dioxo-pyridin-4-ylmethyl-6,7-dihydro-5H-thiazolo[3,2-c]pyrimidine-2-carboxylic acid benzylamide;
6-Benzyl-8-methyl-5,7-dioxo-6,7-dihydro-5H-thiazolo[3,2-c]pyrimidine-2-carboxylic acid(pyridin-4-ylmethyl)-amide;
6-Benzyl-8-methyl-5,7-dioxo-6,7-dihydro-5H-thiazolo[3,2-c]pyrimidine-2-carboxylic acid 4-methoxy-benzylamide;
6-(4-Methoxy-benzyl)-8-methyl-5,7-dioxo-6,7-dihydro-5H-thiazolo[3,2-c]pyrimidine-2-carboxylic acid 4-methoxy-benzylamide;
8-Methyl-5,7-dioxo-6-pyridin-4-ylmethyl-6,7-dihydro-5H-thiazolo[3,2-c]pyrimidine-2-carboxylic acid 4-methoxy-benzylamide;
6-Benzyl-8-methyl-5,7-dioxo-6,7-dihydro-5H-thiazolo[3,2-c]pyrimidine-2-carboxylic acid 3,4-dimethoxy-benzylamide;
6-(4-Methanesulfonyl-benzyl)-8-methyl-5,7-dioxo-6,7-dihydro-5H-thiazolo[3,2-c]pyrimidine-2-carboxylic acid 3,4-dimethoxy-benzylamide;
8-Methyl-5,7-dioxo-6-(4-sulfamoyl-benzyl)-6,7-dihydro-5H-thiazolo[3,2-c]pyrimidine-2-carboxylic acid 3,4-dimethoxy-benzylamide;
6-4-Diethylsulfamoyl-benzyl)-8-methyl-5,7-dioxo-6,7-dihydro-5H-thiazolo[3,2-c]pyrimidine-2-carboxylic acid 3,4-dimethoxy-benzylamide;
8-Methyl-5,7-dioxo-6-pyridin-3-ylmethyl-6,7-dihydro-5H-thiazolo[3,2-c]pyrimidine-2carboxylic acid 3,4-dimethoxy-benzylamide;
8-Methyl-5,7-dioxo-6-pyridin-2-ylmethyl-6,7-dihydro-5H-thiazolo[3,2-c]pyrimidine-2-carboxylic acid 3,4-dimethoxy-benzylamide;
6-Benzyl-8-methyl-5,7-dioxo-6,7-dihydro-5H-thiazolo[3,2-c]pyrimidine-2-carboxylic acid 3-methoxy-benzylamide;
6-(3-Methoxy-benzyl)-8-methyl-5,7-dioxo-6,7-dihydro-5H-thiazolo[3,2-c]pyrimidine-2-carboxylic acid 3-methoxy-benzylamide;
6(3-Methoxy-benzyl)-8-methyl-5,7-dioxo-6,7-dihydro-5H-thiazolo[3,2-c]pyrimidine-2-carboxylic acid(benzo[1,3]dioxol-5-ylmethyl)amide;
6Benzo[1,3]dioxol-5-ylmethyl-8-methyl-5,7-dioxo-6,7-dihydro-5H-thiazolo[3,2-c]pyrimidine-2-carboxylic acid(benzo[1,3]dioxol-5-ylmethyl)-amide;
6-Benzyl-8-methyl-5,7-dioxo-6,7-dihydro-5H-thiazolo[3,2-c]pyrimidine-2-carboxylic acid 4-methylsulfanyl-benzylamide;
6-Benzyl-8-methyl-5,7-dioxo-6,7-dihydro-5H-thiazolo[3,2-c]pyrimidine-2-carboxylic acid 3,4-dichlorobenzylamide;
6-Benzyl-8-methyl-5,7-dioxo-6,7-dihydro-5H-thiazolo[3,2-c]pyrimidine-2-carboxylic acid 4-methoxybenzylamide;
6-Benzyl-8-methyl-5,7-dioxo-6,7-dihydro-5H-thiazolo[3,2-c]pyrimidine-2-carboxylic acid benzylamide;
6-(4-Pyridylmethy)-5,7-dioxo-6,7-dihydro-5H-thiazolo[3,2c]pyrimidine-2-carboxylic acid benzylamide hydrochloride;
6-(4-Chlorobenzyl)-5,7-dioxo-6,7-dihydro-5H-thiazolo[3,2-c]pyrimidine-2-carboxylic acid 3,4-dichlorobenzylamide;
6-(4-Chlorobenzyl)-5,7-dioxo-6,7-dihydro-5H-thiazolo[3,2-c]pyrimidine-2-carboxylic acid benzylamide;
6-(3,4-Dichlorobenzyl)-5,7-oxo-6,7-dihydro-5H-thiazolo[3,2-c]pyrimidine-2-carboxylic acid benzylamide;
6-Benzoyl-5,7-dioxo-6,7-dihydro-5H-thiazolo[3,2-c]pyrimidine-2-carboxylic acid benzylamide;
6-Benzyl-8-methyl-5,7-dioxo-6,7-dihydro-5H-thiazolo[3,2-c]pyrimidine-2-carboxylic acid 3-fluoro-benzylamide;
6-Benzyl-5,7-dioxo-6,7-dihydro-5H-thiazolo[3,2-c]pyrimidine-2-carboxylic acid(pyridin-4-ylmethyl)-amide hydrochloride;
6-Benzyl-5,7-dioxo-6,7-dihydro-5H-thiazolo[3,2-c]pyrimidine-2-carboxylic acid biphenyl-4-ylamide;
6-Benzyl-8-formyl-5,7-dioxo-6,7-dihydro-5H-thiazolo[3,2-c]pyrimidine-2-carboxylic acid 4-fluoro-benzylamide; and
6-Benzyl-8-hydroxymethyl-5,7-dioxo-6,7-dihydro-5H-thiazolo[3,2-c]pyrimidine-2-carboxylic acid 4-fluoro-benzylamide.

17. A compound or a pharmaceutically acceptable salt thereof, selected from the group consisting of:
4-[2-(4-Methoxy-benzylcarbamoyl)-8-methyl-5,7-dioxo-7H-thiazolo[3,2-c]pyrimidin-6-ylmethyl]-benzoic acid methyl ester;
4-[2-(3-Fluoro-benzylcarbamoyl)-8-methyl-5,7-dioxo-7H-thiazolo[3,2-c]pyrimidin-6-ylmethyl]-benzoic acid methyl ester;
4-[2-(4-Fluoro-benzylcarbamoyl)-8-methyl-5,7-dioxo-7H-thiazolo[3,2-c]pyrimidin-6-ylmethyl]-benzoic acid methyl ester.
6-Benzyl-5,7-dioxo-6,7-dihydro-5H-thiazolo[3,2-c]pyrimidine-2-carboxylic acid benzyl ester,
6-Benzyl-8-methyl-5,7-dioxo-6,7-dihydro-5H-thiazolo[3,2-c]pyrimidine-2-carboxylic acid benzyl ester;
6-Benzyl-3-methyl-5,7-dioxo-6,7-dihydro-5H-thiazolo[3,2-c]pyrimidine-2-carboxylic acid methyl ester;
6-Benzyl-8-methyl-5,7-dioxo-6,7-dihydro-5H-thiazolo[3,2-c]pyrimidine-2-carboxylic acid methyl ester;
6-Benzyl-8-methyl-5,7-dioxo-6,7-dihydro-5H-thiazolo[3,2-c]pyrimidine-2-carboxylic acid pyridin-4-ylmethyl ester;
6-Benzyl-5,7-dioxo-6,7-dihydro-5H-thiazolo[3,2-c]pyrimidine-2-carboxylic acid pyridin-4-ylmethyl ester;
8-Methyl-5,7-dioxo-6-pyridin-4-ylmethyl-6,7-dihydro-5H-thiazolo[3,2-c]pyrimidine-2-carboxylic acid 4-methoxy-benzyl ester, and
6-Benzyl-3-methyl-5,7-dioxo-6,7-dihydro-5H-thiazolo[3,2-c]pyrimidine-2-carboxylic acid benzyl ester.

18. A compound or a pharmaceutically acceptable salt thereof, selected from the group consisting of:
6-Benzoyl-thiazolo[3,2-c]pyrimidine-5,7-dione;
6-(4-Chlorobenzyl)-thiazolo[3,2-c]pyrimidine-5,7-dione;
6-Pyridin-4-ylmethyl-thiazolo]3,2-c]pyrimidine-5,7-dione;
8-Methyl-thiazolo[3,2-c]pyrimidine-5,7-dione;
8-Methyl-5,7-dioxo-6,7-dihydro-5H-thiazolo[3,2-c]pyrimidine-2-carboxylic acid;
6-Benzyl-8-methyl-5,7-dioxo-6,7-dihydro-5H-thiazolo[3,2-c]pyrimidine-carboxylic acid;
4-(8-Methyl-5,7-dioxo-7H-thiazolo[3,2-c]pyrimidin-6-yl-methyl)-benzoic acid tert-butyl ester, and
8-Methyl-6-[4-(Morpholine-4-sulfonyl)benzyl]-thiazolo[3,2-c]pyrimidine-5,7-dione.

19. A compound or a pharmaceutically acceptable salt thereof, selected from the group consisting of:
6-(4-Bromo-benzyl)-8-methyl-5,7-dioxo-6,7-hydo-5H-thiazolo[3,2-c]pyrimidine-2-carboxylic acid(2-amino-pyridin-4-ylmethyl)-amide;
6-(4Chloro-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(4-Fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(3-Bromo-4-fluoro-benzyl)-8-methyl-5,7-dioxo-6,7dihydro-5H-thiazolo[3,2-c]pyrimidine-2-carboxylic acid(2-amino-pyridin-4-ylmethyl)-amide;
6-(3-Bromo-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(3,4-Dichloro-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(4-Bromo-3-fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(3-Chloro-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(3-Fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;
6(3,4-Dibromo-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(4-Bromo-3-chloro-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(3,4-Difluoro-benzyl)-8-methyl-5,7-dioxo-6,7-dihydro-5H-thiazolo[3,2c]pyrimidine-2-carboxylic acid(2-amino-pyridin-4-ylmethyl)-amide;
6-(3-Bromo-4-chloro-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(3-Chloro-4-fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(4-Chloro-3-fluoro-benzyl)-8-methyl-5,7-dioxo-6,7-dihydro-5H-thiazolo[3,2-c]pyrimidine-2-carboxylic acid(2-amino-pyridin-4-ylmethyl)-amide;
6-(4-Bromo-benzyl)-8-methyl-5,7-dioxo-6,7-dihydro-5H-thiazolo[3,2-c]pyrimidine-2-carboxylic acid(2-ethoxy-pyridin-4-ylmethyl)-amide;
6-(4-Chloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;
6-(4-Fluoro-benzyl)-8-methyl-5,7-dioxo-6,7-dihydro-5H-thiazolo[3,2-]pyrimidine-2-carboxylic acid(2-ethoxy-pyridin-4-ylmethyl)amide;
6-(3-Bromo-4-fluoro-benzyl)-8-methyl-5,7-dioxo-6,7-dihydro-5H-thiazolo[3,2-c]pyrimidine-2-carboxylic acid(2ethoxy-pyridin-4-ylmethyl)-amide;
6(3-Bromo-benzyl)-8-methyl-5,7-dioxo-6,7-dihydro-5H-thiazolo[3,2c]pyrimidine-2-carboxylic acid(2-ethoxy-pyridin-4-ylmethyl)-amide;
6-(3,4-Dichloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;
6-(4-Bromo-3-fluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;
6-(3-Chloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;
6-(3-Fluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;
6-(3,4-Dibromo-benzyl)-8-methyl-5,7-dioxo-6,7-dihydro-5H-thiazolo[3,2-]pyrimidine-2-carboxylic acid(2-ethoxy-pyridin-4-ylmethyl)-amide;
6(4-Bromo-3-chloro-benzyl)-8-methyl-5,7-dioxo-6,7-dihydro-5H-thiazolo[3,2c]pyrimidine-2-carboxylic acid(2-ethoxy-pyridin-4-ylmethyl)amide;
6-(3,4-Difluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;
6-(3-Bromo-4-chloro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;
6-(3-Chloro-4-fluoro-benzyl)8-methyl-5,7-dioxo-6,7-dihydro-5H-thiazolo[3,2c]pyrimidine-2-carboxylic acid(2-ethoxy-pyridin-4-ylmethyl)-amide;
6-(4-Chloro-3-fluoro-benzyl)-8-methyl-5,7-dioxo-6,7-dihydro-5H-thiazolo[3,2-c]pyrimidine-2-carboxylic acid(2-ethoxy-pyridin-4-ylmethyl)-amide;
6-(4-Bromo-benzyl)-8-methyl-5,7-dioxo-6,7-dihydro-5H-thiazolo[3,2-c]pyrimidine-2-carboxylic acid(6-hydroxy-pyridin-3-ylmethyl)-amide;
6-(4-Chloro-benzyl-8-methyl-5,7-dioxo-6,7-dihydro-5H-thiazolo[3,2-c]pyrimidine-2-carboxylic acid(6-hydroxy-pyridin-3-ylmethyl)-amide;
6-(4-Fluoro-benzyl)-8-methyl-5,7-dioxo-6,7-dihydro-5H-thiazolo[3,2-c]pyrimidine-2-carboxylic acid(6-hydroxy-pyridin-3-ylmethyl)amide;
6-(3-Bromo-fluoro-benzyl)-8-methyl-5,7-dioxo-6,7-dihydro-5H-thiazolo[3,2-c]pyrimidine-2-carboxylic acid(6-hydroxy-pyridin-3-ylmethyl)amide;
6-(3-Bromo-benzyl)-8-methyl-5,7-dioxo-6,7-dihydro-5H-thiazolo[3,2-c]pyrimidine-2-carboxylic acid(6-hydroxy-pyridin-3-ylmethyl)-amide;
6-(3,4-Dichloro-benzyl)-8-methyl-5,7-dioxo-6,7-dihydro-5H-thiazolo[3,2c]pyrimidine-2-carboxylic acid(6-hydroxy-pyridin-3-ylmethyl)-amide;
6-(4-Bromo-3-fluoro-benzyl)-8-methyl-5,7-dioxo-6,7-hydro-5H-thiazolo[3,2-c]pyrimidine-2-carboxylic acid(6-hydroxy-pyridin-3-ylmethyl)amide;
6-(3-Chloro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;
6-(3-Fluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;
6-(3,4-Dibromo-benzyl)-8-methyl-5,7-dioxo-6,7-dihydro-5H-thiazolo[3,2-c]pyrimidine-2-carboxylic acid(6-hydroxy-pyridin-3-ylmethyl)-amide;
6-(4-Bromo-3-chloro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;
6-(3,4-Difluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;
6-(3-Bromo-4-chloro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;
6-(3-Chloro-4-fluoro-benzyl)-8-methyl-5,7-dioxo-6,7-dihydro-5H-thiazolo[3,2-c]pyrimidine2-carboxylic acid(6-hydroxy-pyridin-3-ylmethyl)-amide;
6-(4-Chloro-3-fluoro-benzyl)-8-methyl-5,7-dioxo-6,7-dihydro-5H-thiazolo[3,2-c]pyrimidine-2-carboxylic acid(6-hydroxy-pyridin-3-ylmethyl)-amide;
6(4-Bromo-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(4-Chloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(4-Fluoro-benzyl)-8-methyl-5,7-dioxo-6,7-dihydro-5H-thiazolo[3,2c]pyrimidine-2-carboxylic acid(2-methoxy-pyridin-4-ylmethyl)-amide;
6-(3-Bromo-4-fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(3-Bromo-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(3,4-Dichloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(4-Bromo-3-fluoro-benzyl)-8-methyl-5,7-dioxo6,7-hydro-5H-thiazolo[3,2-c]pyrimidine-2-carboxylic acid(2-methoxy-pyridin-4-ylmethyl)-amide;
6-(3-Chloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(3-Fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(3,4-Dibromo-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(4-Bromo-3-chloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(3,4-Difluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(3-Bromo-4-chloro-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(3-Chloro-4-fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amide;
6-(4-Chloro-3-fluoro-benzyl)-8-methyl-5,7-dioxo-6,7-dihydro-5H-thiazolo[3,2-c]pyrimidine-2-carboxylic acid(2-methoxy-pyridin-4-ylmethyl)-amido;
6-(4-Bromo-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(4-Chloro-benzyl)-8-methyl-5,7-dioxo-6,7-dihydro-5H-thiazolo[3,2c]pyrimidine-2-carboxylic acid(2-methyl-pyridin-4-ylmethyl)-amide;
6-(4-Fluoro-benzyl)-8-methyl-5,7-dioxo-6,7-dihydro-5H-thiazolo[3,2c]pyrimidine-2-carboxylic acid(2-methyl-pyridin-4-ylmethyl)-amide;
6-(3-Bromo-4-fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(3-Bromo-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(3,4-Dichloro-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(4-Bromo-3-fluoro-benzyl)-8-methyl-5,7A-dioxo-6,7-dihydro-5H-thiazolo[3,2-c]pyrimidine-2-carboxylic acid(2-methyl-pyridin-4-ylmethyl)-amide;
6-(3-Chloro-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(3Fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(3,4-Dibromo-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(4-Bromo-3-chloro-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(3,4-Difluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(3-Bromo-4-chloro-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(3-Chloro-4-fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(4-Chloro-3-fluoro-benzyl)-8-methyl-5,7-dioxo-6,7-dihydro-5H-thiazolo[3,2-c]pyrimidine-2-carboxylic acid(2-methyl-pyridin-4-ylmethyl)-amide;
6-(4-Bromo-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(4-Chloro-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(4-Fluoro-benzyl)-8-methyl-5,7-dioxo-6,7-dihydro-5H-thiazolo[3,2-]pyrimidine-2-carboxylic acid(2-methylamino-pyridin-4-ylmethyl)-amide;
6-(3-Bromo-4-fluoro-benzyl)-8-methyl-5,7-dioxo-6,7-dihydro-5H-thiazolo[3,2-c]pyrimidine-2-carboxylic acid(2-methylamino-pyridin-4-ylmethyl1amide;
6-(3-Bromo-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(3,4Dichloro-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(4-Bromo-3-fluoro-benzyl)-8-methyl-5,7-dioxo-6,7-dihydro-5H-thiazolo[3,2-c]pyrimidine-2-carboxylic acid(2-methylamino-pyridin4-ylmethyl)-amide;
6-(3-Chloro-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(3-Fluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(3,4-Dibromo-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(4-Bromo-3chloro-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(3,4-Difluoro-benzyl)-8-methyl-5,7-dioxo-6,7-dihydro-5H-thiazolo[3,2-c]pyrimidine-2-carboxylic acid(2-methylamino-pyridin-4-ylmethyl)amide;
6-(3-Chloro-4-fluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(4-Chloro-3-fluoro-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(4-Chloro-3-bromo-benzyl)-8-methyl-5,7-dioxo-6,7-dihydro-5H-thiazolo[3,2-c]pyrimidine-2-carboxylic acid(2-methylamino-pyridin-4-ylmethyl)-amide;
6-(4-Bromo-benzyl)-8-methyl-5,7-dioxo-6,7-dihydro-5H-thiazolo[3,2-c]pyrimidine-2-carboxylic acid(pyridin-3-ylmethyl)-amide;
6-(4-Chloro-benzyl)-8-methyl-5,7-dioxo-6,7-dihydro-5H-thiazolo[3,2-c]pyrimidine-2-carboxylic acid(pyridin-3-ylmethyl)-amide;
6-(4-Fluoro-benzyl)-8-methyl-5,7-dioxo-6,7-dihydro-5H-thiazolo[3,2-c]pyrimidine-2-carboxylic acid(pyridin-3-ylmethyl)-amide;
6-(3-Bromo-4-fluoro-benzyl)-8-methyl-5,7-dioxo-6,7-dihydro-5H-thiazolo[3,2-c]pyrimidine-2-carboxylic acid(pyridin-3-ylmethyl)-amide;
6-(3-Bromo-benzyl)-8-methyl-5,7-dioxo-6,7-dihydro-5H-thiazolo[3,2-c]pyrimidine-2-carboxylic acid(pyridin-3-ylmethyl)-amide;
6-(3,4-Dichloro-benzyl)-8-methyl-5,7-dioxo-6,7-dihydro-5H-thiazolo[3,2-c]pyrimidine-2-carboxylic acid(pyridin-3-ylmethyl)-amide;
6-(4-Bromo-3-fluoro-benzyl)-8-methyl-5,7-dioxo-6,7-dihydro-5H-thiazolo[3,2-c]pyrimidine-2-carboxylic acid(pyridin-3-ylmethyl)-amide;
6-(3-Chloro-benzyl)-8-methyl-5,7-dioxo-6,7-dihydro-5H-thiazolo[3,2-c]pyrimidine-2-carboxylic acid(pyridin-3-ylmethyl)-amide;
6-(3-Fluoro-benzyl)-8-methyl-5,7-dioxo-6,7-dihydro-5H-thiazolo[3,2-c]pyrimidine-2-carboxylic acid(pyridin-3-ylmethyl)-amide;
6-(3,4-Dibromo-benzyl)-8-methyl-5,7-dioxo-6,7-dihydro-5H-thiazolo[3,2-c]pyrimidine-2-carboxylic acid(pyridin-3-ylmethyl)-amide;
6-(4-Bromo-3-chloro-benzyl)-8-methyl-5,7-dioxo-6,7-dihydro-5H-thiazolo[3,2-c]pyrimidine-2-carboxylic acid(pyridin-3-ylmethyl)-amide;
6-(3,4-Difluoro-benzyl)-8-methyl-5,7-dioxo-6,7-dihydro-5H-thiazolo[3,2-c]pyrimidine-2-carboxylic acid(pyridin-3-ylmethyl)-amide;
6-(3-Bromo-4-chloro-benzyl)-8-methyl-5,7-dioxo-6,7-dihydro-5H-thiazolo[3,2-c]pyrimidine-2-carboxylic acid(pyridin-3-ylmethyl)-amide;
6-(3-Chloro-4-fluoro-benzyl)-8-methyl-5,7-dioxo-6,7-dihydro-5H-thiazolo[3,2-c]pyrimidine-2-carboxylic acid(pyridin-3-ylmethyl)-amide;
6-(4-Chloro-3-fluoro-benzyl)-8-methyl-5,7-dioxo-6,7-dihydro-5H-thiazolo[3,2-c]pyrimidine-2-carboxylic acid(pyridin-3-ylmethyl)-amide;
6-(4-Bromo-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(4-Chloro-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(4-Fluoro-benzyl)-8-methyl-5,7-dioxo-6,7-dihydro-5H-thiazolo[3,2-c]pyrimidine-2-carboxylic acid(6amino-pyridin-3-ylmethyl)-amide;
6-(3-Bromo-4-fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(3-Bromo-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(3,4-Dichloro-benzyl)-8-methyl-5,7-dioxo-6,7-dihydro-5H-thiazolo[3,2-c]pyrimidine-2-carboxylic acid(6-amino-pyridin-3-ylmethyl)amide;
6-(4-Bromo-3-fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(3-Chloro-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(3-Fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(3,4-Dibromo-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(4-Bromo-3-chloro-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(3,4-Difluoro-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(3-Bromo-4-chloro-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(3-Chloro-4-fluoro-benzyl)-8-methyl-5,7-dioxo-6,7-dihydro-5H-thiazolo[3,2-c]pyrimidine-2-carboxylic acid(6-amino-pyridin-3-ylmethyl)-amide;
6-(4-Chloro-3-fluoro-benzyl)-8-methyl-5,7-dioxo-6,7-dihydro-5H-thiazolo[3,2-c]pyrimidine-2-carboxylic acid(6-amino-pyridin-3-ylmethyl)amide;
6-(4-Bromo-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(4-Chloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(4-Fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(3-Bromo-4-fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(3-Bromo-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(3,4-Dichloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(4-Bromo-3-fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(3-Chloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(3-Fluoro-benzyl)-8-methyl-5,7-dioxo-6,7-dihydro-5H-thiazolo-[3,2-c]pyrimidine-2-carboxylic acid(6-ethoxy-pyridin-3-ylmethyl)amide:
6-(3,4-Dibromo-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(4-Bromo-3-chloro-benzyl)-8-methyl-5,7-dioxo-6,7-dihydro-5H-thiazolo[3,2-c]pyrimidine-2-carboxylic acid(6-ethoxy-pyridin-3-ylmethyl)amide;
6-(3,4-Difluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(3-Bromo-4-chloro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(3-Chloro-4-fluoro-benzyl)-8-methyl-5,7-dioxo-6,7-dihydro-5H-thiazolo[3,2-c]pyrimidine-2-carboxylic acid(6-ethoxy-pyridin-3-ylmethyl)amide;
6-(4-Chloro-3-fluoro-benzyl)-8-methyl-5,7-dioxo-6,7-dihydro-5H-thiazolo[3,2-c]pyrimidine-2-carboxylic acid(6-ethoxy-pyridin-3-ylmethyl)-amide;
6-(4-Bromo-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(4-Chloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(4-Fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(3-Bromo-fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(3-Bromo-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(3,4-Dichloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(4-Bromo-3-fluoro-benzyl)-8methyl-5,7-dioxo-6,7-dihydro-5H-thiazolo[3,2-c]pyrimidine-2-carboxylic acid(6-methoxy-pyridin-3-ylmethyl)-amide;
6-(3-Chloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(3-Fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(3,4-Dibrorno-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(4-Bromo-3-chloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(3,4-Difluoro-benzyl)-8-methyl-5,7-dioxo-6,7-dihydro-5H-thiazolo[3,2-c]pyrimidine-2-carboxylic acid(6methoxy-pyridin-3-ylmethyl)-amide;
6-(3-Bromo-4-chloro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(3-Chloro-4-fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(4-Chloro-3-fluoro-benzyl)-8-methyl-5,7-dioxo-6,7-dihydro-5H-thiazolo[3,2-c]pyrimidine-2-carboxylic acid(6-methoxy-pyridin-3-ylmethyl)-amide;
6-(4-Bromo-benzyl)-8-methyl-5,7-dioxo-6,7-dihydro-5H-thiazolo[3,2-c]pyrimidine-2-carboxylic acid(6-methyl-pyridin-3-ylmethyl)amide;
6-(4-Chloro-benzyl)-8-methyl-5,7-dioxo-6,7-dihydro-5H-thiazolo[3,2-c]pyrimidine-2-carboxylic acid(6-methyl-pyridin-3-ylmethyl-amide;
6-(4-Fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(3-Bromo-4-fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(3-Bromo-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(3,4-Dichloro-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(4-Bromo-3-fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(3-Chloro-benzyl)-8-methyl-5,7dioxo-6,7-dihydro-5H-thiazolo[3,2-c]pyrimidine-2-carboxylic acid(6-methyl-pyridin-3-ylmethyl)-amide;
6-(3-Fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(3,4-Dibromo-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(4-Bromo-3-chloro-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(3,4-Difluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(3-Bromo-4chloro-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(3-Chloro-4-fluoro-benzyl)-8-methyl-5,7-dioxo-6,7-dihydro-5H-thiazolo[3,2-c]pyrimidine-2-carboxylic acid(6-methyl-pyridin-3-ylmethyl)-amide;
6-(4-Chloro-3-fluoro-benzyl)-8methyl-5,7-dioxo-6,7-dihydro-5H-thiazolo[3,2-c]pyrimidine-2-carboxylic acid(6-methyl-pyridin-3-ylmethyl)-amide;
6-(4-Cyano-benzyl)-8-methyl-5,7-dioxo-6,7-dihydro-5H-thiazolo[3,2-c]pyrimidine-2-carboxylic acid(2-methoxy-pyridin-4-ylmethyl)-amide; and
6-(4-Isopropylsulfamoyl-benzyl)-8-methyl-5,7-dioxo-6,7-dihydro-5H-thiazolo[3,2-c]pyrimidine-2-carboxylic acid(2-methoxy-pyridin-4-ylmethyl)-amide.

20. A compound or a pharmaceutically acceptable salt thereof, selected from the group consisting of:
8-Methyl-5,7-dioxo-6-(3-oxo-3-phenyl-propyl)-6,7-dihydro-5H-thiazolo[3,2-c]pyrimidine-2-carboxylic acid 4-fluoro-benzylamide;
8-Methyl-6-(1-phenylethyl) 5,7-dioxo-6,7-dihydro-5H-thiazolo[3,2-c]pyrimidine-2-carboxylic acid 4-fluorobenzylamide;
8-Methyl-5,7-dioxo-6-(2-phenylmethanesulfonyl-ethyl)-6,7-dihydro-5H-thiazolo[3,2-c]pyrimidine-2-carboxylic acid 4-fluorobenzylamide;
6-(5-Cyano-pentyl)-8-Methyl-5,7-dioxo-6,7-dihydro-5H-thiazolo[3,2-c]pyrimidine-2-carboxylic acid 4-fluorobenzylamide;
6-(E)-But-2-enyl-8-Methyl-5,7-dioxo-6,7-dihydro-5H-thiazolo[3,2-c]pyrimidine-2-carboxylic acid 4-fluorobenzylamide;
8-Methyl-5,7-dioxo-6-(E)-pent-2-enyl-6,7-dihydro-5H-thiazolo[3,2-c]pyrimidine-2-carboxylic acid 4-fluorobenzylamide;
6-sec-Butyl-8-Methyl-5,7-dioxo-6,7-dihydro-5H-thiazolo[3,2-c]pyrimidine-2-carboxylic acid 4-fluorobenzylamide;
8-Methyl-6-(2-methyl-allyl)-5,7-dioxo-6,7-dihydro-5H-thiazolo[3,2-]pyrimidine-2-carboxylic acid 4-fluorobenzylamide;
6-(1-Ethyl-propyl)-8-Methyl-5,7-dioxo-6,7-dihydro-5H-thiazolo[3,2-c]pyrimidine-2-carboxylic acid 4-fluorobenzylamide;
8-Methyl-5,7-dioxo-6-pent-2-ynyl-6,7-dihydro-5H-thiazolo[3,2-c]pyrimidine-2-carboxylic acid 4-fluorobenzylamide;
6-(2-Benzensulfonyl-ethyl)-8-Methyl-5,7-dioxo-6,7-dihydro-5H-thiazolo[3,2-c]pyrimidine-2-carboxylic acid 4-fluorobenzylamide;
8-Methyl-6-(3-methyl-but-2-enyl)-5,7-dioxo-6,7-dihydro-5H-thiazolo[3,2-c]pyrimidine-2-carboxylic acid 4-fluorobenzylamide;
6-[2-(4-Fluoro-benzensulfonyl)-ethyl]-8-Methyl-5,7-dioxo-6,7-dihydro-5H-thiazolo[3,2-c]pyrimidine-2-carboxylic acid 4-fluorobenzylamide;
6-[3-(4-Fluoro-phenyl)-3-oxo-propyl]-8-Methyl-5,7-dioxo-6,7-dihydro-5H-thiazolo[3,2-c]pyrimidine-2-carboxylic acid 4-fluorobenzylamide;
8-Methyl-5,7-dioxo-6-{2-[(1-phenyl-methanoyl)-amino]-ethyl}-6,7-dihydro-5H-thiazolo[3,2-c]pyrimidine-2-carboxylic acid 4-fluorobenzylamide;
8-Methyl-5,7-dioxo-6-(2-phenoxy-ethyl)-6,7-dihydro-5H-thiazolo[3,2-c]pyrimidine-2-carboxylic acid 4-fluorobenzylamide; and
{5-[2-(4-Fluoro-benzylcarbamoyl)-8-methyl-5,7-dioxo-7H-thiazolo[3,2-c]pyrimidine-6-ylmethyl]-isoxazol-3-yl]}-carbamic acid methyl.

21-34. (canceled)

35. A compound or a pharmaceutically acceptable salt thereof, selected from the group consisting of:
6-Benzyl-5,7-dioxo-6,7-dihydro-5H-thiazolo[3,2-c]pyrimidine-2-carboxylic acid benzyl ester;
6-Benzyl-8-methyl-5,7-dioxo-6,7-dihydro-5H-thiazolo[3,2-c]pyrimidine-2-carboxylic acid benzyl ester;
6-Benzyl-8-methyl-5,7-dioxo-6,7-dihydro-5H-thiazolo[3,2-c]pyrimidine-2-carboxylic acid benzylamide;
6-Benzyl-8-methyl-5,7-dioxo-6,7-dihydro-5H-thiazolo[3,2-c]pyrimidine-2-carboxylic acid 3-fluoro-benzylamide;
6-(3-Fluoro-benzyl)-8-methyl-5,7-dioxo-6,7-dihydro-5H-thiazolo[3,2-c]pyrimidine-2-carboxylic acid 3-fluoro-benzylamide;
6-(3,4-Dichloro-benzyl)-8-methyl-5,7-dioxo-6,7-dihydro-5H-thiazolo[3,2-c]pyrimidine-2-carboxylic acid benzylamide; p1 6-(3,4-Dichloro-benzyl)-8-methyl-5,7-dioxo-6,7-dihydro-5H-thiazolo[3,2-c]pyrimidine-2-carboxylic acid 4-methyl-benzylamide;
8-Methyl-5,7-dioxo-6-pyridin-4-ylmethyl-6,7-dihydro-5H-thiazolo[3,2-c]pyrimidine-2-carboxylic acid benzylamide;
6-Benzyl-8-methyl-5,7-dioxo-6,7-dihydro-5H-thiazolo[3,2-c]pyrimidine-2-carboxylic acid(pyridin-4-ylmethyl)-amide;
6-Benzyl-8-methyl-5,7-dioxo-6,7-dihydro-5H-thiazolo[3,2-c]pyrimidine-2-carboxylic acid pyridin-4-ylmethyl ester;
6-Benzyl-5,7-dioxo-6,7-dihydro-5H-thiazolo[3,2-c]pyrimidine-2-carboxylic acid pyridin-4-ylmethyl ester;
6-Benzyl-8-methyl-5,7-dioxo-6,7-dihydro-5H-thiazolo[3,2-c]pyrimidine-2-carboxylic acid 4-methoxy-benzylamide;
6-(4-Methoxy-benzyl)-8-methyl-5,7-dioxo-6,7-dihydro-5H-thiazolo[3,2-c]pyrimidine-2-carboxylic acid 4-methoxy-benzylamide;
8-Methyl-5,7-dioxo-6-pyridin-4-ylmethyl-6,7-dihydro-5H-thiazolo[3,2-c]pyrimidine-2-carboxylic acid 4-methoxy-benzylamide;
8-Methyl-5,7-dioxo-6-pyridin-4-ylmethyl-6,7-dihydro-5H-thiazolo[3,2-c]pyrimidine-2-carboxylic acid 4-methoxy-benzyl ester;
6-Benzyl-8-methyl-5,7-dioxo-6,7-dihydro-5H-thiazolo[3,2-c]pyrimidine-2-carboxylic acid 3,4-dimethoxy-benzylamide;
6-(4Methanesulfonyl-benzyl)-8-methyl-5,7-dioxo-6,7-dihydro-5H-thiazolo[3,2-c]pyrimidine-2-carboxylic acid 3,4-dimethoxy-benzylamide,
8-Methyl-5,7-dioxo-6-(4-sulfamoyl-benzyl)6,7-dihydro-5H-thiazolo[3,2-c]pyrimidine-2-carboxylic acid 3,4dimethoxy-benzylamide;
6-(4-Dimethylsulfamoyl-benzyl)-8-methyl-5,7-dioxo-6,7-dihydro-5H-thiazolo[3,2-c]pyrimidine-2-carboxylic acid 3,4-dimethoxy-benzylamide;
8-Methyl-5,7-dioxo-6-pyridin-3-ylmethyl-6,7-dihydro-5H-thiazolo[3,2-c]pyrimidine-2-carboxylic acid 3,4-dimethoxy-benzylamide;
8-Methyl-5,7-dioxo-6-pyridin-2-ylmethyl-6,7-dihydro-5H-thiazolo[3,2-c]pyrimidine-2-carboxylic acid 3,4-dimethoxy-benzylamide;
6-Benzyl-8-methyl-5,7-dioxo-6,7-dihydro-5H-thiazolo[3,2-c]pyrimidine-2-carboxylic acid 3-methoxy-benzylamide;
6-(3-Methoxy-benzyl)-8-methyl-5,7-dioxo-6,7-dihydro-5H-thiazolo[3,2-c]pyrimidine-2-carboxylic acid 3-methoxy-benzylamide,
6-(3-Methoxy-benzyl)-8-methyl-5,7-dioxo-6,7-dihydro-5H-thiazolo[3,2-c]pyrimidine-2-carboxylic acid(benzo[1,3]dioxol-5-ylmethyl)amide;
6-Benzo[1,3]dioxol-5-ylmethyl-8-methyl-5,7-dioxo-6,7-dihydro-5H-thiazolo[3,2-c]pyrimidine-2-carboxylic acid(benzo[1,3]dioxol-5-ylmethyl)-amide;
6-Benzyl-8-methyl-5,7-dioxo-6,7-dihydro-5H-thiazolo[3,2-c]pyrimidine-2-carbothioic acid benzylamide;
6-Benzyl-8-methyl-5,7-dioxo-6,7-dihydro-5H-thiazolo[3,2-c]pyrimidine-2-carbothioic acid 4methoxy-benzylamide;
6-Benzyl-8-methyl-5,7-dioxo-6,7-dihydro-5H-thiazolo[3,2-c]pyrimidine-2-carboxylic acid 4-methylsulfanyl-benzylamide;
6-Benzyl-3-methyl-5,7-dioxo-6,7-dihydro-5H-thiazolo[3,2-c]pyrimidine-2-carboxylic acid benzyl ester;
6-Benzyl-8-methyl-5,7-dioxo-6,7-dihydro-5H-thiazolo[3,2-c]pyrimidine-2-carboxylic acid 3,4-dichlorobenzylamide;
6-Benzyl-8-methyl-5,7-dioxo-6,7-dihydro-5H-thiazolo[3,2-c]pyrimidine-2-carboxylic acid 4-methoxybenzylamide;
6-Benzyl-8-methyl-5,7-dioxo-6,7-dihydro-5H-thiazolo[3,2-c]pyrimidine-2-carboxylic acid benzylamide;
6-(4-Pyridylmethy)-5,7-dioxo-6,7-dihydro-5H-thiazolo[3,2-c]pyrimidine-2-carboxylic acid benzylamide hydrochloride;
6-(4-Chlorobenzyl)-5,7-dioxo-6,7-dihydro-5H-thiazolo[3,2-c]pyrimidine-2-carboxylic acid 3,4-dichlorobenzylamide;
6-(4-Chlorobenzyl)-5,7-dioxo-6,7-dihydro-5H-thiazolo[3,2-c]pyrimidine-2-carboxylic acid benzylamide, 6-(3,4-Dichloro-benzyl)-5,7-dioxo-6,7-dihydro-5H-thiazolo[3,2-c]pyrimidine-2-carboxylic acid benzylamide;
6-Benzoyl-5,7-dioxo-6,7-dihydro-5H-thiazolo[3,2-c]pyrimidine-2-carboxylic acid benzylamide;
6-Benzyl-8-methyl-5,7-dioxo-6,7-dihydro-5H-thiazolo[3,2-c]pyrimidine-2-carboxylic acid 3-fluoro-benzylamide;
6-Benzyl-5,7-dioxo-6,7-dihydro-5H-thiazolo[3,2-c]pyrimidine-2-carboxylic acid(pyridin-4-ylmethyl)-amide hydrochloride;
6-Benzyl-2-(3-phenyl-propionyl)-thiazolo[3,2-c]pyrimidine-5,7dione;
6-Benzyl-2-(hydroxy-phenyl-methyl)-thiazolo[3,2-c]pyrimidine-5,7-dione;
6-Benzyl-5,7-dioxo-6,7-dihydro-5H-thiazolo[3,2-c]pyrimidine-2-carboxylic acid biphenyl-4-ylamide;
6-Benzyl-2-(1-hydroxy-3-phenyl-propyl)-thiazolo[3,2-c]pyrimidine-5,7-dione;
6-Benzyl-5,7-dioxo-2,3,6,7-tetrahydro-5H-thiazolo[3,2-c]pyrimidine-2-carboxylic acid benzyl ester,
6-Benzyl-5,7-oxo-2,3,6,7-tetrahydro-5H-thiazolo[3,2-c]pyrimidine-2-carboxylic acid benzyl ester;
6-Benzyl-5,7-dioxo-2,3,6,7-tetrahydro-5H-thiazolo[3,2-c]pyrimidine-2-carboxylic acid pyridin-4-ylmethyl ester hydrochloride;
6-Benzyl-5,7-dioxo-2,3,6,7-tetrahydro-5H-thiazolo[3,2-c]pyrimidine-2-carboxylic acid;
6-Benzyl-5,7-dioxo-2,3,6,7-tetrahydro-5H-thiazolo[3,2-c]pyrimidine-2-carboxylic acid pyridin-4-ylmethyl ester hydrochloride;
6-Benzyl-5,7-dioxo-2,3,6,7-tetrahydro-5H-thiazolo[3,2-]pyrimidine-2-carboxylic acid(pyridin-4-ylmethyl)amide; and
6-Benzyl-1,5,7-trioxo-1,2,3,5,6,7-hexahydro-114-thiazolo[3,2-c]pyrimidine-3carboxylic acid benzyl ester.

36-44. (canceled)
